(19) United States
(12) Patent Application Publication
(10) Pub. No.: US 2026/0167859 A1
(43) Pub. Date: Jun. 18, 2026

(54) ORGANIC COMPOUND AND ORGANIC LIGHT-EMITTING DEVICE COMPRISING SAME

(71) Applicant: P&H TECH Co., Ltd, Yongin-si (KR)

(72) Inventors: Seo-Yong HYUN, Yongin-si (KR); Gwan-hee PARK, Yongin-si (KR)

(73) Assignee: P&H TECH Co., Ltd, Yongin-si (KR)

(21) Appl. No.: 18/683,304

(22) PCT Filed: Sep. 27, 2021
(86) PCT No.: PCT/KR2021/013096

§ 371 Date: Feb. 26, 2026

(30) Foreign Application Priority Data
Aug. 13, 2021 (KR) 10-2021-0107035

(51) Int. Cl. C09K 11/02; C07D 207/27; C07D 401/14; H10K 50/858

(52) U.S. Cl. CPC C09K 11/025; C07D 207/27; C07D 401/14; H10K 50/858

(57) ABSTRACT

The present invention relates to an organic compound and an organic light-emitting device comprising same. The organic compound is used in a light efficiency improving layer included in an organic light-emitting device to increase the light efficiency of the device so as to be able to implement excellent device characteristics such as low voltage driving, excellent color purity, and improved luminescence efficiency of the device. Same can be industrially and usefully used for various lighting and display devices.

### TECHNICAL FIELD

The present invention relates to an organic compound that is employed as a material for a light efficiency improving layer (capping layer) provided in an organic light-emitting device, and an organic light-emitting device that employs the same, thus achieving greatly improved luminescent properties such as low-voltage driving of the device and excellent luminous efficiency.

### BACKGROUND ART

The organic light-emitting device may be formed even on a transparent substrate, and may be driven at a low voltage of 10 V or less compared to a plasma display panel or an inorganic electroluminescence (EL) display. In addition, the device consumes relatively little power and has good color representation. The device may display three colors of green, blue, and read, and thus has recently become a subject of intense interest as a next-generation display device.

However, in order for such an organic light-emitting device to exhibit the aforementioned characteristics, the materials constituting an organic layer in the device, such as hole injecting materials, hole transport materials, light emitting materials, electron transport materials, and electron injecting materials, are prerequisites for the support by stable and efficient materials. However, the development of a stable and efficient organic layer material for an organic light-emitting device has not yet been sufficiently made.

Thus, further improvements in terms of efficiency and life characteristics are required for good stability, high efficiency, long lifetime, and large size of organic light-emitting devices. Particularly, there is a strong need to develop materials constituting each organic layer of organic light-emitting devices.

In addition, recently, research aimed at improving the characteristics of organic light-emitting devices by changes in the performance of each organic layer material, as well as a technique for improving the color purity and enhancing the luminous efficiency by optimizing the optical thickness between an anode and a cathode are considered as one of the crucial factors for improving the device performance. As an example of this method, an increase in light efficiency and excellent color purity are achieved by using a capping layer on an electrode.

### DISCLOSURE

### Technical Problem

Thus, the present invention has been made in an effort to provide a novel organic compound which may be employed in a light efficiency improving layer (capping layer) provided in an organic light-emitting device to implement excellent luminescent properties such as low-voltage driving of the device and improved luminous efficiency, and an organic light-emitting device including the same.

### Technical Solution

An aspect of the present invention provides an organic compound represented by Formula I below.

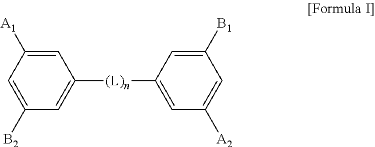

Characteristic structures of Formula I above and specific compounds, L, A₁ to A₂, and B₁ to B₂ implemented thereby will be described below.

Another aspect of the present invention provides an organic light-emitting device including a first electrode, a second electrode, and one or more organic layers arranged between the first and second electrodes, wherein the organic light-emitting device further includes a light efficiency improving layer (capping layer) formed on at least one side opposite to the organic layer among the upper or lower portions of the first electrode and the second electrode, and the light efficiency improving layer includes an organic compound represented by Formula I above.

### Advantageous Effects

According to the present invention, an organic compound is employed as a material for a light efficiency improving layer provided in an organic light-emitting device to implement low-voltage driving of the organic light-emitting device and improved luminescent properties such as excellent luminous efficiency, color purity, etc., and thus can be effectively used in various display devices.

### BEST MODE FOR CARRYING OUT INVENTION

Hereinafter, the present invention will be described in more detail.

The present invention relates to an organic compound represented by Formula I below which is employed as a material for a light efficiency improving layer provided in an organic light-emitting device to implement low-voltage driving of the device and luminescent properties such as excellent luminescence efficiency, color purity, etc.

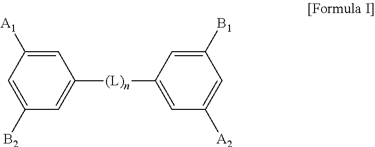

In Formula I above,
(i) L is a single bond or is selected from a substituted or unsubstituted arylene group having 6 to 30 carbon atoms and a substituted or unsubstituted heteroarylene group having 3 to 30 carbon atoms, n is an integer of 0 to 2, and when the n is 2, a plurality of L are the same as or different from each other.
(ii) A₁ and A₂ are the same as or different from each other, and each independently selected from structures shown in Structural Formula 1 below, and ‘*’ indicates a linking site.

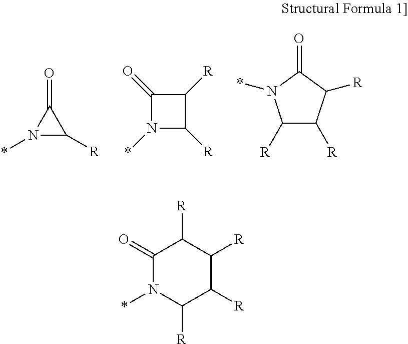

In Structural Formula 1 above,
in each structure, R is each independently selected from hydrogen, deuterium, a cyano group, a halogen group, a substituted or unsubstituted alkyl group having 1 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted cycloalkyl group having 3 to 20 carbon atoms, a substituted or unsubstituted aryl group having 6 to 30 carbon atoms, a substituted or unsubstituted heteroaryl group having 2 to 30 carbon atoms, and in each structure of Structural Formula 1, a plurality of R are the same as or different from each other.
(iii) B₁ and B₂ are the same as or different from each other, and each independently selected from hydrogen, deuterium, a cyano group, a halogen group, a substituted or unsubstituted alkyl group having 1 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted cycloalkyl group having 3 to 20 carbon atoms, a substituted or unsubstituted aryl group having 6 to 30 carbon atoms, a substituted or unsubstituted heteroaryl group having 2 to 30 carbon atoms, Structural Formula 1 below, and Structural Formula 2 below.

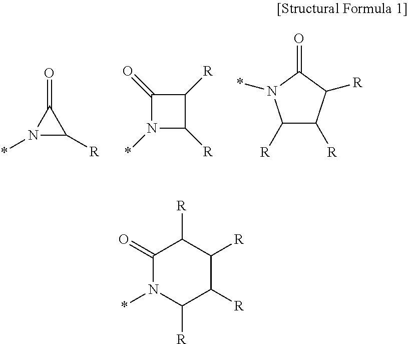

In Structural Formula 1 above,

in each structure, R is each independently selected from hydrogen, deuterium, a cyano group, a halogen group, a substituted or unsubstituted alkyl group having 1 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted cycloalkyl group having 3 to 20 carbon atoms, a substituted or unsubstituted aryl group having 6 to 30 carbon atoms, and a substituted or unsubstituted heteroaryl group having 2 to 30 carbon atoms, and in each structure of Structural Formula 1, a plurality of R are the same as or different from each other.

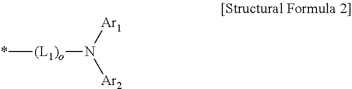

In Structural Formula 2 above,
L₁ is a single bond or selected from a substituted or unsubstituted arylene group having 6 to 30 carbon atoms and a substituted or unsubstituted heteroarylene group having 3 to 30 carbon atoms, o is an integer of 0 to 2, and when the o is 2, a plurality of L₁ are the same as or different from each other.

Ar₁ and Ar₂ are the same as or different from each other and each independently selected from a substituted or unsubstituted aryl group having 6 to 30 carbon atoms and a substituted or unsubstituted heteroaryl group having 2 to 30 carbon atoms.
(iv) Meanwhile, the ‘substituted or unsubstituted’ in the definition of R, B₁, B₂, L, L₁, Ar₁ and Ar₂ above means substitution of R, B₁, B₂, L, L₁, Ar₁ and Ar₂ with one or two or more substituents selected from the group consisting of deuterium, a halogen group, a cyano group, a nitro group, a hydroxy group, a silyl group, an alkyl group, an amine group, a halogenated alkyl group, a deuterated alkyl group, a cycloalkyl group, a heterocycloalkyl group, an alkoxy group, a halogenated alkoxy group, a deuterated alkoxy group, an aryl group, a heteroaryl group, an alkylsilyl group and an arylsilyl group, substitution with a substituent to which two or more of the substituents are linked, or having no substituent.

For specific examples, the substituted arylene group means that a phenyl group, a biphenyl group, a naphthalene group, a fluorenyl group, a pyrenyl group, a phenanthrenyl group, a perylene group, a tetracenyl group, and an anthracenyl group are substituted with other substituents.

In addition, the substituted heteroaryl group means that a pyridyl group, a thiophenyl group, a triazine group, a quinoline group, a phenanthroline group, an imidazole group, a thiazole group, an oxazole group, a carbazole group and a condensate heteroring group thereof, for example, a benzquinoline group, a benzimidazole group, a benzoxazole group, a benzthiazole group, a benzcarbazole group, a dibenzothiophenyl group, and a dibenzofuran group are substituted with other substituents.

In an embodiment of the present invention, examples of the substituents will be described in detail below, but are not limited thereto.

In an embodiment of the present invention, the alkyl groups may be straight or branched. The number of carbon atoms in the alkyl groups is not particularly limited but is preferably from 1 to 20. Specific examples of the alkyl groups include, but are not limited to, methyl, ethyl, propyl, n-propyl, isopropyl, butyl, n-butyl, isobutyl, tert-butyl, sec-butyl, 1-methylbutyl, 1-ethylbutyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethylpropyl, 1,1-dimethylpropyl, isohexyl, 2-methylpentyl, 4-methylhexyl, and 5-methylhexyl groups.

In an embodiment of the present invention, the alkoxy groups may be straight or branched. The number of carbon atoms in the alkoxy groups is not particularly limited but is preferably from 1 to 20 as long as steric hindrance is avoided. Specific examples of the alkoxy groups include, but are not limited to, methoxy, ethoxy, n-propoxy, isopropoxy, i-propyloxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, and p-methylbenzyloxy groups.

In an embodiment of the present invention, the deuterated alkyl group or alkoxy group and the halogenated alkyl group or alkoxy group mean an alkyl group or alkoxy group in which the above alkyl group or alkoxy group is substituted with deuterium or a halogen group.

In an embodiment of the present invention, the aryl groups may be monocyclic or polycyclic. The number of carbon atoms in the aryl groups is not particularly limited but is preferably from 6 to 30. Examples of the monocyclic aryl groups include phenyl, biphenyl, terphenyl, and stilbene groups but the scope of the present invention is not limited thereto. Examples of the polycyclic aryl groups include naphthyl, anthracenyl, phenanthrenyl, pyrenyl, perylenyl, tetracenyl, chrysenyl, fluorenyl, acenaphathcenyl, triphenylene, and fluoranthrene groups, but the scope of the present invention is not limited thereto.

In addition, in an embodiment of the present invention, the fluorenyl groups refer to structures in which two cyclic organic compounds are linked through one atom, and examples thereof include

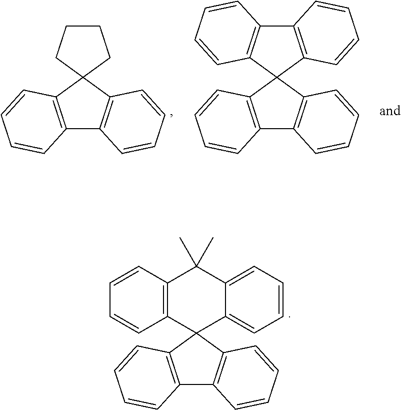

In an embodiment of the present invention, the fluorenyl groups include open structures in which one of the two cyclic organic compounds linked through one atom is cleaved, and examples thereof include

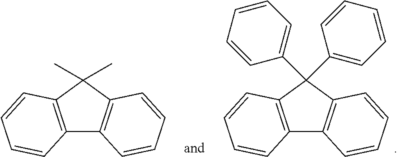

In addition, carbon atoms of the ring may be substituted with any one or more heteroatoms selected from among N, S and O, and examples thereof include

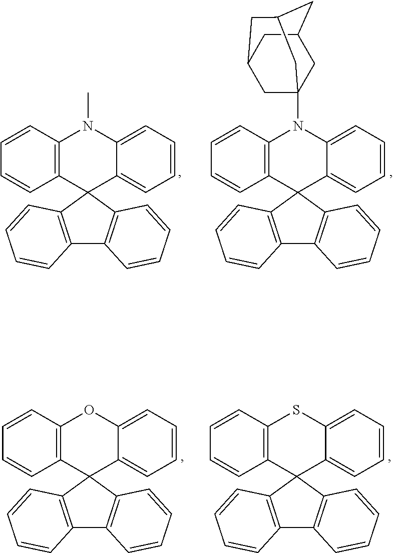

and the like.

In an embodiment of the present invention, the heteroaryl groups refer to heterocyclic groups containing heteroatoms selected from O, N, and S. The number of carbon atoms is not particularly limited, but preferably from 2 to 30. In an embodiment of the present invention, specific examples thereof include, but are not limited to, thiophene, furan, pyrrole, imidazole, thiazole, oxazole, oxadiazole, triazole, pyridyl, bipyridyl, pyrimidyl, triazine, triazole, acridyl, pyridazine, pyrazinyl, quinolinyl, quinazoline, quinoxalinyl, phthalazinyl, pyridopyrimidinyl, pyridopyrazinyl, pyrazinopyrazinyl, isoquinoline, indole, carbazole, benzoxazole, benzimidazole, benzothiazole, benzocarbazole, benzothiophene, dibenzothiophene, benzofuranyl, dibenzofuranyl, phenanthroline, thiazolyl, isoxazolyl, oxadiazolyl, thiadiazolyl, benzothiazolyl, phenothiazinyl, phenoxazine, and phenothiazine groups.

In an embodiment of the present invention, the amine group may be —NH₂, an alkylamine group, an arylamine group, a heteroaryl amino group, an arylheteroarylamine group, etc., the aryl (heteroaryl)amine group means an amine substituted with an aryl group and/or heteroaryl group, and the alkylamine group means an amine substituted with an alkyl group. Examples of the aryl (heteroaryl)amine group include a substituted or unsubstituted mono aryl (heteroaryl)amine group, a substituted or unsubstituted diaryl (heteroaryl)amine group, or a substituted or unsubstituted triaryl (heteroaryl)amine group, wherein the aryl group and the heteroaryl group in the aryl (heteroaryl)amine group are the same as the definition of the aryl group and the heteroaryl group, and the alkyl group in the alkylamine group is also the same as the definition of the alkyl group.

For example, the arylamine group includes a phenylamine group, a naphthylamine group, a biphenylamine group, an anthracenylamine group, a 3-methyl-phenylamine group, a 4-methyl-naphthylamine group, and a 2-methyl-biphenyl amine group, a 9-methyl-anthracenylamine group, a diphenyl amine group, a phenylnaphthylamine group, a ditolylamine group, a phenyltolylamine group, a triphenylamine group, etc., but is not limited thereto.

In an embodiment of the present invention, the silyl group is an unsubstituted silyl group or a silyl group substituted with an alkyl group, an aryl group, and the like, and specific examples of the silyl group include trimethylsilyl, triethylsilyl, triphenylsilyl, trimethoxysilyl, dimethoxyphenylsilyl, diphenylmethylsilyl, diphenylvinylsilyl, methylcyclobutylsilyl, dimethylfurylsilyl, and the like, but are not limited thereto.

Specific examples of the halogen groups as substituents used in an embodiment of the present invention include fluorine (F), chlorine (Cl), and bromine (Br).

In an embodiment of the present invention, a cycloalkyl group refers to a monocyclic, polycyclic and spiro alkyl radical, includes the same, and preferably contains a cyclic carbon atom having 3 to 20 carbon atoms, and includes cyclopropyl, cyclopentyl, cyclohexyl, bicycloheptyl, spirodecyl, spiroundecyl, adamantyl, and the like, and the cycloalkyl group may be arbitrarily substituted.

In an embodiment of the present invention, the heterocycloalkyl group refers to an aromatic or non-aromatic cyclic radical containing one or more heteroatoms, and includes the same, and one or more heteroatoms are selected from among O, S, N, P, B, Si, and Se, preferably O, N or S, and specifically, in the case of including N, the one or more heteroatoms may be aziridine, pyrrolidine, piperidine, azepane, azocane, and the like.

The organic compound according to the present invention represented by Formula I may be used as a material for the light efficiency improving layer (capping layer) provided in the organic light-emitting device due to its structural specificity.

Preferred specific examples of the organic compound represented by Formula I according to the present invention include the following compounds but are not limited thereto.

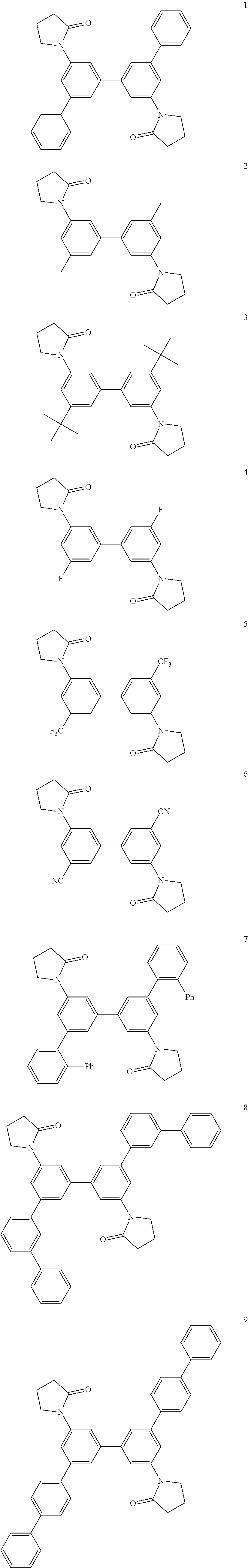

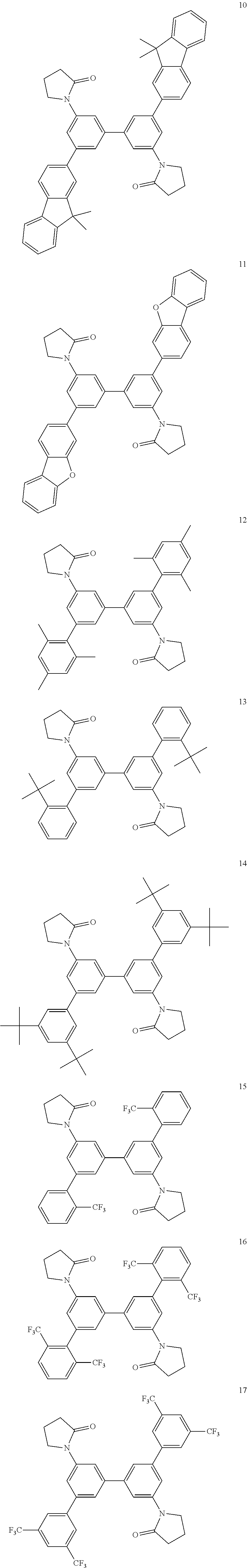

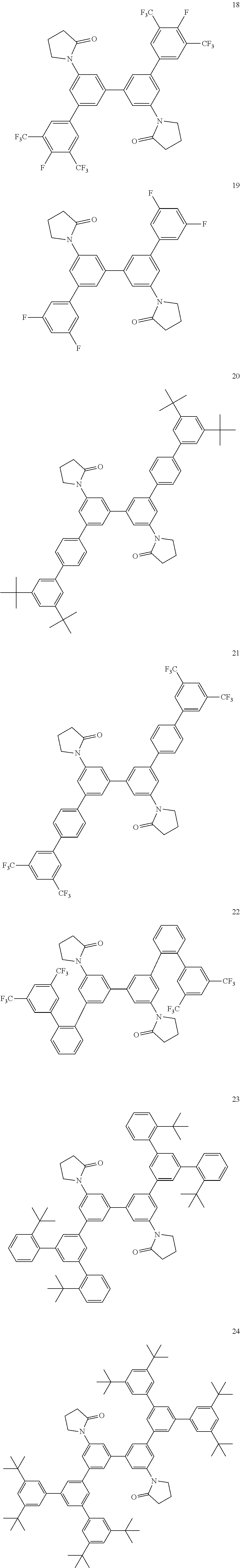

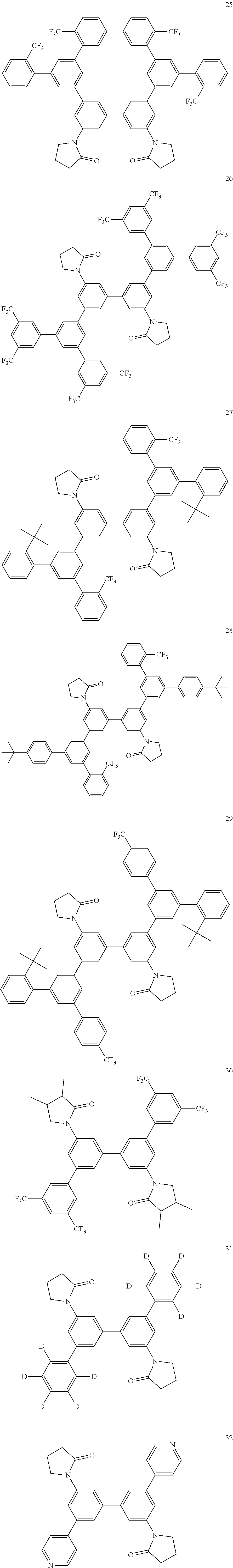

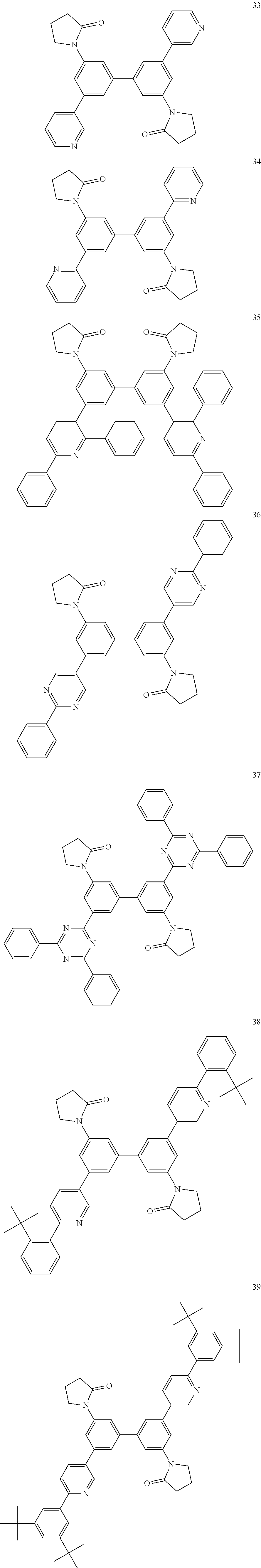

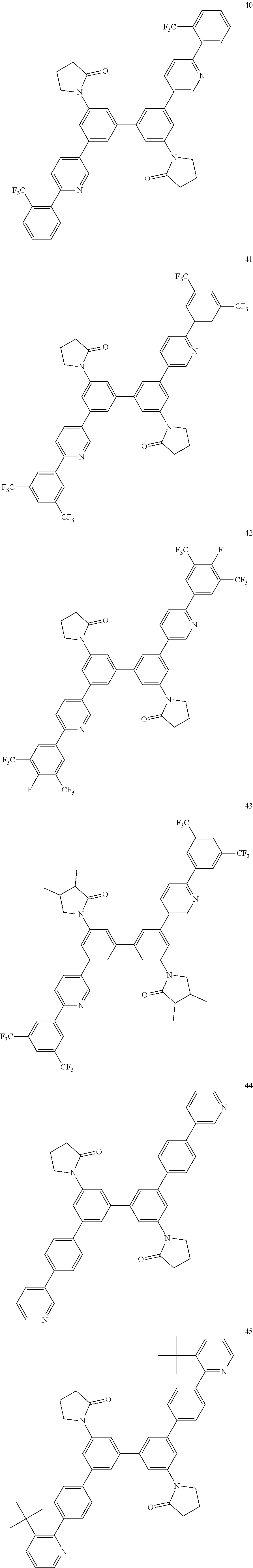

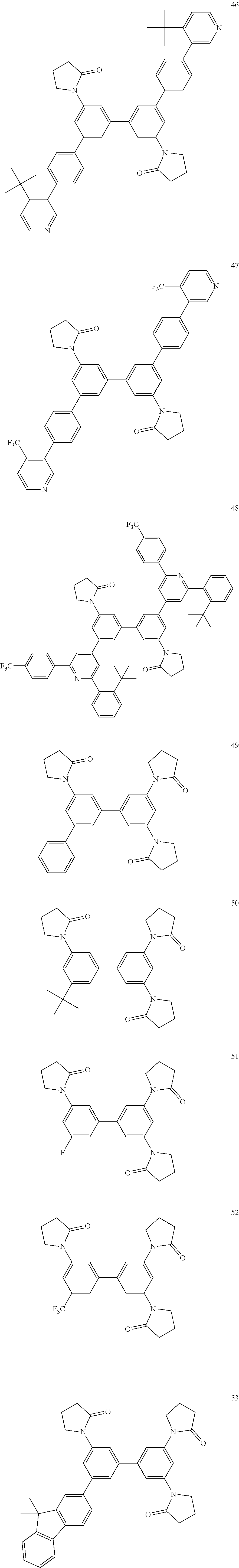

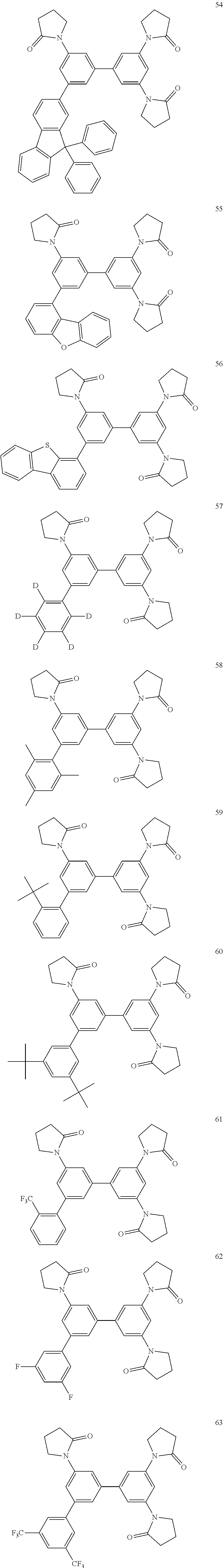

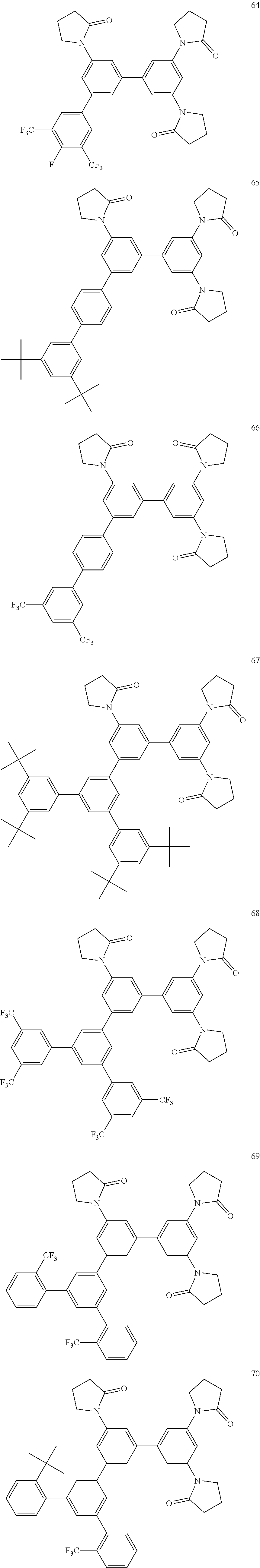

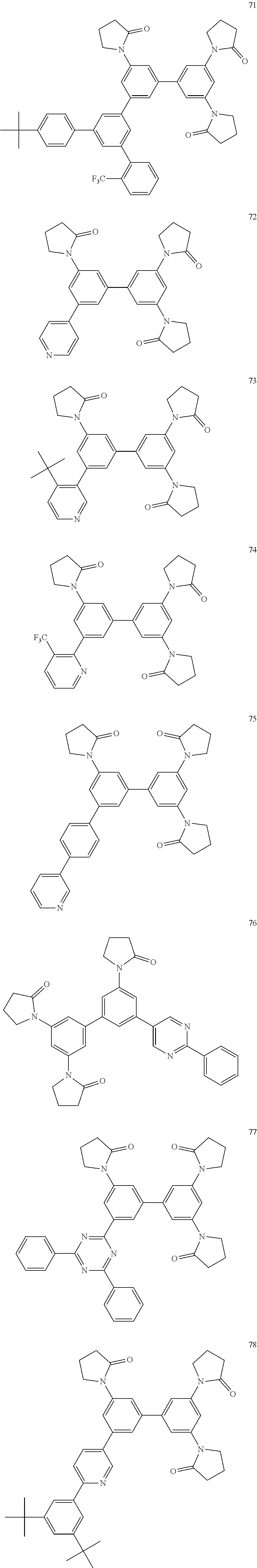

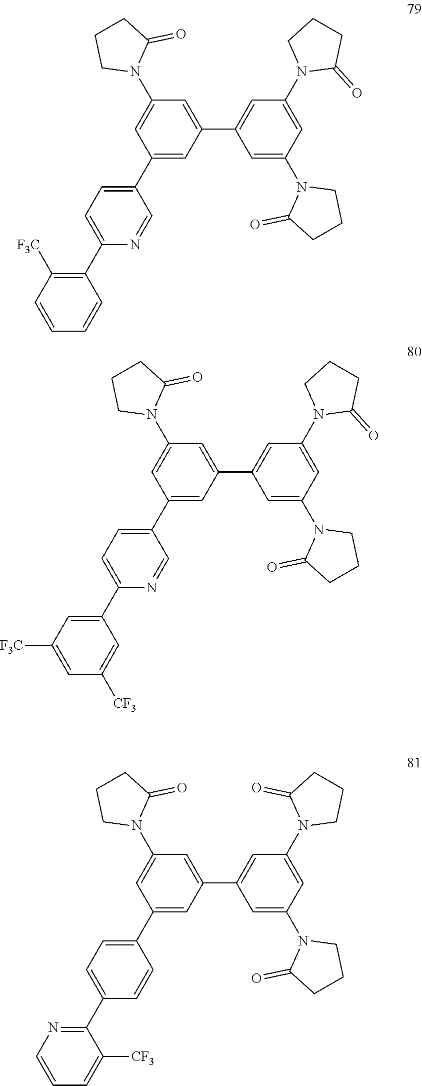 [SMILES: O=C1CCCN1c1cc(-c2ccc(-c3ccccc3C(F)(F)F)nc2)cc(-c2cc(N3CCCC3=O)cc(N3CCCC3=O)c2)c1 ; O=C1CCCN1c1cc(-c2ccc(-c3cc(C(F)(F)F)cc(C(F)(F)F)c3)nc2)cc(-c2cc(N3CCCC3=O)cc(N3CCCC3=O)c2)c1 ; O=C1CCCN1c1cc(-c2ccc(-c3ncccc3C(F)(F)F)cc2)cc(-c2cc(N3CCCC3=O)cc(N3CCCC3=O)c2)c1]

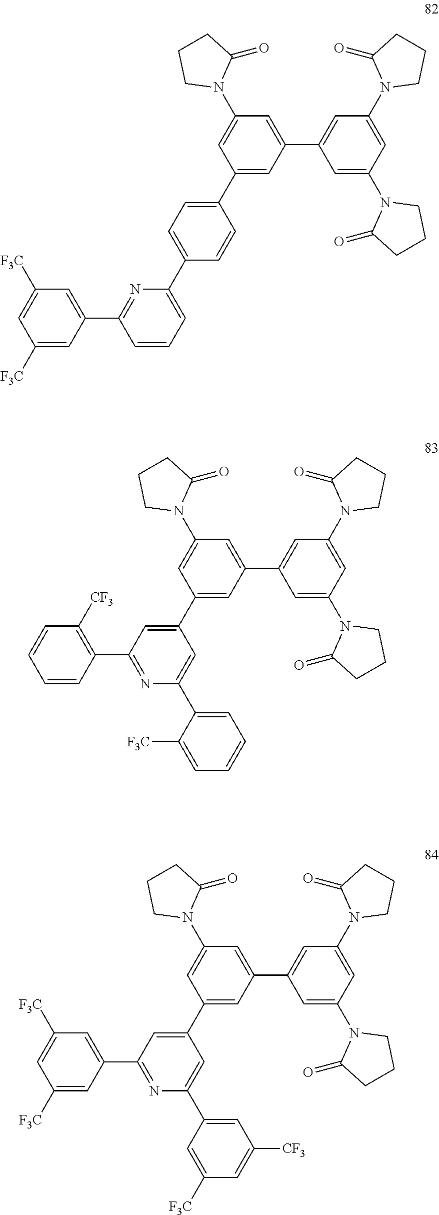 [SMILES: O=C1CCCN1c1cc(-c2ccc(-c3cccc(-c4cc(C(F)(F)F)cc(C(F)(F)F)c4)n3)cc2)cc(-c2cc(N3CCCC3=O)cc(N3CCCC3=O)c2)c1 ; O=C1CCCN1c1cc(-c2cc(N3CCCC3=O)cc(N3CCCC3=O)c2)cc(-c2cc(-c3ccccc3C(F)(F)F)nc(-c3ccccc3C(F)(F)F)c2)c1 ; O=C1CCCN1c1cc(-c2cc(N3CCCC3=O)cc(N3CCCC3=O)c2)cc(-c2cc(-c3cc(C(F)(F)F)cc(C(F)(F)F)c3)nc(-c3cc(C(F)(F)F)cc(C(F)(F)F)c3)c2)c1]

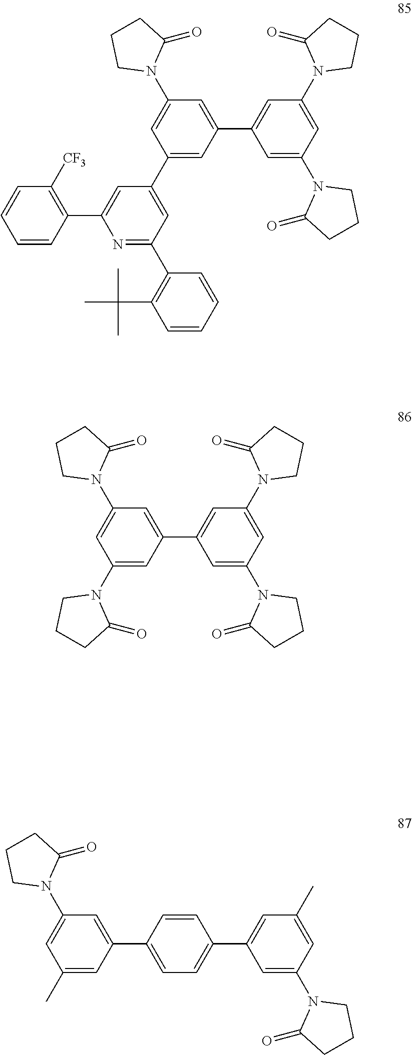 [SMILES: CC(C)(C)c1ccccc1-c1cc(-c2cc(-c3cc(N4CCCC4=O)cc(N4CCCC4=O)c3)cc(N3CCCC3=O)c2)cc(-c2ccccc2C(F)(F)F)n1 ; O=C1CCCN1c1cc(-c2cc(N3CCCC3=O)cc(N3CCCC3=O)c2)cc(N2CCCC2=O)c1 ; Cc1cc(-c2ccc(-c3cc(C)cc(N4CCCC4=O)c3)cc2)cc(N2CCCC2=O)c1]

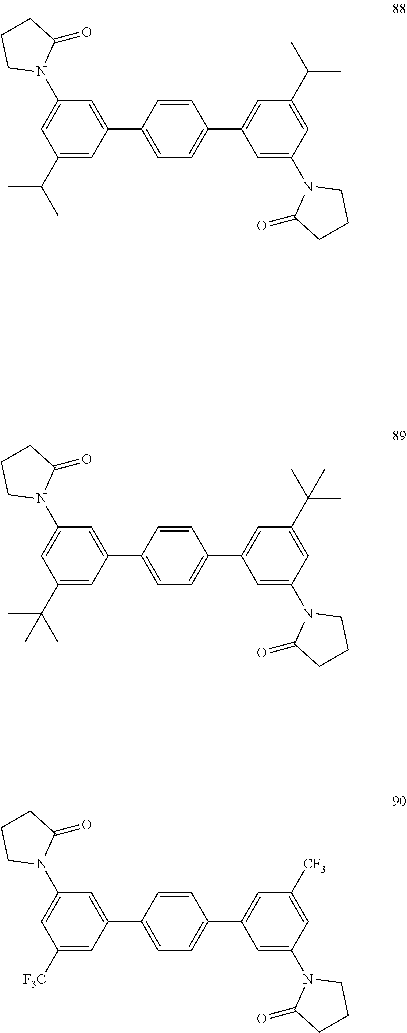 [SMILES: CC(C)c1cc(-c2ccc(-c3cc(C(C)C)cc(N4CCCC4=O)c3)cc2)cc(N2CCCC2=O)c1 ; CC(C)(C)c1cc(-c2ccc(-c3cc(N4CCCC4=O)cc(C(C)(C)C)c3)cc2)cc(N2CCCC2=O)c1 ; O=C1CCCN1c1cc(-c2ccc(-c3cc(N4CCCC4=O)cc(C(F)(F)F)c3)cc2)cc(C(F)(F)F)c1]

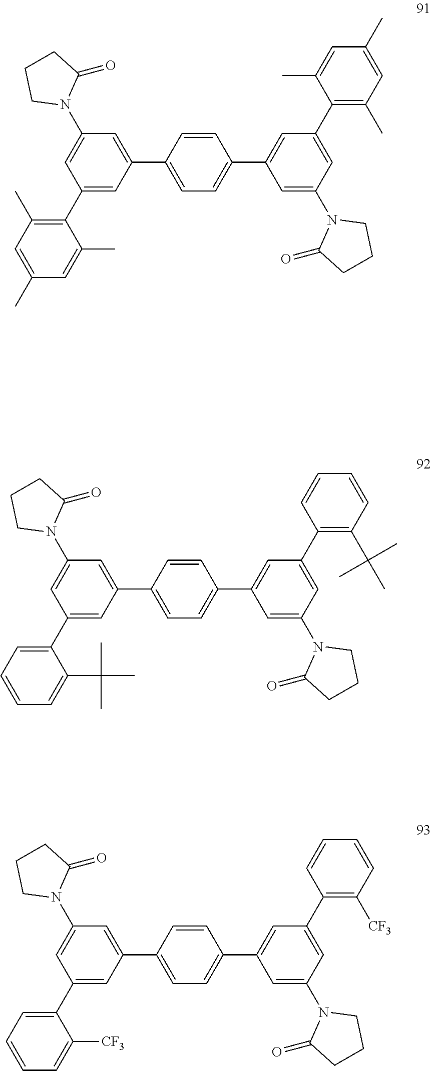 [SMILES: Cc1cc(C)c(-c2cc(-c3ccc(-c4cc(-c5c(C)cc(C)cc5C)cc(N5CCCC5=O)c4)cc3)cc(N3CCCC3=O)c2)c(C)c1 ; CC(C)(C)c1ccccc1-c1cc(-c2ccc(-c3cc(-c4ccccc4C(C)(C)C)cc(N4CCCC4=O)c3)cc2)cc(N2CCCC2=O)c1 ; O=C1CCCN1c1cc(-c2ccc(-c3cc(-c4ccccc4C(F)(F)F)cc(N4CCCC4=O)c3)cc2)cc(-c2ccccc2C(F)(F)F)c1]

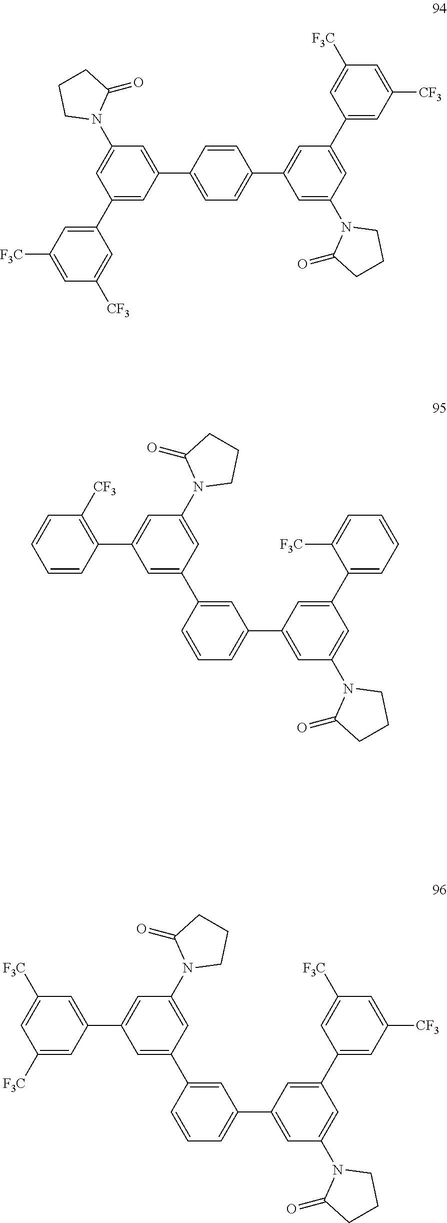 [SMILES: O=C1CCCN1c1cc(-c2ccc(-c3cc(-c4cc(C(F)(F)F)cc(C(F)(F)F)c4)cc(N4CCCC4=O)c3)cc2)cc(-c2cc(C(F)(F)F)cc(C(F)(F)F)c2)c1 ; O=C1CCCN1c1cc(-c2cccc(-c3cc(-c4ccccc4C(F)(F)F)cc(N4CCCC4=O)c3)c2)cc(-c2ccccc2C(F)(F)F)c1 ; O=C1CCCN1c1cc(-c2cccc(-c3cc(-c4cc(C(F)(F)F)cc(C(F)(F)F)c4)cc(N4CCCC4=O)c3)c2)cc(-c2cc(C(F)(F)F)cc(C(F)(F)F)c2)c1]

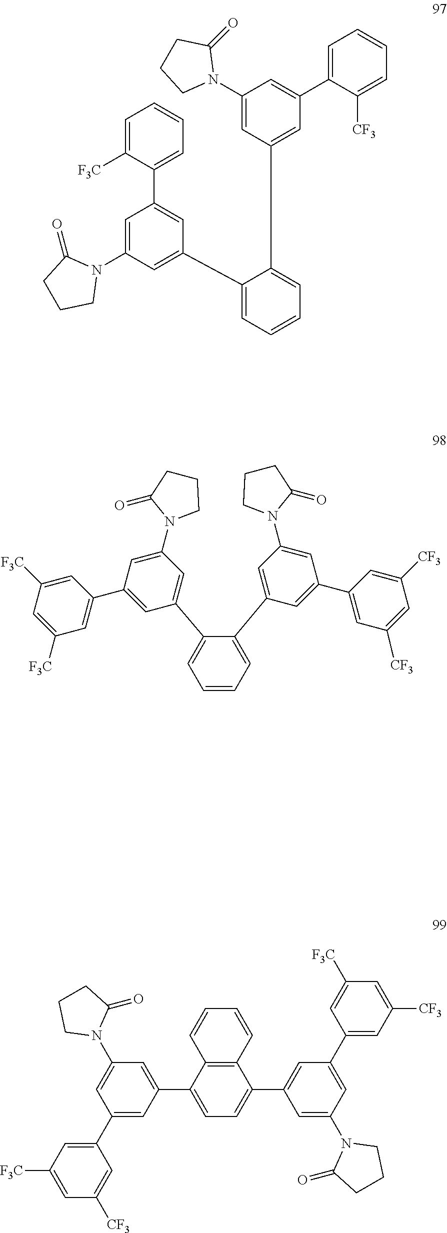 [SMILES: O=C1CCCN1c1cc(-c2ccccc2-c2cc(-c3ccccc3C(F)(F)F)cc(N3CCCC3=O)c2)cc(-c2ccccc2C(F)(F)F)c1 ; O=C1CCCN1c1cc(-c2cc(C(F)(F)F)cc(C(F)(F)F)c2)cc(-c2ccccc2-c2cc(-c3cc(C(F)(F)F)cc(C(F)(F)F)c3)cc(N3CCCC3=O)c2)c1 ; O=C1CCCN1c1cc(-c2cc(C(F)(F)F)cc(C(F)(F)F)c2)cc(-c2ccc(-c3cc(-c4cc(C(F)(F)F)cc(C(F)(F)F)c4)cc(N4CCCC4=O)c3)c3ccccc23)c1]

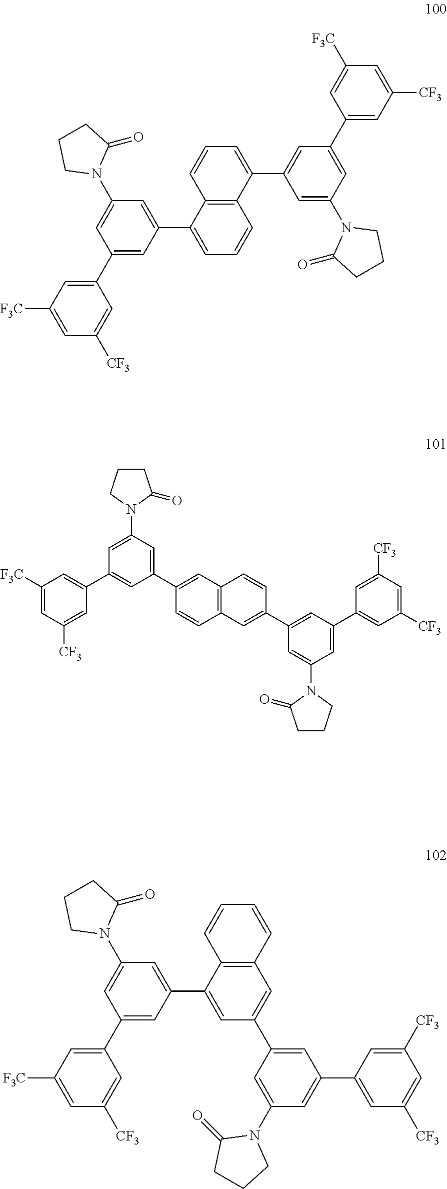 [SMILES: O=C1CCCN1c1cc(-c2cc(C(F)(F)F)cc(C(F)(F)F)c2)cc(-c2cccc3c(-c4cc(-c5cc(C(F)(F)F)cc(C(F)(F)F)c5)cc(N5CCCC5=O)c4)cccc23)c1 ; O=C1CCCN1c1cc(-c2cc(C(F)(F)F)cc(C(F)(F)F)c2)cc(-c2ccc3cc(-c4cc(-c5cc(C(F)(F)F)cc(C(F)(F)F)c5)cc(N5CCCC5=O)c4)ccc3c2)c1 ; O=C1CCCN1c1cc(-c2cc(C(F)(F)F)cc(C(F)(F)F)c2)cc(-c2cc(-c3cc(-c4cc(C(F)(F)F)cc(C(F)(F)F)c4)cc(N4CCCC4=O)c3)c3ccccc3c2)c1]

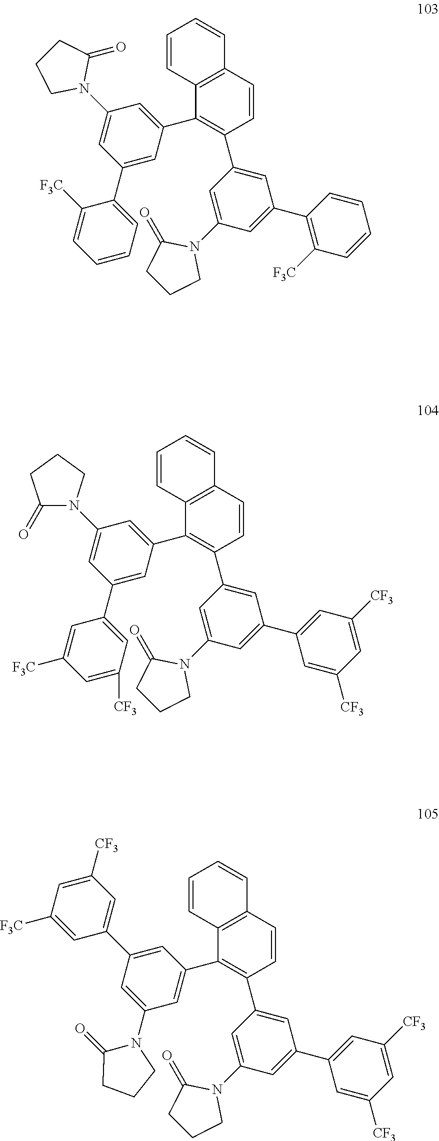 [SMILES: O=C1CCCN1c1cc(-c2ccccc2C(F)(F)F)cc(-c2ccc3ccccc3c2-c2cc(-c3ccccc3C(F)(F)F)cc(N3CCCC3=O)c2)c1 ; O=C1CCCN1c1cc(-c2cc(C(F)(F)F)cc(C(F)(F)F)c2)cc(-c2ccc3ccccc3c2-c2cc(-c3cc(C(F)(F)F)cc(C(F)(F)F)c3)cc(N3CCCC3=O)c2)c1 ; O=C1CCCN1c1cc(-c2cc(C(F)(F)F)cc(C(F)(F)F)c2)cc(-c2ccc3ccccc3c2-c2cc(-c3cc(C(F)(F)F)cc(C(F)(F)F)c3)cc(N3CCCC3=O)c2)c1]

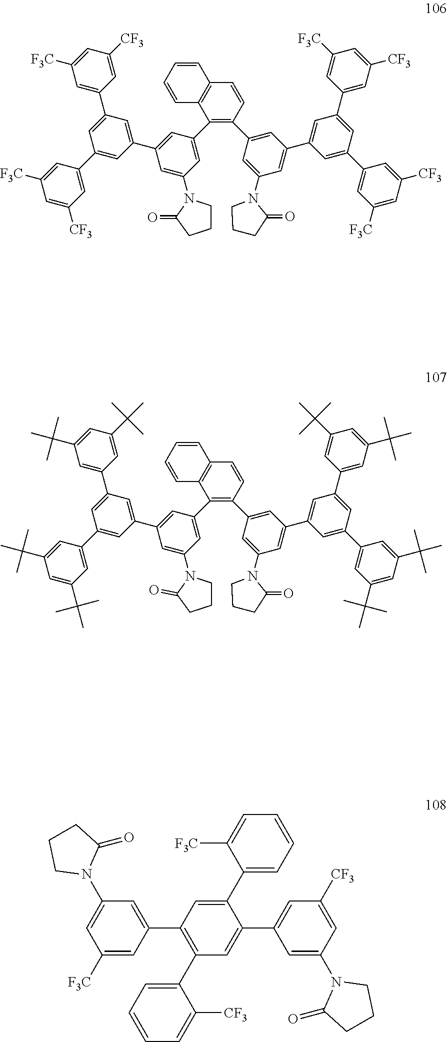

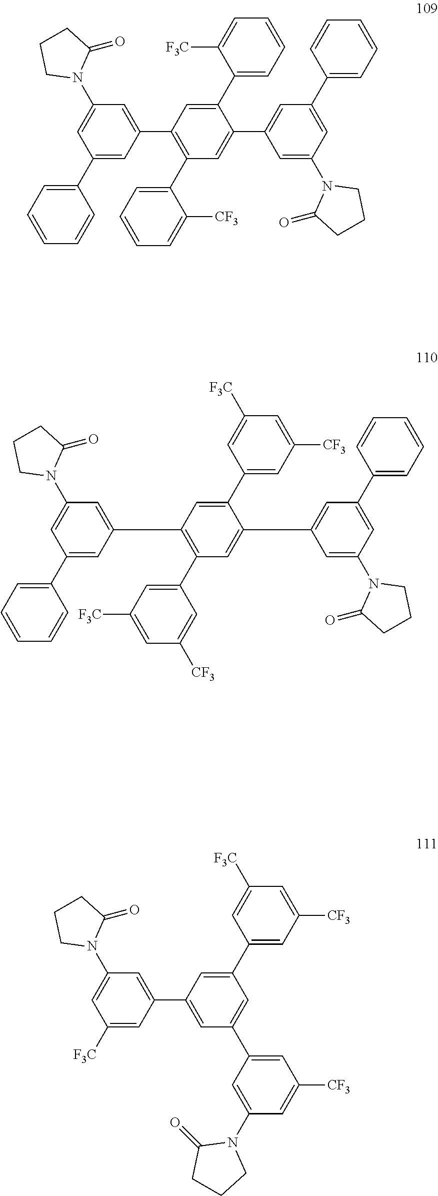 [SMILES: O=C1CCCN1c1cc(-c2ccccc2)cc(-c2cc(-c3ccccc3C(F)(F)F)c(-c3cc(-c4ccccc4)cc(N4CCCC4=O)c3)cc2-c2ccccc2C(F)(F)F)c1 ; O=C1CCCN1c1cc(-c2ccccc2)cc(-c2cc(-c3cc(C(F)(F)F)cc(C(F)(F)F)c3)c(-c3cc(-c4ccccc4)cc(N4CCCC4=O)c3)cc2-c2cc(C(F)(F)F)cc(C(F)(F)F)c2)c1 ; O=C1CCCN1c1cc(-c2cc(-c3cc(N4CCCC4=O)cc(C(F)(F)F)c3)cc(-c3cc(C(F)(F)F)cc(C(F)(F)F)c3)c2)cc(C(F)(F)F)c1]

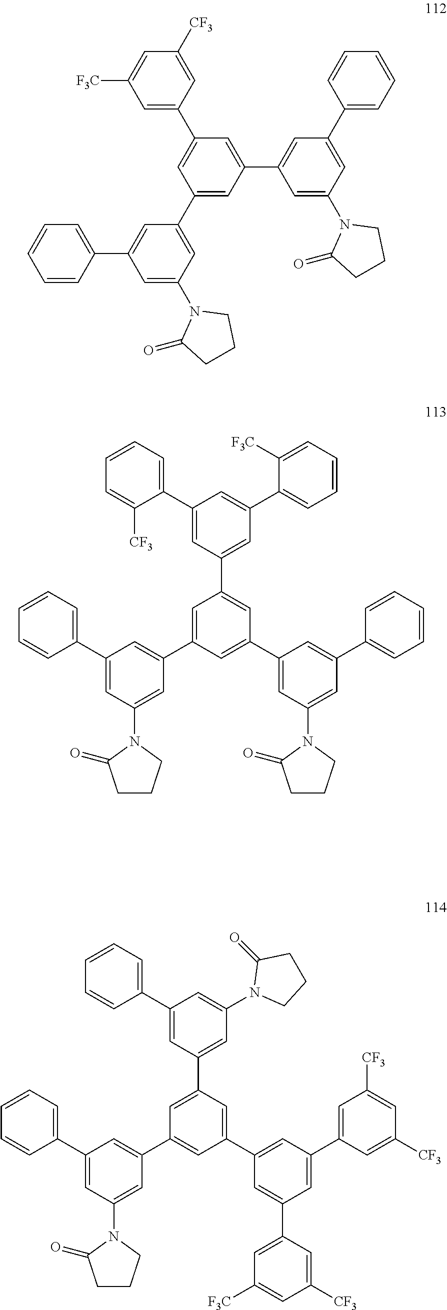 [SMILES: O=C1CCCN1c1cc(-c2ccccc2)cc(-c2cc(-c3cc(-c4ccccc4)cc(N4CCCC4=O)c3)cc(-c3cc(C(F)(F)F)cc(C(F)(F)F)c3)c2)c1 ; O=C1CCCN1c1cc(-c2ccccc2)cc(-c2cc(-c3cc(-c4ccccc4C(F)(F)F)cc(-c4ccccc4C(F)(F)F)c3)cc(-c3cc(-c4ccccc4)cc(N4CCCC4=O)c3)c2)c1 ; O=C1CCCN1c1cc(-c2ccccc2)cc(-c2cc(-c3cc(-c4cc(C(F)(F)F)cc(C(F)(F)F)c4)cc(-c4cc(C(F)(F)F)cc(C(F)(F)F)c4)c3)cc(-c3cc(-c4ccccc4)cc(N4CCCC4=O)c3)c2)c1]

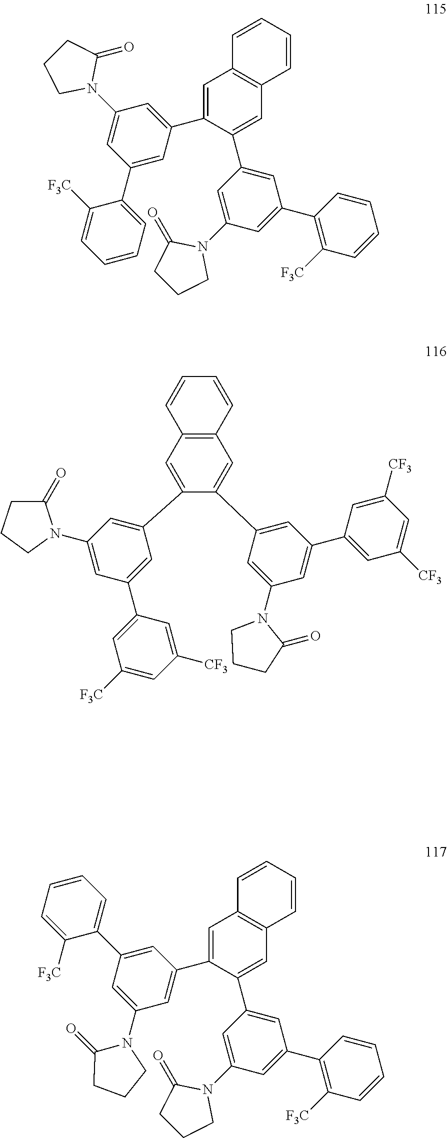 [SMILES: O=C1CCCN1c1cc(-c2cc3ccccc3cc2-c2cc(-c3ccccc3C(F)(F)F)cc(N3CCCC3=O)c2)cc(-c2ccccc2C(F)(F)F)c1 ; O=C1CCCN1c1cc(-c2cc(C(F)(F)F)cc(C(F)(F)F)c2)cc(-c2cc3ccccc3cc2-c2cc(-c3cc(C(F)(F)F)cc(C(F)(F)F)c3)cc(N3CCCC3=O)c2)c1 ; O=C1CCCN1c1cc(-c2cc3ccccc3cc2-c2cc(-c3ccccc3C(F)(F)F)cc(N3CCCC3=O)c2)cc(-c2ccccc2C(F)(F)F)c1]

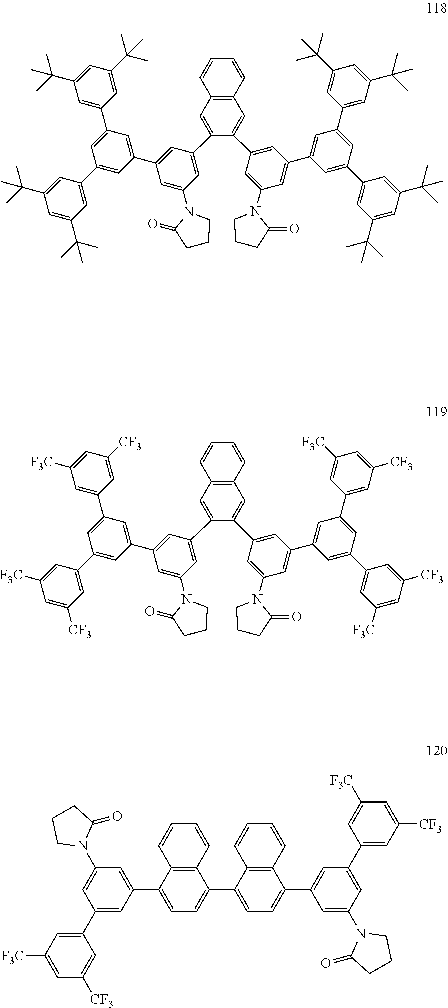

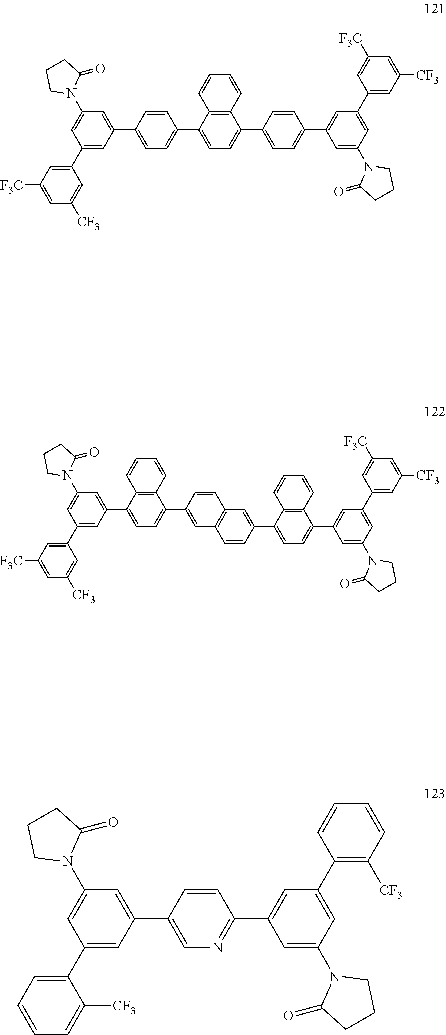 [SMILES: O=C1CCCN1c1cc(-c2ccc(-c3ccc(-c4ccc(-c5cc(-c6cc(C(F)(F)F)cc(C(F)(F)F)c6)cc(N6CCCC6=O)c5)cc4)c4ccccc34)cc2)cc(-c2cc(C(F)(F)F)cc(C(F)(F)F)c2)c1 ; O=C1CCCN1c1cc(-c2cc(C(F)(F)F)cc(C(F)(F)F)c2)cc(-c2ccc(-c3ccc4cc(-c5ccc(-c6cc(-c7cc(C(F)(F)F)cc(C(F)(F)F)c7)cc(N7CCCC7=O)c6)c6ccccc56)ccc4c3)c3ccccc23)c1 ; O=C1CCCN1c1cc(-c2ccc(-c3cc(-c4ccccc4C(F)(F)F)cc(N4CCCC4=O)c3)nc2)cc(-c2ccccc2C(F)(F)F)c1]

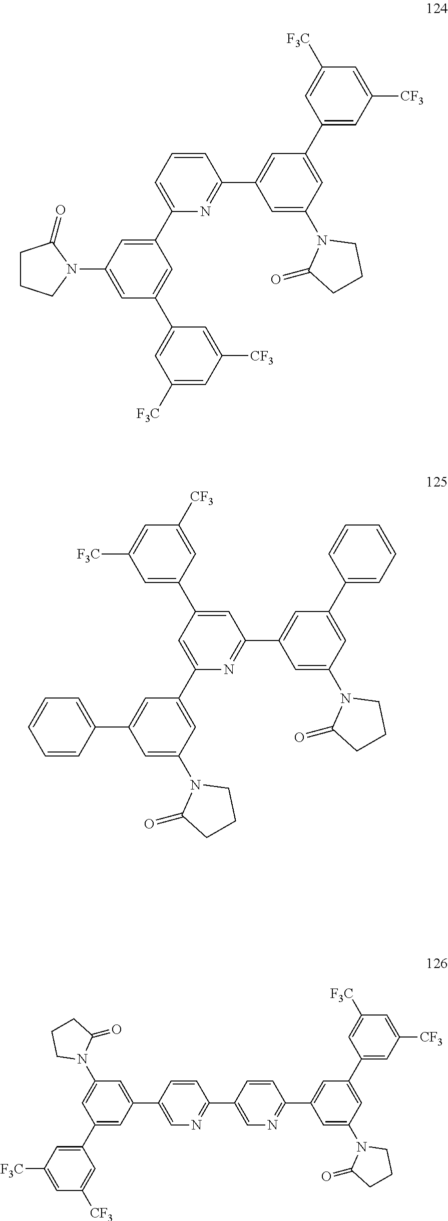 [SMILES: O=C1CCCN1c1cc(-c2cc(C(F)(F)F)cc(C(F)(F)F)c2)cc(-c2cccc(-c3cc(-c4cc(C(F)(F)F)cc(C(F)(F)F)c4)cc(N4CCCC4=O)c3)n2)c1 ; O=C1CCCN1c1cc(-c2ccccc2)cc(-c2cc(-c3cc(C(F)(F)F)cc(C(F)(F)F)c3)cc(-c3cc(-c4ccccc4)cc(N4CCCC4=O)c3)n2)c1 ; O=C1CCCN1c1cc(-c2ccc(-c3ccc(-c4cc(-c5cc(C(F)(F)F)cc(C(F)(F)F)c5)cc(N5CCCC5=O)c4)nc3)nc2)cc(-c2cc(C(F)(F)F)cc(C(F)(F)F)c2)c1]

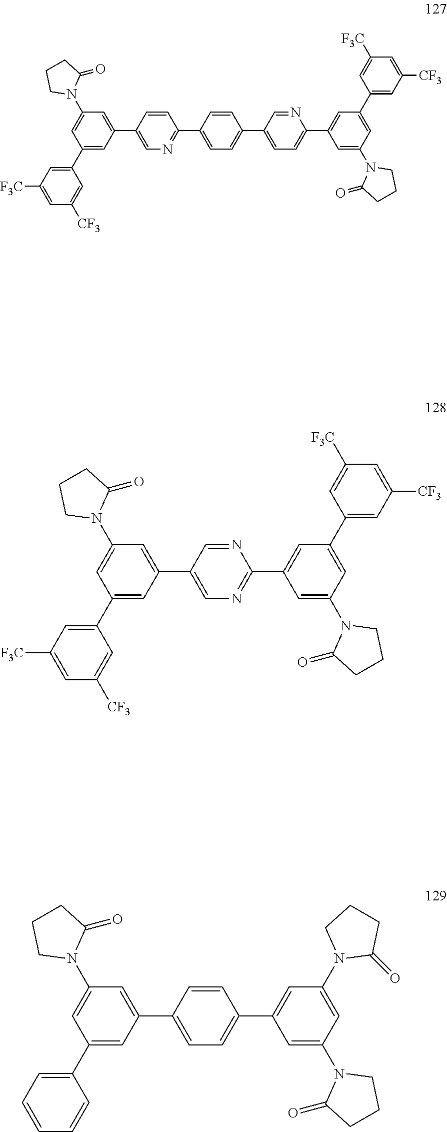 [SMILES: O=C1CCCN1c1cc(-c2ccc(-c3ccc(-c4ccc(-c5cc(-c6cc(C(F)(F)F)cc(C(F)(F)F)c6)cc(N6CCCC6=O)c5)nc4)cc3)nc2)cc(-c2cc(C(F)(F)F)cc(C(F)(F)F)c2)c1 ; O=C1CCCN1c1cc(-c2cnc(-c3cc(-c4cc(C(F)(F)F)cc(C(F)(F)F)c4)cc(N4CCCC4=O)c3)nc2)cc(-c2cc(C(F)(F)F)cc(C(F)(F)F)c2)c1 ; O=C1CCCN1c1cc(-c2ccccc2)cc(-c2ccc(-c3cc(N4CCCC4=O)cc(N4CCCC4=O)c3)cc2)c1]

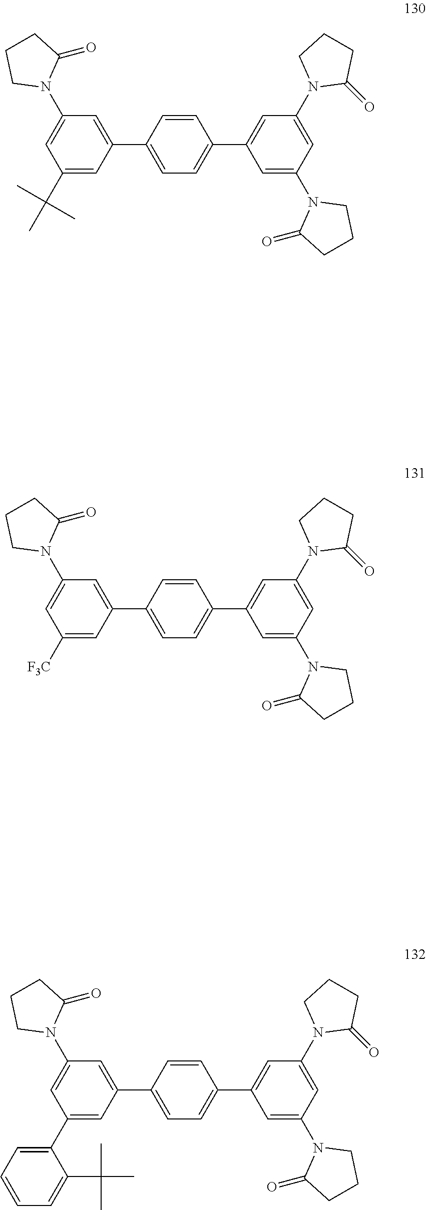 [SMILES: CC(C)(C)c1cc(-c2ccc(-c3cc(N4CCCC4=O)cc(N4CCCC4=O)c3)cc2)cc(N2CCCC2=O)c1 ; O=C1CCCN1c1cc(-c2ccc(-c3cc(N4CCCC4=O)cc(C(F)(F)F)c3)cc2)cc(N2CCCC2=O)c1 ; CC(C)(C)c1ccccc1-c1cc(-c2ccc(-c3cc(N4CCCC4=O)cc(N4CCCC4=O)c3)cc2)cc(N2CCCC2=O)c1]

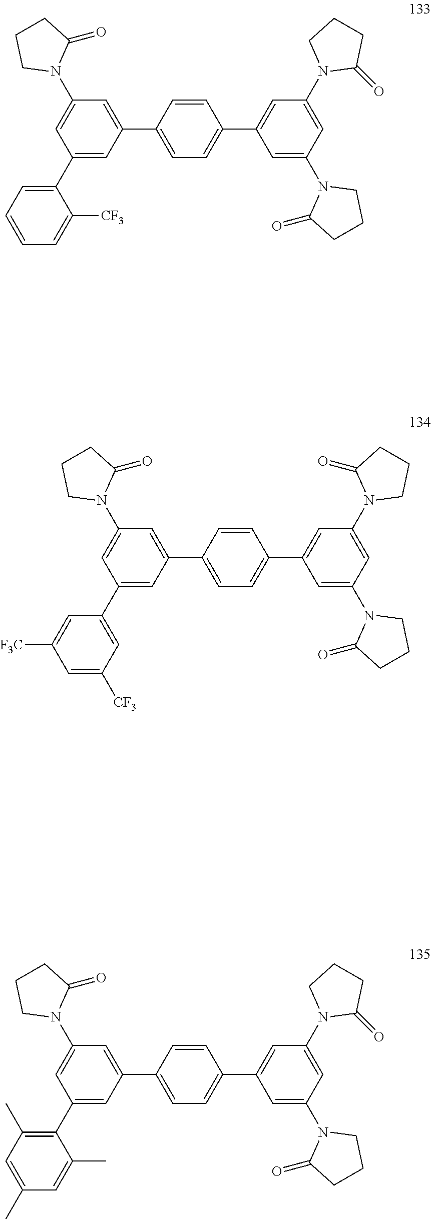 [SMILES: O=C1CCCN1c1cc(-c2ccc(-c3cc(N4CCCC4=O)cc(N4CCCC4=O)c3)cc2)cc(-c2ccccc2C(F)(F)F)c1 ; O=C1CCCN1c1cc(-c2ccc(-c3cc(N4CCCC4=O)cc(N4CCCC4=O)c3)cc2)cc(-c2cc(C(F)(F)F)cc(C(F)(F)F)c2)c1 ; Cc1cc(C)c(-c2cc(-c3ccc(-c4cc(N5CCCC5=O)cc(N5CCCC5=O)c4)cc3)cc(N3CCCC3=O)c2)c(C)c1]

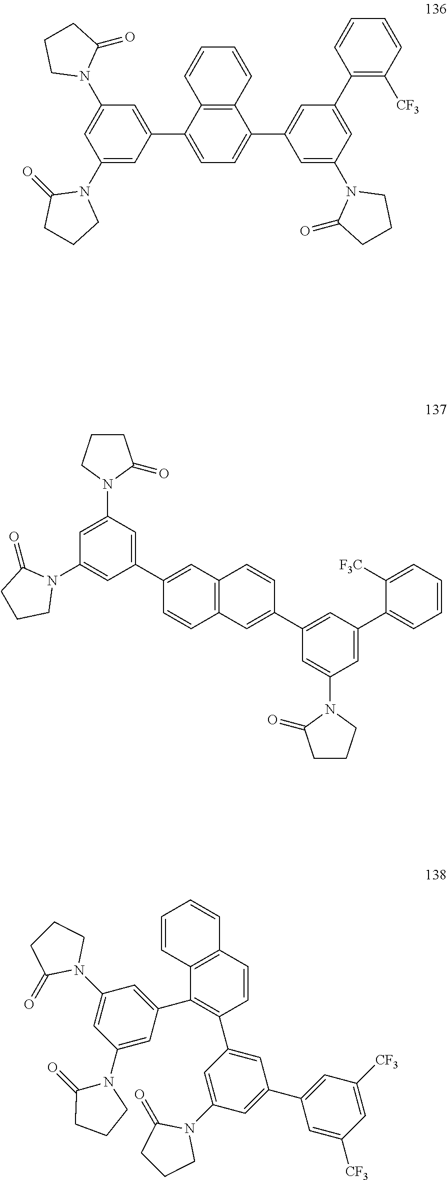 [SMILES: O=C1CCCN1c1cc(-c2ccccc2C(F)(F)F)cc(-c2ccc(-c3cc(N4CCCC4=O)cc(N4CCCC4=O)c3)c3ccccc23)c1 ; O=C1CCCN1c1cc(-c2ccc3cc(-c4cc(N5CCCC5=O)cc(N5CCCC5=O)c4)ccc3c2)cc(-c2ccccc2C(F)(F)F)c1 ; O=C1CCCN1c1cc(-c2cc(C(F)(F)F)cc(C(F)(F)F)c2)cc(-c2ccc3ccccc3c2-c2cc(N3CCCC3=O)cc(N3CCCC3=O)c2)c1]

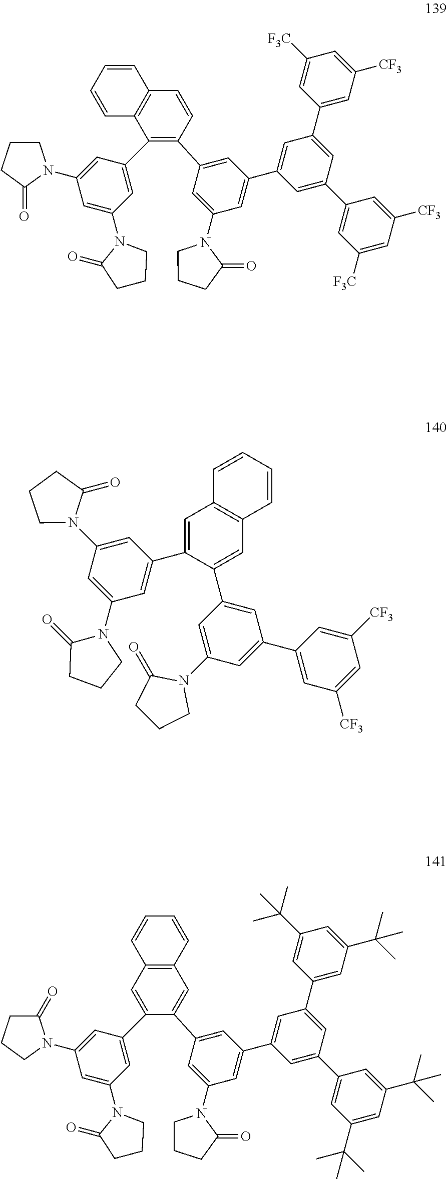 [SMILES: O=C1CCCN1c1cc(-c2cc(-c3cc(C(F)(F)F)cc(C(F)(F)F)c3)cc(-c3cc(C(F)(F)F)cc(C(F)(F)F)c3)c2)cc(-c2ccc3ccccc3c2-c2cc(N3CCCC3=O)cc(N3CCCC3=O)c2)c1 ; O=C1CCCN1c1cc(-c2cc(C(F)(F)F)cc(C(F)(F)F)c2)cc(-c2cc3ccccc3cc2-c2cc(N3CCCC3=O)cc(N3CCCC3=O)c2)c1 ; CC(C)(C)c1cc(-c2cc(-c3cc(-c4cc5ccccc5cc4-c4cc(N5CCCC5=O)cc(N5CCCC5=O)c4)cc(N4CCCC4=O)c3)cc(-c3cc(C(C)(C)C)cc(C(C)(C)C)c3)c2)cc(C(C)(C)C)c1]

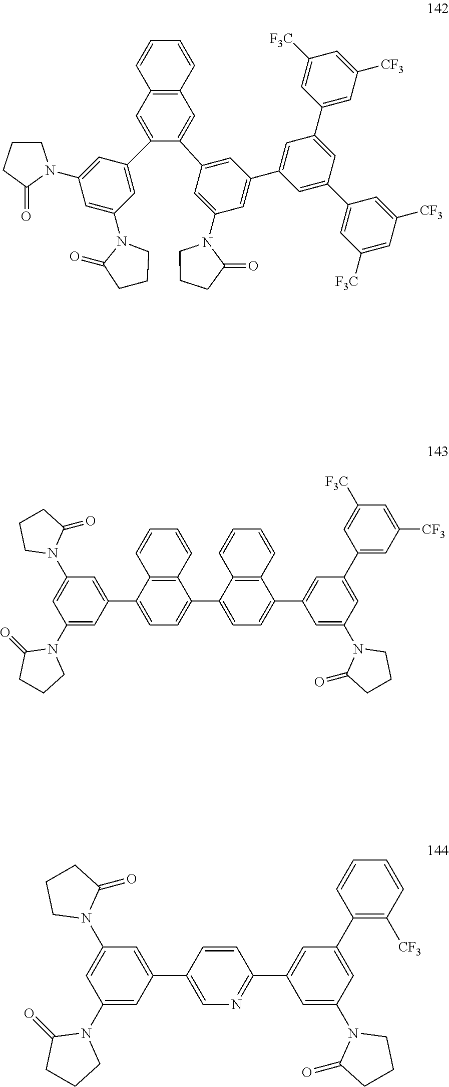 [SMILES: O=C1CCCN1c1cc(-c2cc(-c3cc(C(F)(F)F)cc(C(F)(F)F)c3)cc(-c3cc(C(F)(F)F)cc(C(F)(F)F)c3)c2)cc(-c2cc3ccccc3cc2-c2cc(N3CCCC3=O)cc(N3CCCC3=O)c2)c1 ; O=C1CCCN1c1cc(-c2cc(C(F)(F)F)cc(C(F)(F)F)c2)cc(-c2ccc(-c3ccc(-c4cc(N5CCCC5=O)cc(N5CCCC5=O)c4)c4ccccc34)c3ccccc23)c1 ; O=C1CCCN1c1cc(-c2ccc(-c3cc(N4CCCC4=O)cc(N4CCCC4=O)c3)cn2)cc(-c2ccccc2C(F)(F)F)c1]

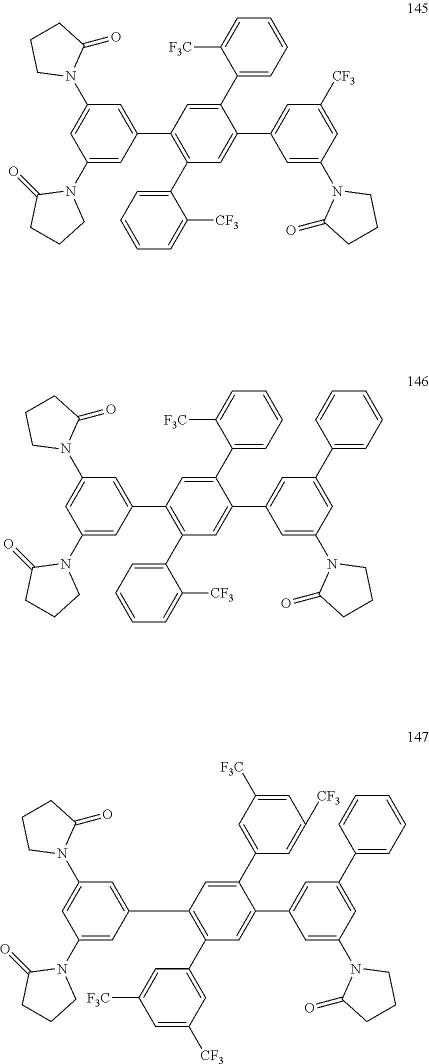 [SMILES: O=C1CCCN1c1cc(-c2cc(-c3ccccc3C(F)(F)F)c(-c3cc(N4CCCC4=O)cc(C(F)(F)F)c3)cc2-c2ccccc2C(F)(F)F)cc(N2CCCC2=O)c1 ; O=C1CCCN1c1cc(-c2ccccc2)cc(-c2cc(-c3ccccc3C(F)(F)F)c(-c3cc(N4CCCC4=O)cc(N4CCCC4=O)c3)cc2-c2ccccc2C(F)(F)F)c1 ; O=C1CCCN1c1cc(-c2ccccc2)cc(-c2cc(-c3cc(C(F)(F)F)cc(C(F)(F)F)c3)c(-c3cc(N4CCCC4=O)cc(N4CCCC4=O)c3)cc2-c2cc(C(F)(F)F)cc(C(F)(F)F)c2)c1]

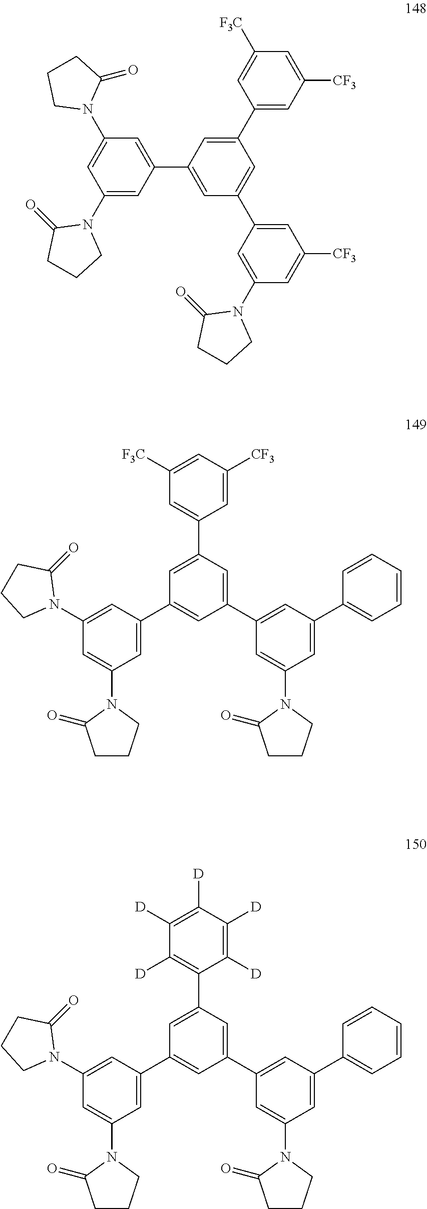 [SMILES: O=C1CCCN1c1cc(-c2cc(-c3cc(N4CCCC4=O)cc(C(F)(F)F)c3)cc(-c3cc(C(F)(F)F)cc(C(F)(F)F)c3)c2)cc(N2CCCC2=O)c1 ; O=C1CCCN1c1cc(-c2ccccc2)cc(-c2cc(-c3cc(N4CCCC4=O)cc(N4CCCC4=O)c3)cc(-c3cc(C(F)(F)F)cc(C(F)(F)F)c3)c2)c1 ; [2H]c1c([2H])c([2H])c(-c2cc(-c3cc(-c4ccccc4)cc(N4CCCC4=O)c3)cc(-c3cc(N4CCCC4=O)cc(N4CCCC4=O)c3)c2)c([2H])c1[2H]]

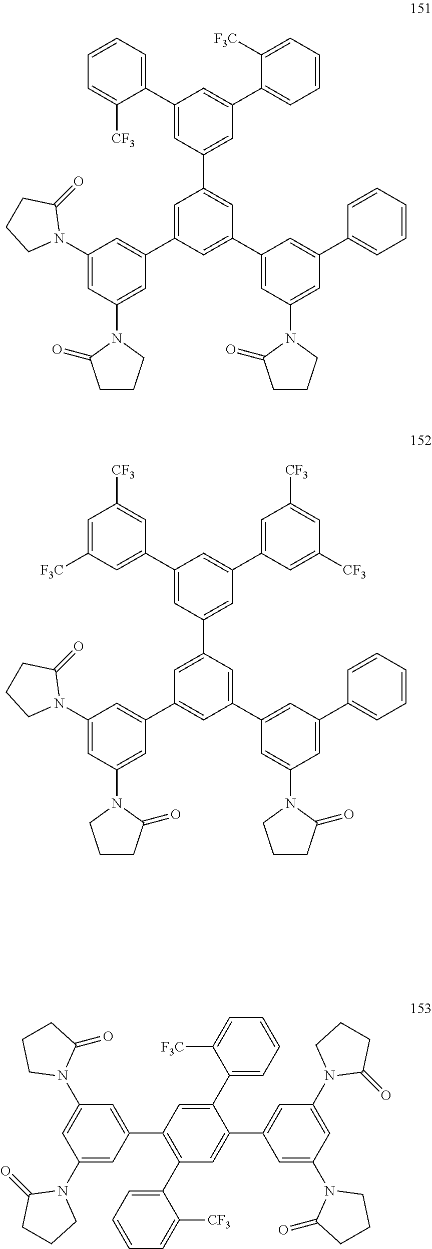 [SMILES: O=C1CCCN1c1cc(-c2ccccc2)cc(-c2cc(-c3cc(-c4ccccc4C(F)(F)F)cc(-c4ccccc4C(F)(F)F)c3)cc(-c3cc(N4CCCC4=O)cc(N4CCCC4=O)c3)c2)c1 ; O=C1CCCN1c1cc(-c2ccccc2)cc(-c2cc(-c3cc(-c4cc(C(F)(F)F)cc(C(F)(F)F)c4)cc(-c4cc(C(F)(F)F)cc(C(F)(F)F)c4)c3)cc(-c3cc(N4CCCC4=O)cc(N4CCCC4=O)c3)c2)c1 ; O=C1CCCN1c1cc(-c2cc(-c3ccccc3C(F)(F)F)c(-c3cc(N4CCCC4=O)cc(N4CCCC4=O)c3)cc2-c2ccccc2C(F)(F)F)cc(N2CCCC2=O)c1]

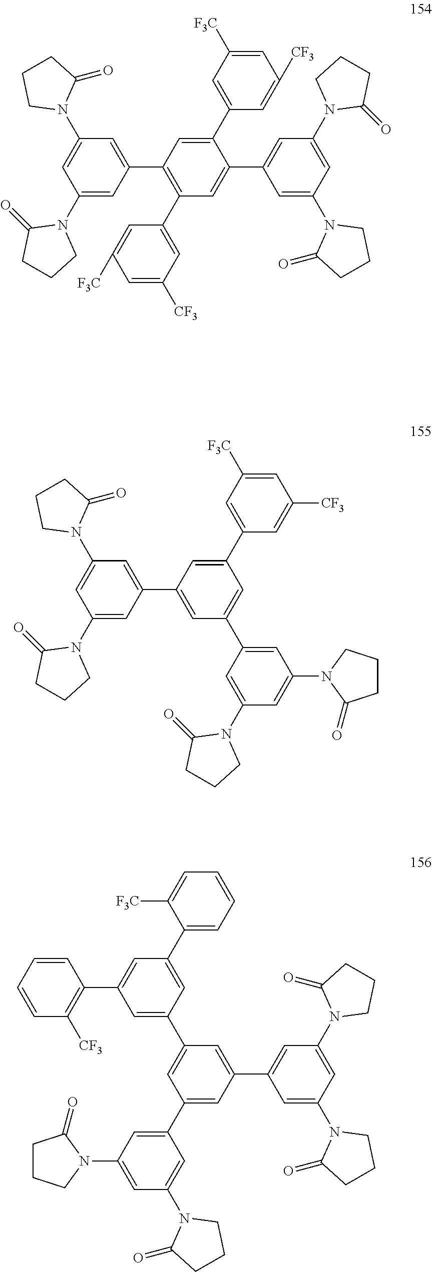 [SMILES: O=C1CCCN1c1cc(-c2cc(-c3cc(C(F)(F)F)cc(C(F)(F)F)c3)c(-c3cc(N4CCCC4=O)cc(N4CCCC4=O)c3)cc2-c2cc(C(F)(F)F)cc(C(F)(F)F)c2)cc(N2CCCC2=O)c1 ; O=C1CCCN1c1cc(-c2cc(-c3cc(N4CCCC4=O)cc(N4CCCC4=O)c3)cc(-c3cc(C(F)(F)F)cc(C(F)(F)F)c3)c2)cc(N2CCCC2=O)c1 ; O=C1CCCN1c1cc(-c2cc(-c3cc(-c4ccccc4C(F)(F)F)cc(-c4ccccc4C(F)(F)F)c3)cc(-c3cc(N4CCCC4=O)cc(N4CCCC4=O)c3)c2)cc(N2CCCC2=O)c1]

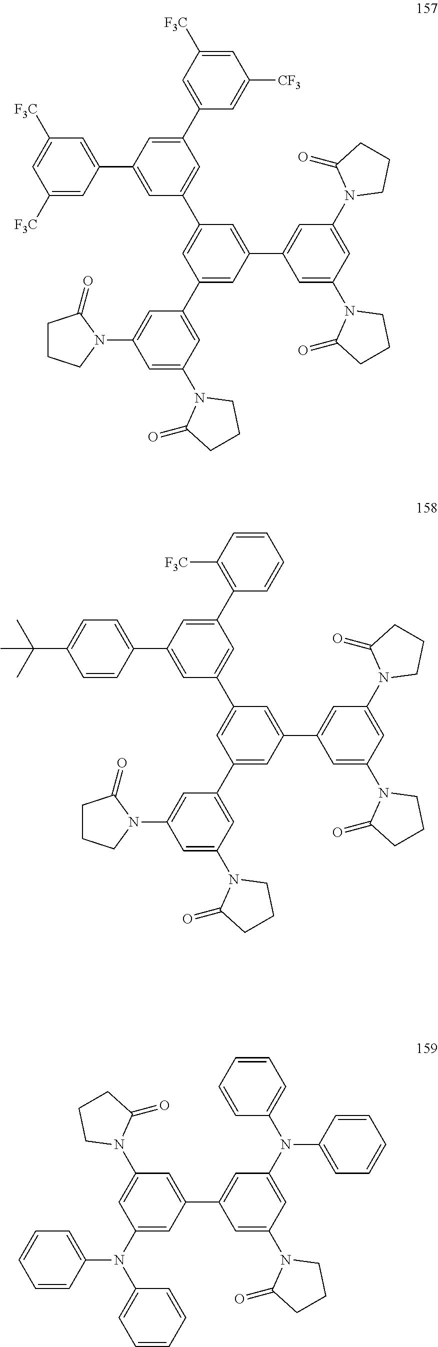 [SMILES: O=C1CCCN1c1cc(-c2cc(-c3cc(-c4cc(C(F)(F)F)cc(C(F)(F)F)c4)cc(-c4cc(C(F)(F)F)cc(C(F)(F)F)c4)c3)cc(-c3cc(N4CCCC4=O)cc(N4CCCC4=O)c3)c2)cc(N2CCCC2=O)c1 ; CC(C)(C)c1ccc(-c2cc(-c3cc(-c4cc(N5CCCC5=O)cc(N5CCCC5=O)c4)cc(-c4cc(N5CCCC5=O)cc(N5CCCC5=O)c4)c3)cc(-c3ccccc3C(F)(F)F)c2)cc1 ; O=C1CCCN1c1cc(-c2cc(N3CCCC3=O)cc(N(c3ccccc3)c3ccccc3)c2)cc(N(c2ccccc2)c2ccccc2)c1]

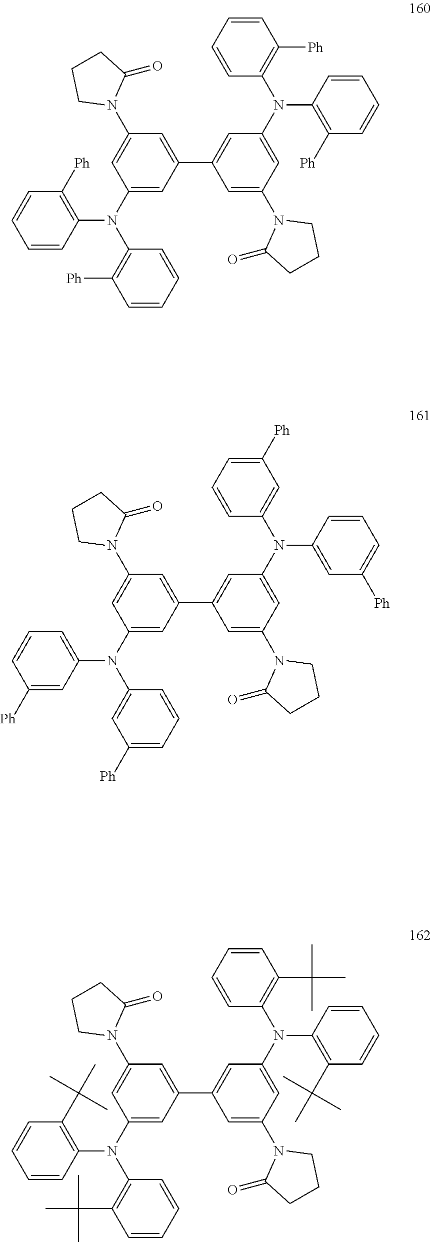 [SMILES: O=C1CCCN1c1cc(-c2cc(N3CCCC3=O)cc(N(c3ccccc3-c3ccccc3)c3ccccc3-c3ccccc3)c2)cc(N(c2ccccc2-c2ccccc2)c2ccccc2-c2ccccc2)c1 ; O=C1CCCN1c1cc(-c2cc(N3CCCC3=O)cc(N(c3cccc(-c4ccccc4)c3)c3cccc(-c4ccccc4)c3)c2)cc(N(c2cccc(-c3ccccc3)c2)c2cccc(-c3ccccc3)c2)c1 ; CC(C)(C)c1ccccc1N(c1cc(-c2cc(N3CCCC3=O)cc(N(c3ccccc3C(C)(C)C)c3ccccc3C(C)(C)C)c2)cc(N2CCCC2=O)c1)c1ccccc1C(C)(C)C]

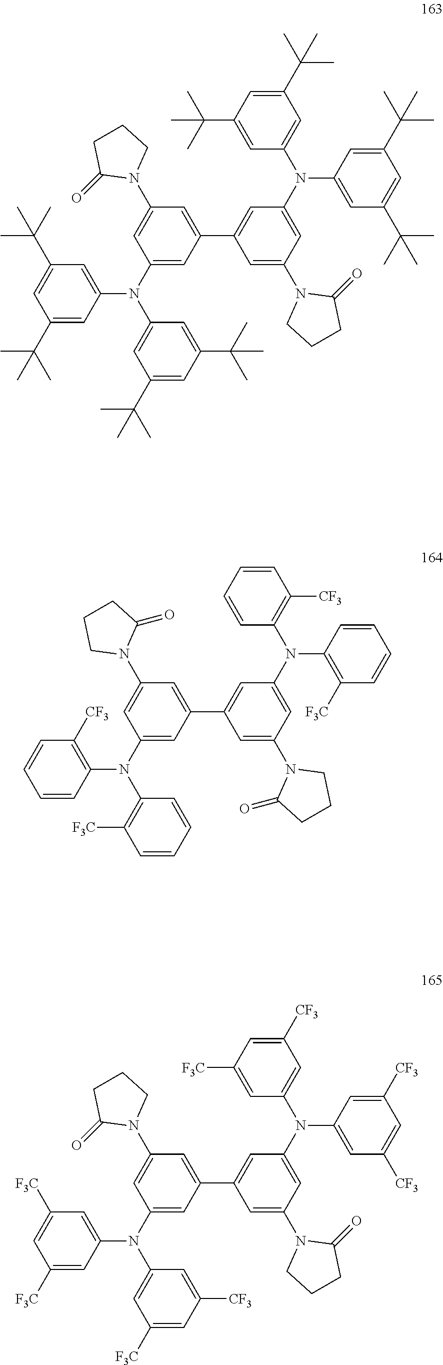 [SMILES: CC(C)(C)c1cc(N(c2cc(-c3cc(N4CCCC4=O)cc(N(c4cc(C(C)(C)C)cc(C(C)(C)C)c4)c4cc(C(C)(C)C)cc(C(C)(C)C)c4)c3)cc(N3CCCC3=O)c2)c2cc(C(C)(C)C)cc(C(C)(C)C)c2)cc(C(C)(C)C)c1 ; O=C1CCCN1c1cc(-c2cc(N3CCCC3=O)cc(N(c3ccccc3C(F)(F)F)c3ccccc3C(F)(F)F)c2)cc(N(c2ccccc2C(F)(F)F)c2ccccc2C(F)(F)F)c1 ; O=C1CCCN1c1cc(-c2cc(N3CCCC3=O)cc(N(c3cc(C(F)(F)F)cc(C(F)(F)F)c3)c3cc(C(F)(F)F)cc(C(F)(F)F)c3)c2)cc(N(c2cc(C(F)(F)F)cc(C(F)(F)F)c2)c2cc(C(F)(F)F)cc(C(F)(F)F)c2)c1]

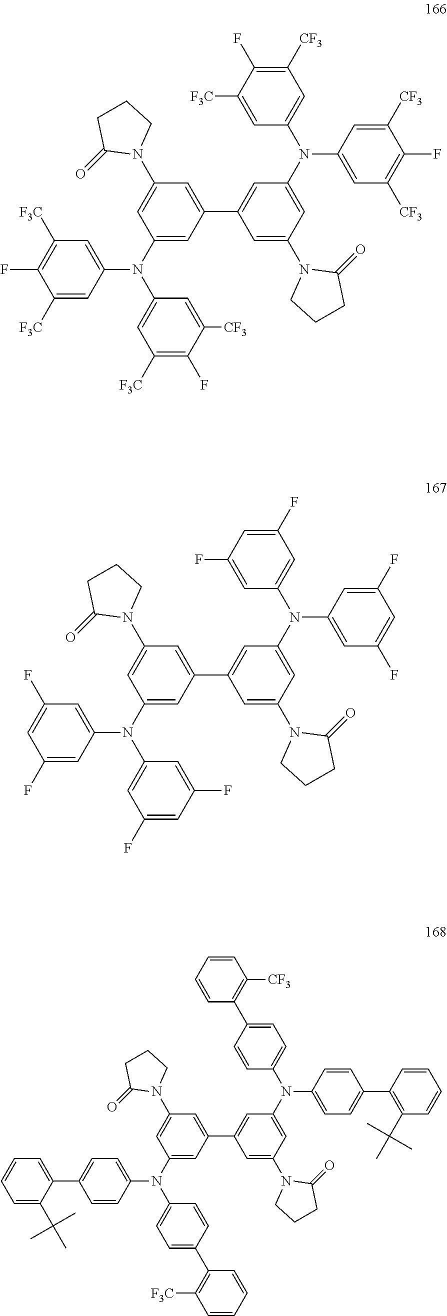 [SMILES: O=C1CCCN1c1cc(-c2cc(N3CCCC3=O)cc(N(c3cc(C(F)(F)F)c(F)c(C(F)(F)F)c3)c3cc(C(F)(F)F)c(F)c(C(F)(F)F)c3)c2)cc(N(c2cc(C(F)(F)F)c(F)c(C(F)(F)F)c2)c2cc(C(F)(F)F)c(F)c(C(F)(F)F)c2)c1 ; O=C1CCCN1c1cc(-c2cc(N3CCCC3=O)cc(N(c3cc(F)cc(F)c3)c3cc(F)cc(F)c3)c2)cc(N(c2cc(F)cc(F)c2)c2cc(F)cc(F)c2)c1 ; CC(C)(C)c1ccccc1-c1ccc(N(c2ccc(-c3ccccc3C(F)(F)F)cc2)c2cc(-c3cc(N4CCCC4=O)cc(N(c4ccc(-c5ccccc5C(C)(C)C)cc4)c4ccc(-c5ccccc5C(F)(F)F)cc4)c3)cc(N3CCCC3=O)c2)cc1]

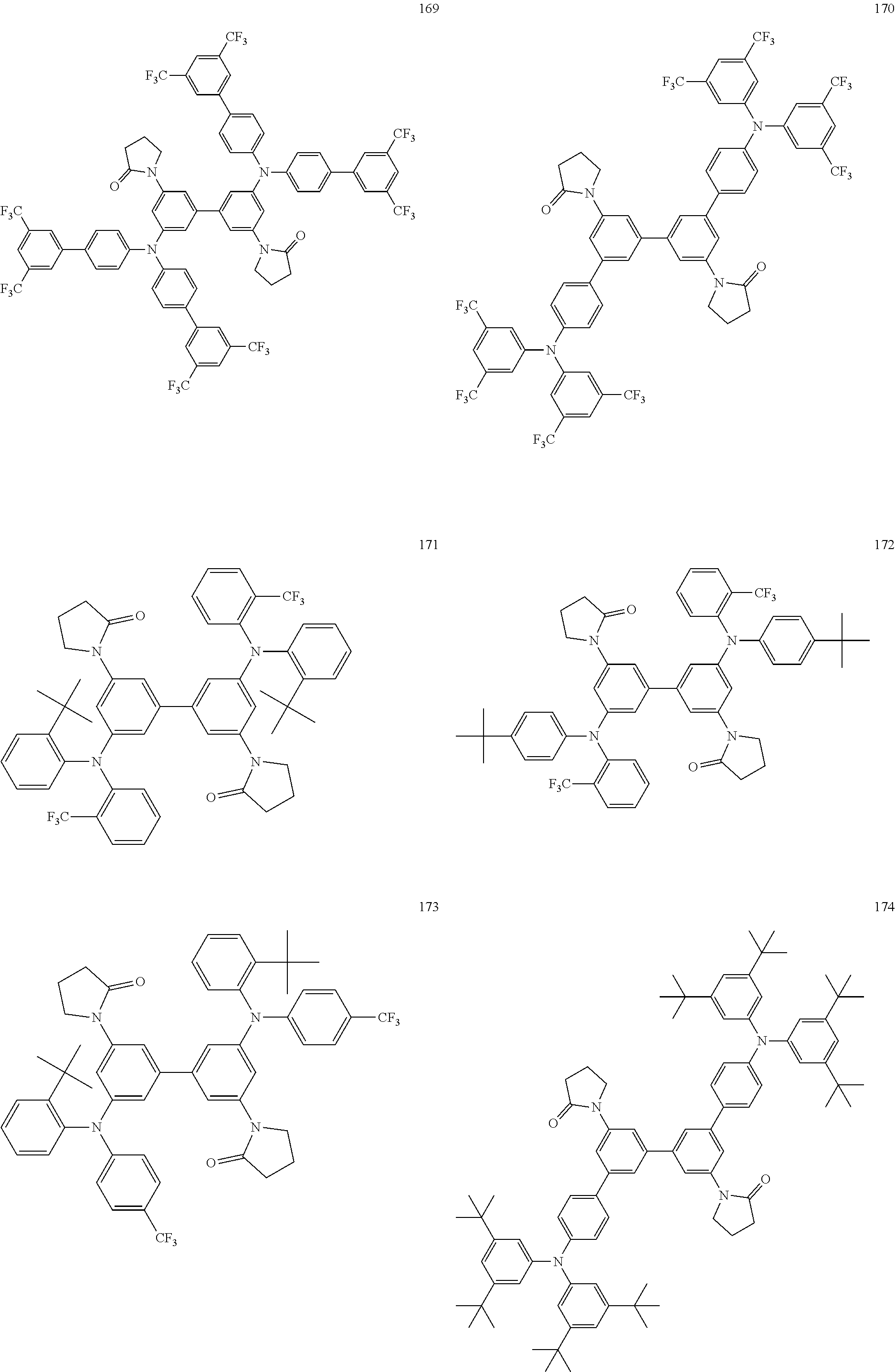

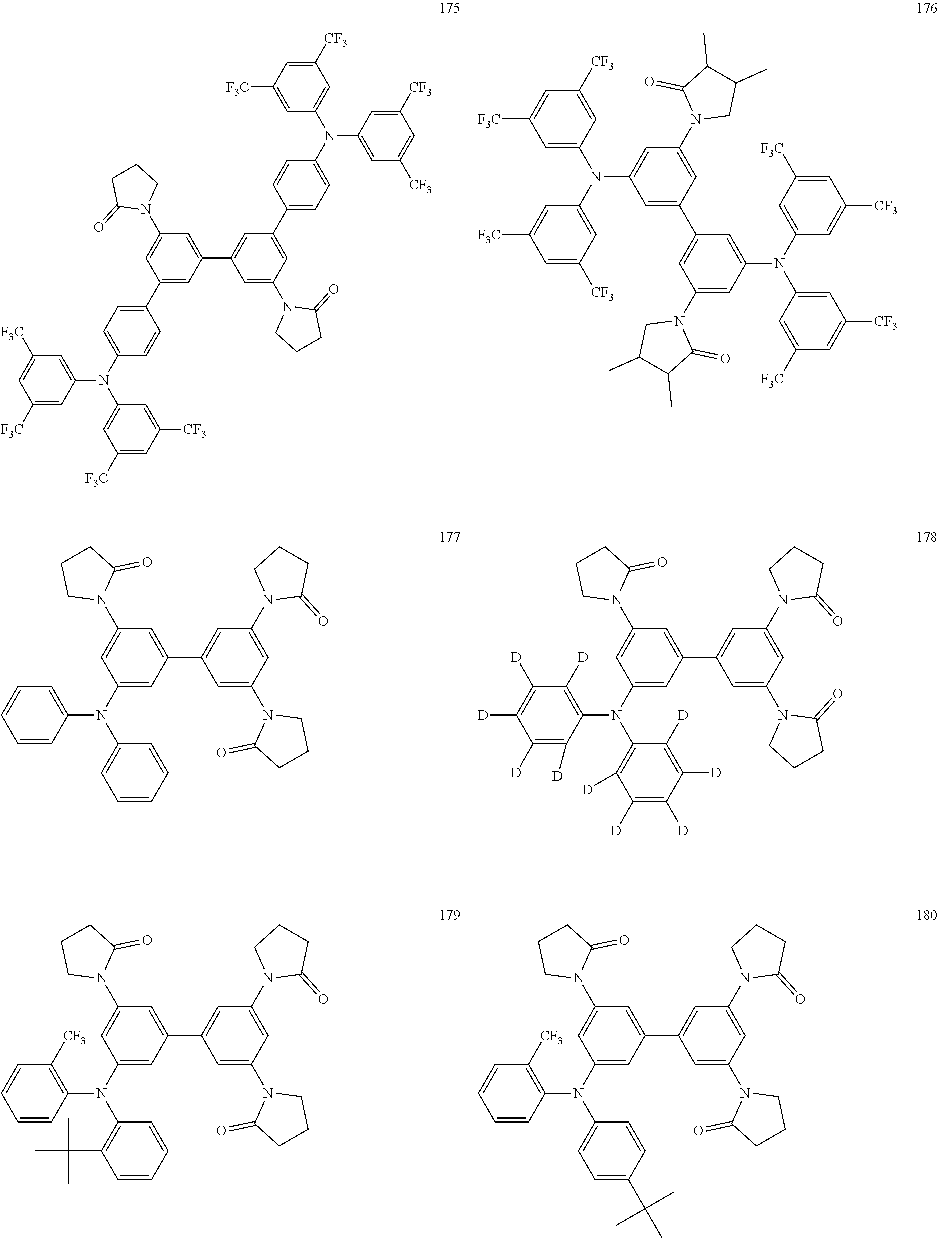

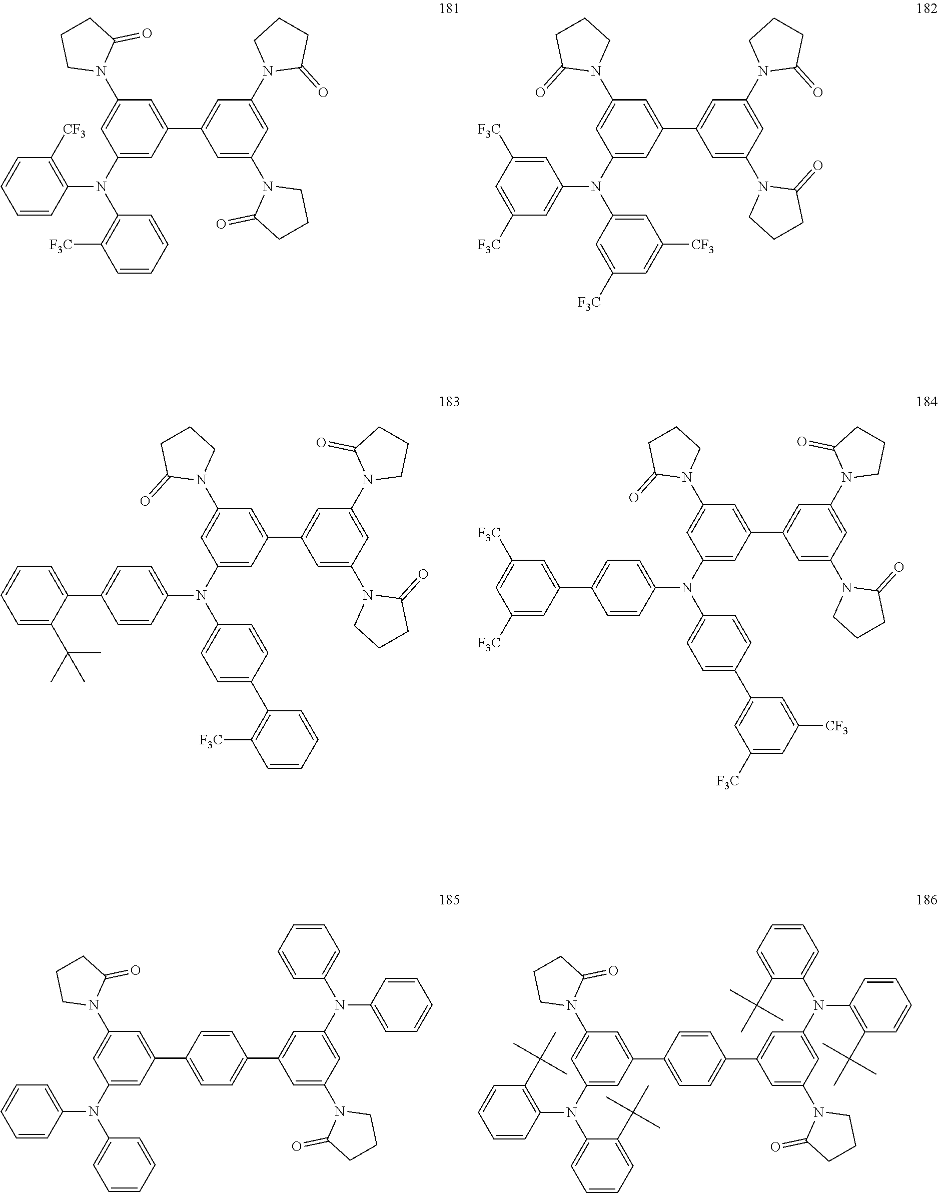

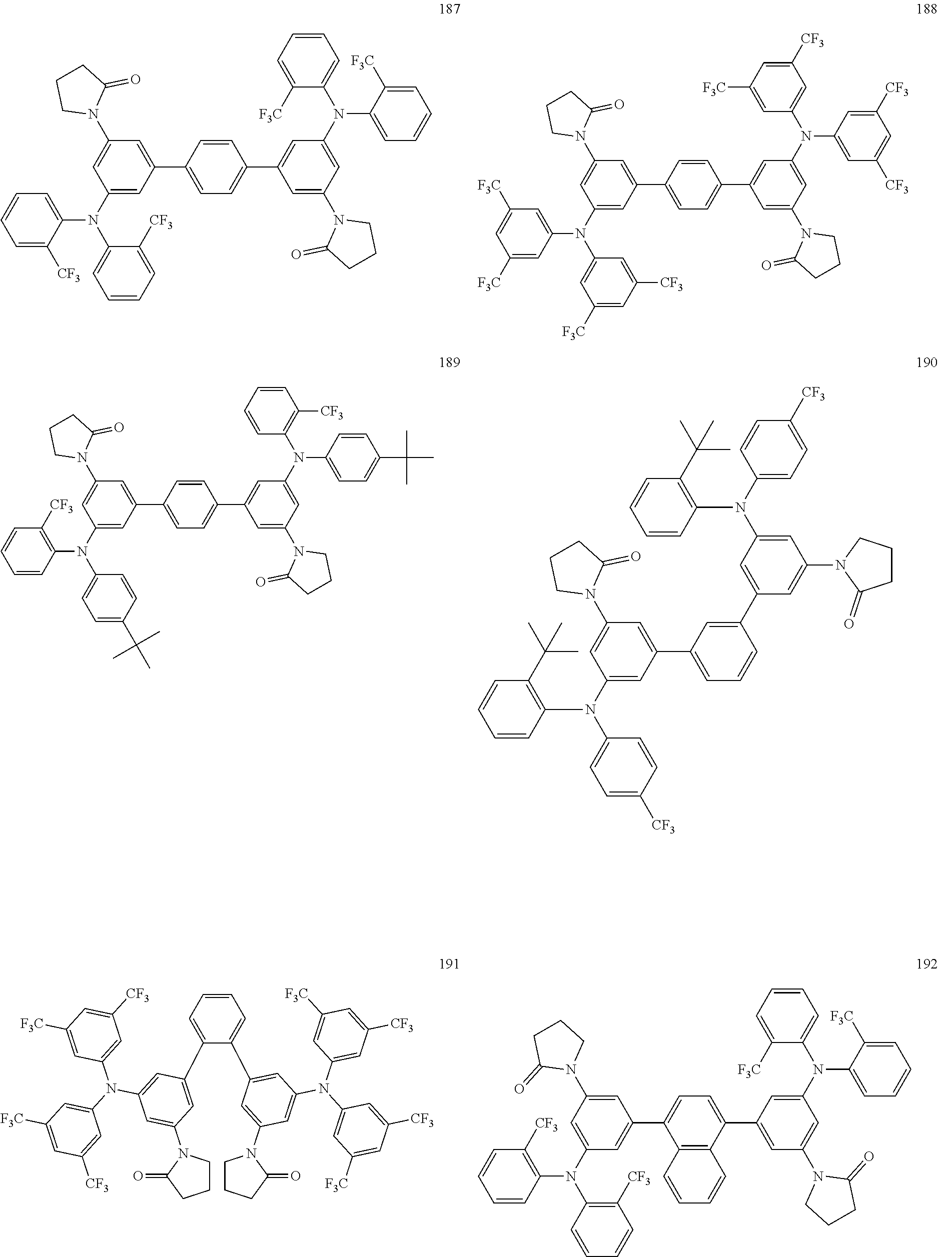

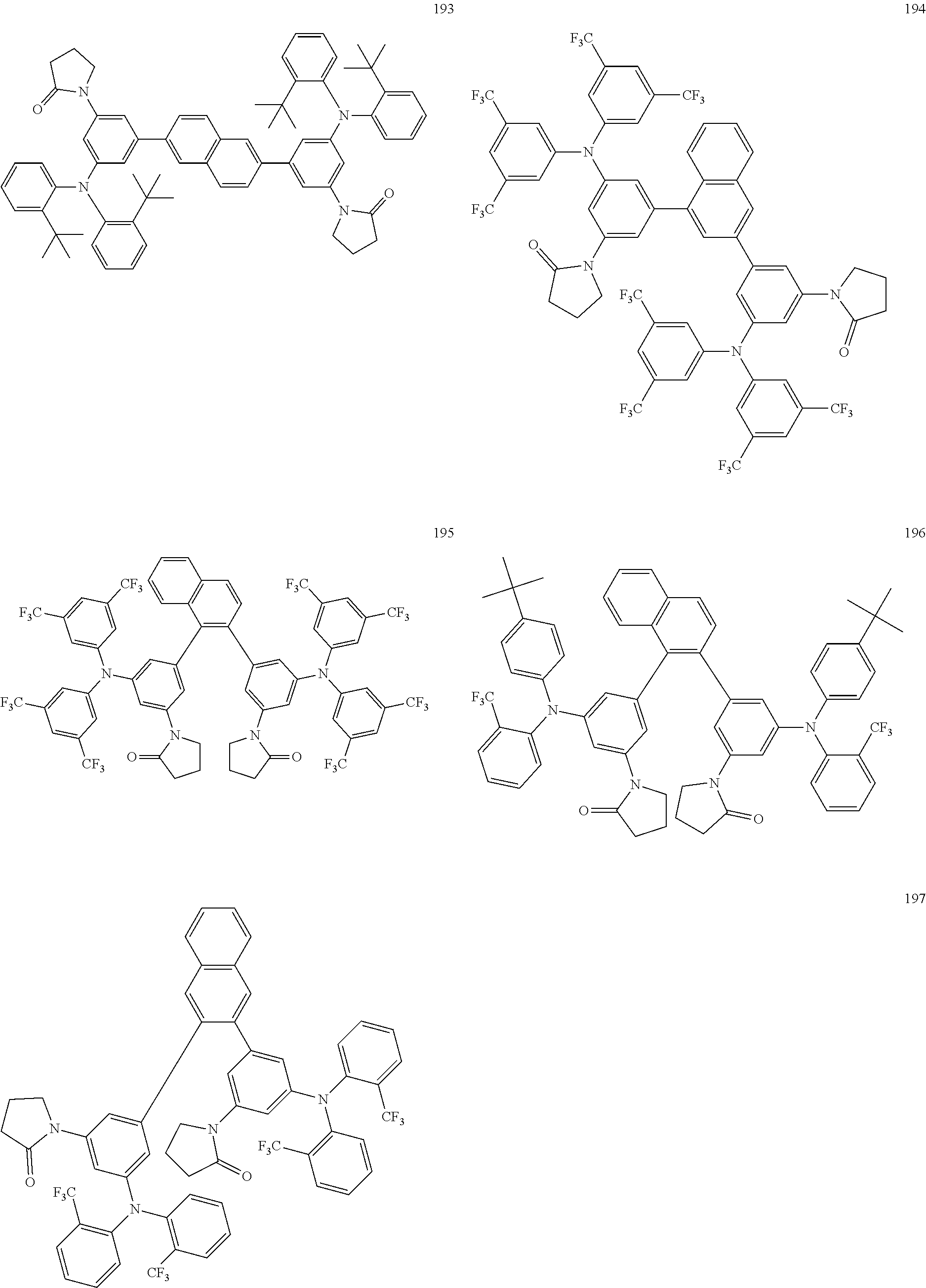

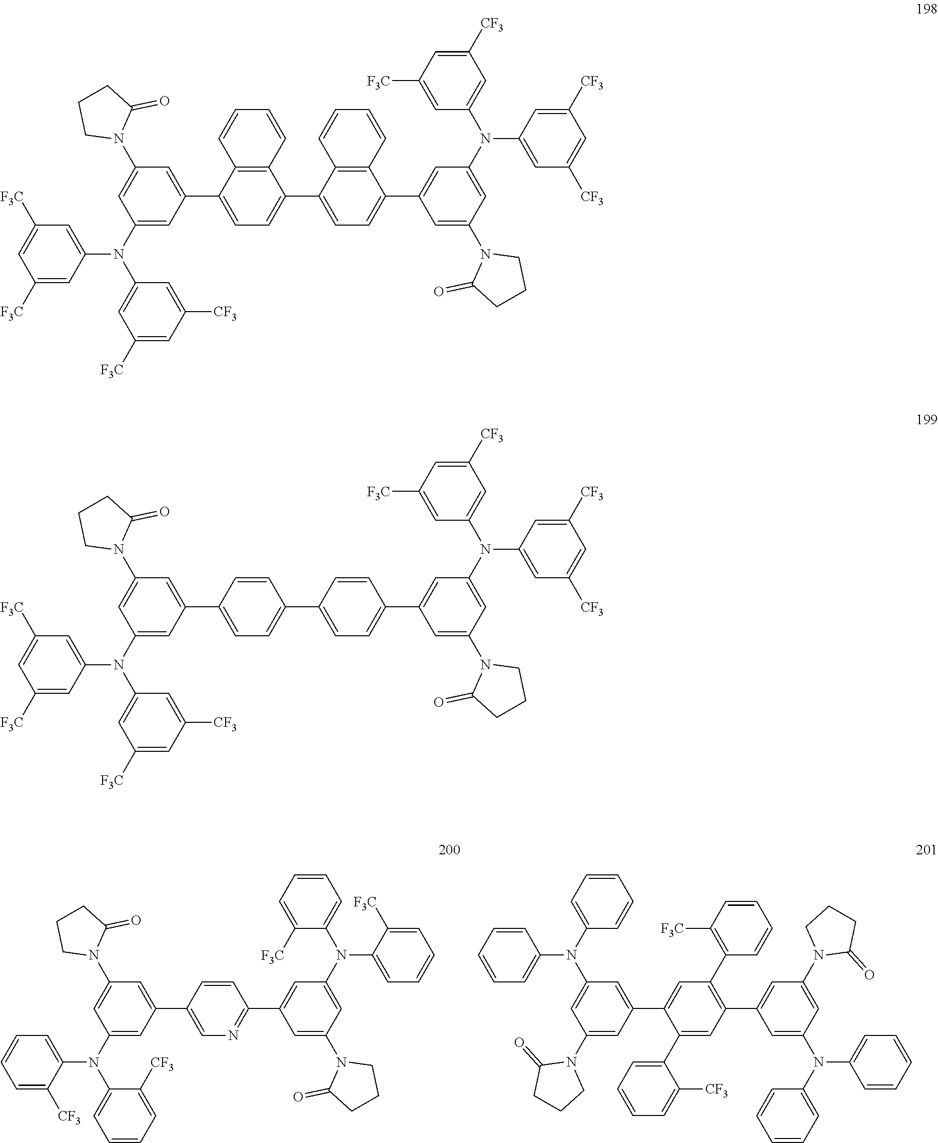

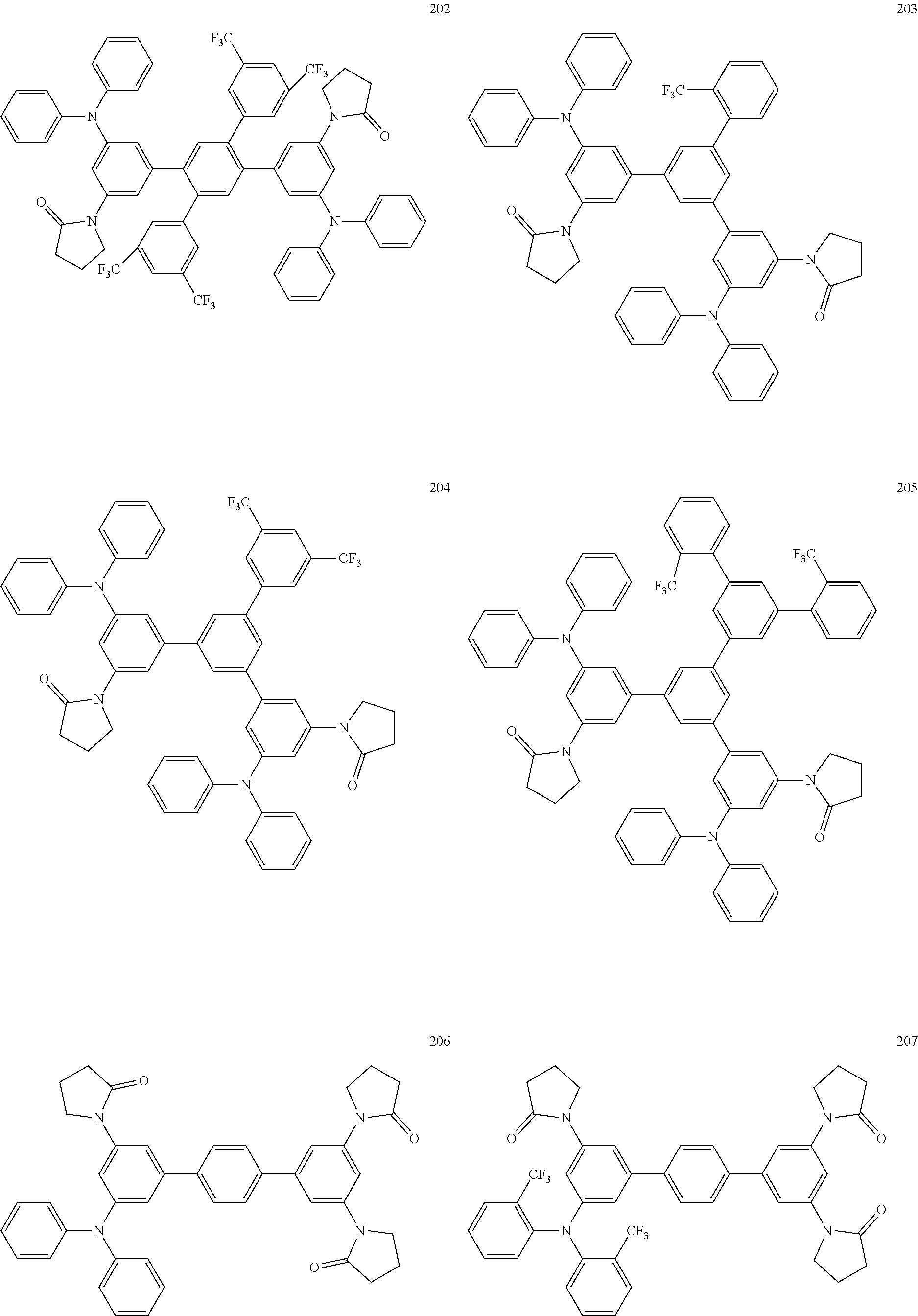

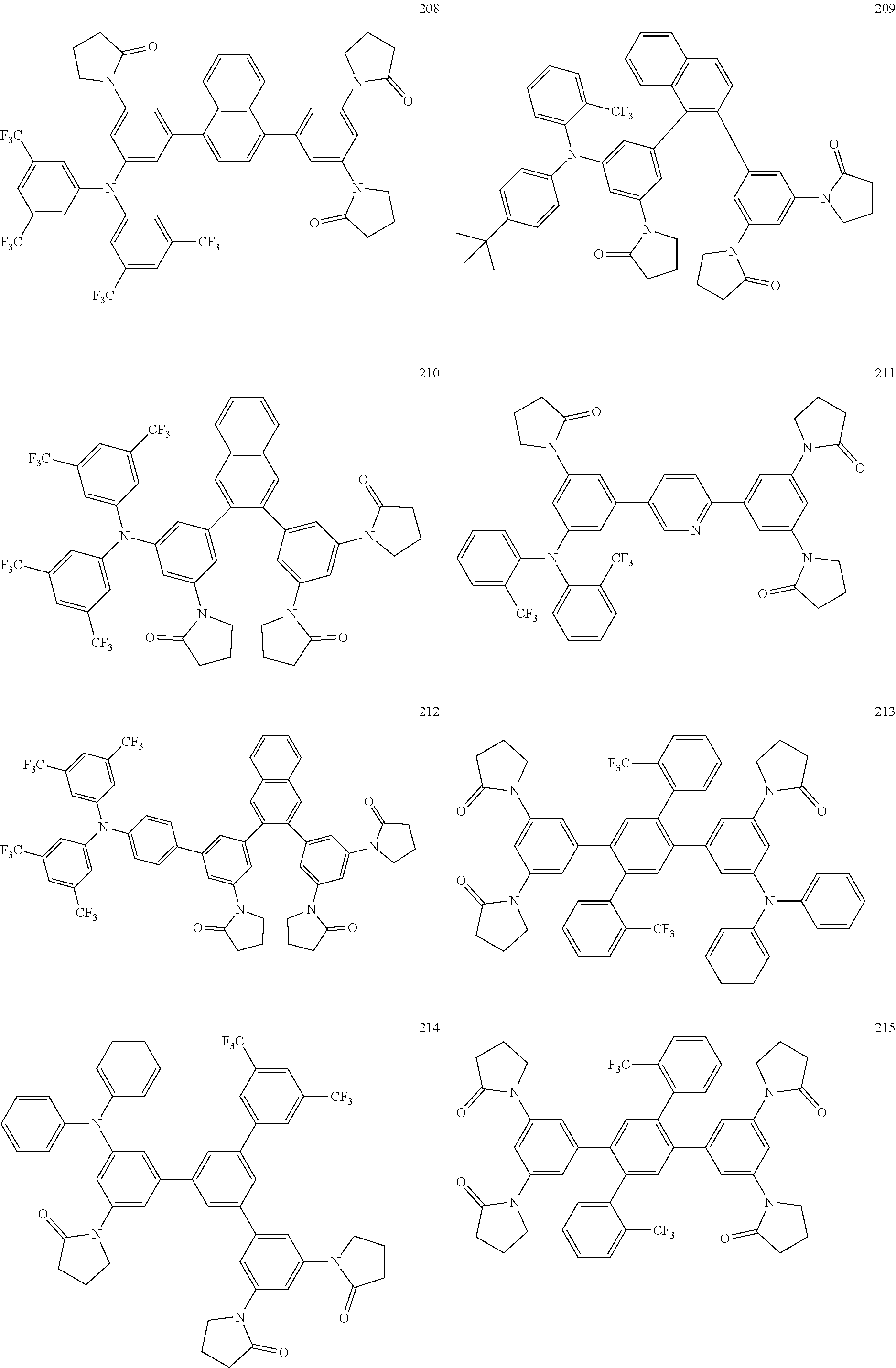

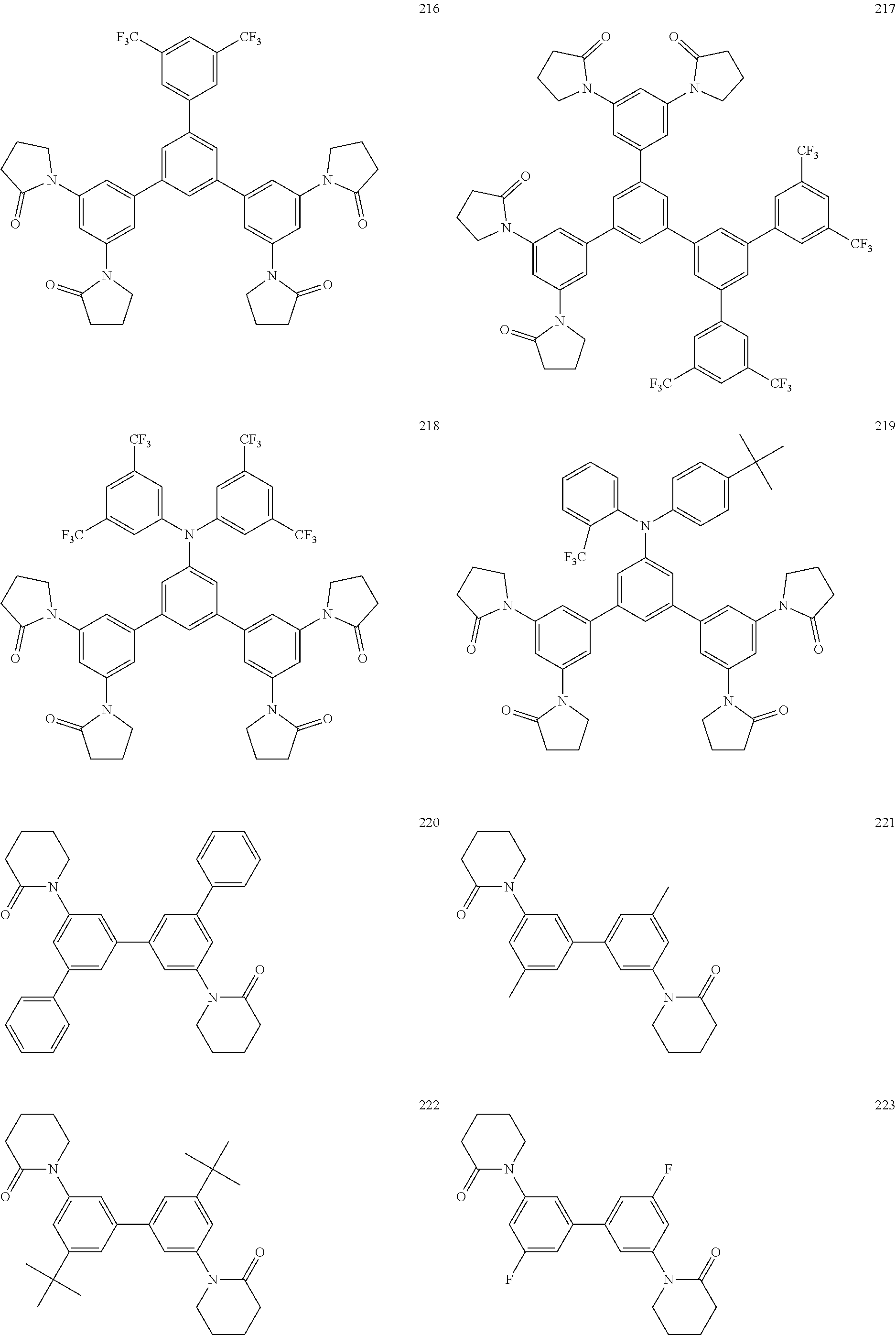

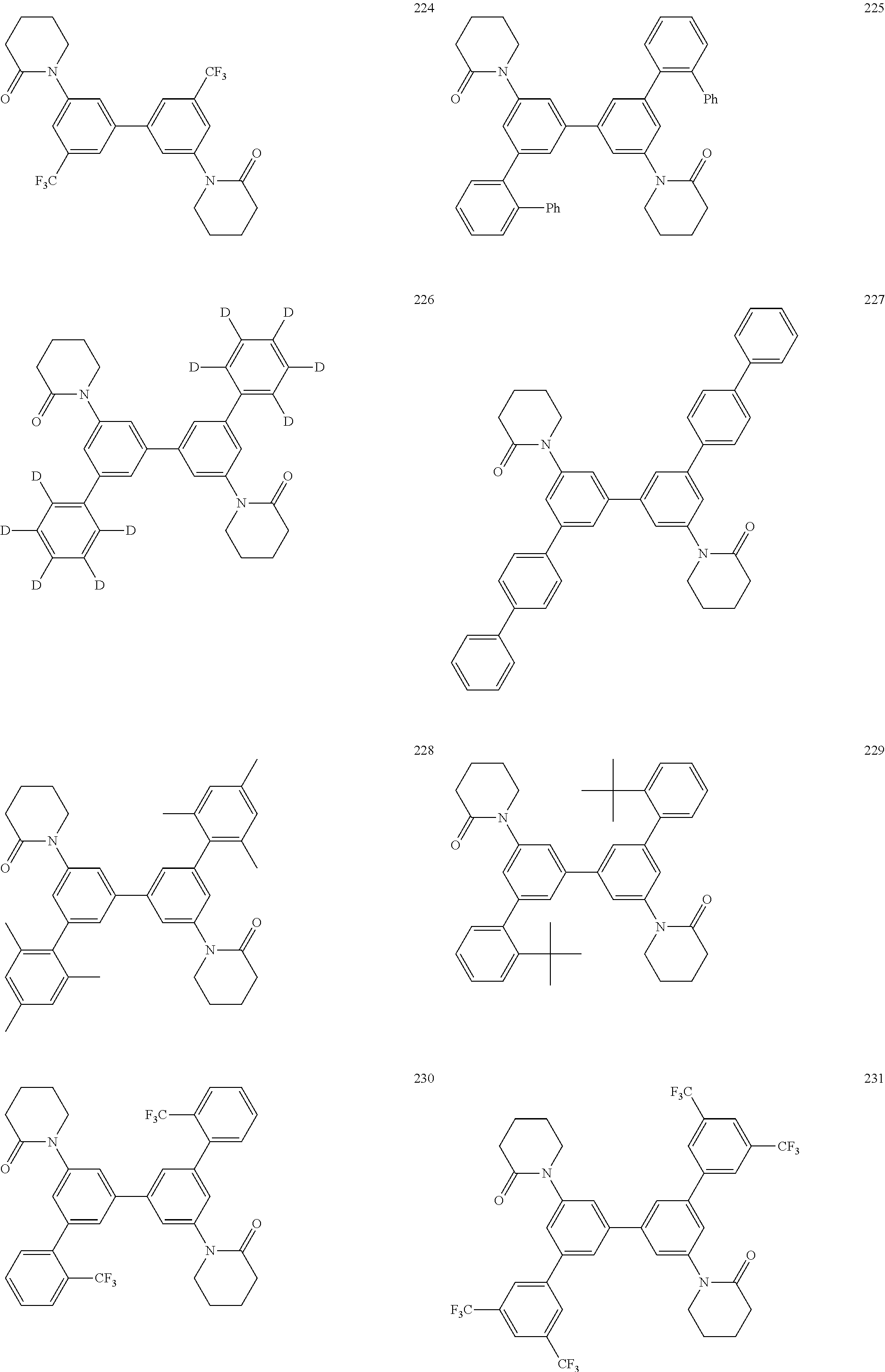

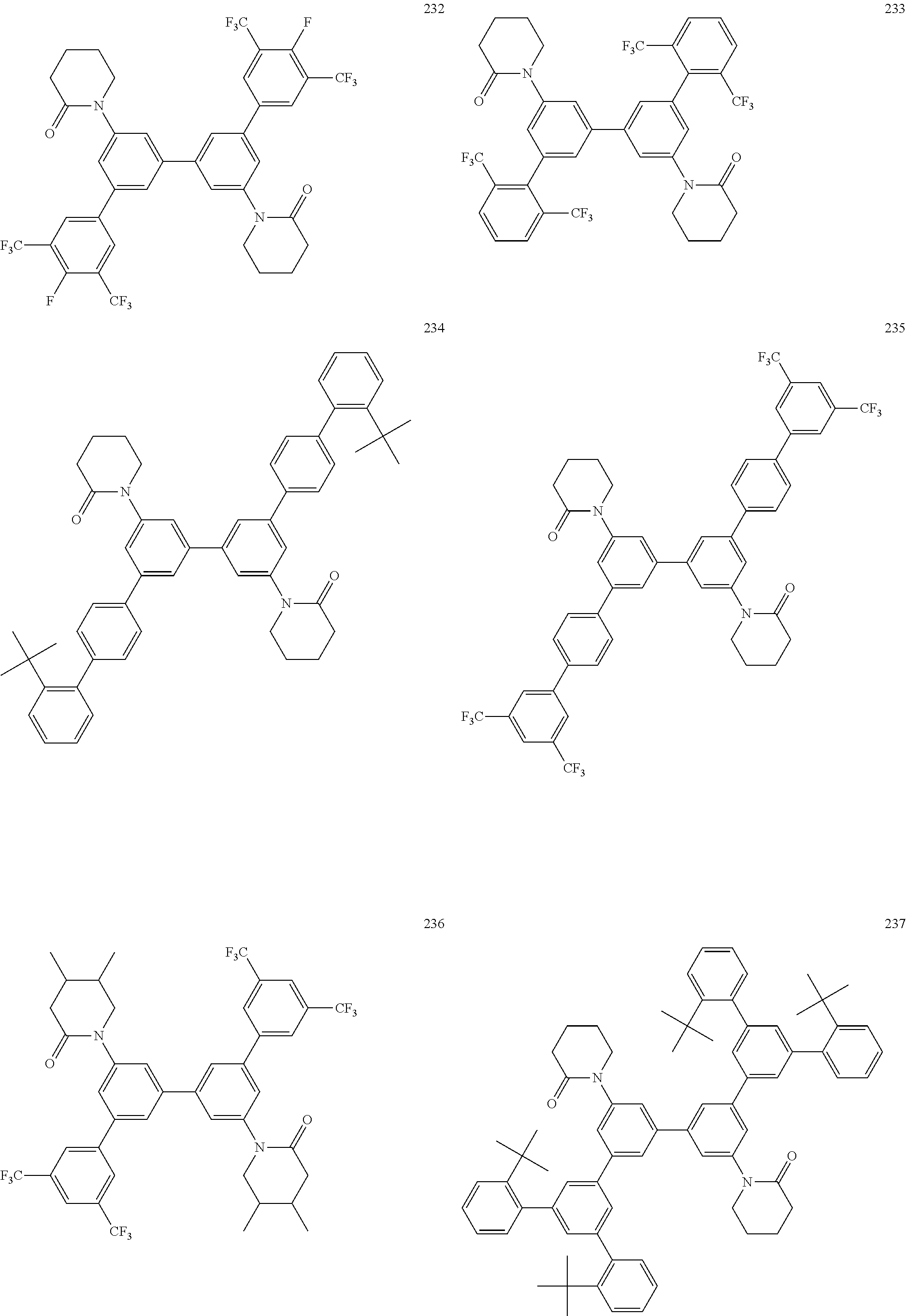

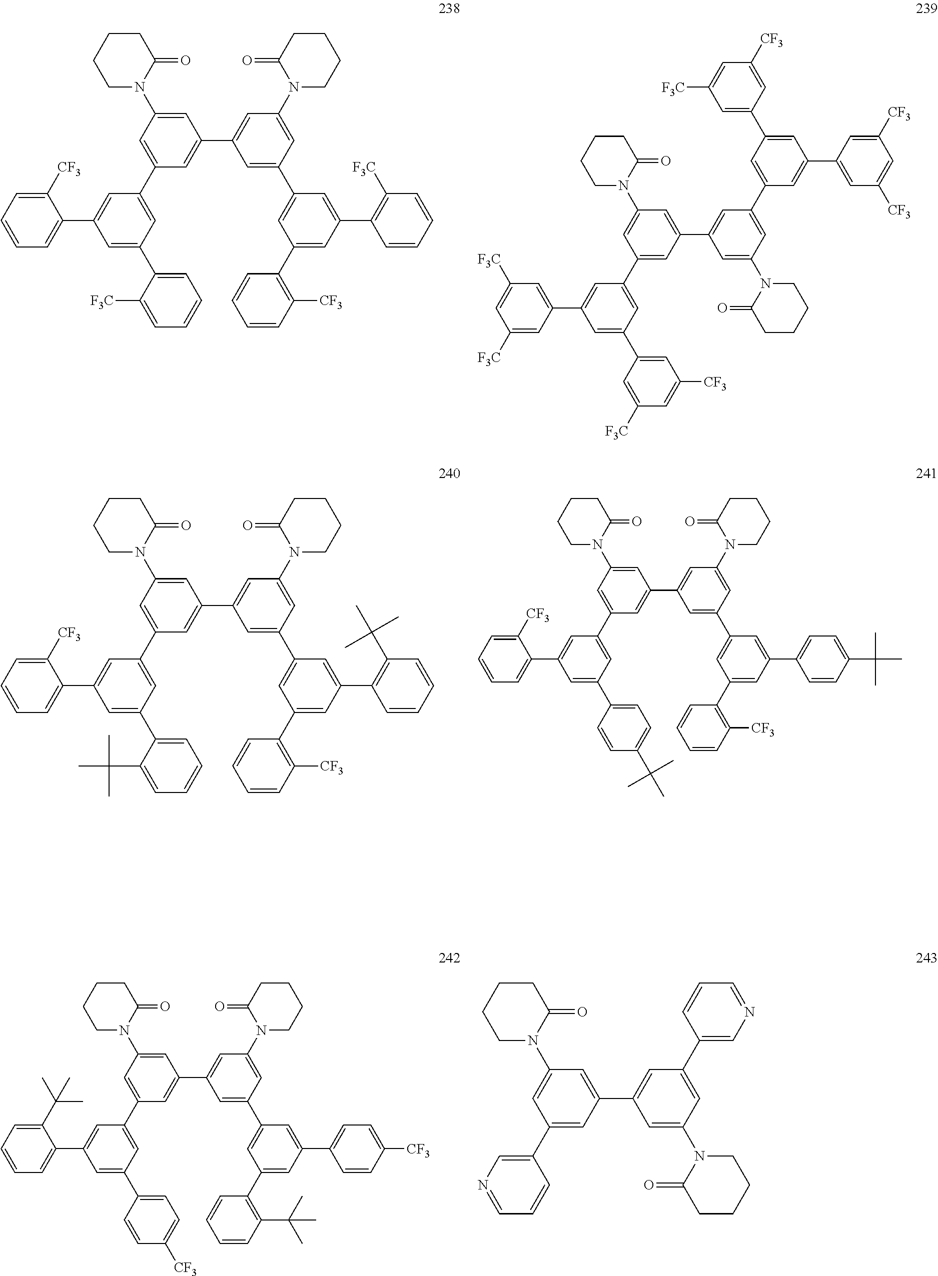

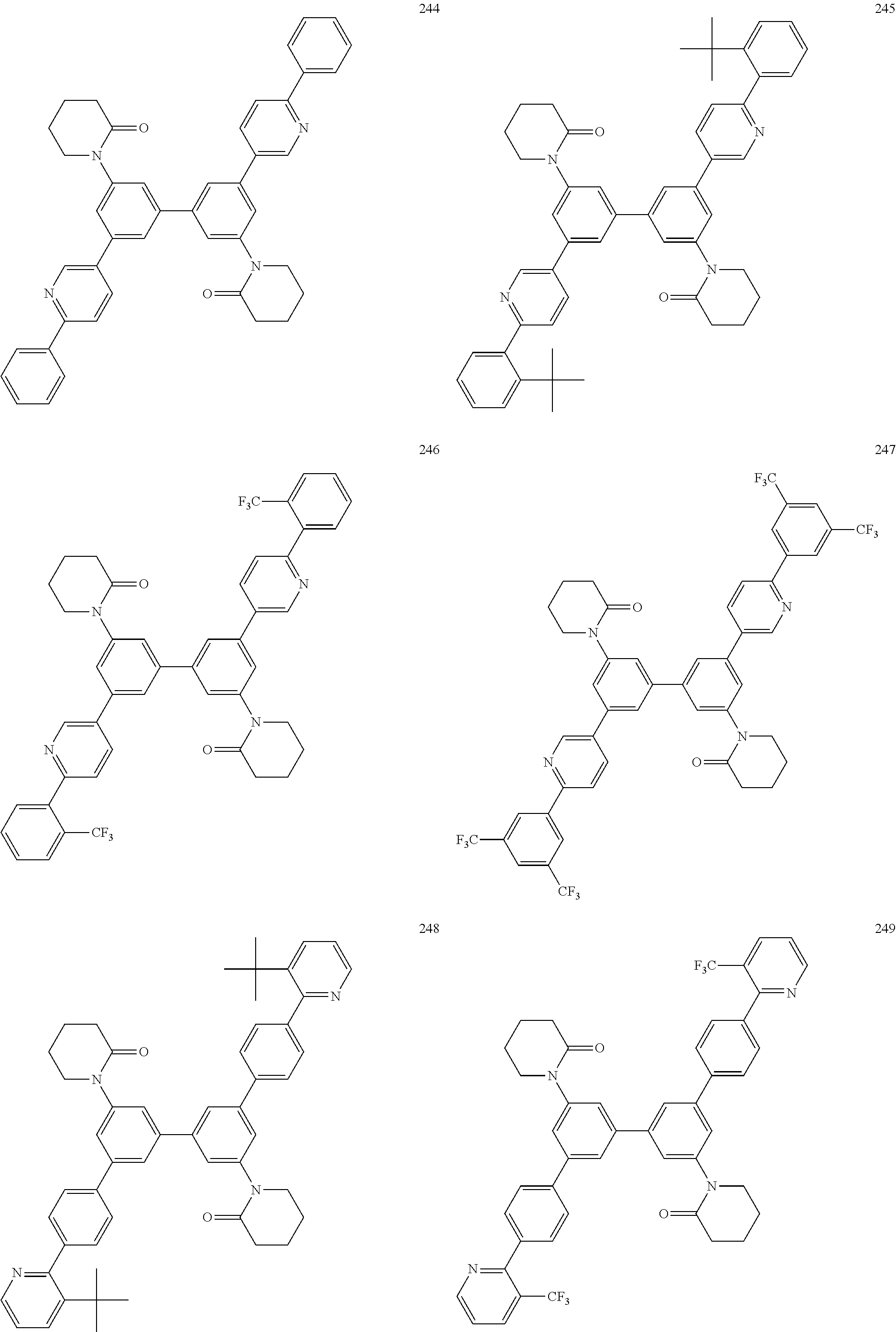

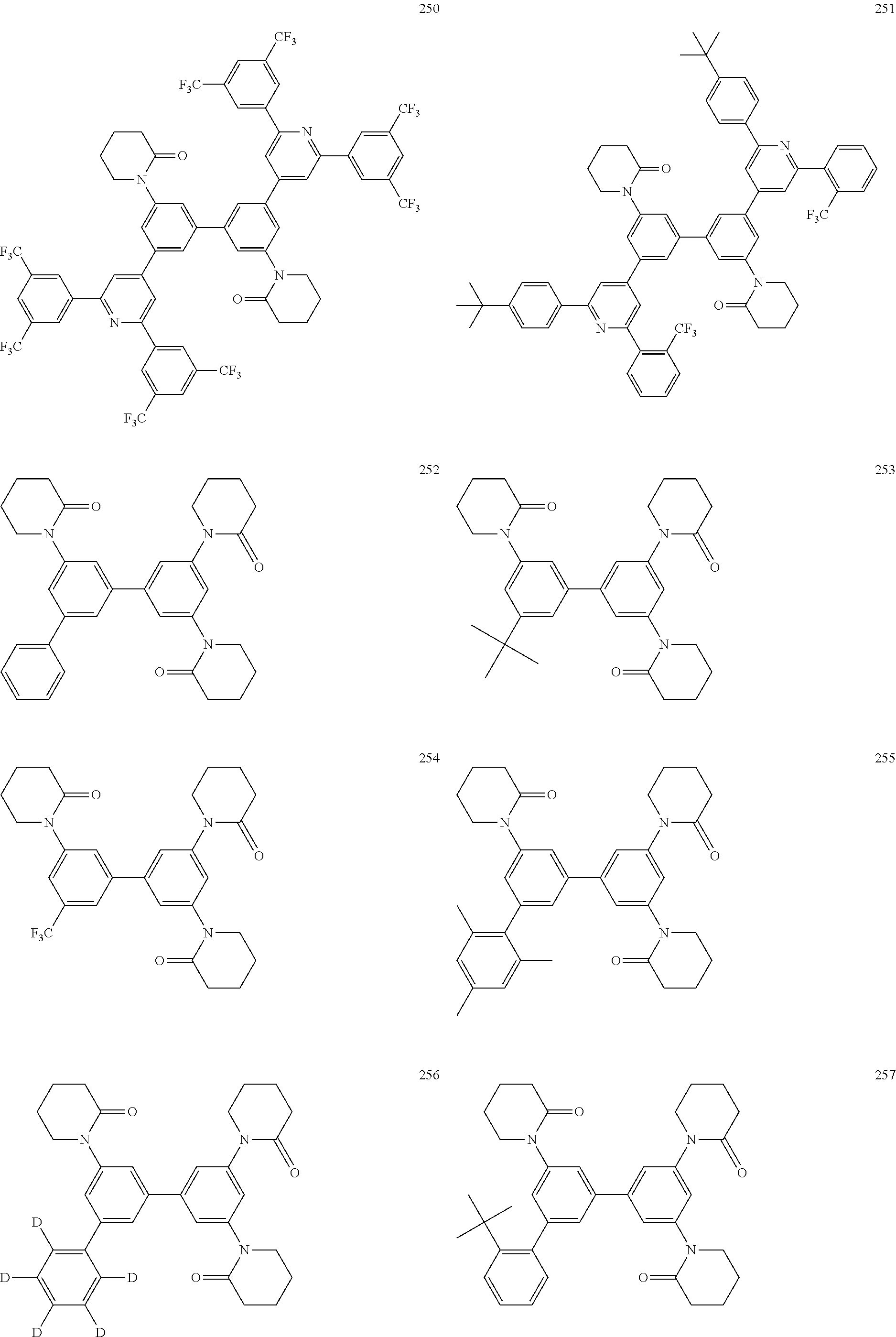

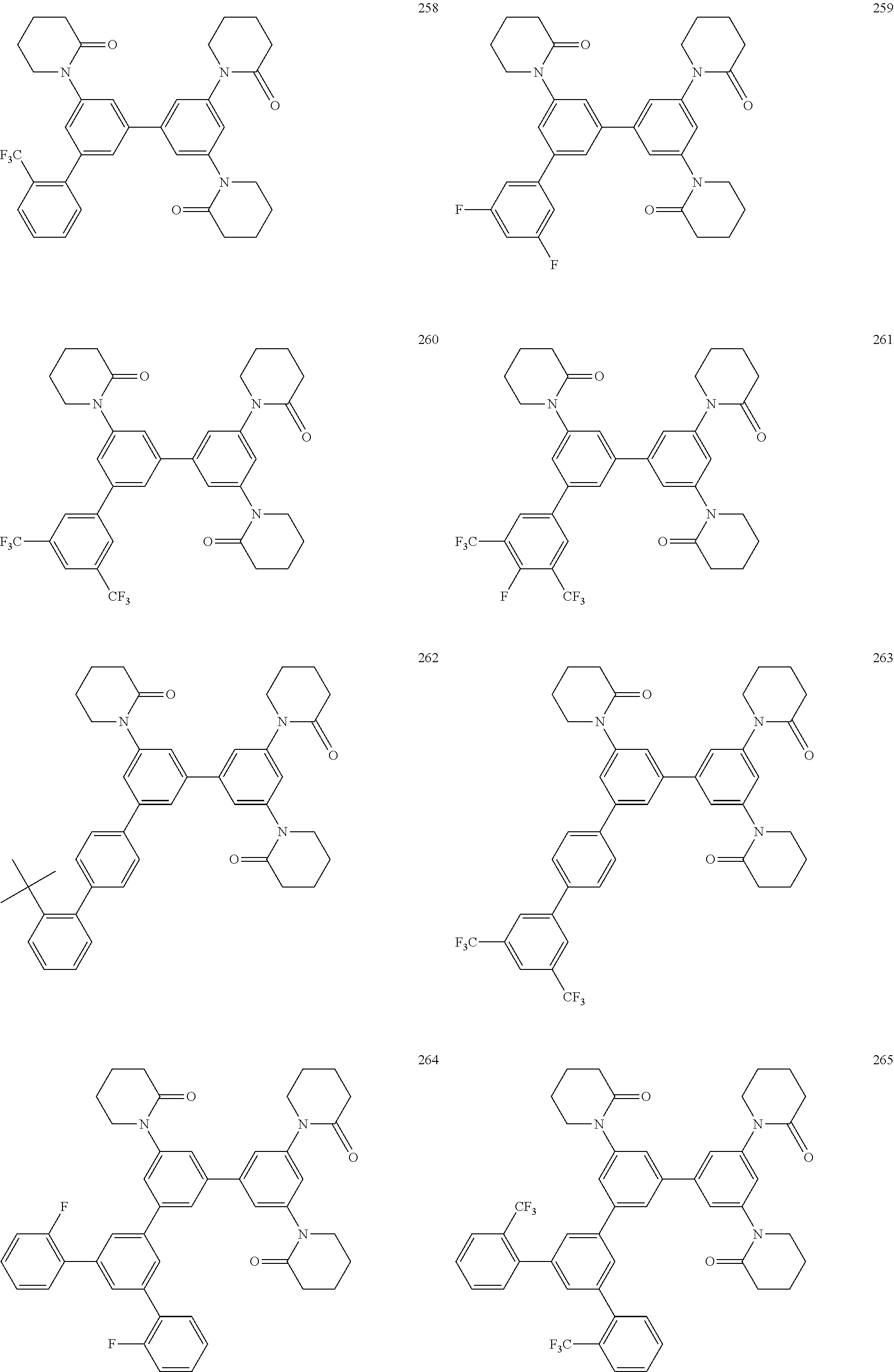

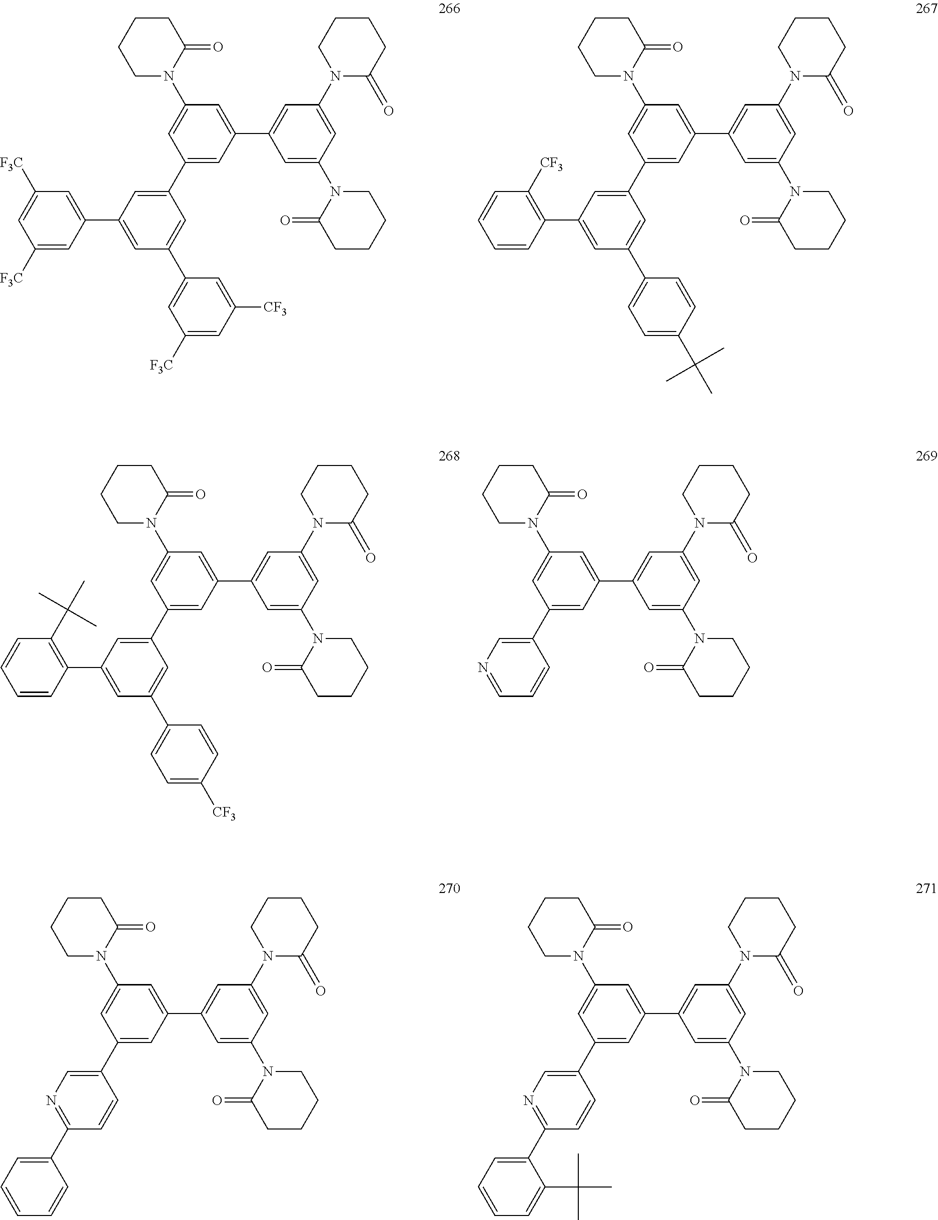

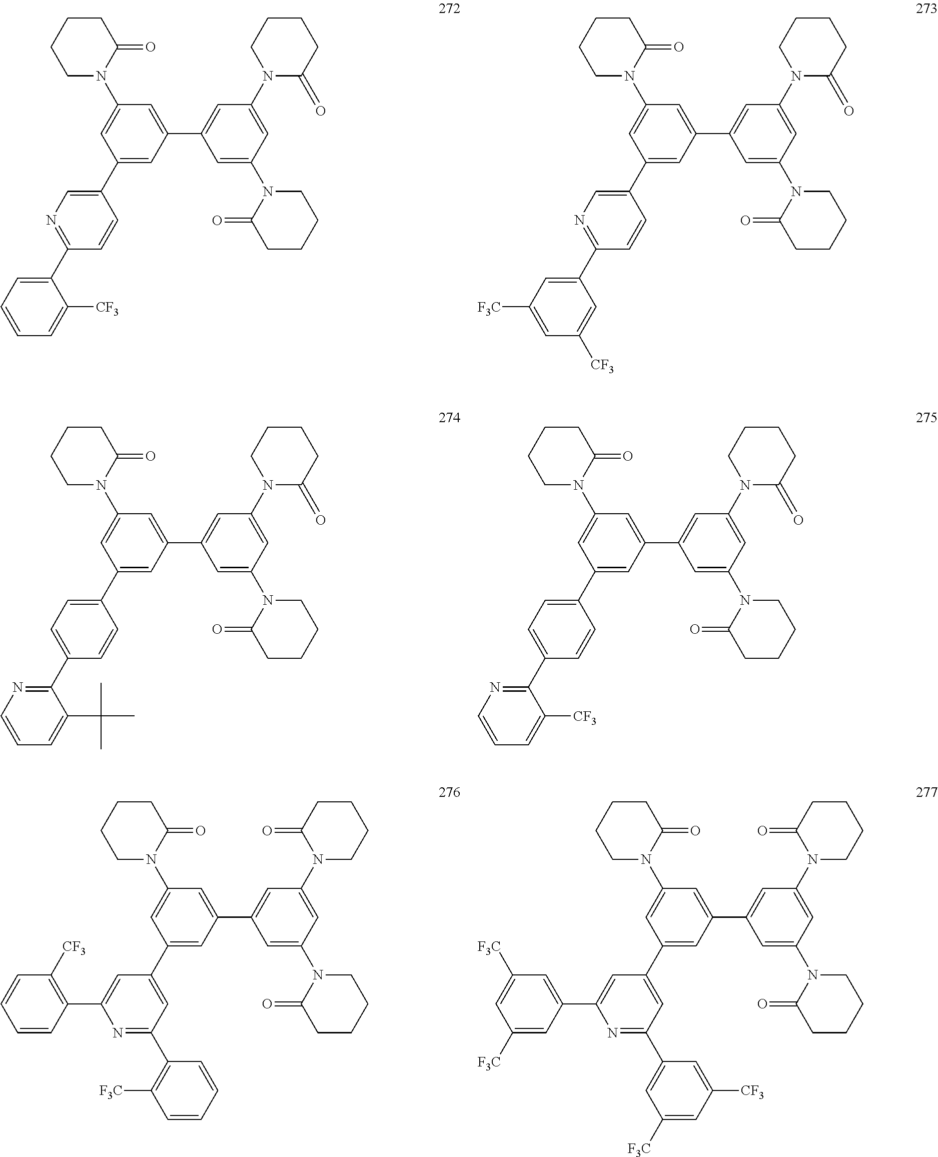

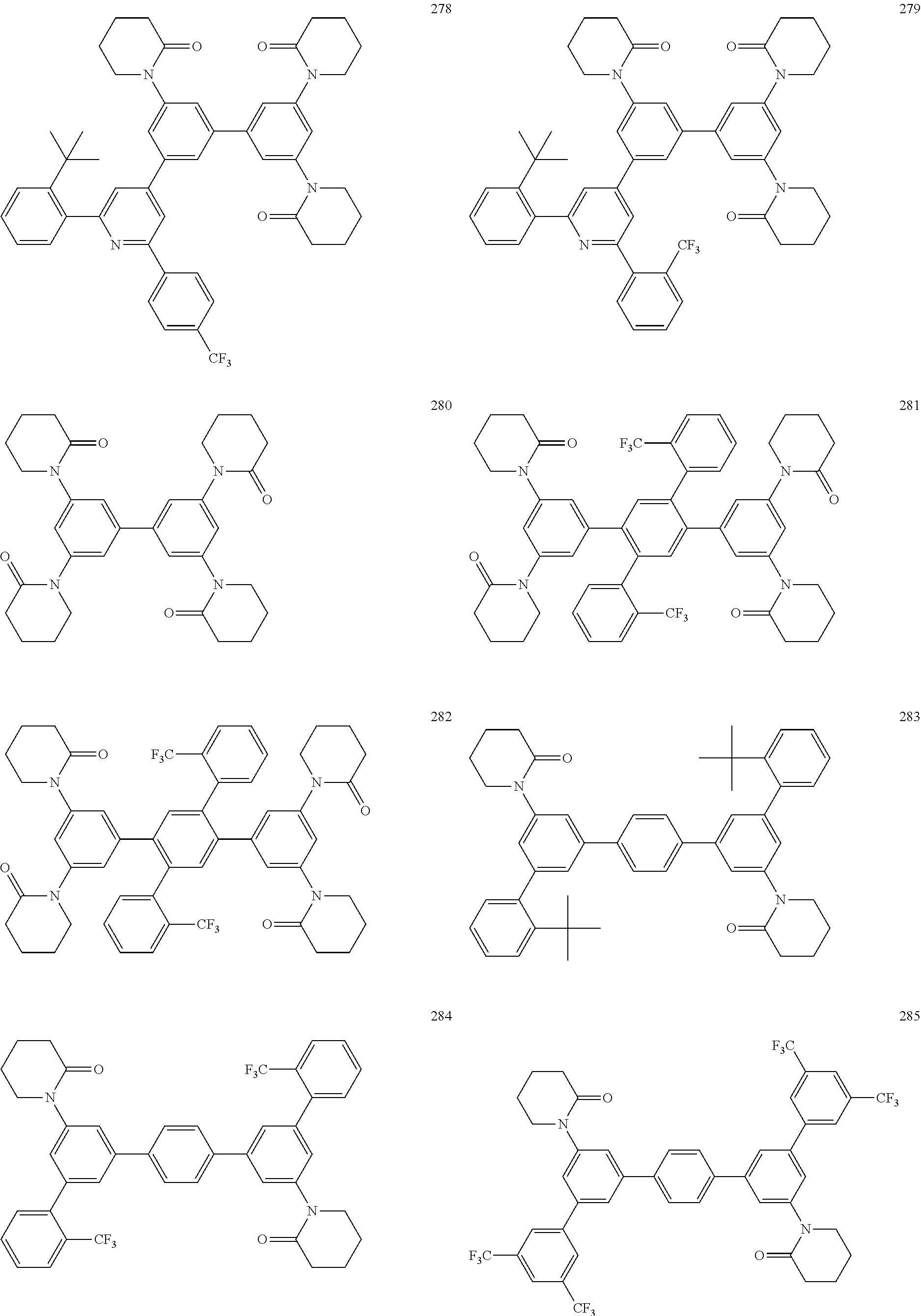

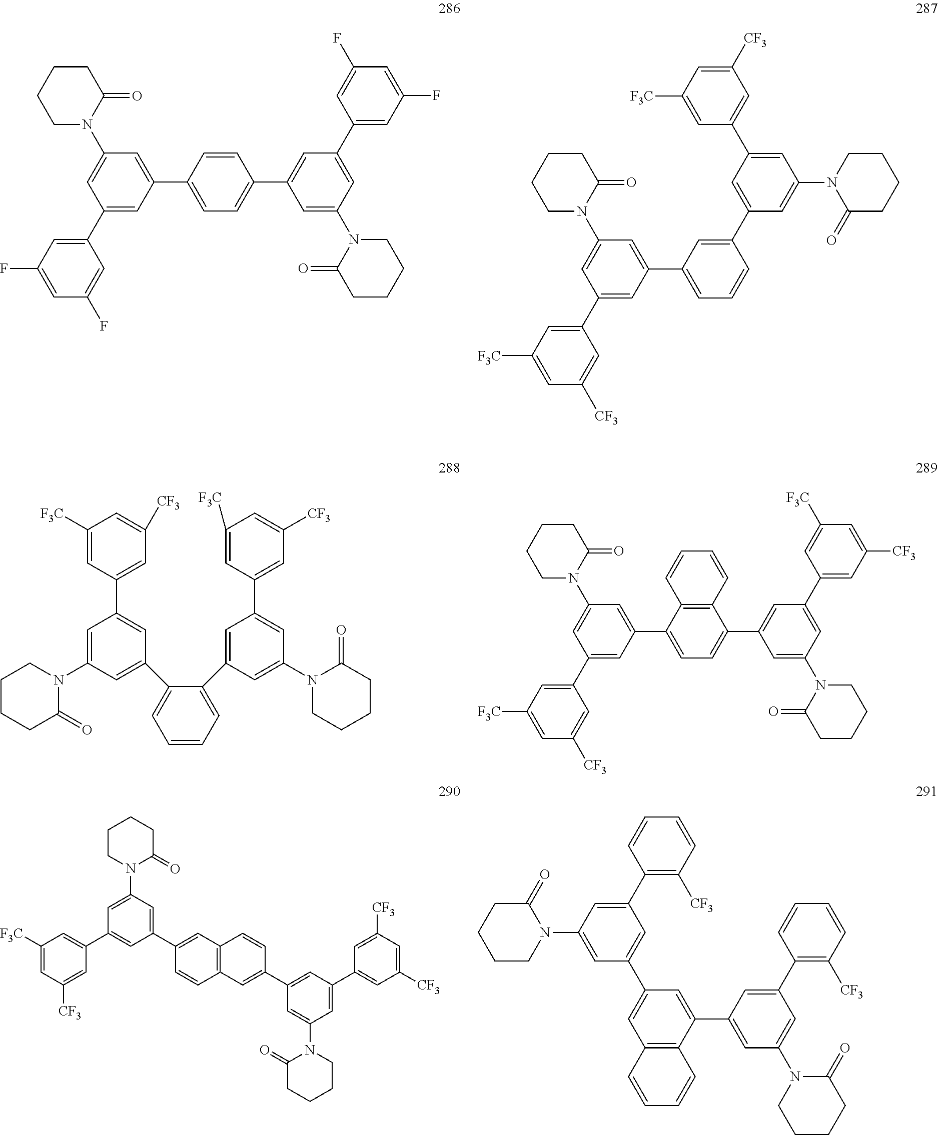

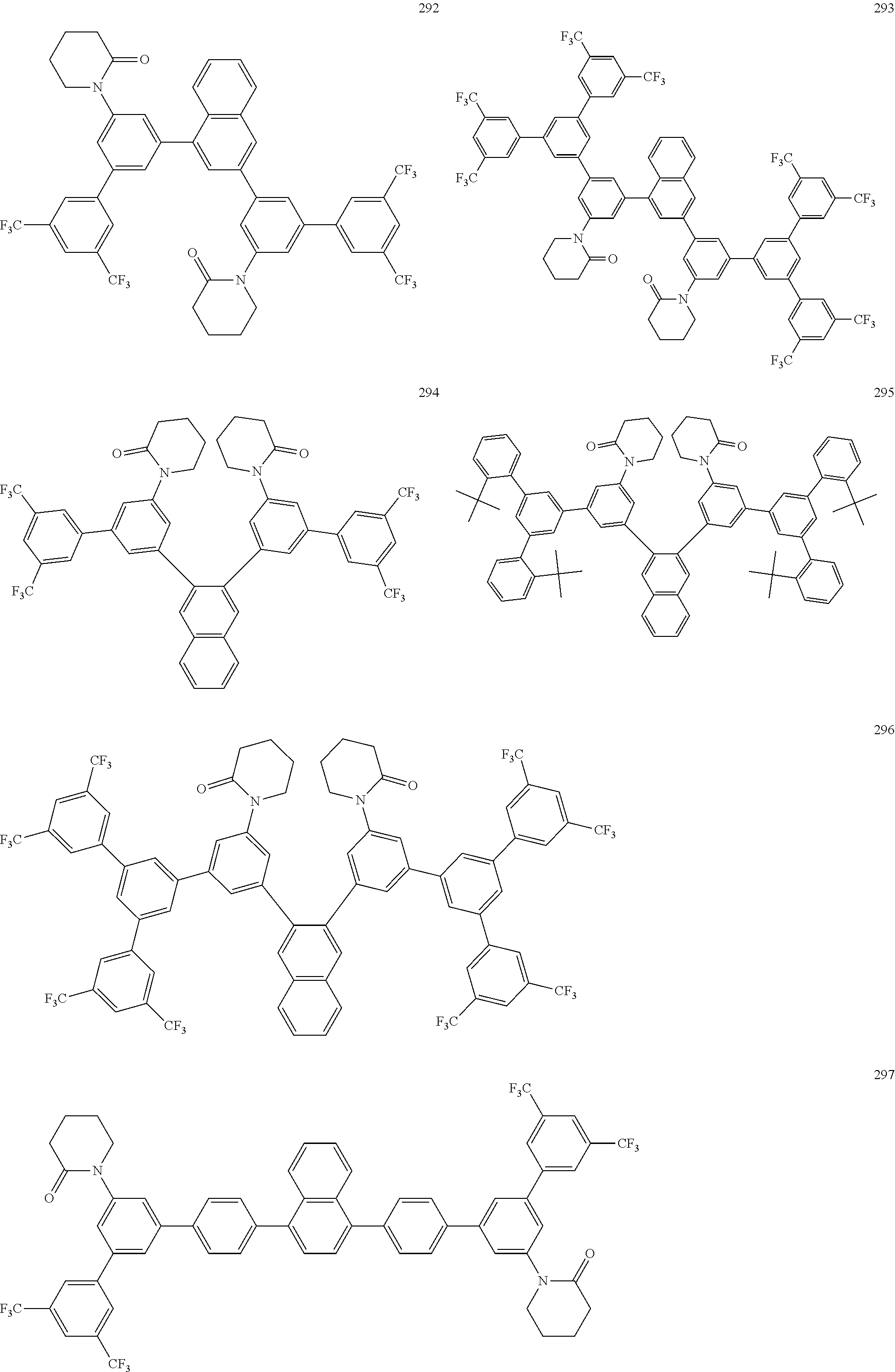

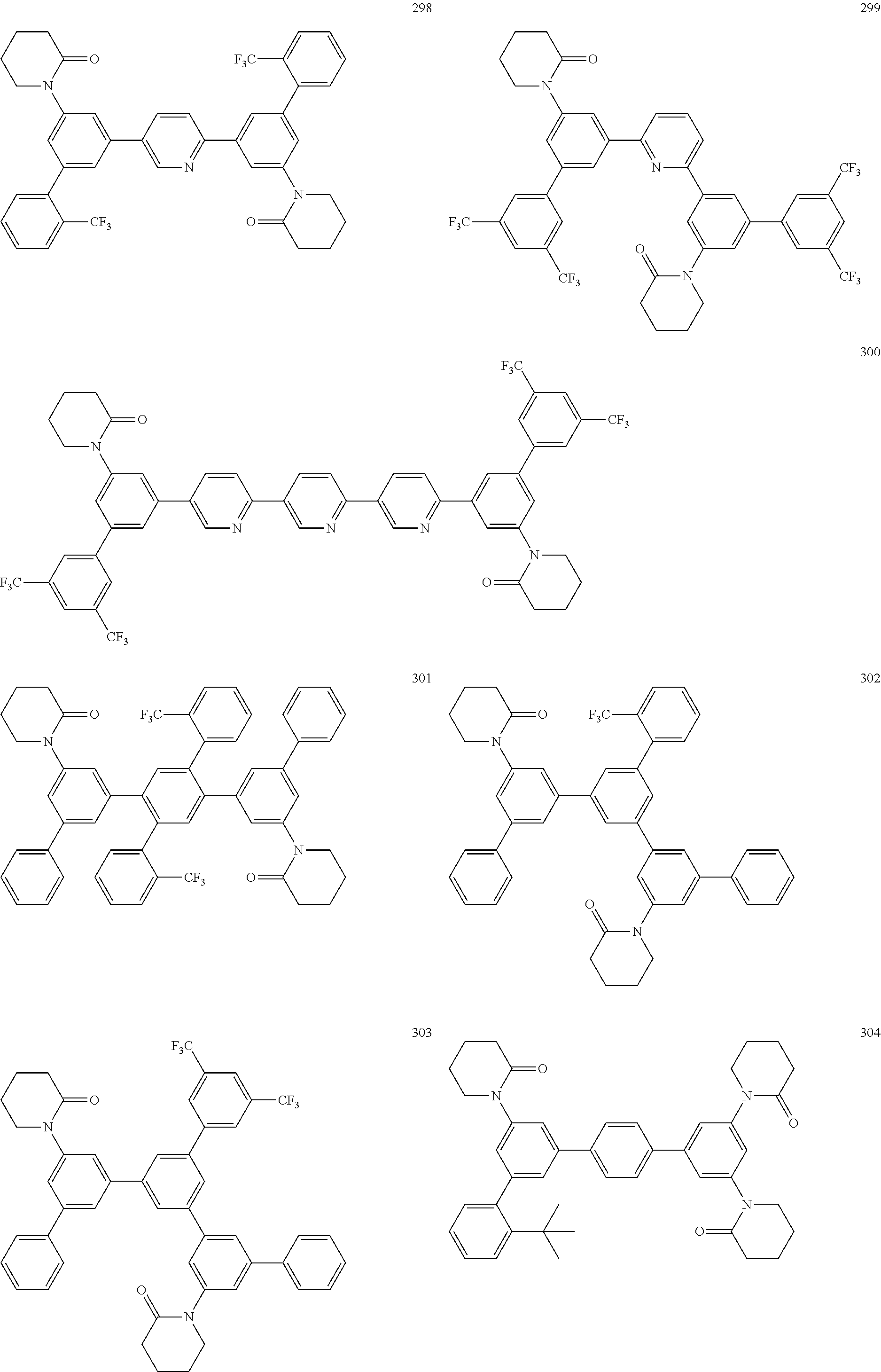

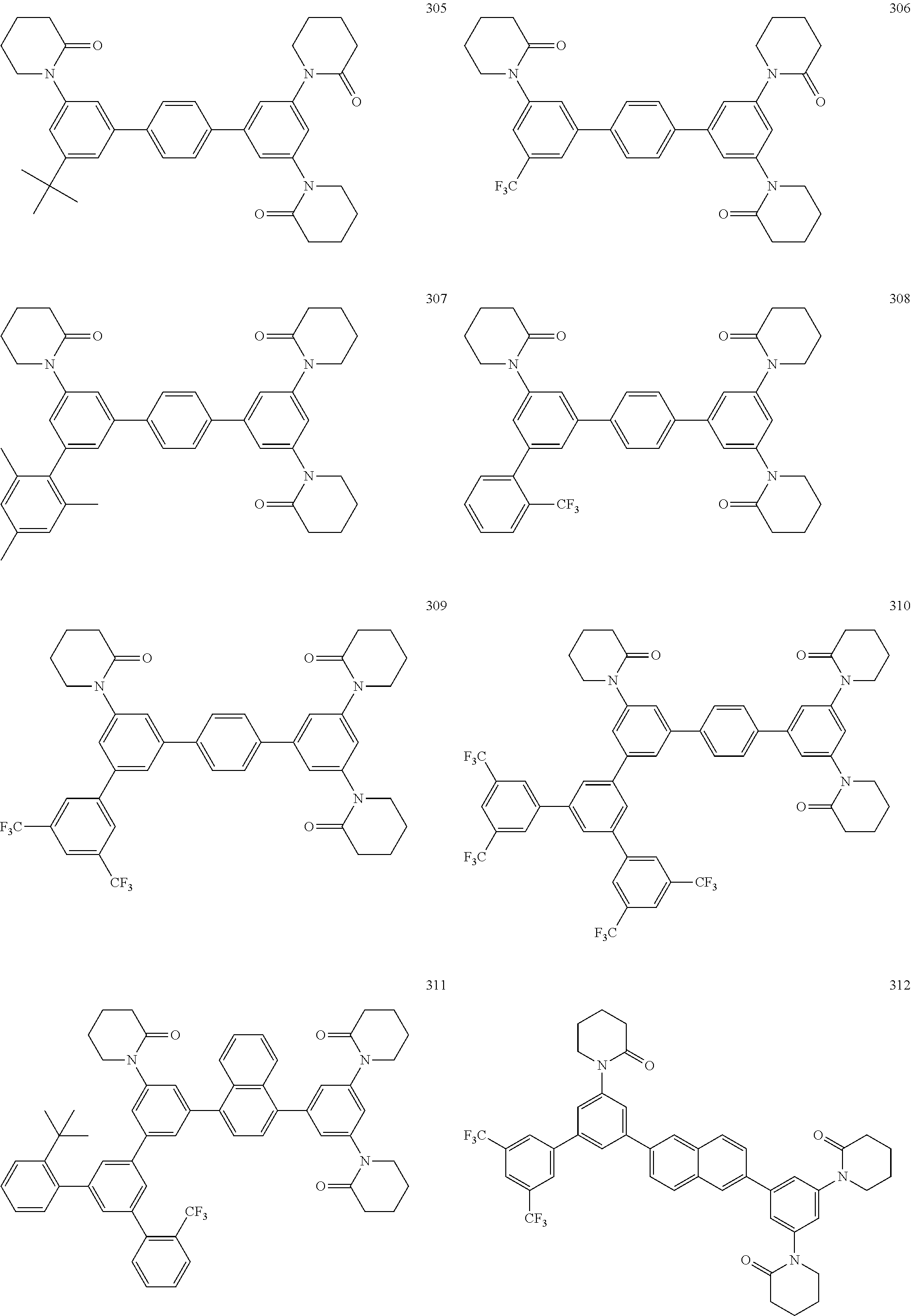

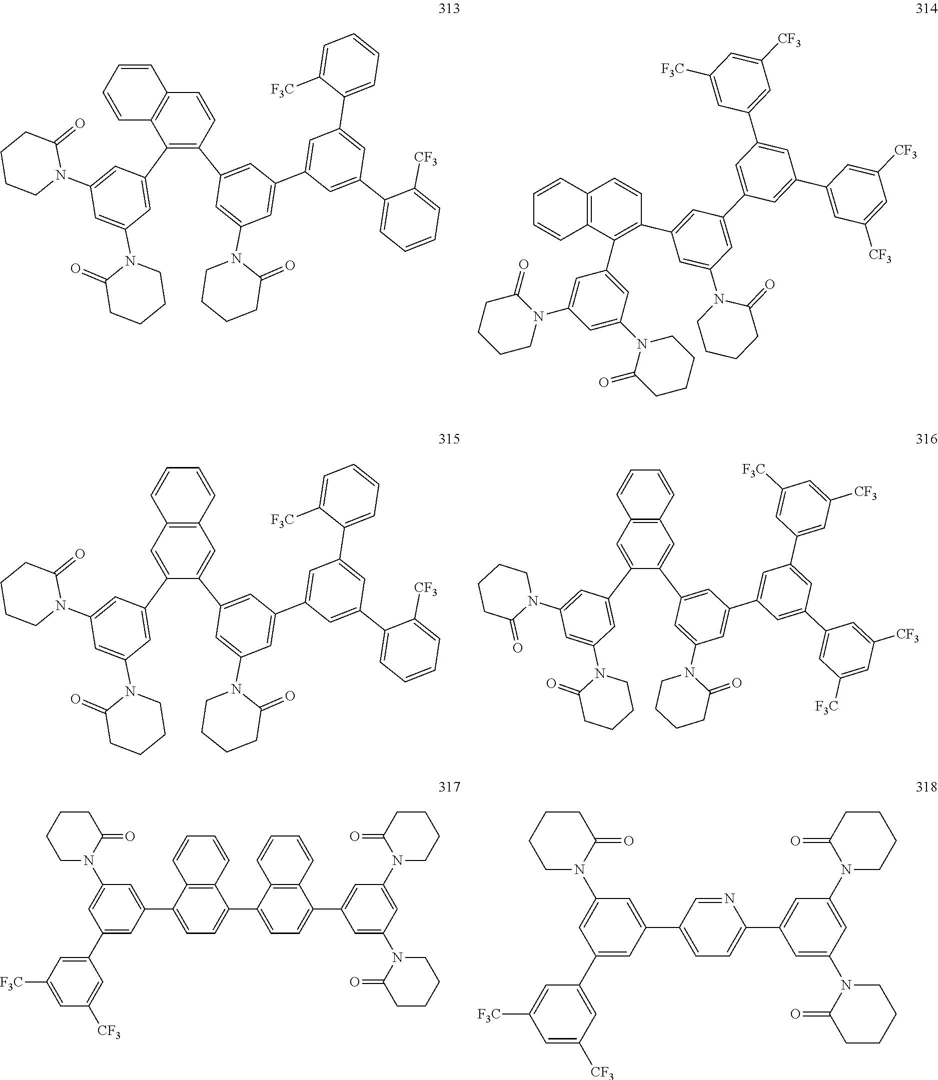

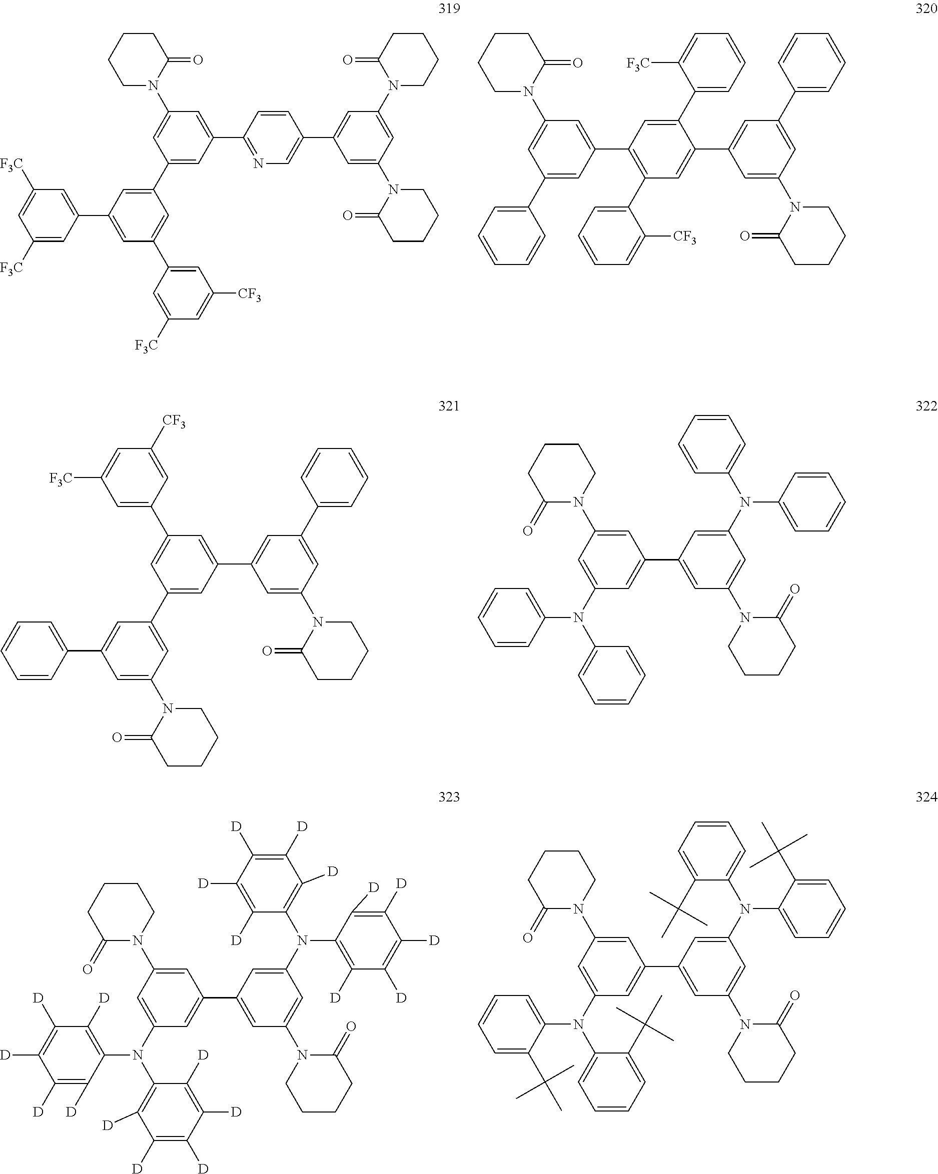

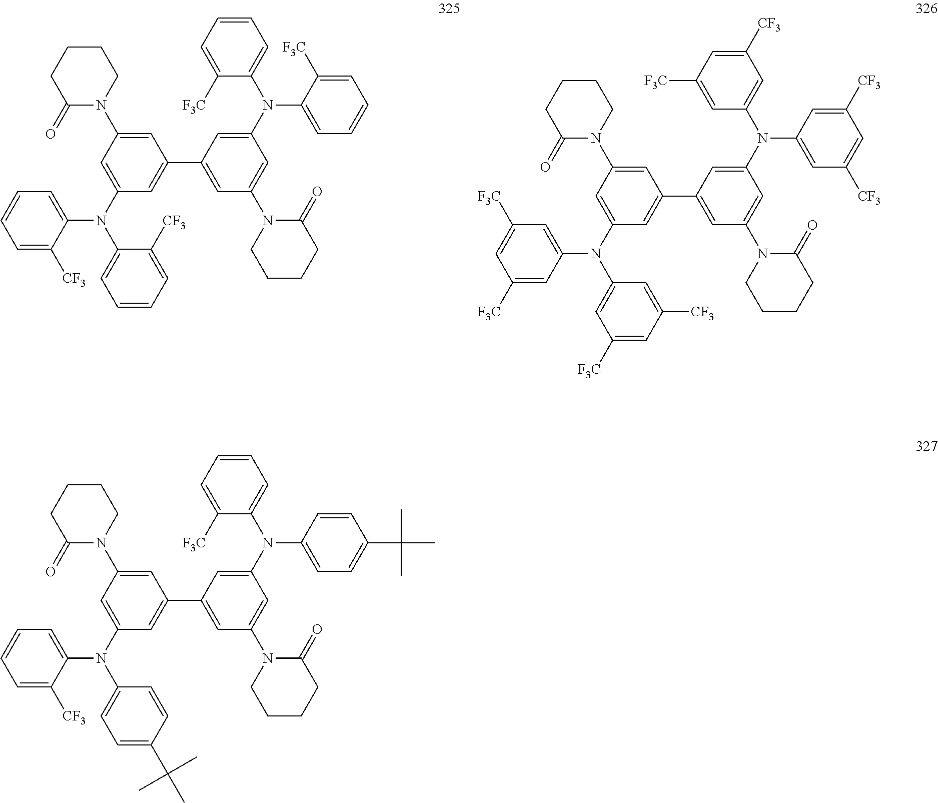 [SMILES: O=C1CCCCN1c1cc(-c2cc(N3CCCCC3=O)cc(N(c3ccccc3C(F)(F)F)c3ccccc3C(F)(F)F)c2)cc(N(c2ccccc2C(F)(F)F)c2ccccc2C(F)(F)F)c1 ; O=C1CCCCN1c1cc(-c2cc(N3CCCCC3=O)cc(N(c3cc(C(F)(F)F)cc(C(F)(F)F)c3)c3cc(C(F)(F)F)cc(C(F)(F)F)c3)c2)cc(N(c2cc(C(F)(F)F)cc(C(F)(F)F)c2)c2cc(C(F)(F)F)cc(C(F)(F)F)c2)c1 ; CC(C)(C)c1ccc(N(c2cc(-c3cc(N4CCCCC4=O)cc(N(c4ccc(C(C)(C)C)cc4)c4ccccc4C(F)(F)F)c3)cc(N3CCCCC3=O)c2)c2ccccc2C(F)(F)F)cc1]

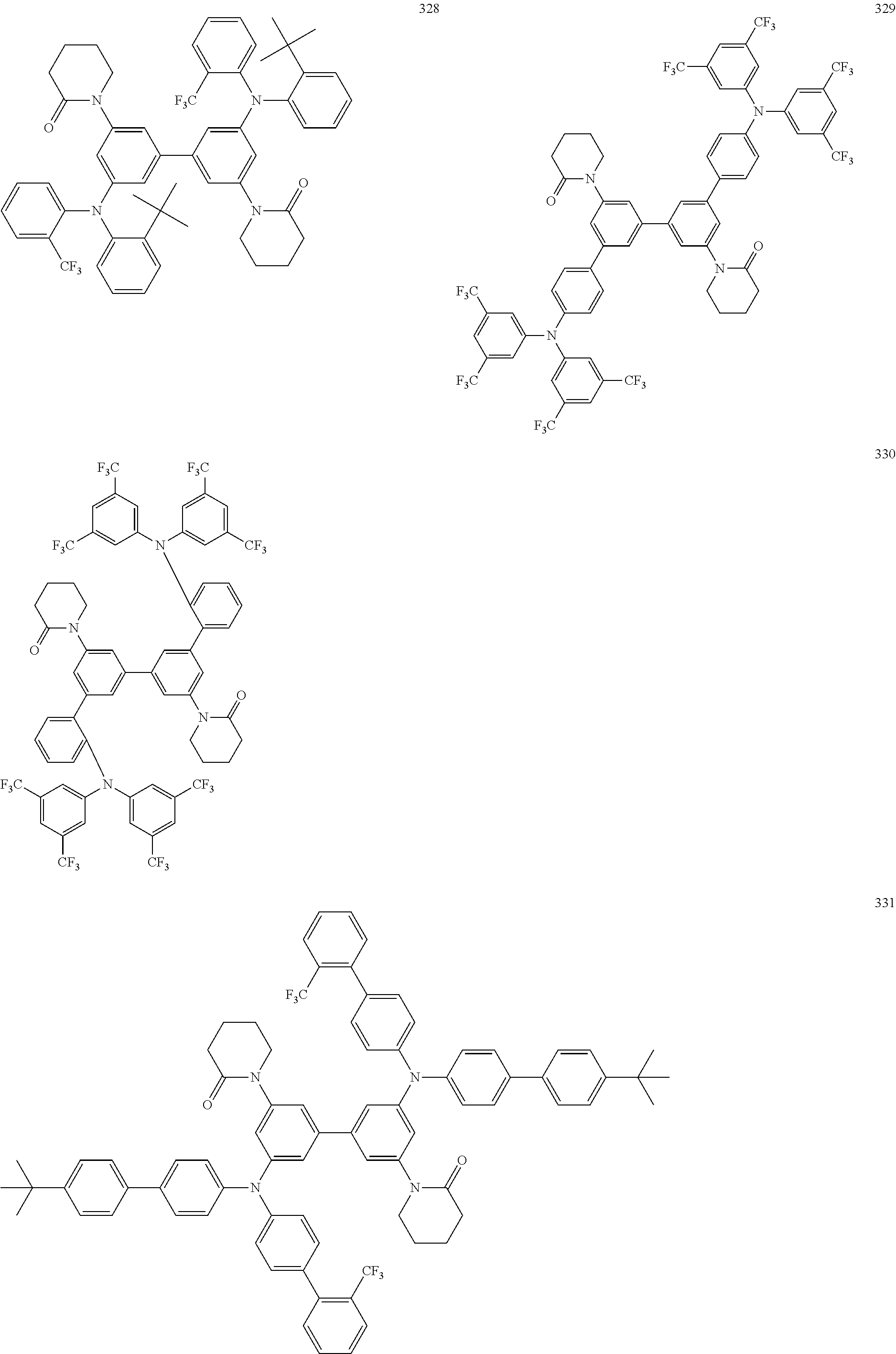

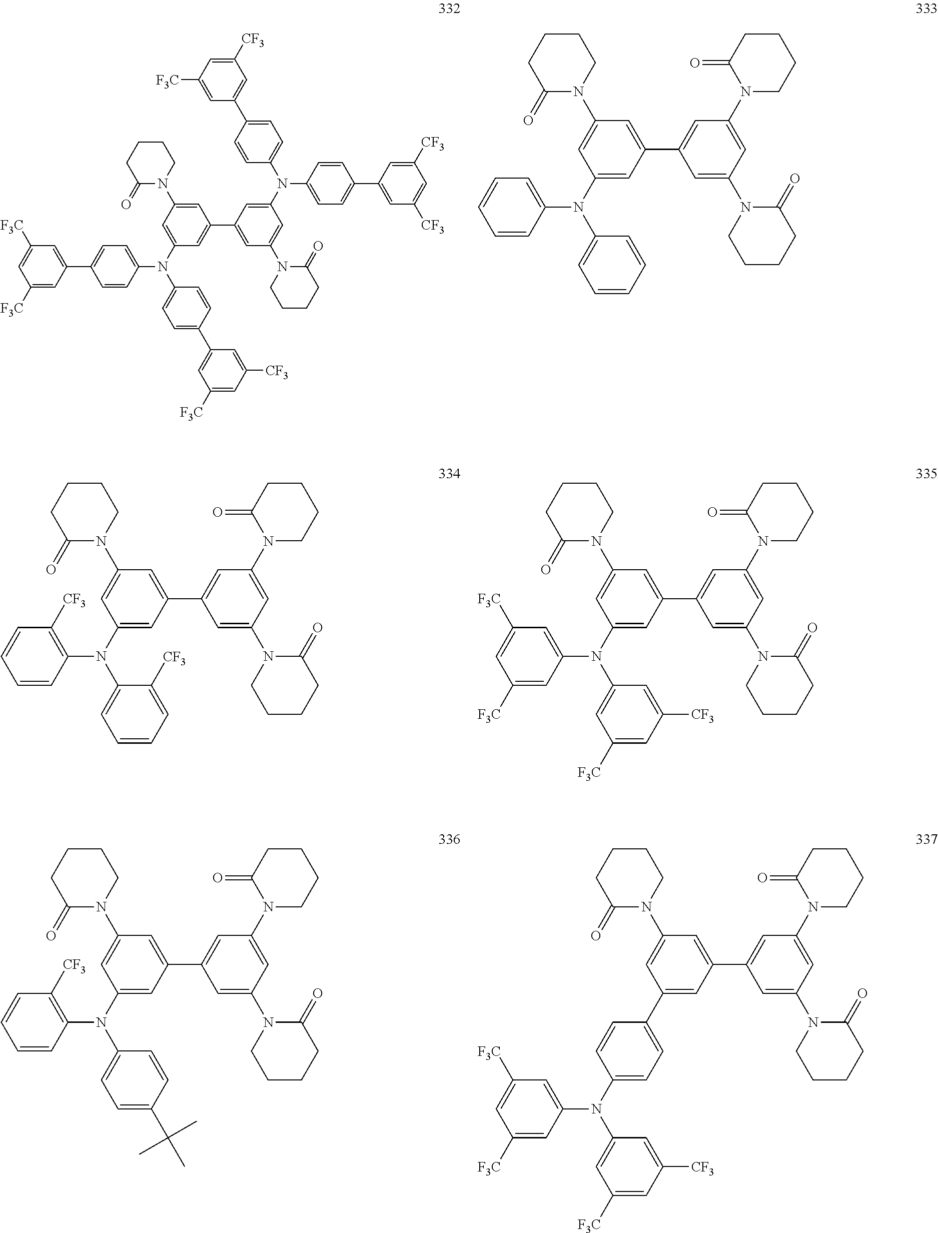

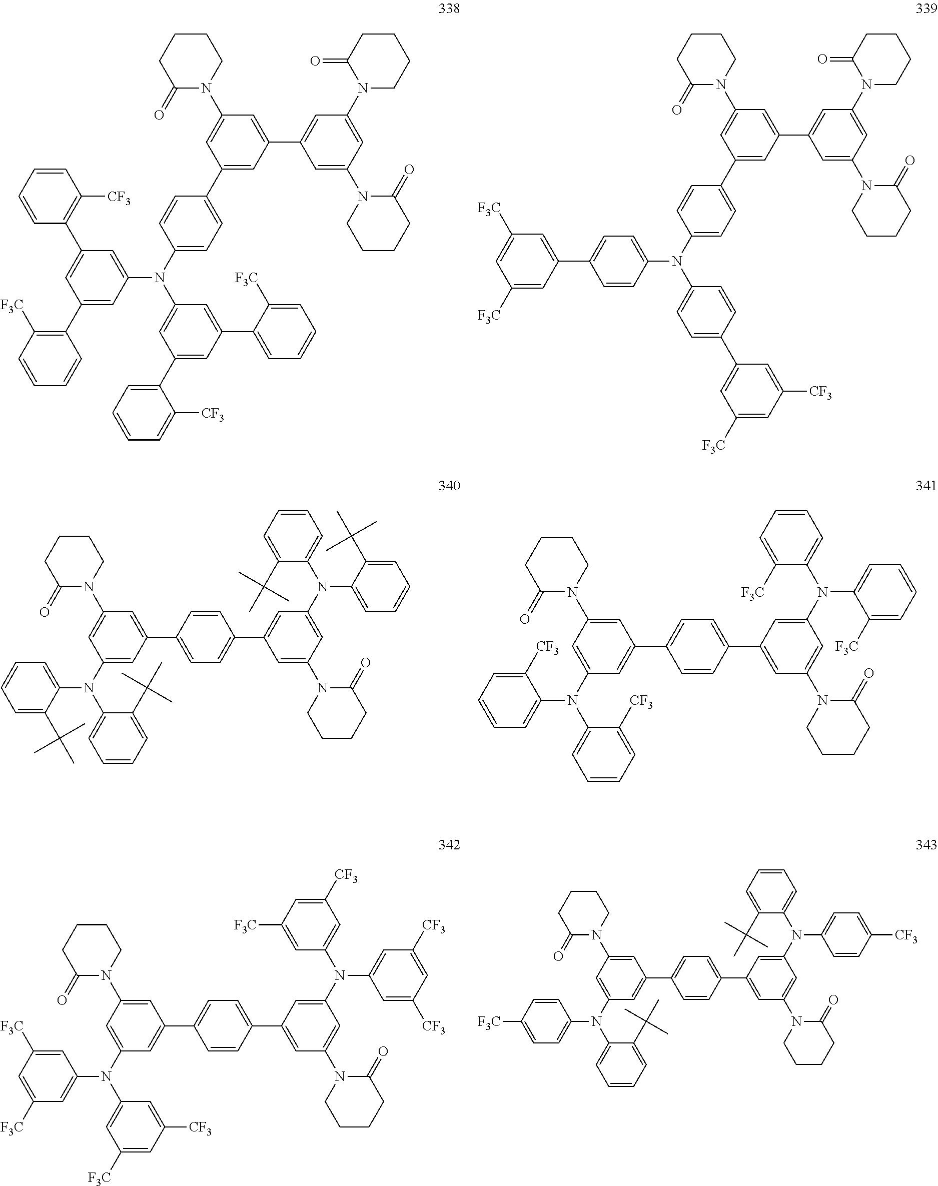

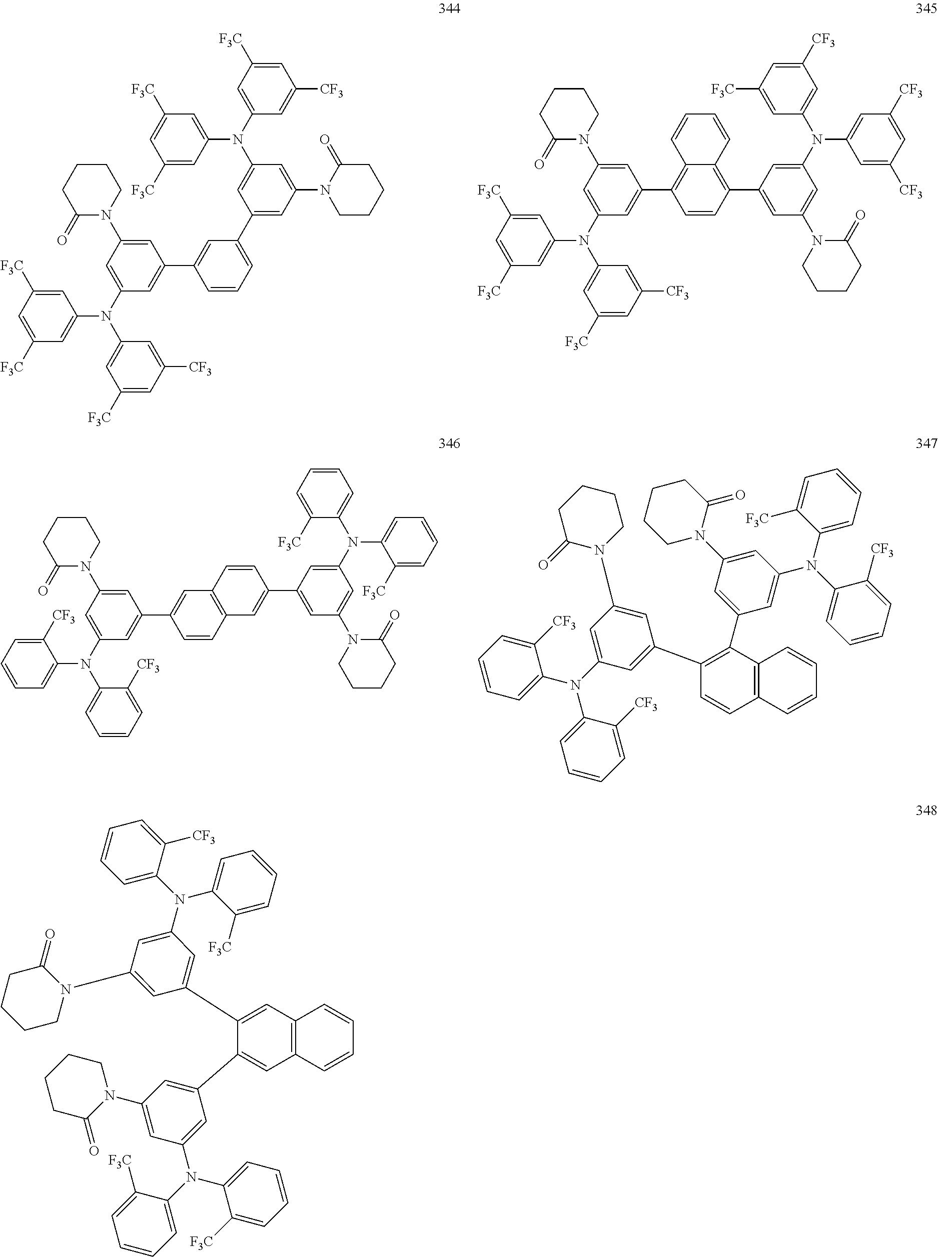

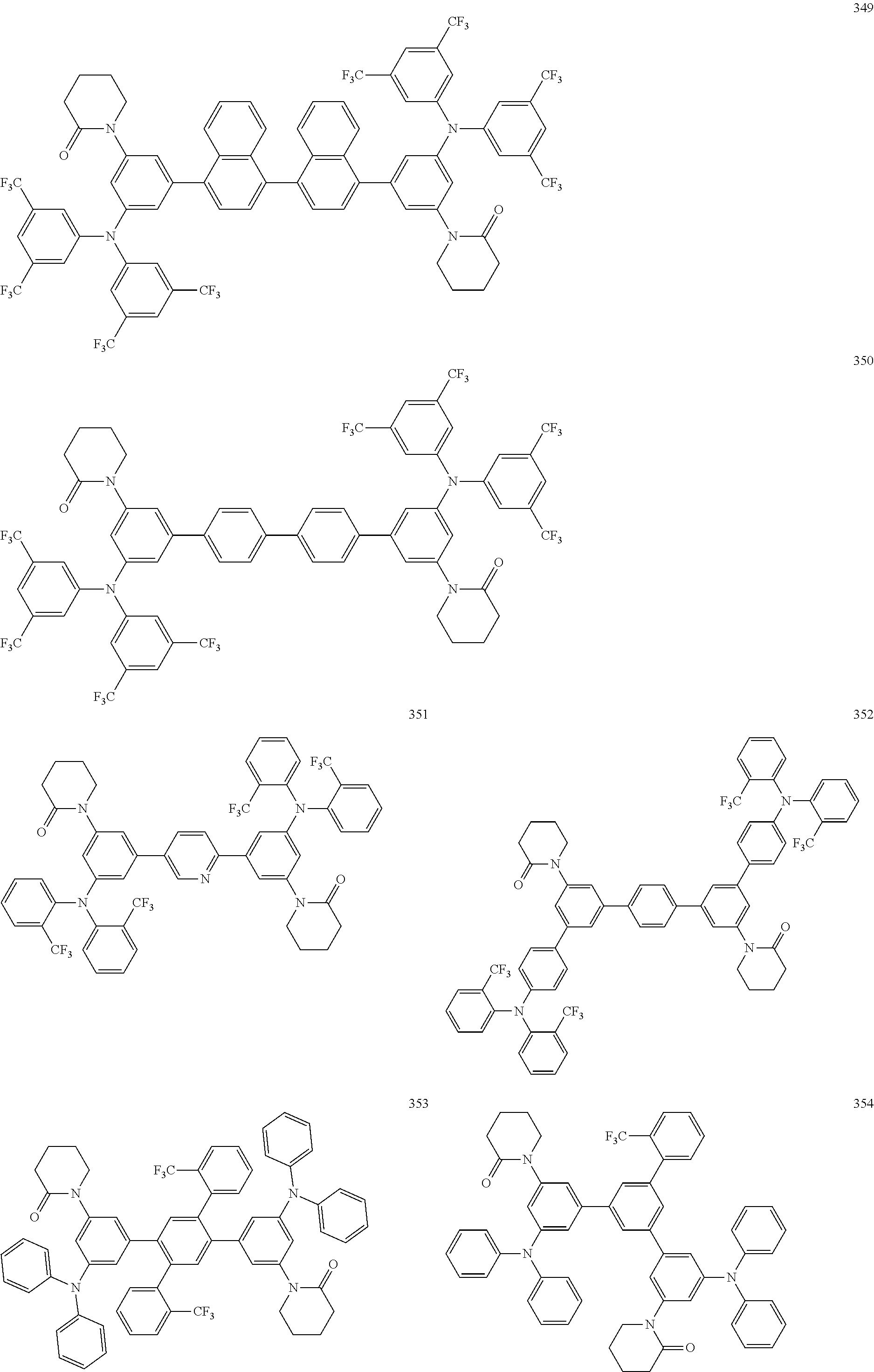

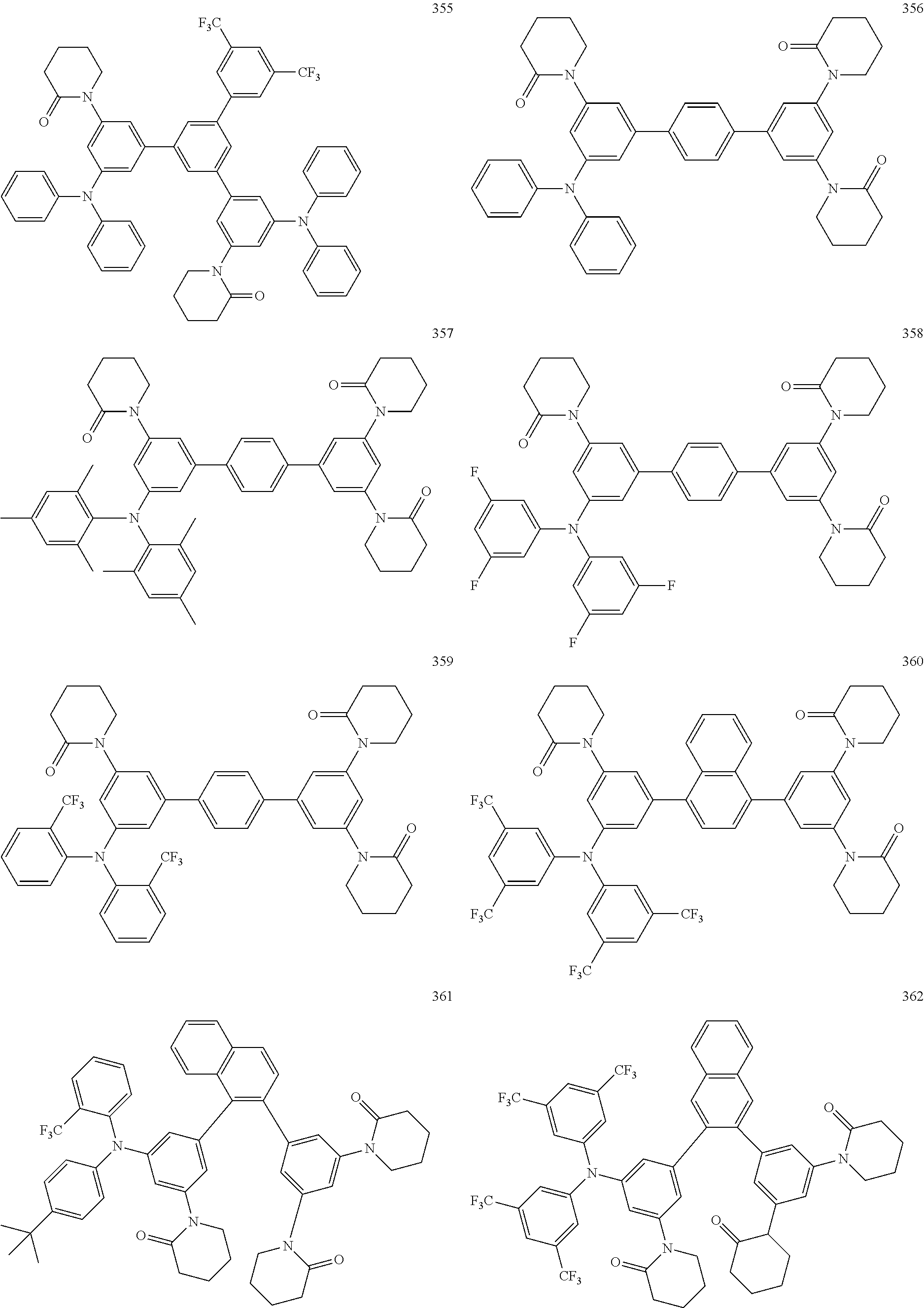

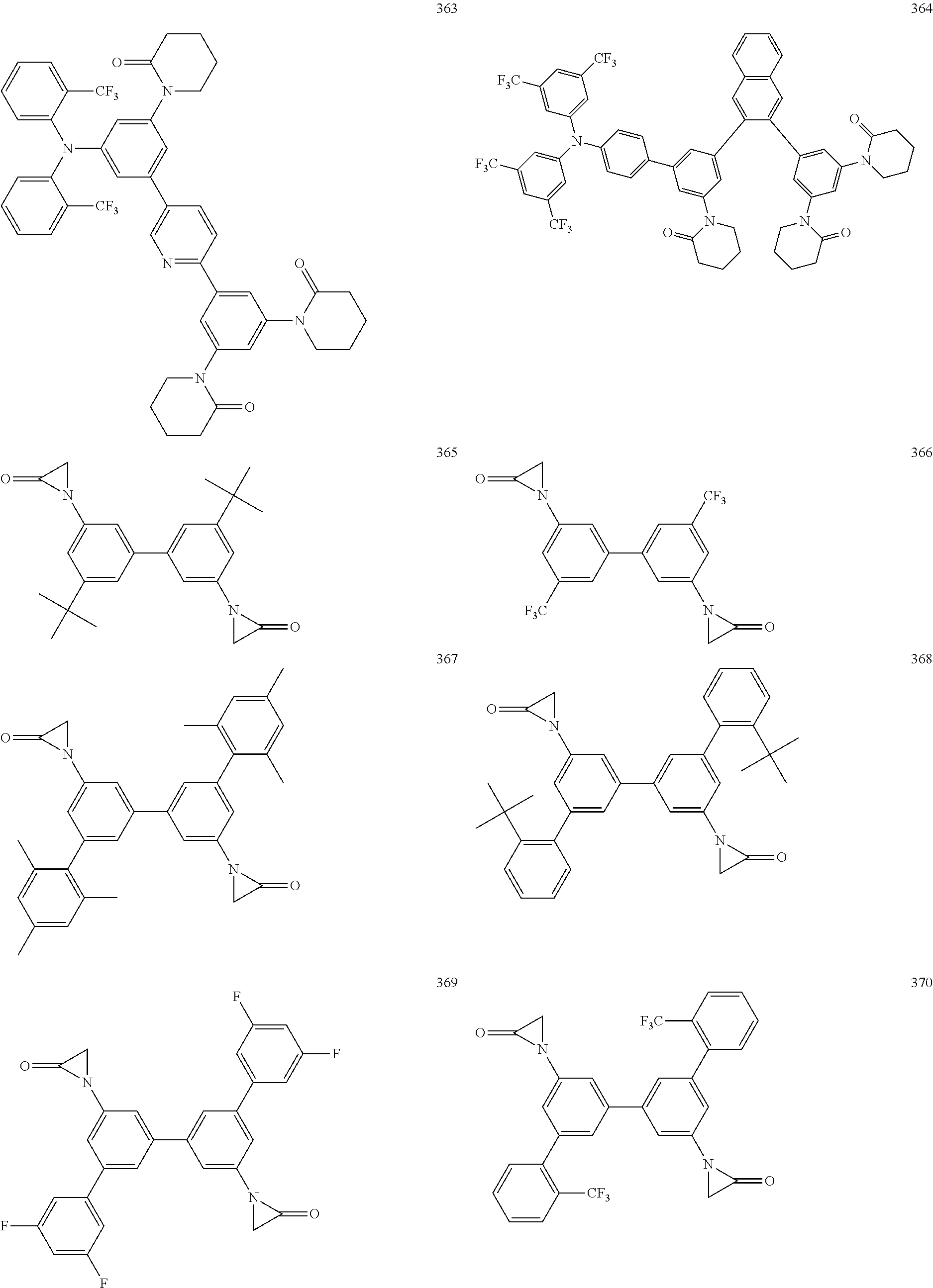

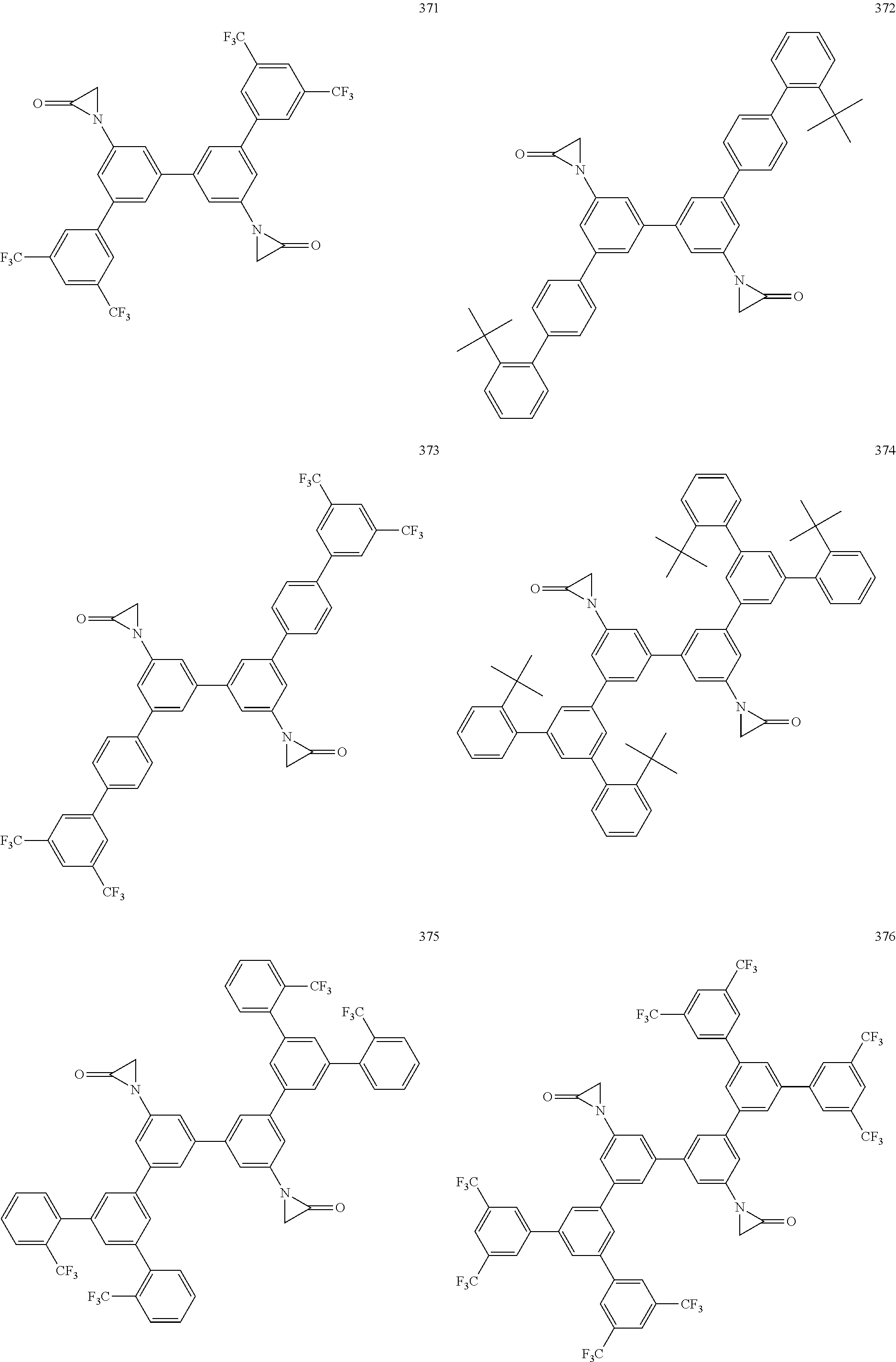

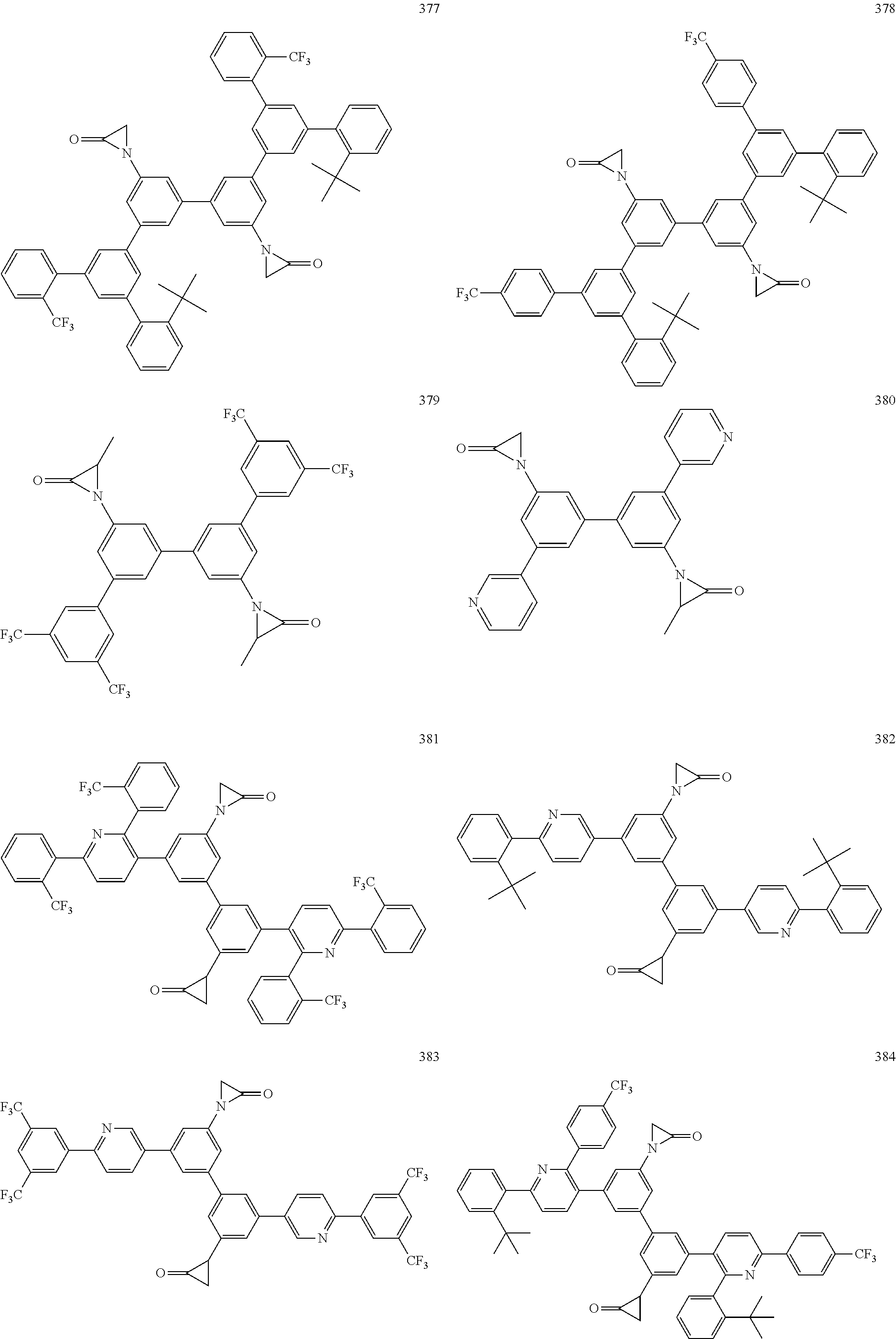

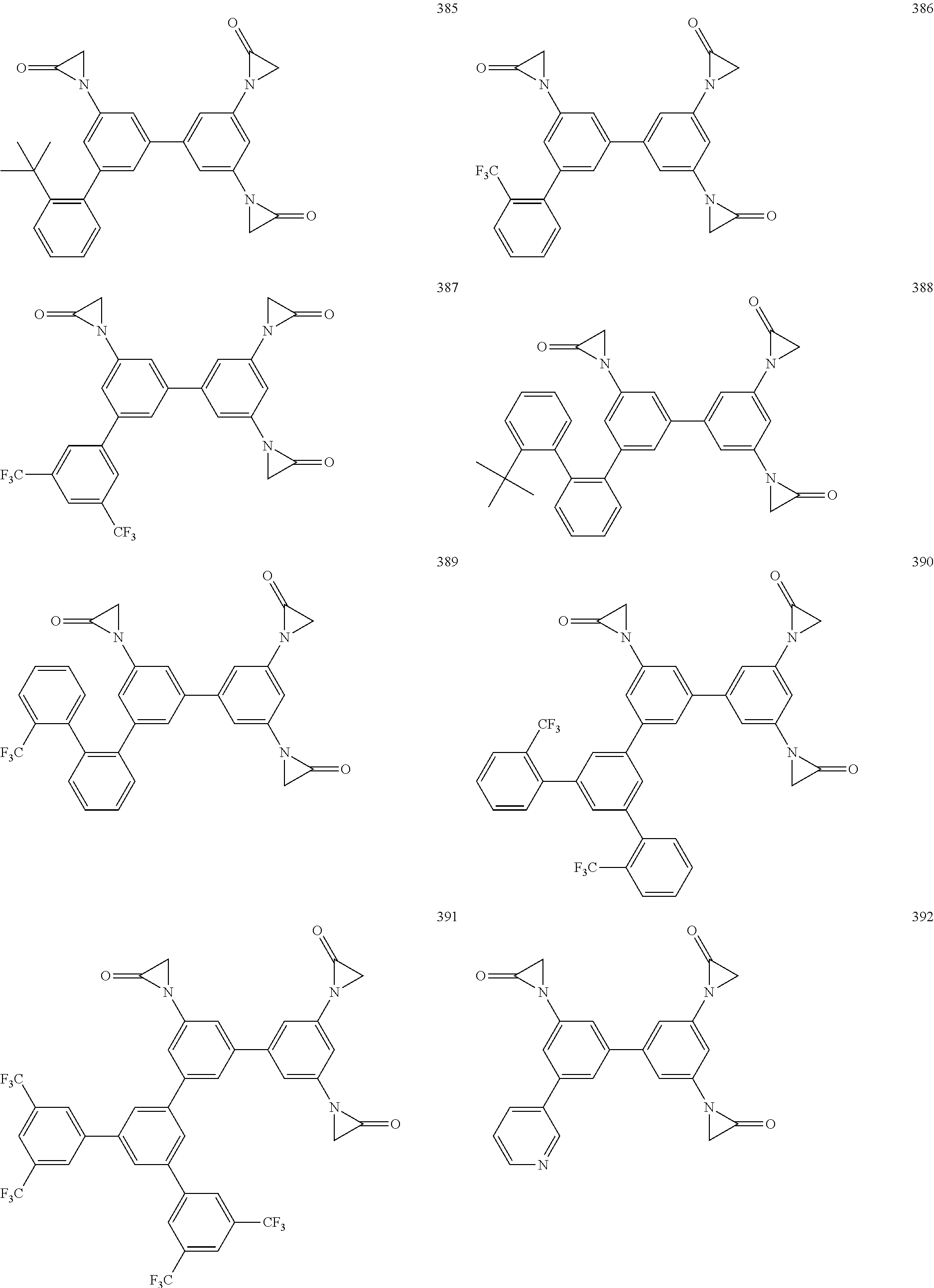

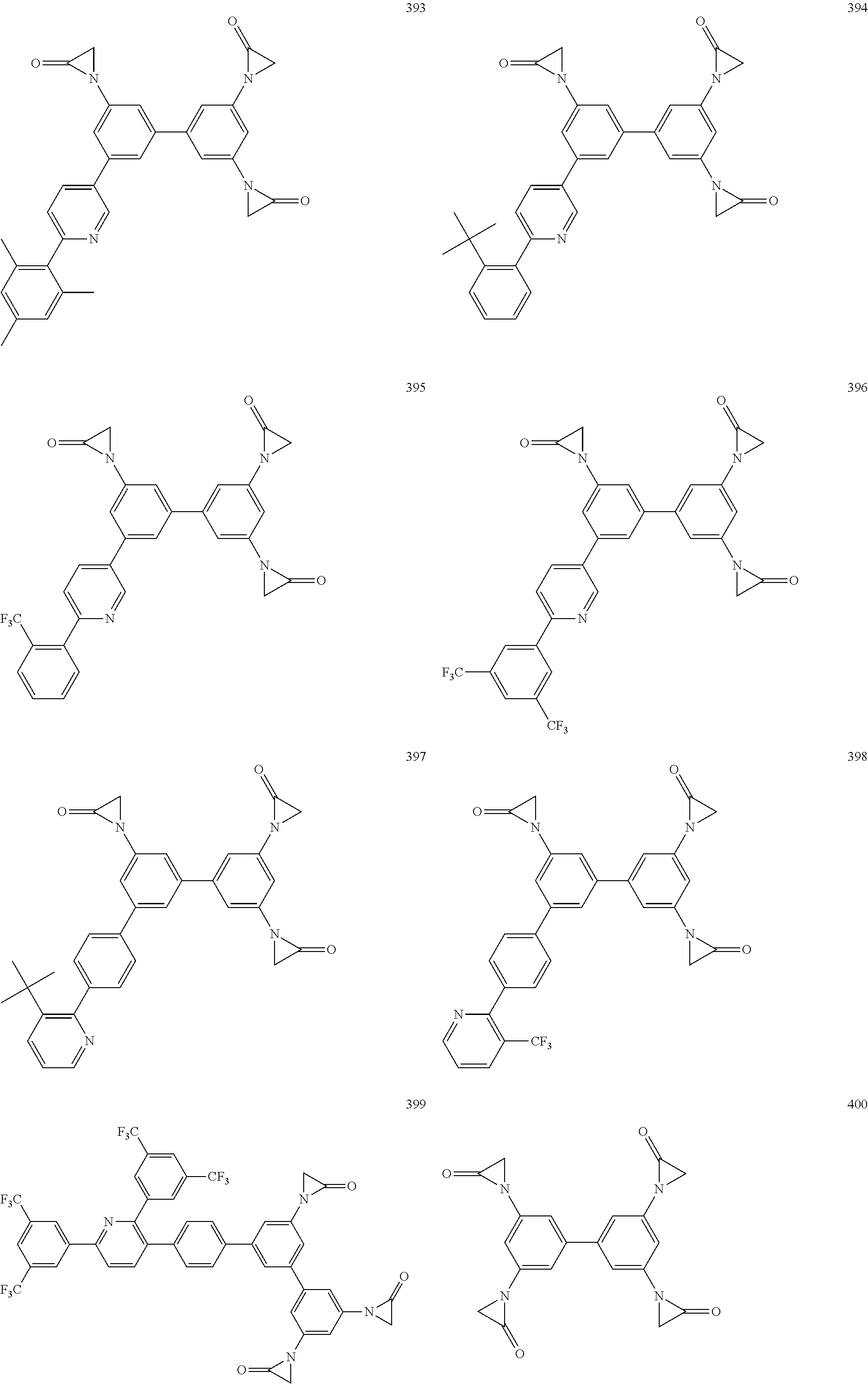

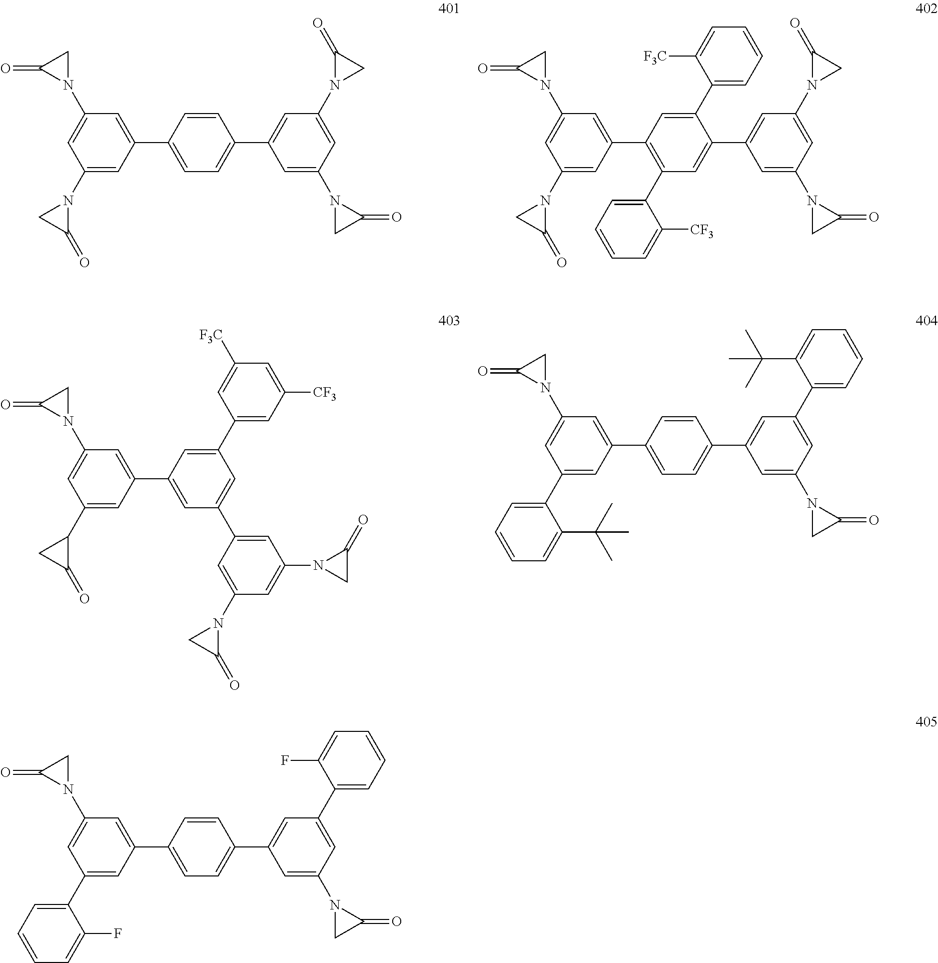 [SMILES: O=C1CN1c1cc(-c2ccc(-c3cc(N4CC4=O)cc(N4CC4=O)c3)cc2)cc(N2CC2=O)c1 ; O=C1CN1c1cc(-c2cc(-c3ccccc3C(F)(F)F)c(-c3cc(N4CC4=O)cc(N4CC4=O)c3)cc2-c2ccccc2C(F)(F)F)cc(N2CC2=O)c1 ; O=C1CC1c1cc(-c2cc(-c3cc(N4CC4=O)cc(N4CC4=O)c3)cc(-c3cc(C(F)(F)F)cc(C(F)(F)F)c3)c2)cc(N2CC2=O)c1 ; CC(C)(C)c1ccccc1-c1cc(-c2ccc(-c3cc(-c4ccccc4C(C)(C)C)cc(N4CC4=O)c3)cc2)cc(N2CC2=O)c1 ; O=C1CN1c1cc(-c2ccc(-c3cc(-c4ccccc4F)cc(N4CC4=O)c3)cc2)cc(-c2ccccc2F)c1]

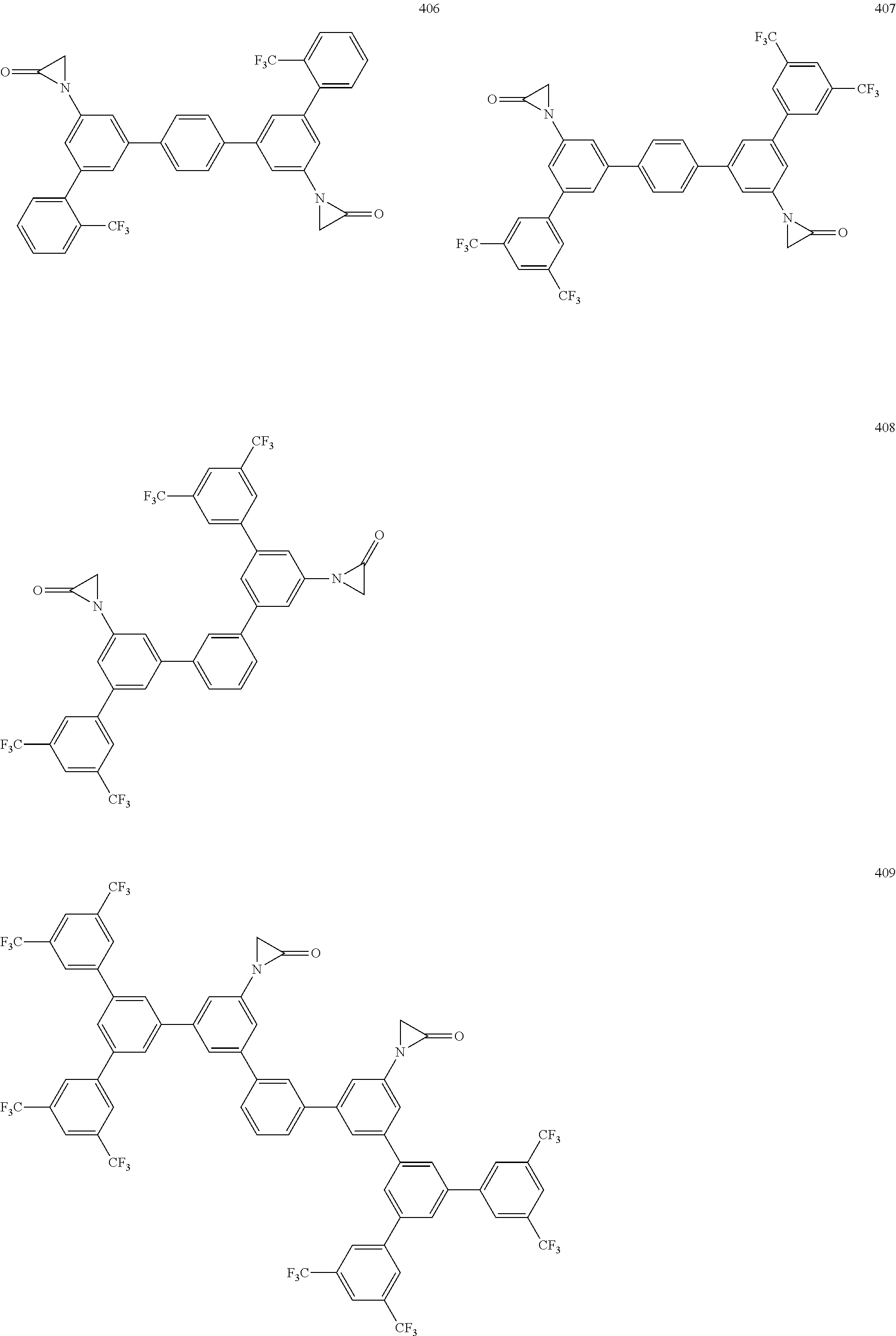

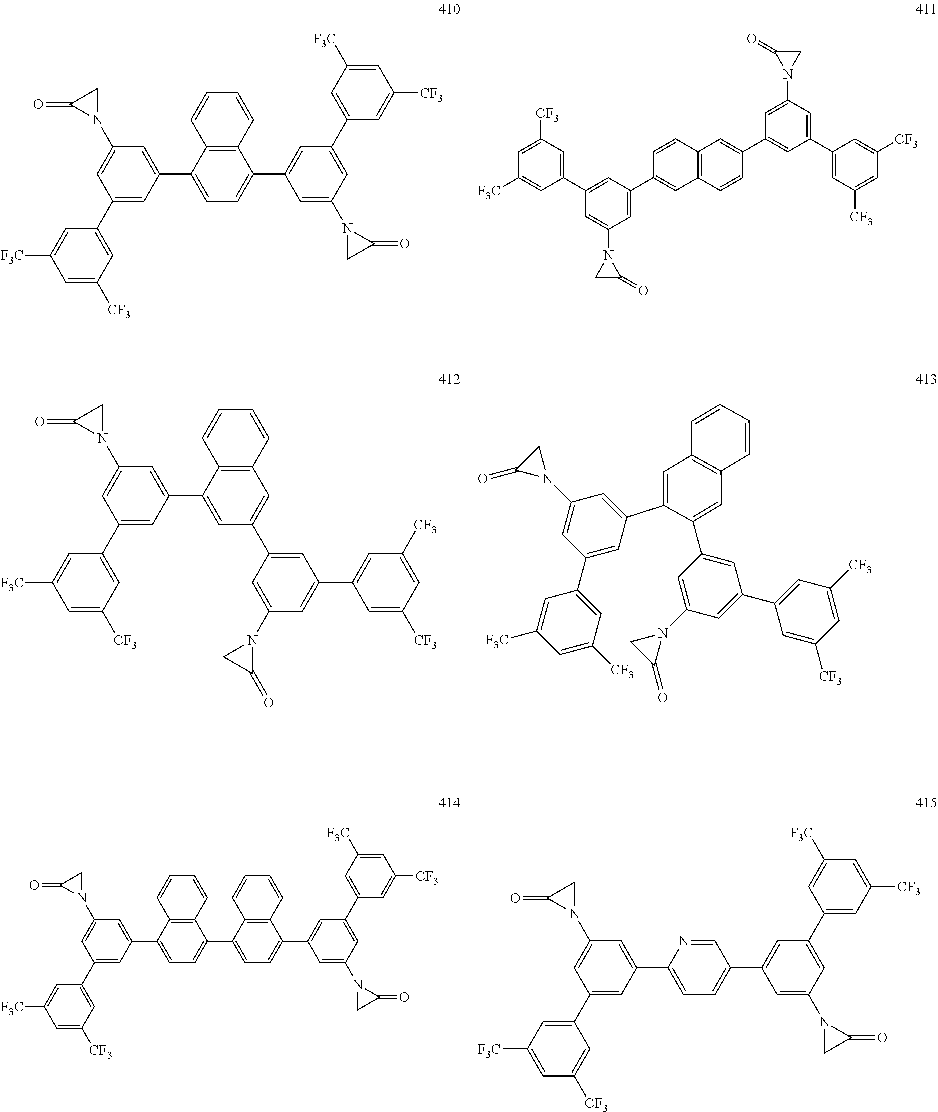

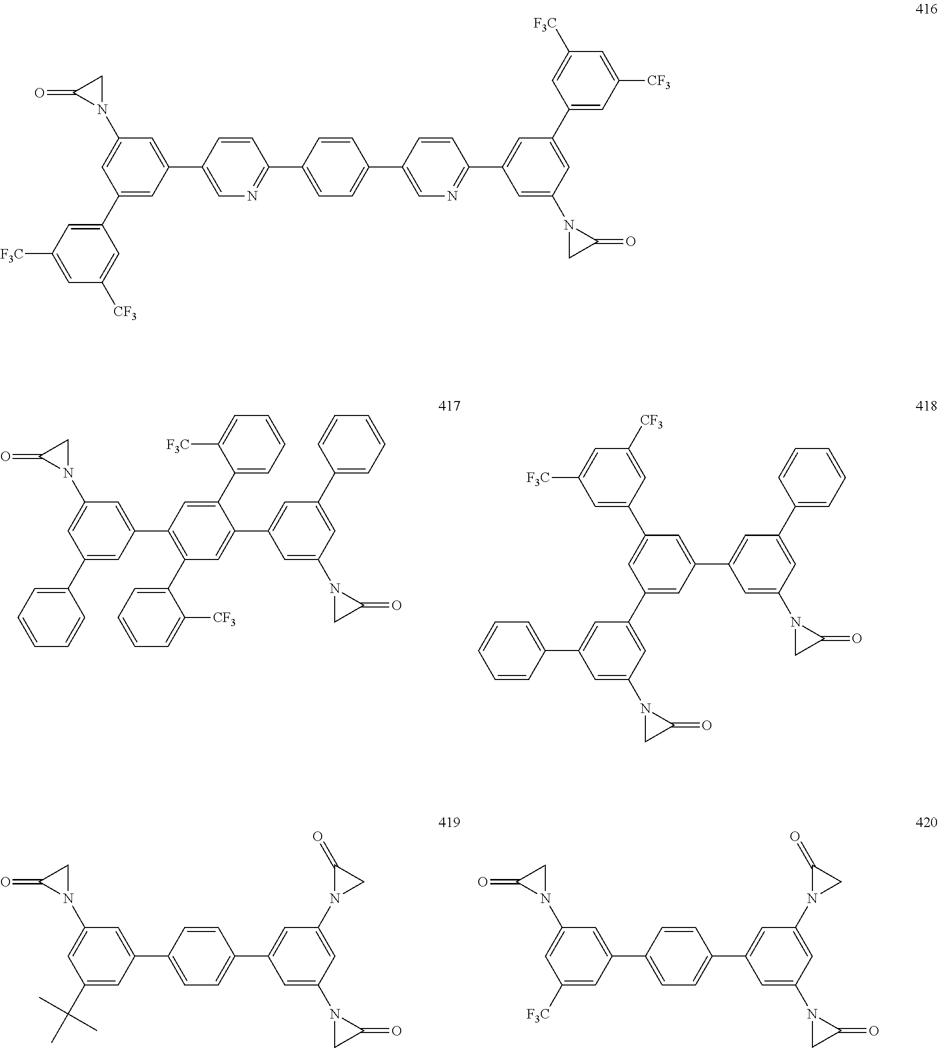 [SMILES: O=C1CN1c1cc(-c2ccc(-c3ccc(-c4ccc(-c5cc(-c6cc(C(F)(F)F)cc(C(F)(F)F)c6)cc(N6CC6=O)c5)nc4)cc3)nc2)cc(-c2cc(C(F)(F)F)cc(C(F)(F)F)c2)c1 ; O=C1CN1c1cc(-c2ccccc2)cc(-c2cc(-c3ccccc3C(F)(F)F)c(-c3cc(-c4ccccc4)cc(N4CC4=O)c3)cc2-c2ccccc2C(F)(F)F)c1 ; O=C1CN1c1cc(-c2ccccc2)cc(-c2cc(-c3cc(-c4ccccc4)cc(N4CC4=O)c3)cc(-c3cc(C(F)(F)F)cc(C(F)(F)F)c3)c2)c1 ; CC(C)(C)c1cc(-c2ccc(-c3cc(N4CC4=O)cc(N4CC4=O)c3)cc2)cc(N2CC2=O)c1 ; O=C1CN1c1cc(-c2ccc(-c3cc(N4CC4=O)cc(C(F)(F)F)c3)cc2)cc(N2CC2=O)c1]

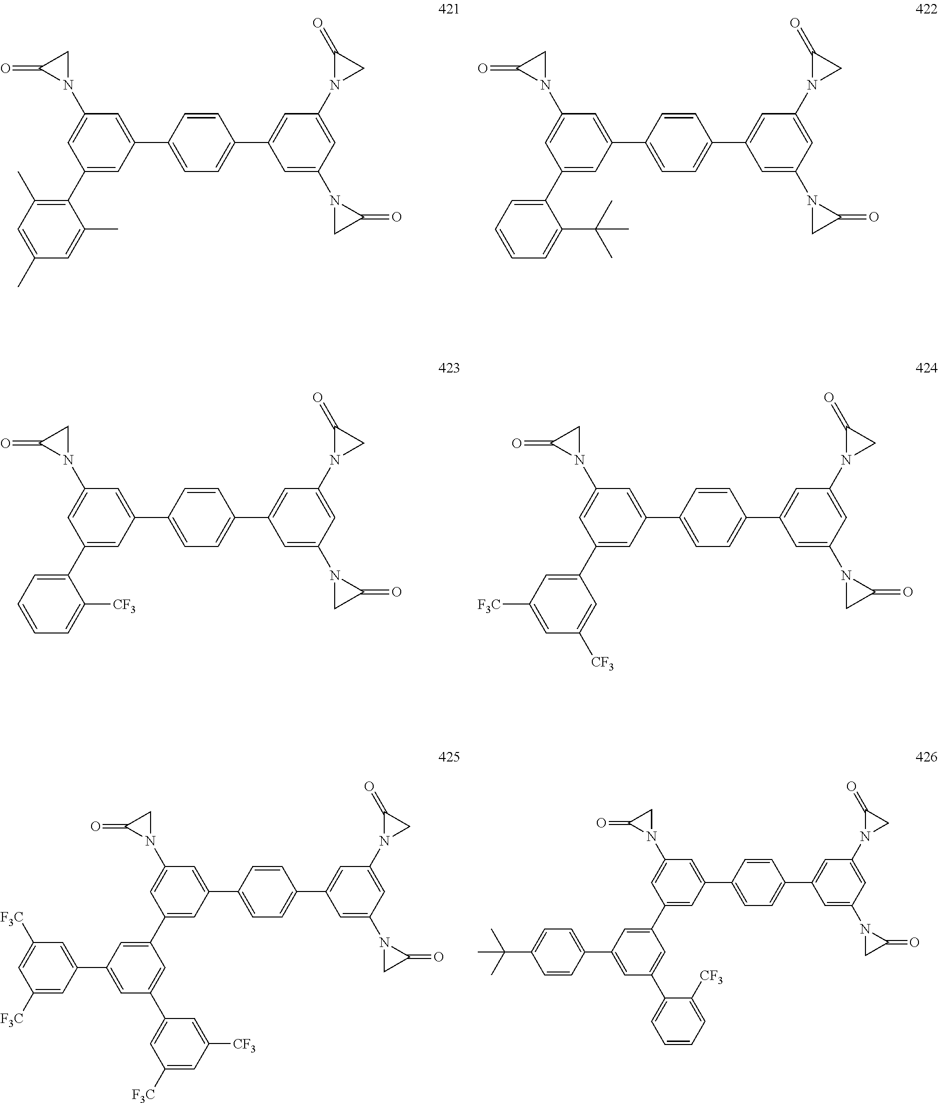

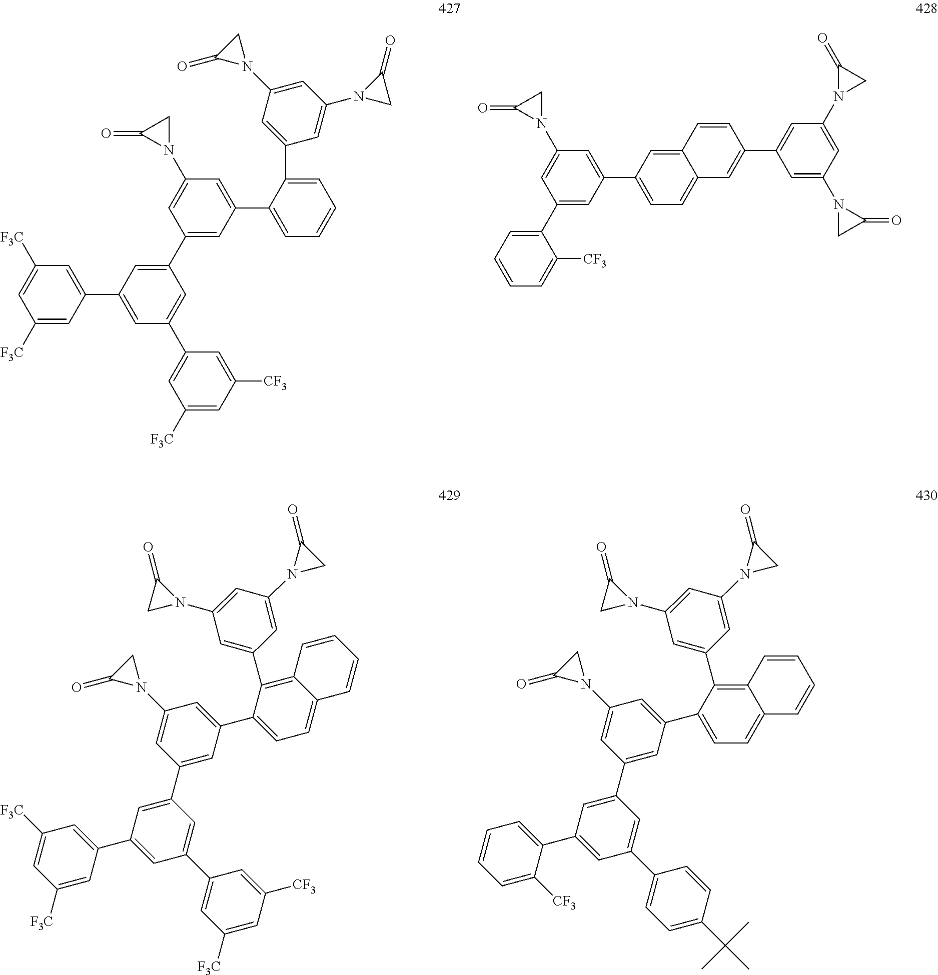 [SMILES: O=C1CN1c1cc(-c2cc(-c3cc(C(F)(F)F)cc(C(F)(F)F)c3)cc(-c3cc(C(F)(F)F)cc(C(F)(F)F)c3)c2)cc(-c2ccccc2-c2cc(N3CC3=O)cc(N3CC3=O)c2)c1 ; O=C1CN1c1cc(-c2ccc3cc(-c4cc(N5CC5=O)cc(N5CC5=O)c4)ccc3c2)cc(-c2ccccc2C(F)(F)F)c1 ; O=C1CN1c1cc(-c2cc(-c3cc(C(F)(F)F)cc(C(F)(F)F)c3)cc(-c3cc(C(F)(F)F)cc(C(F)(F)F)c3)c2)cc(-c2ccc3ccccc3c2-c2cc(N3CC3=O)cc(N3CC3=O)c2)c1 ; CC(C)(C)c1ccc(-c2cc(-c3cc(-c4ccc5ccccc5c4-c4cc(N5CC5=O)cc(N5CC5=O)c4)cc(N4CC4=O)c3)cc(-c3ccccc3C(F)(F)F)c2)cc1]

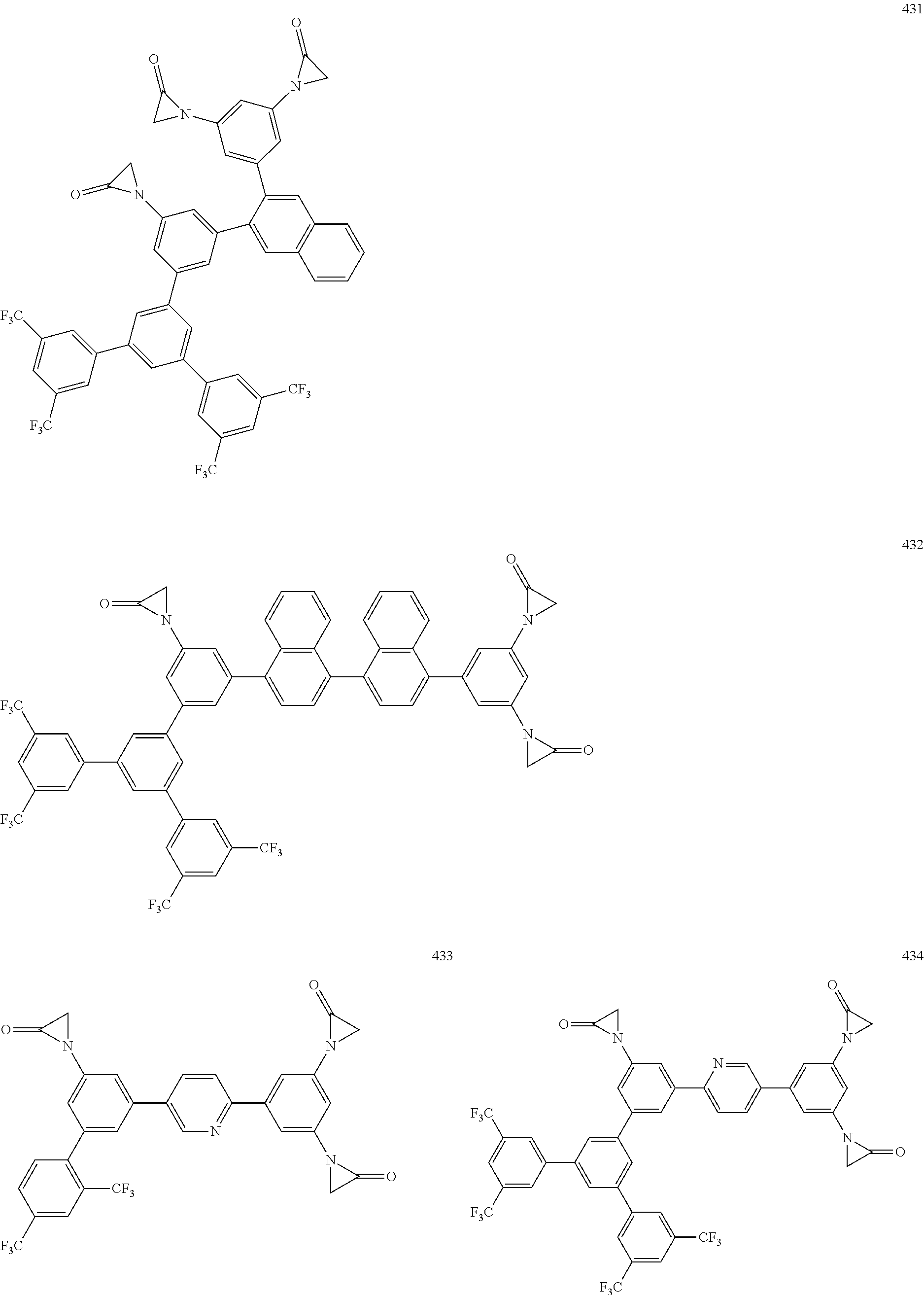

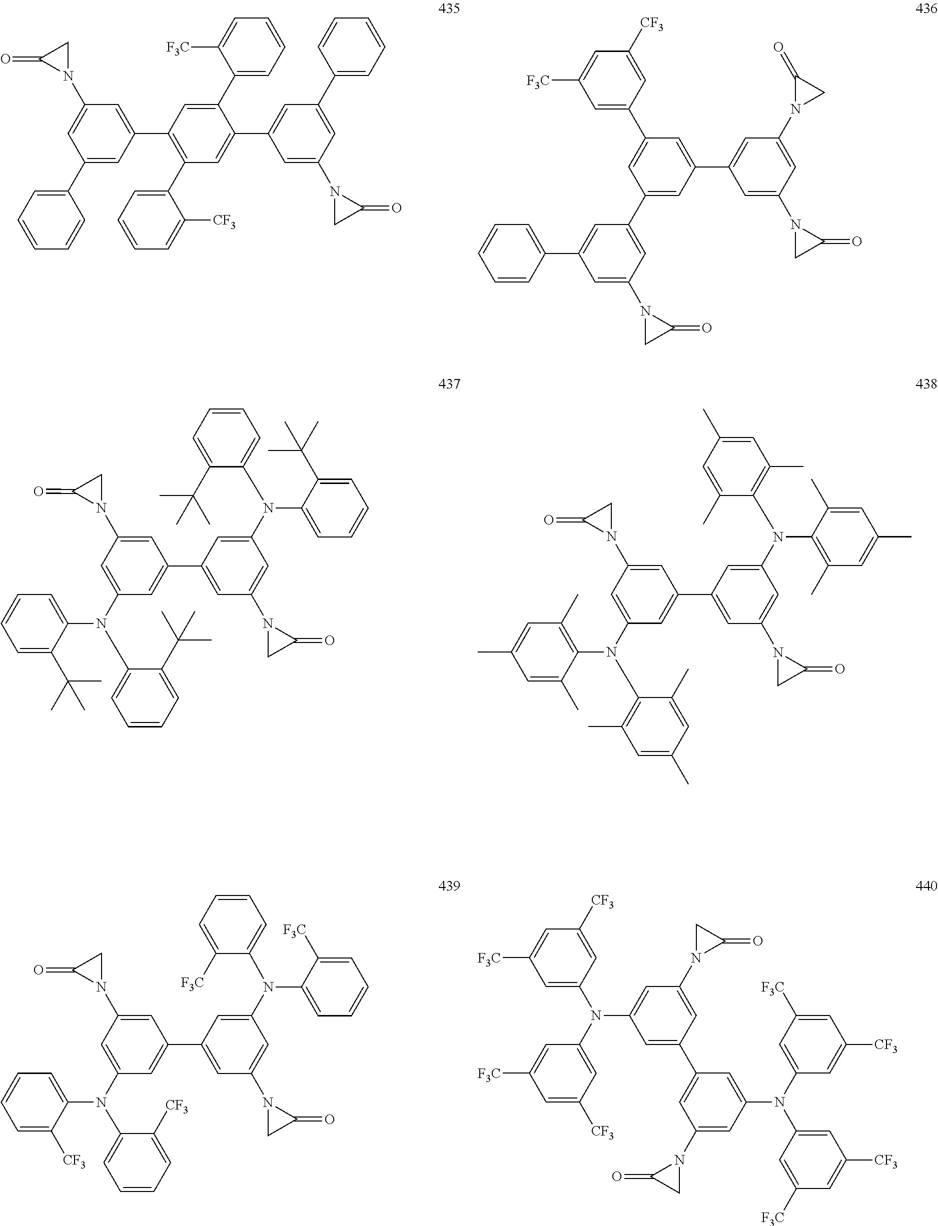

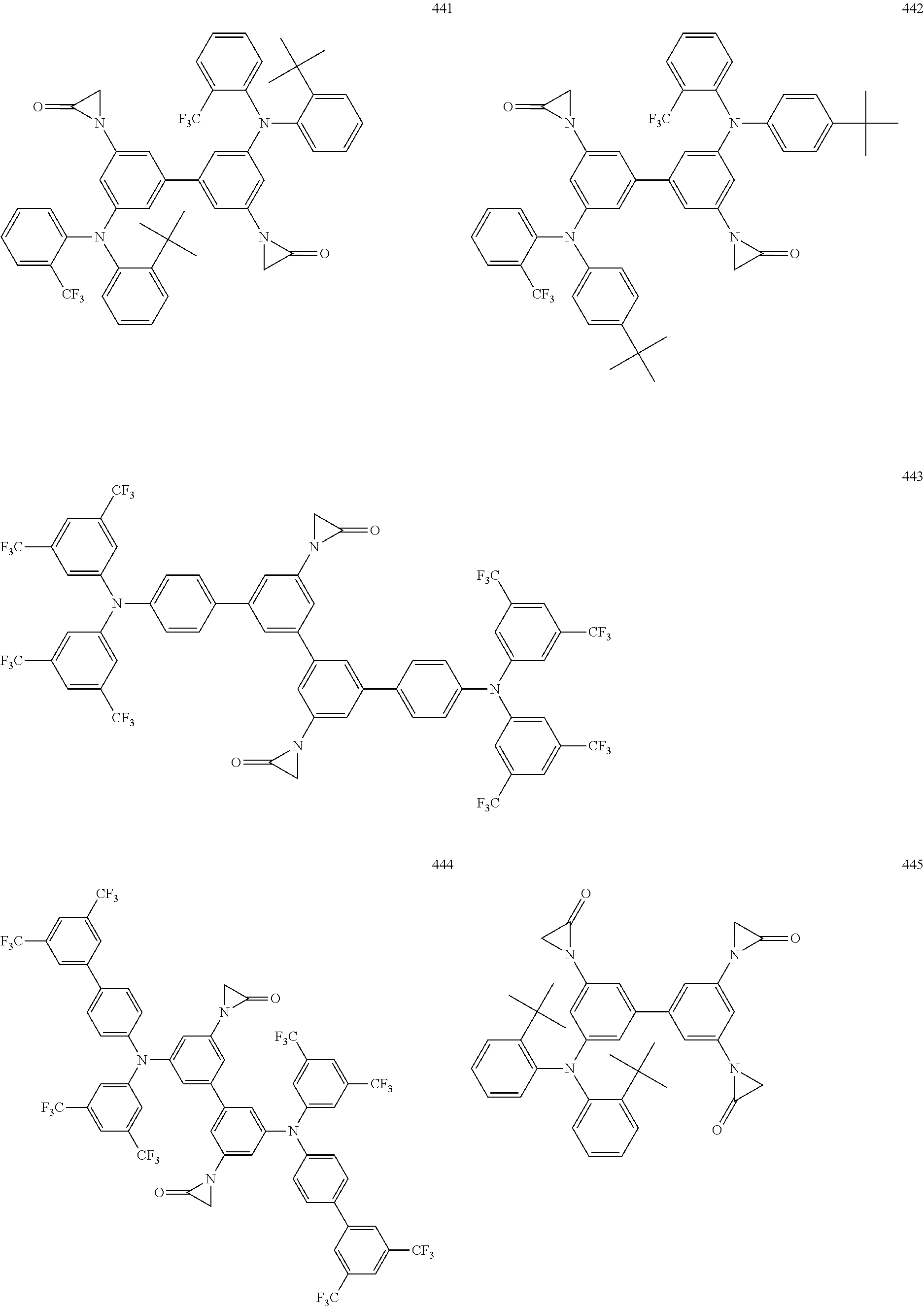

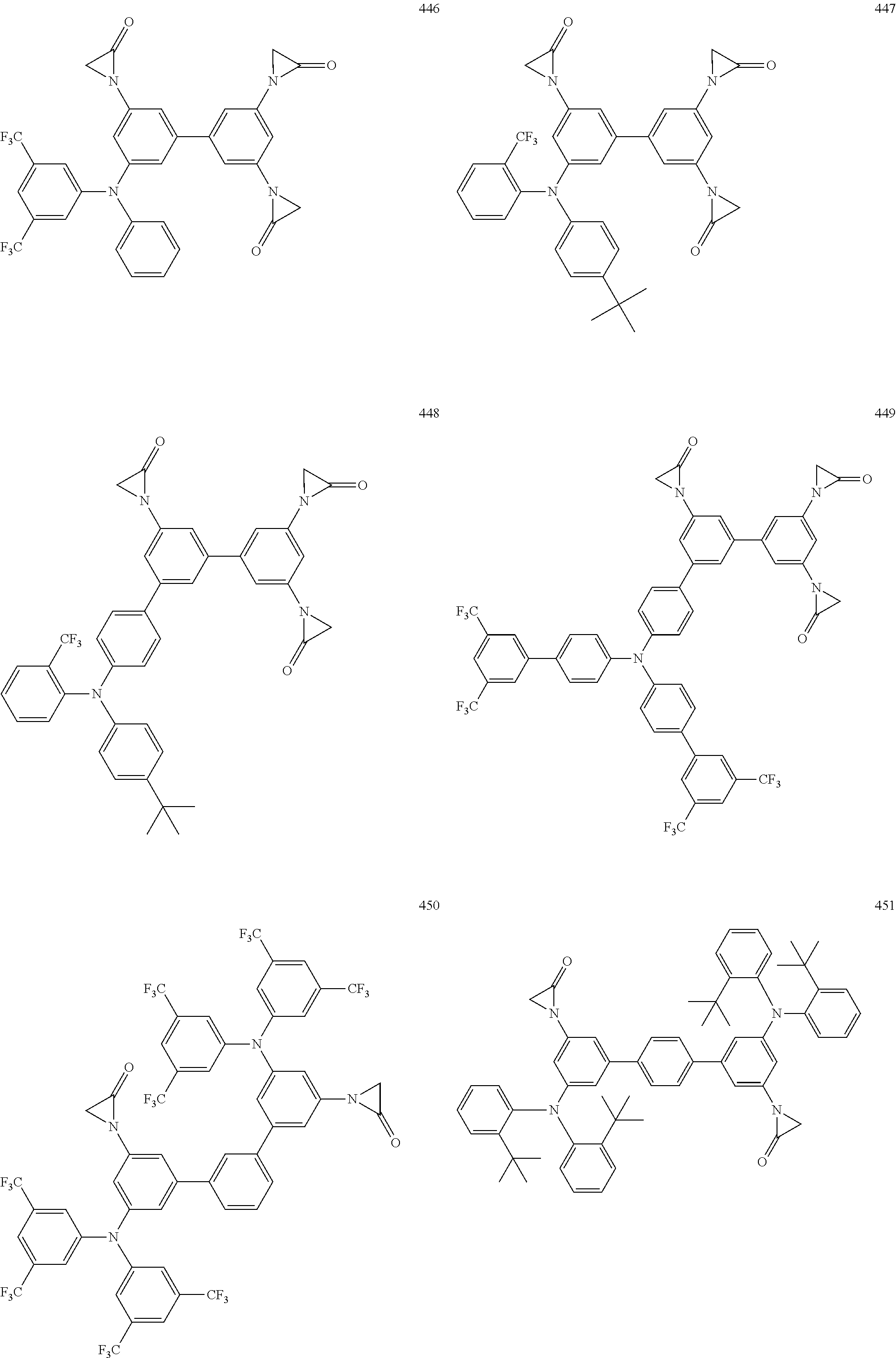

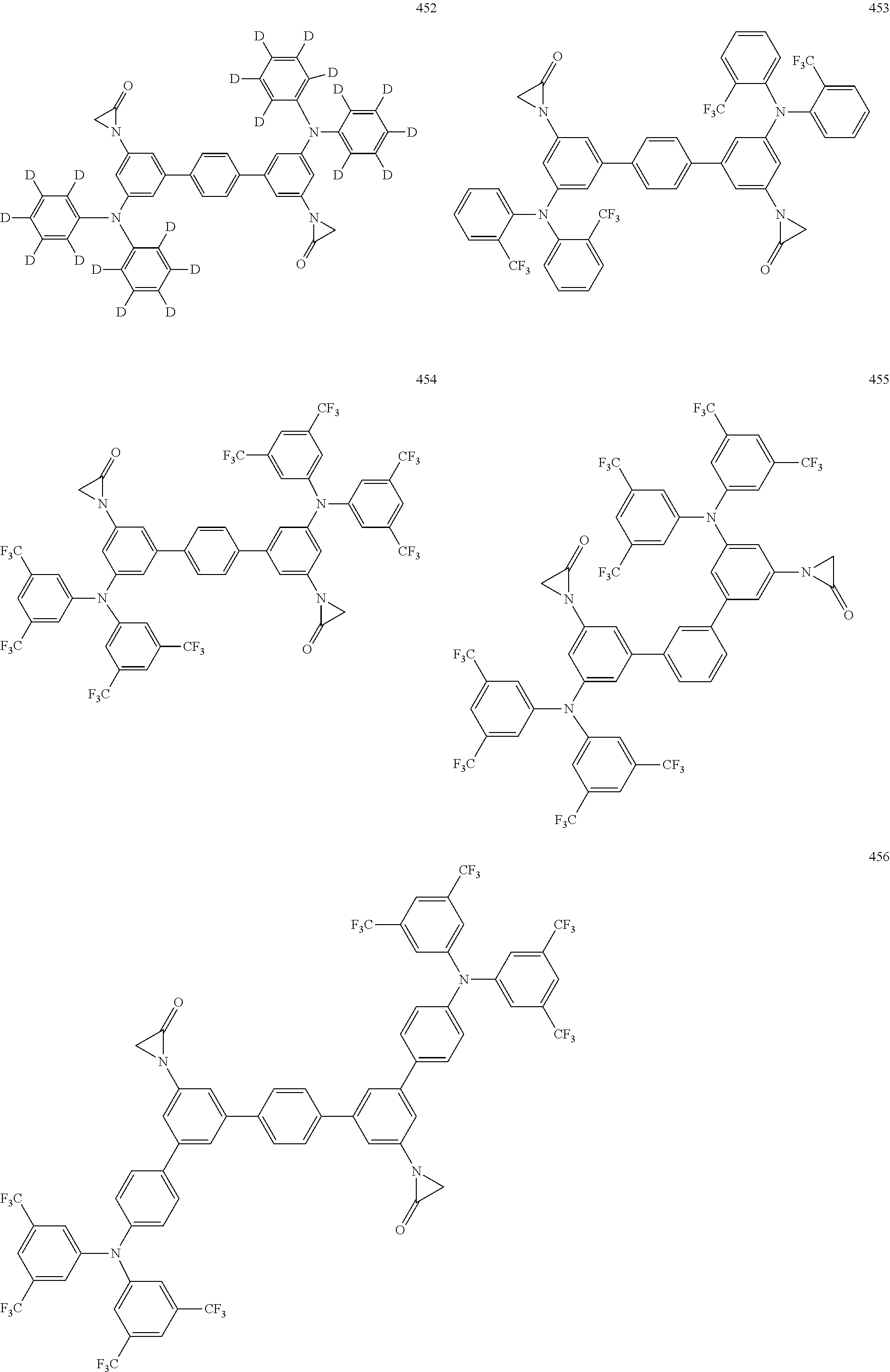

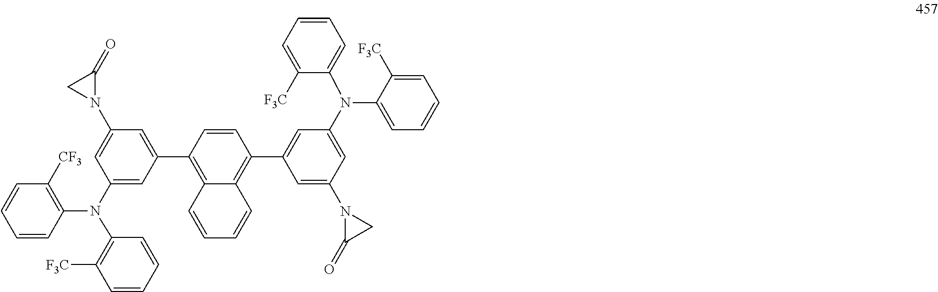 [SMILES: O=C1CN1c1cc(-c2ccc(-c3cc(N4CC4=O)cc(N(c4ccccc4C(F)(F)F)c4ccccc4C(F)(F)F)c3)c3ccccc23)cc(N(c2ccccc2C(F)(F)F)c2ccccc2C(F)(F)F)c1]

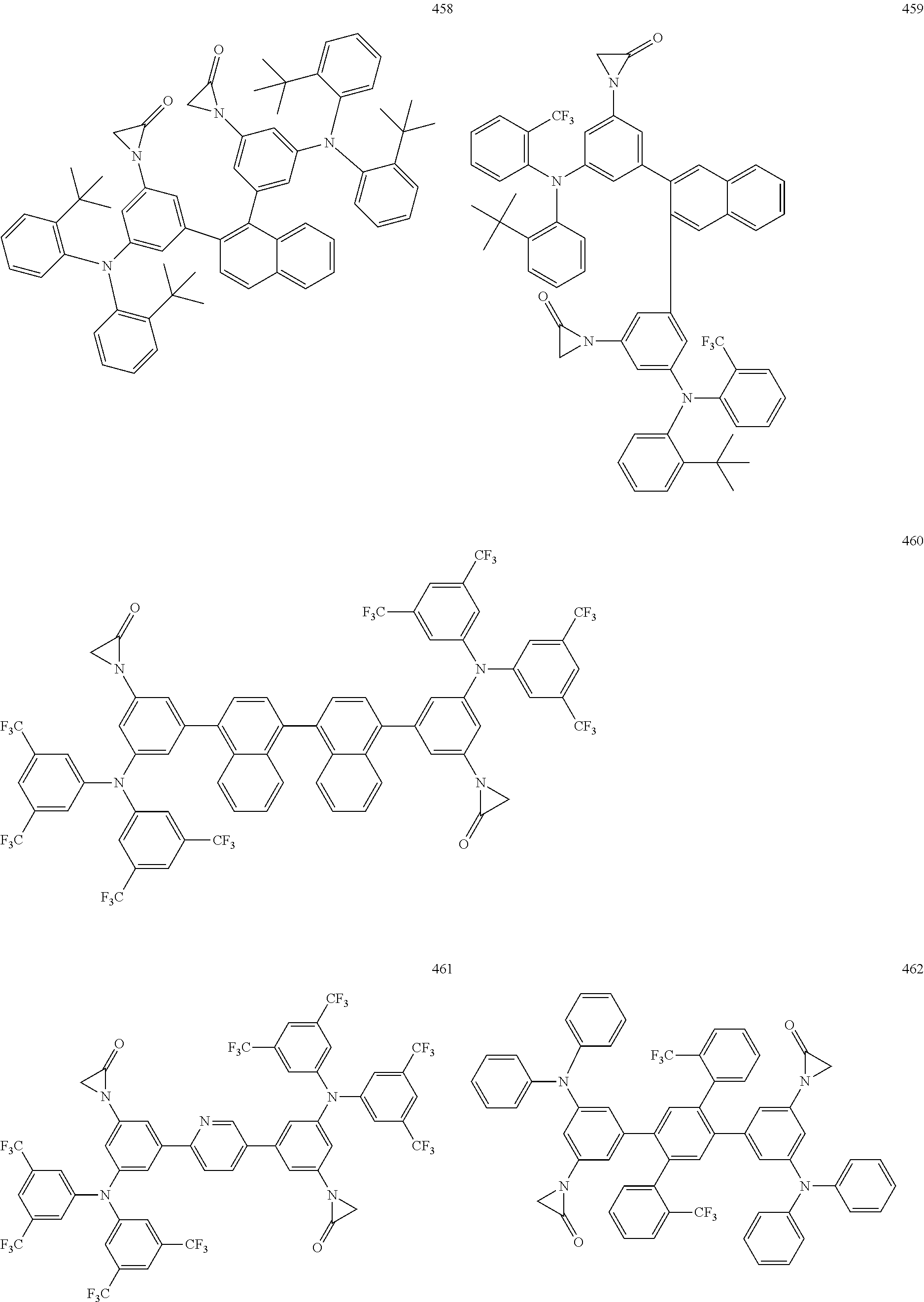

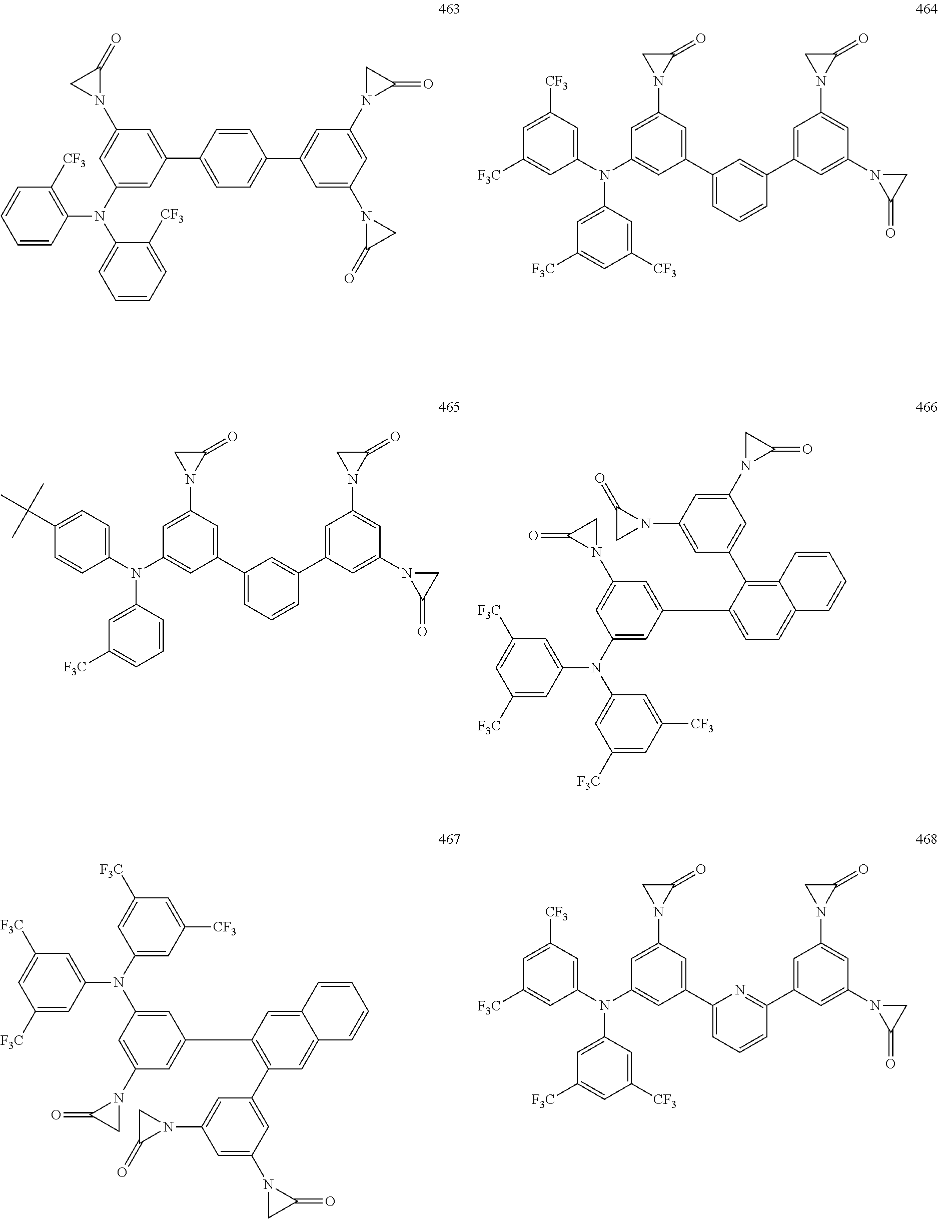

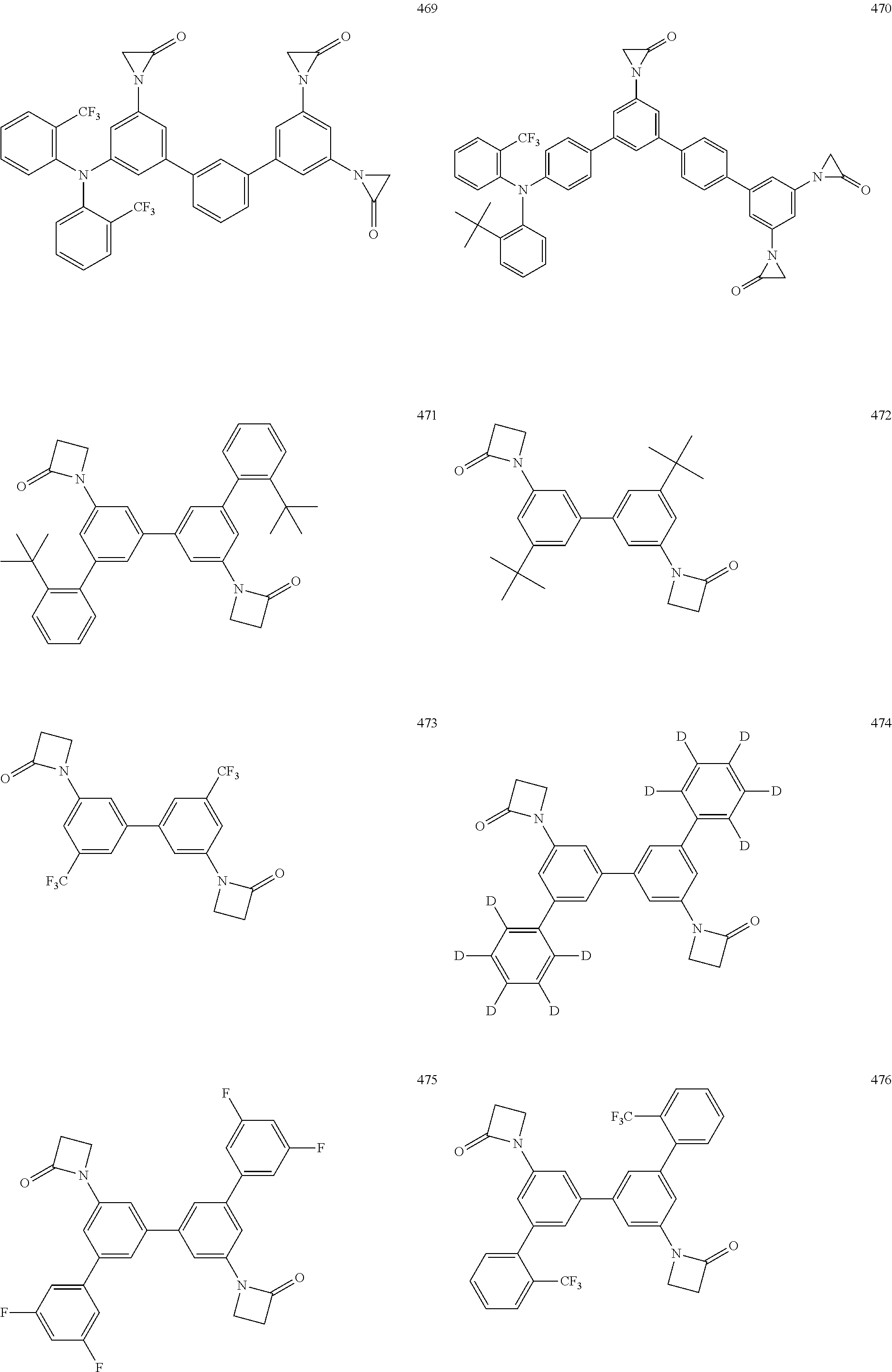

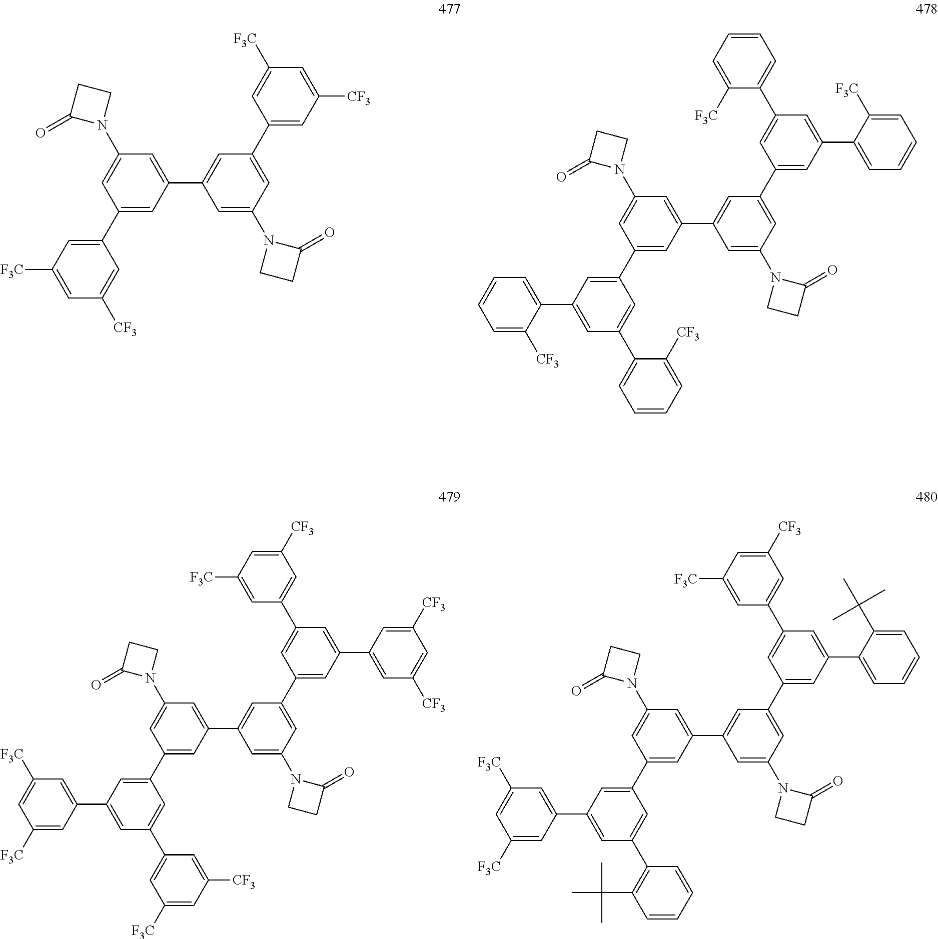

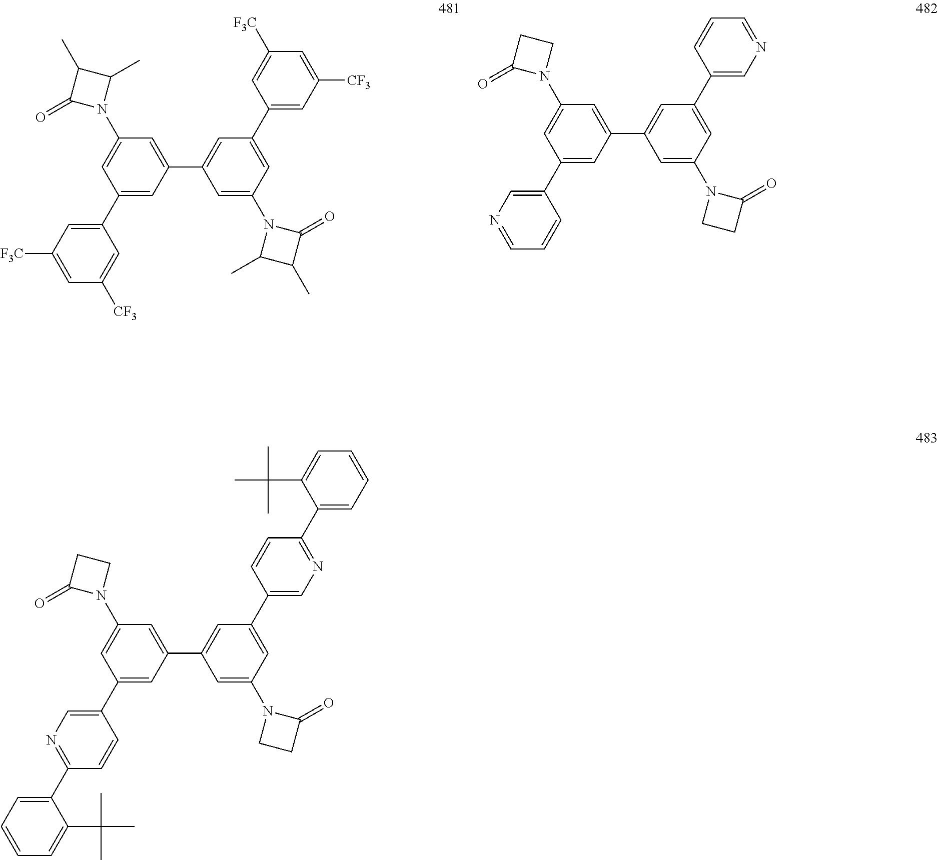 [SMILES: CC1C(=O)N(c2cc(-c3cc(-c4cc(C(F)(F)F)cc(C(F)(F)F)c4)cc(N4C(=O)C(C)C4C)c3)cc(-c3cc(C(F)(F)F)cc(C(F)(F)F)c3)c2)C1C ; O=C1CCN1c1cc(-c2cccnc2)cc(-c2cc(-c3cccnc3)cc(N3CCC3=O)c2)c1 ; CC(C)(C)c1ccccc1-c1ccc(-c2cc(-c3cc(-c4ccc(-c5ccccc5C(C)(C)C)nc4)cc(N4CCC4=O)c3)cc(N3CCC3=O)c2)cn1]

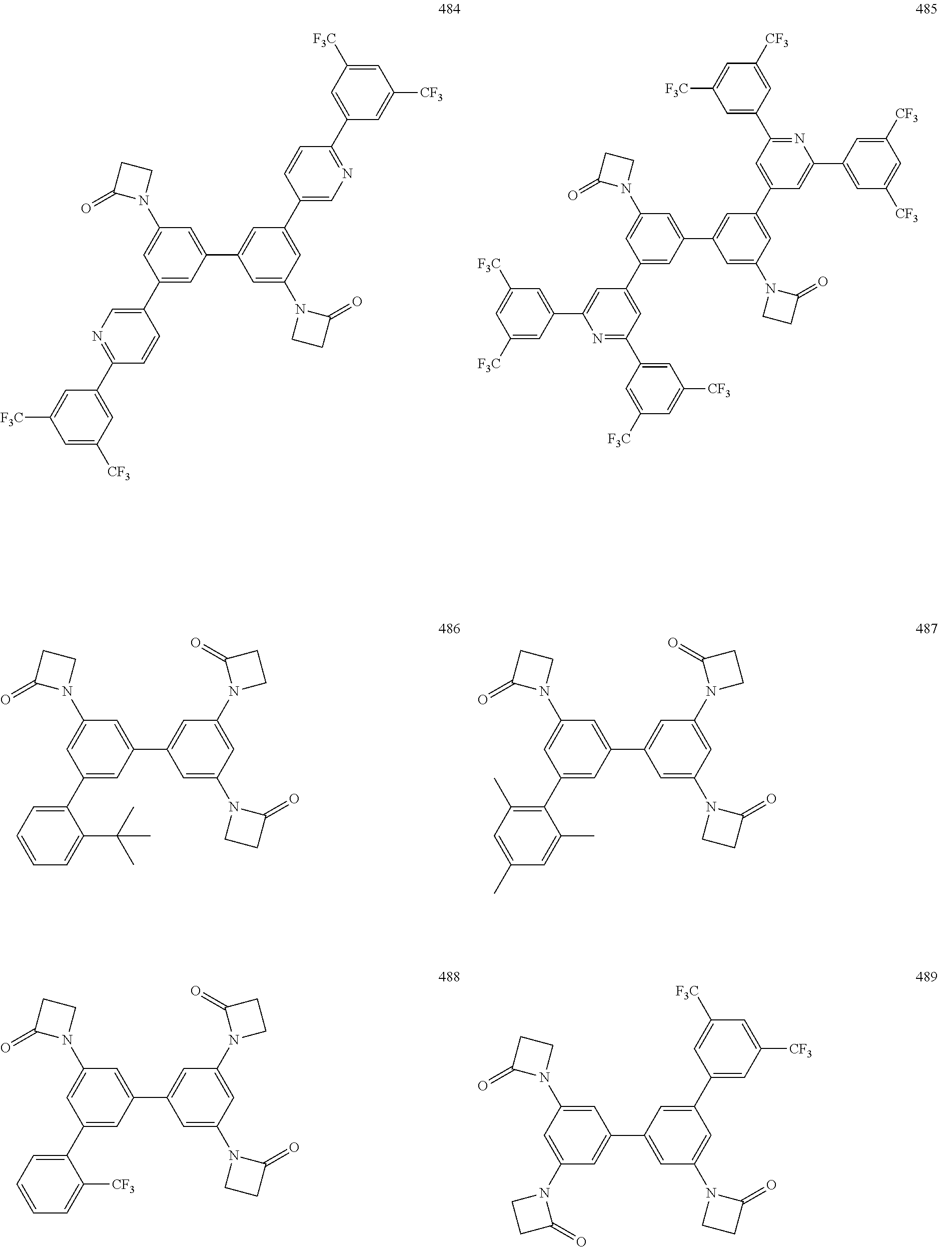

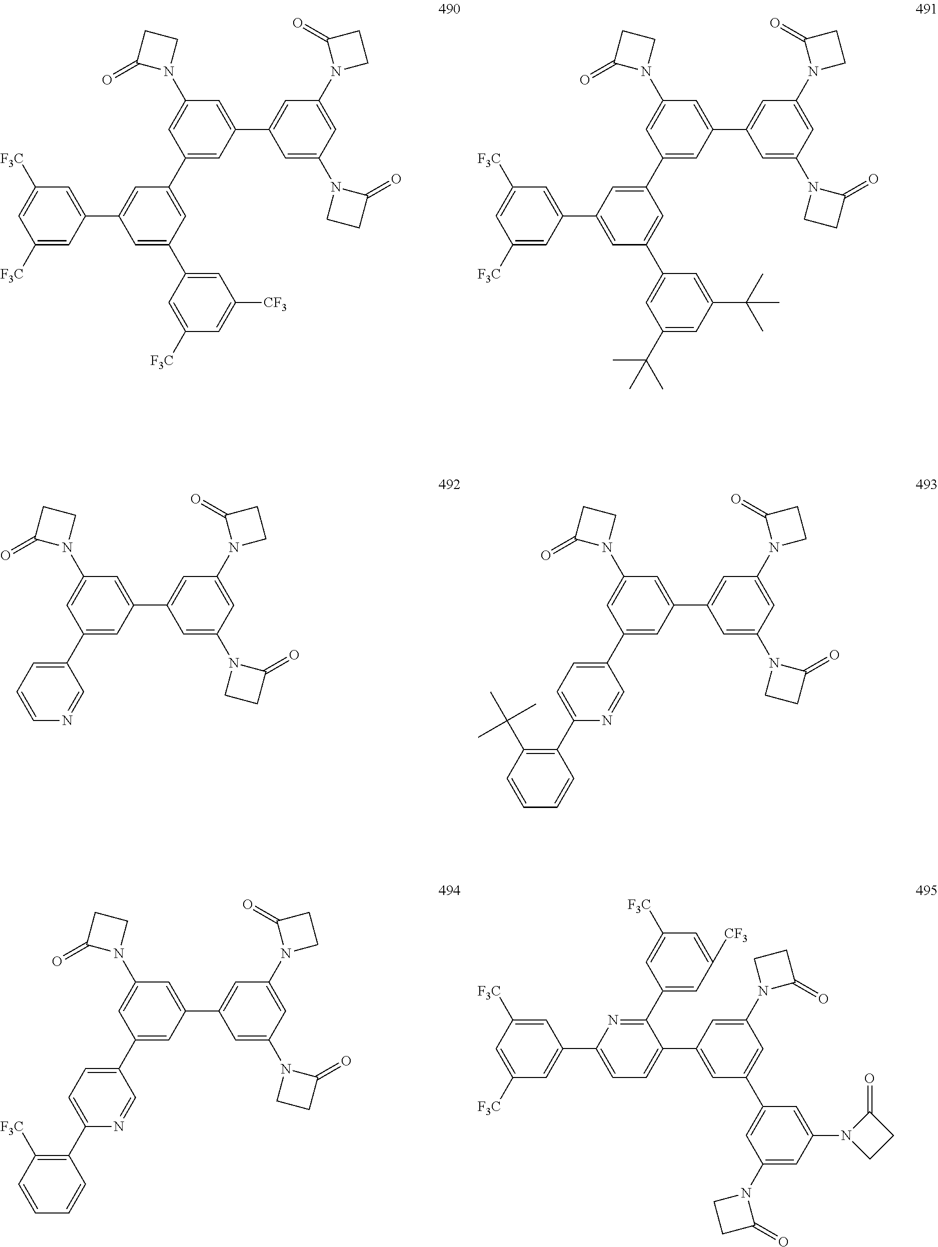

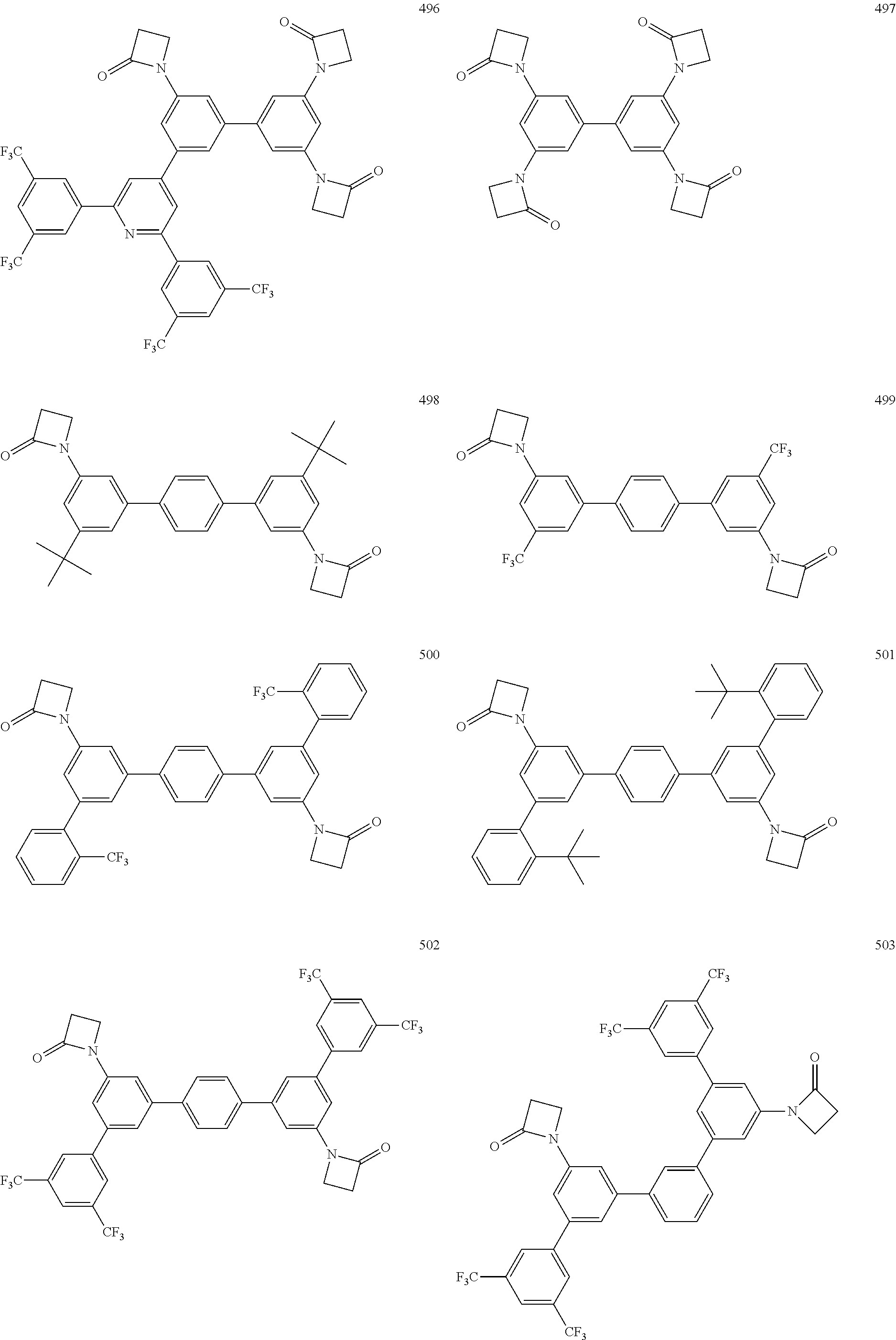

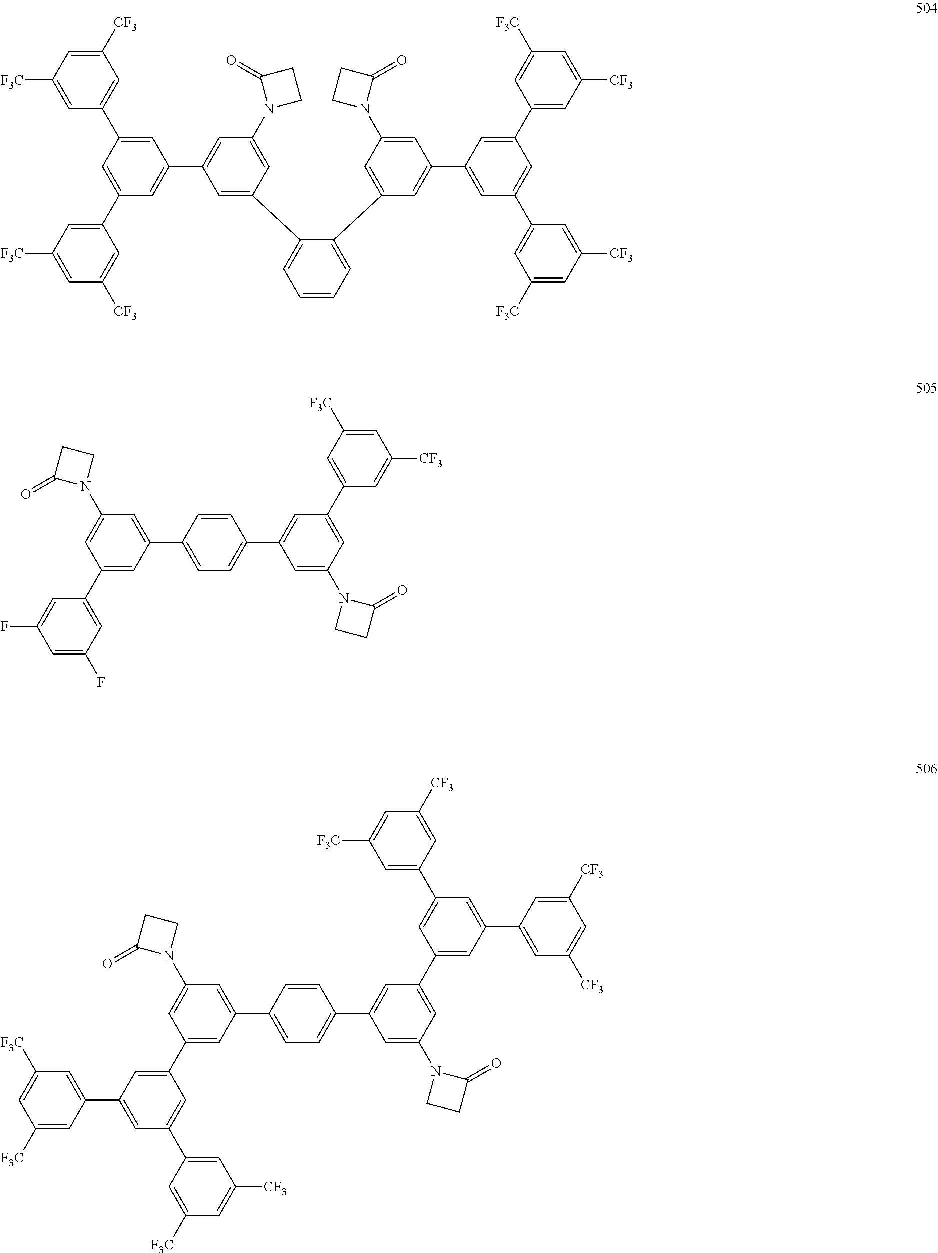 [SMILES: O=C1CCN1c1cc(-c2cc(-c3cc(C(F)(F)F)cc(C(F)(F)F)c3)cc(-c3cc(C(F)(F)F)cc(C(F)(F)F)c3)c2)cc(-c2ccccc2-c2cc(-c3cc(-c4cc(C(F)(F)F)cc(C(F)(F)F)c4)cc(-c4cc(C(F)(F)F)cc(C(F)(F)F)c4)c3)cc(N3CCC3=O)c2)c1 ; O=C1CCN1c1cc(-c2ccc(-c3cc(-c4cc(C(F)(F)F)cc(C(F)(F)F)c4)cc(N4CCC4=O)c3)cc2)cc(-c2cc(F)cc(F)c2)c1 ; O=C1CCN1c1cc(-c2ccc(-c3cc(-c4cc(-c5cc(C(F)(F)F)cc(C(F)(F)F)c5)cc(-c5cc(C(F)(F)F)cc(C(F)(F)F)c5)c4)cc(N4CCC4=O)c3)cc2)cc(-c2cc(-c3cc(C(F)(F)F)cc(C(F)(F)F)c3)cc(-c3cc(C(F)(F)F)cc(C(F)(F)F)c3)c2)c1]

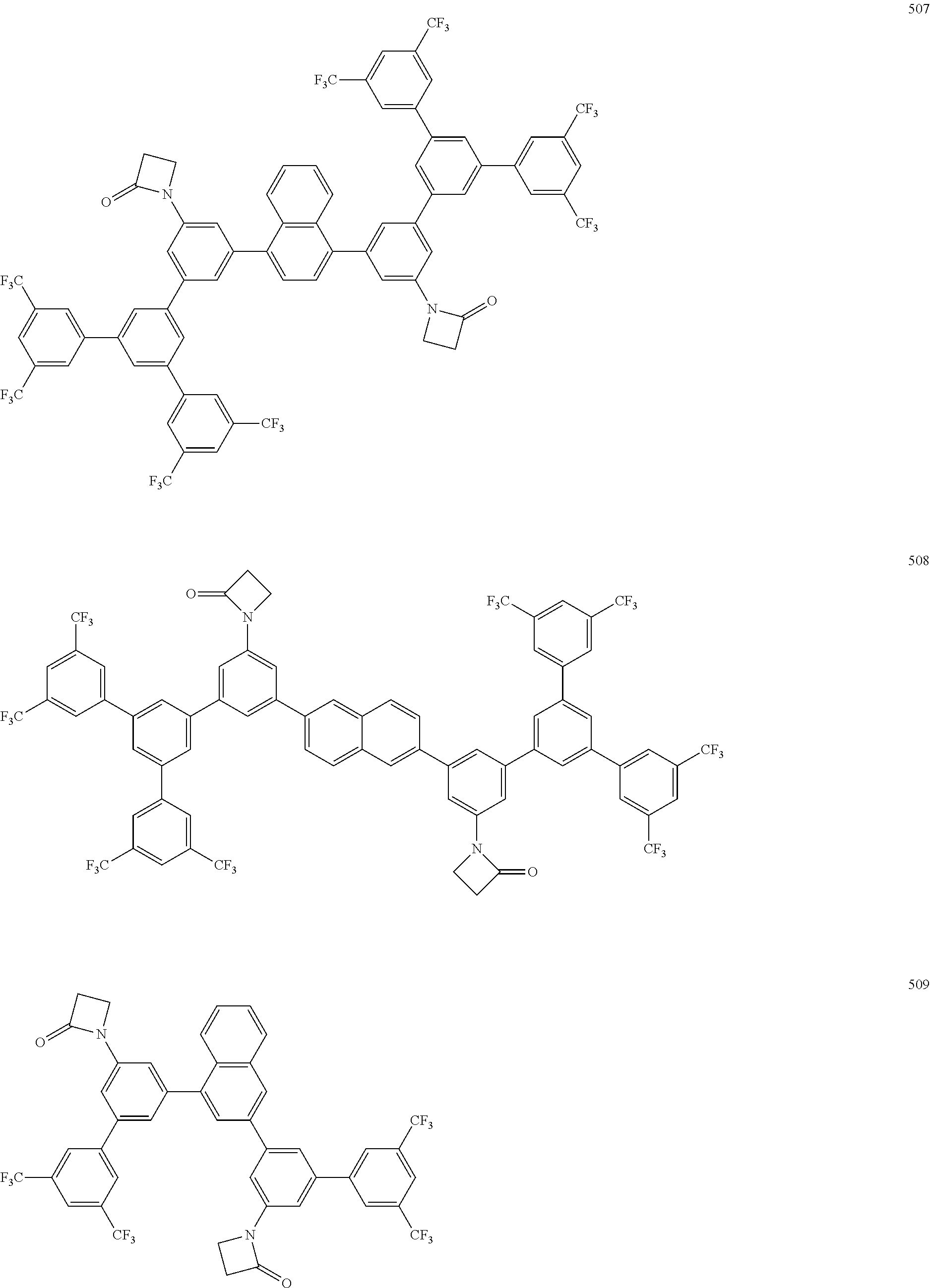

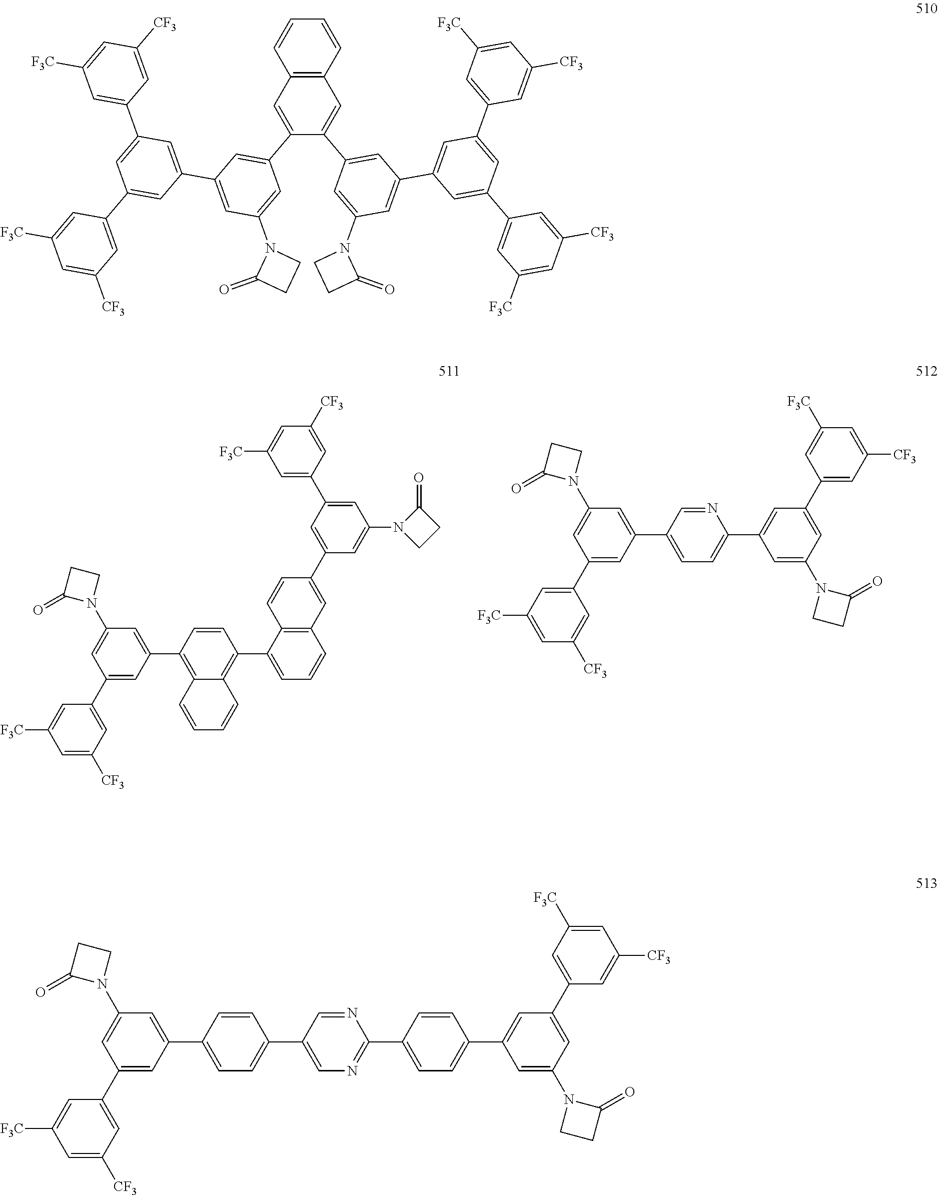

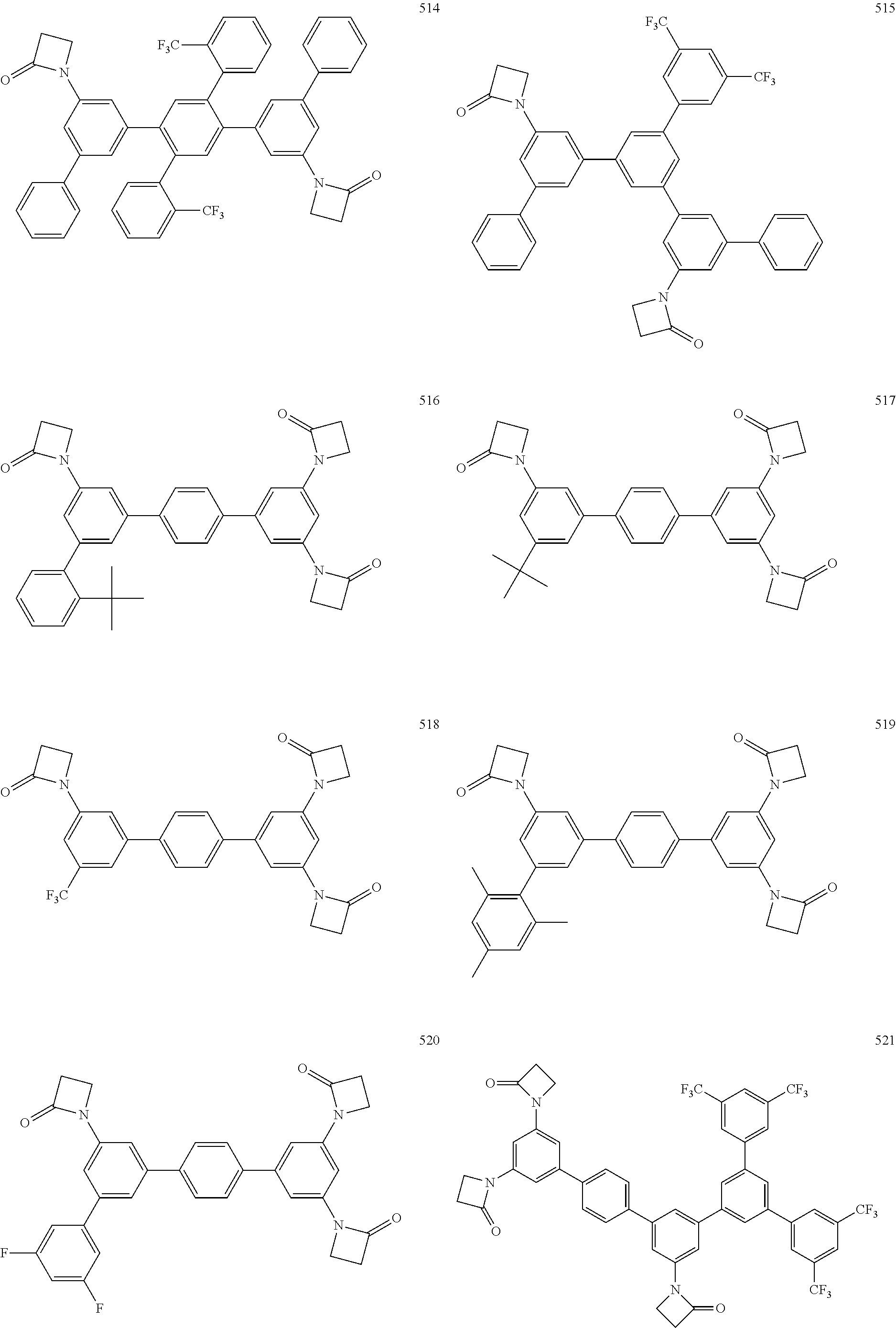

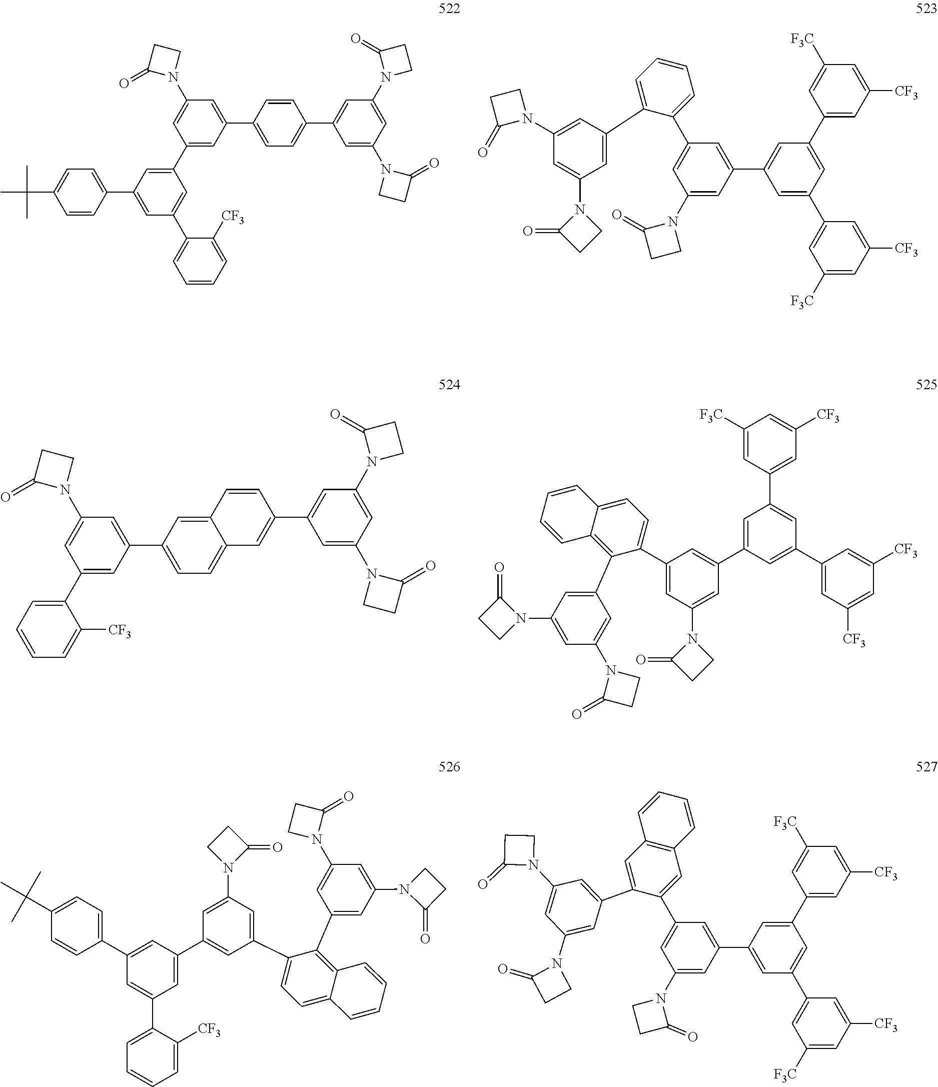

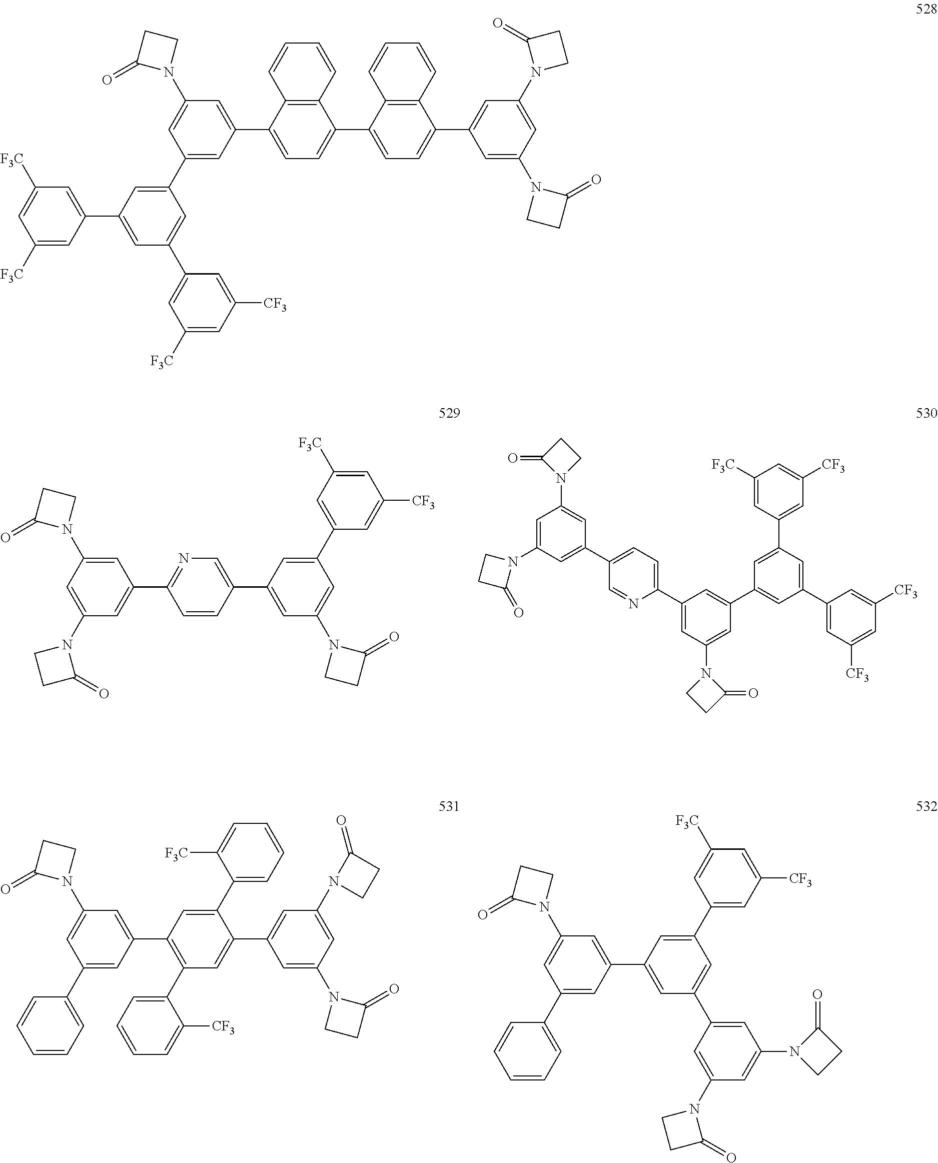

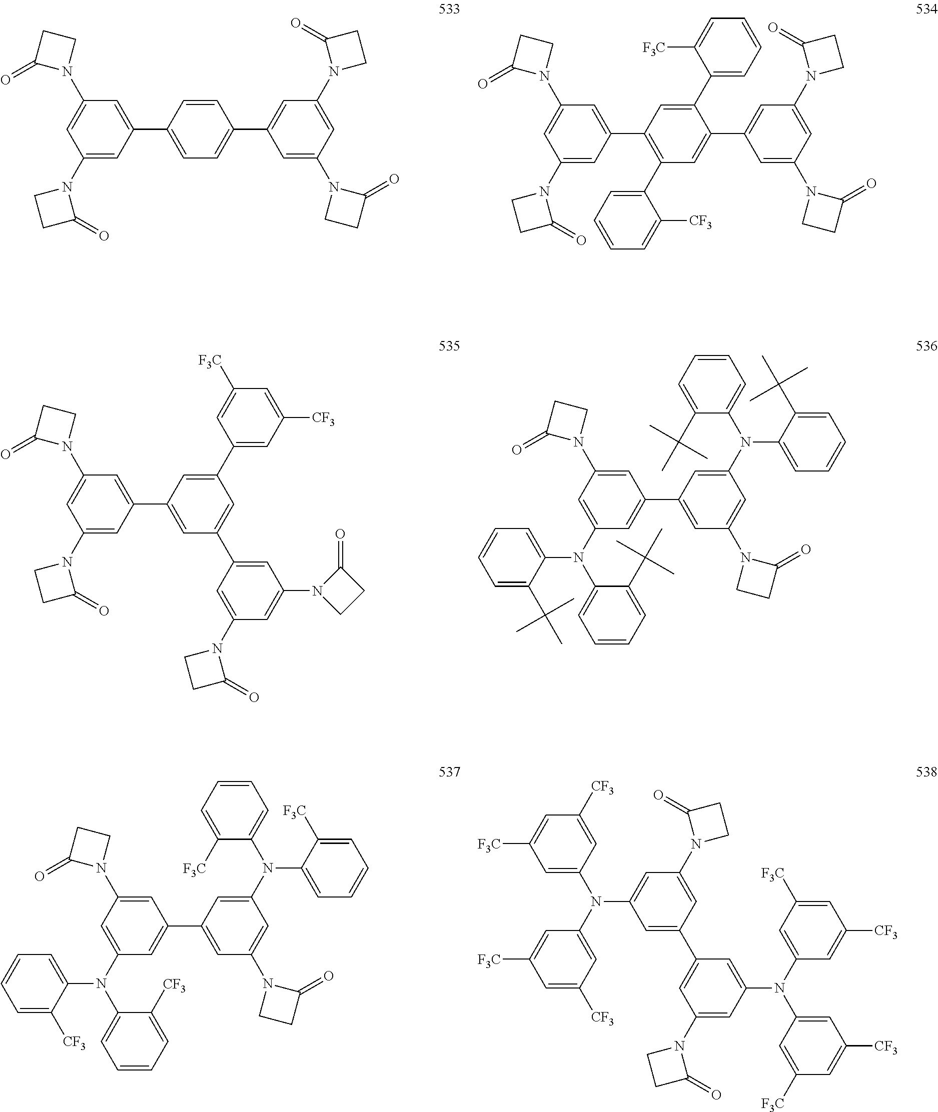

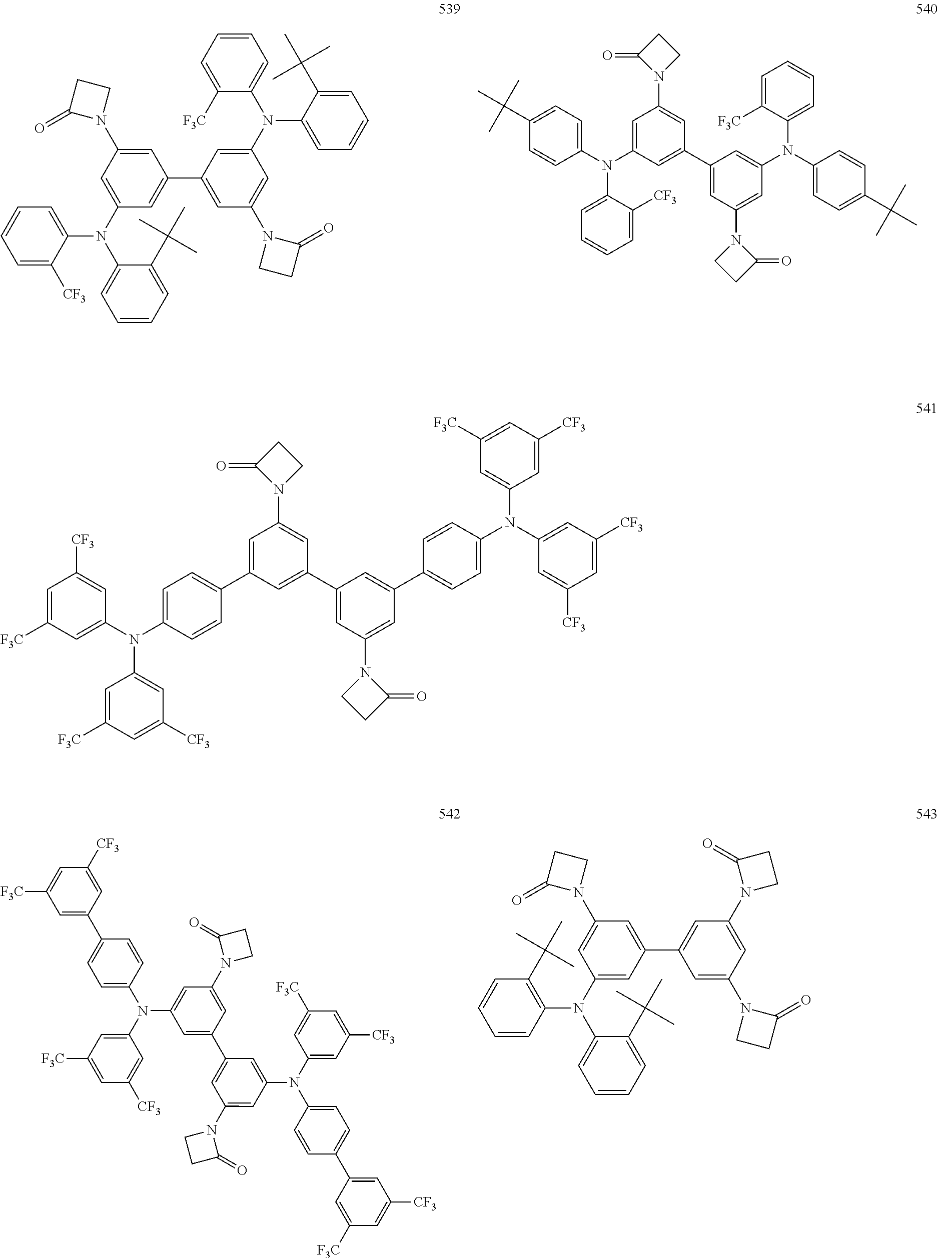

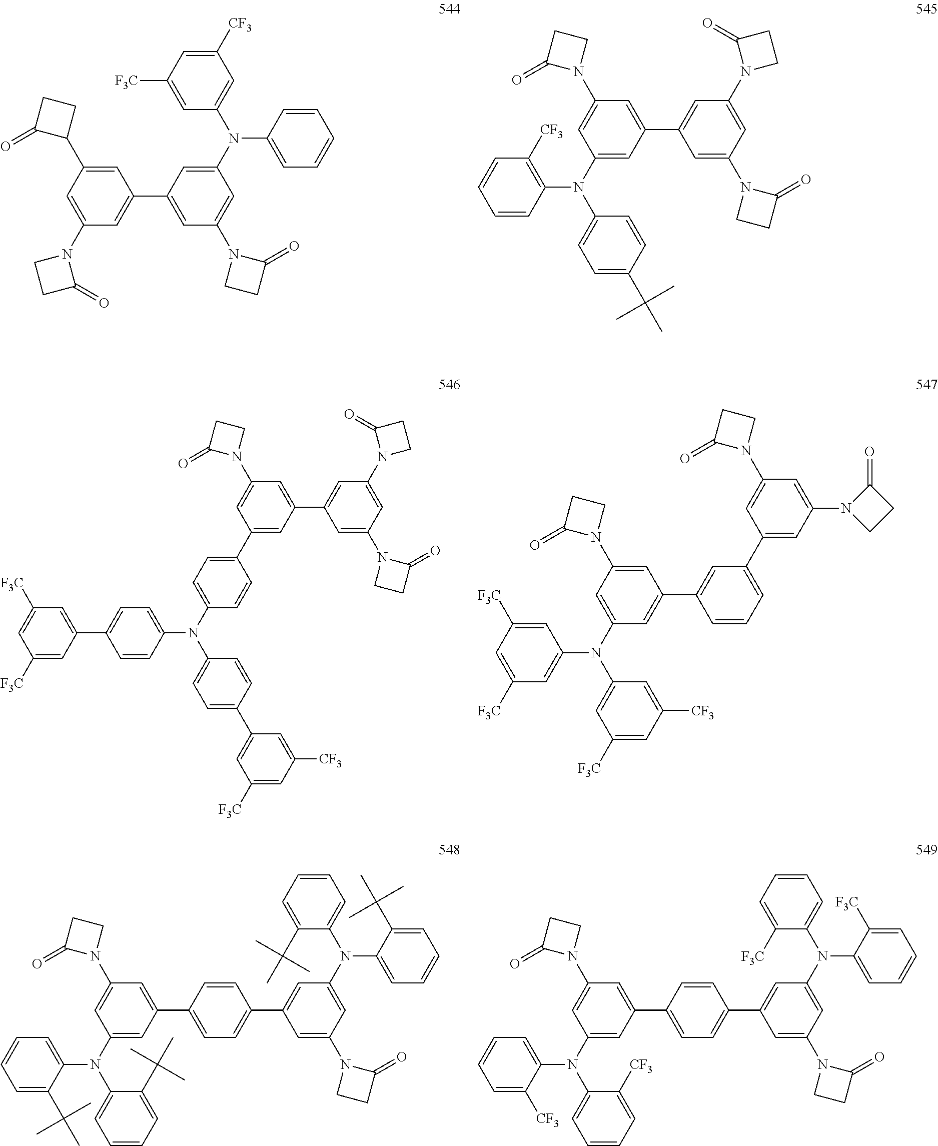

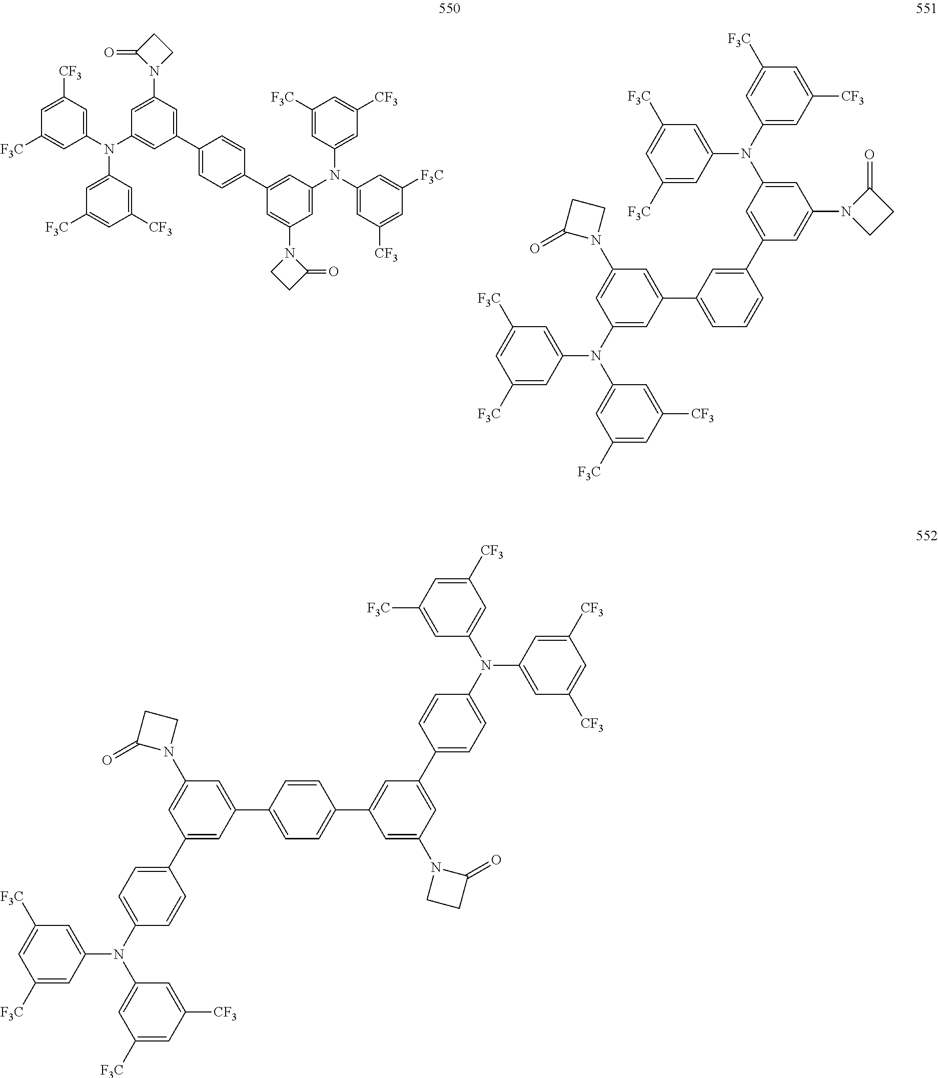

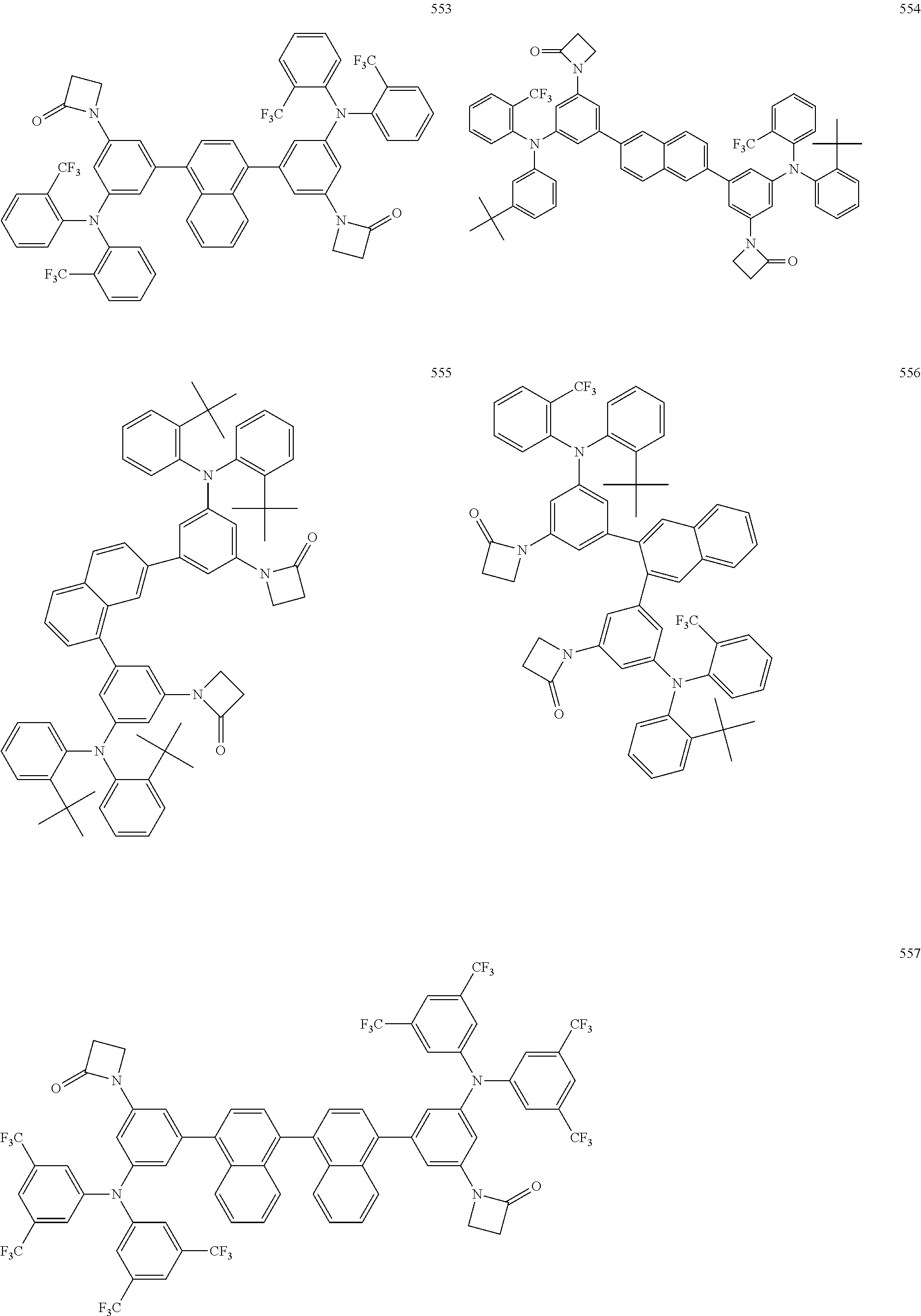

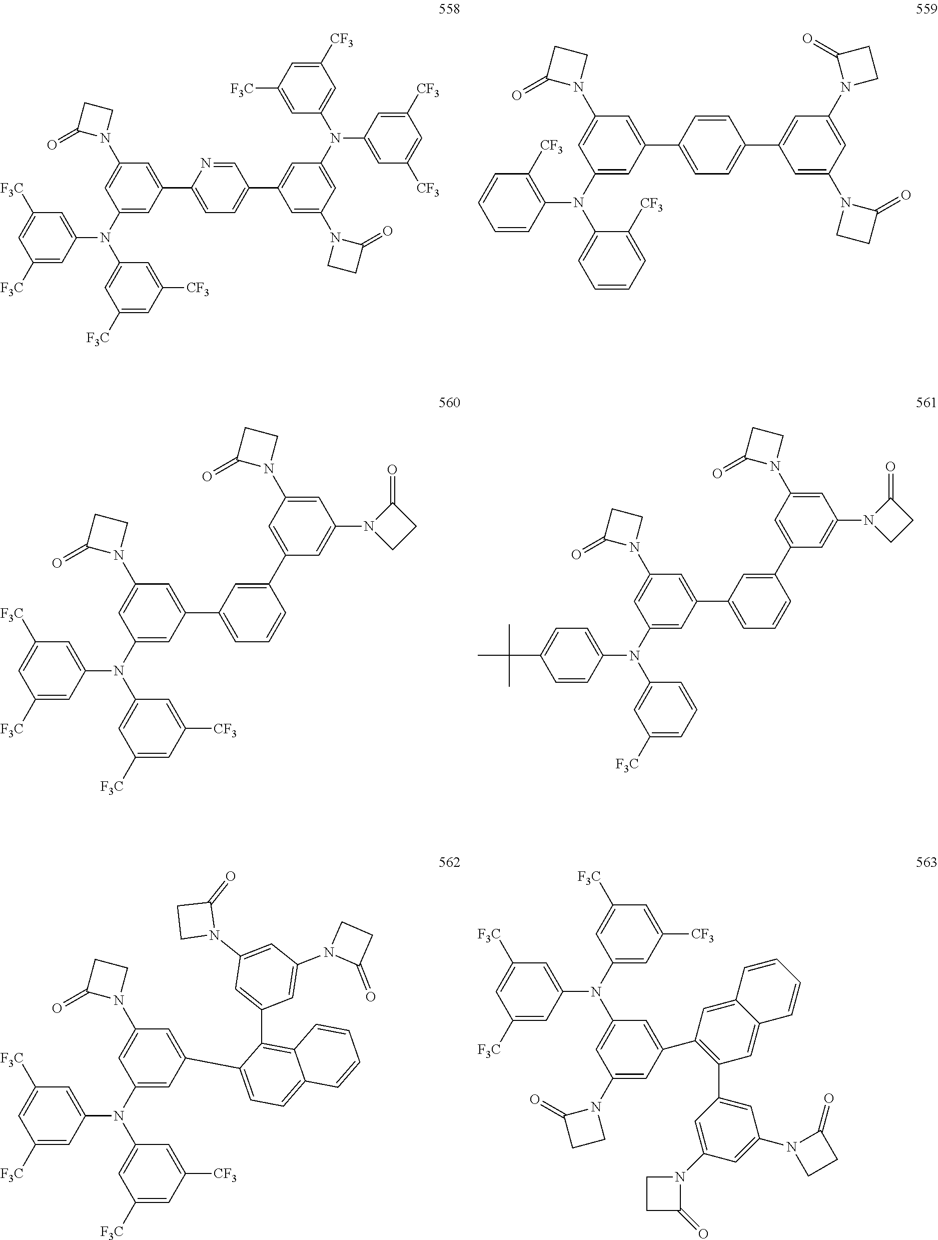

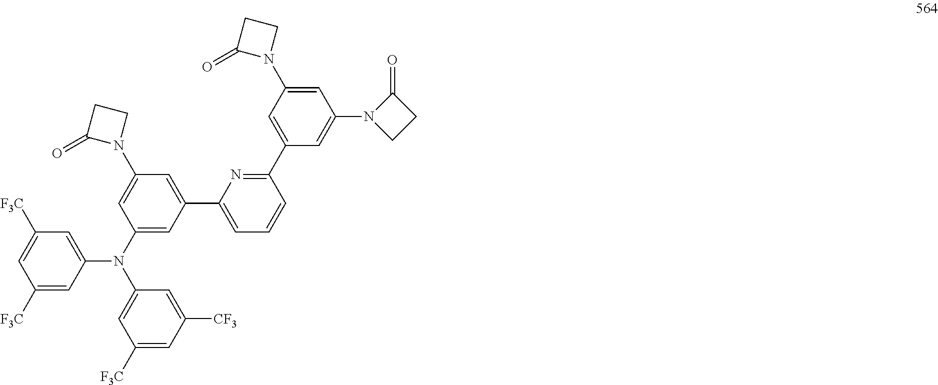 [SMILES: O=C1CCN1c1cc(-c2cccc(-c3cc(N4CCC4=O)cc(N(c4cc(C(F)(F)F)cc(C(F)(F)F)c4)c4cc(C(F)(F)F)cc(C(F)(F)F)c4)c3)n2)cc(N2CCC2=O)c1]

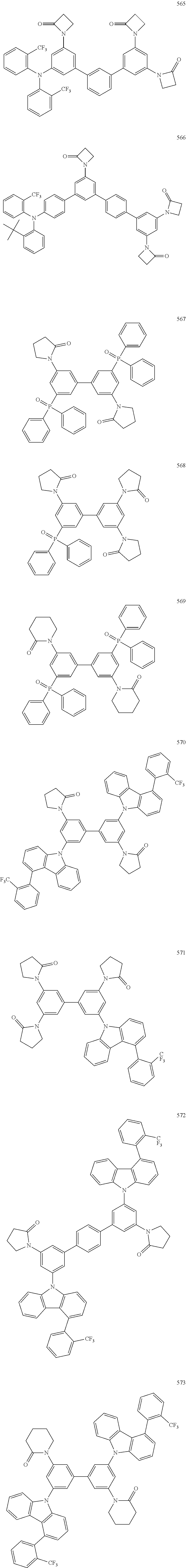

As such, the organic compound according to the present invention can synthesize organic compounds with various properties using moieties with unique properties. As a result, when the organic compound according to the present invention is applied to the light efficiency improving layer provided in the organic light-emitting device, it is possible to further improve the luminescent properties such as luminous efficiency, etc. of the device.

In addition, the compound of an embodiment of the present invention may be applied to a device according to a general method for manufacturing an organic light-emitting device.

An organic light-emitting device according to an embodiment of the present invention may include a first electrode, a second electrode, and an organic layer arranged therebetween. The organic light-emitting device may be manufactured using a general device manufacturing method and material, except that the organic compound of an embodiment of the present invention is used to form the organic layer of the device.

The organic layer of the organic light-emitting device according to an embodiment of the present invention may have a monolayer structure or a multilayer structure in which two or more organic layers are stacked. For example, the structure of the organic layers may include a hole injecting layer, a hole transport layer, a light emitting layer, an electron transport layer, an electron injecting layer, an electron blocking layer, a hole blocking layer, and a light efficiency improving layer (capping layer). The number of the organic layers is not limited and may be increased or decreased.

In addition, the organic light-emitting device may include a substrate, a first electrode (anode), an organic layer, a second electrode (cathode), and a light efficiency improving layer, and the light efficiency improving layer may be formed under the first electrode (bottom emission) or on the second electrode (top emission).

When the light efficiency improving layer is formed on the second electrode (top emission), light from the light emitting layer is emitted to the cathode and passes through the light efficiency improving layer (CPL) formed using the compound according to an embodiment of the present invention having a relatively high refractive index, so that the wavelength of the light is amplified, resulting in an increase in luminous efficiency.

Preferred structures of the organic layers of the organic light-emitting device according to an embodiment of the present invention will be explained in more detail in the examples to be described later.

In addition, the organic light-emitting device of an embodiment of the present invention may be manufactured by depositing a metal, a conductive metal oxide or an alloy thereof on a substrate by a physical vapor deposition (PVD) method such as sputtering or e-beam evaporation to form an anode, forming organic layers including a hole injecting layer, a hole transport layer, a light emitting layer, and an electron transport layer thereon, and depositing a cathode material thereon.

In addition to the above methods, the organic light-emitting device may be fabricated by depositing a cathode material, organic layer materials, and an anode material in this order on a substrate. The organic layers may have a multilayer structure including a hole injecting layer, a hole transport layer, a light emitting layer, and an electron transport layer, but is not limited thereto and may have a monolayer structure. In addition, the organic layers may be manufactured in a smaller number of layers by a solvent process using various polymer materials rather than by a deposition process, such as spin coating, dip coating, doctor blading, screen printing, inkjet printing or thermal transfer.

As the anode material, a material having a high work function is generally preferred for easy injection of holes into the organic layers. Specific examples of anode materials suitable for use in an embodiment of the present invention include, but are not limited to: metals such as vanadium, chromium, copper, zinc, and gold and alloys thereof; metal oxides such as zinc oxide, indium oxide, indium thin oxide (ITO), and indium zinc oxide (IZO); combinations of metals and oxides such as ZnO:Al and SnO₂:Sb; and conductive polymers such as poly(3-methylthiophene), poly[3,4-(ethylene-1,2-dioxy)thiophene](PEDT), polypyrrole, and polyaniline.

As the cathode material, a material having a low work function is generally preferred for easy injection of electrons into the organic layers. Specific examples of suitable cathode materials include, but are not limited to: metals such as magnesium, calcium, sodium, potassium, titanium, indium, yttrium, lithium, gadolinium, aluminum, silver, tin, and lead and alloys thereof; and multilayer structure materials such as LiF/Al and LiO₂/Al.

The hole injecting material is preferably a material that may receive holes injected from the anode at low voltage. The highest occupied molecular orbital (HOMO) of the hole injecting material is preferably between the work function of the anode material and the HOMO of the adjacent organic layer. Specific examples of hole injecting materials include, but are not limited to, metal porphyrin, oligothiophene, arylamine-based organic materials, hexanitrile hexaazatriphenylene, quinacridone-based organic materials, perylene-based organic materials, anthraquinone, polyaniline, and polythiophene-based conductive polymers.

The hole transport material is a material that may receive holes transported from the anode or the hole injecting layer and may transfer the holes to the light emitting layer. A material with high hole mobility is suitable. Specific examples thereof include arylamine-based organic materials, conductive polymers, and block copolymers consisting of conjugated and non-conjugated segments. The use of the organic compound according to an embodiment of the present invention ensures further improved low-voltage driving characteristics, high luminous efficiency, and life characteristics of the device.

The light emitting material is a material that may receive and recombine holes from the hole transport layer and electrons from the electron transport layer to emit light in the visible ray area. A material with high quantum efficiency for fluorescence and phosphorescence is preferred. Specific examples thereof include, but are not limited to, 8-hydroxyquinoline aluminum complex (Alq₃), carbazole-based compounds, dimerized styryl compounds, BAlq, 10-hydroxybenzoquinoline-metal compounds, benzoxazole-based compounds, benzthiazole-based compounds, and benzimidazole-based compounds, poly(p-phenylenevinylene) (PPV)-based polymers, spiro compounds, polyfluorene, and rubrene.

The electron transport material is a material that may receive electrons injected from the cathode and may transfer the electrons to the light emitting layer. A material with high electron mobility is suitable. Specific examples thereof include, but are not limited to, 8-hydroxyquinoline Al complex, Alq₃ complexes, organic radical compounds, hydroxyflavone-metal complexes.

The organic light-emitting device according to an embodiment of the present invention may be of a top emission, bottom emission or dual emission type according to the materials used.

In addition, the organic compound according to an embodiment of the present invention may perform its function even in organic electronic devices, including organic solar cells, organic photoconductors, and organic transistors, based on a similar principle to that applied to the organic light-emitting device.

### MODE FOR CARRYING OUT INVENTION

Hereinafter, the present invention will be explained in more detail with reference to the preferred examples. However, these examples are provided for illustrative purposes and do not serve to limit the scope of the invention. It will be obvious to those skilled in the art that various modifications and changes are possible without departing from the scope and technical spirit of the present invention.

### Synthesis Example 1: Synthesis of Compound 1

### (1) Preparation Example 1: Synthesis of Intermediate 1-1

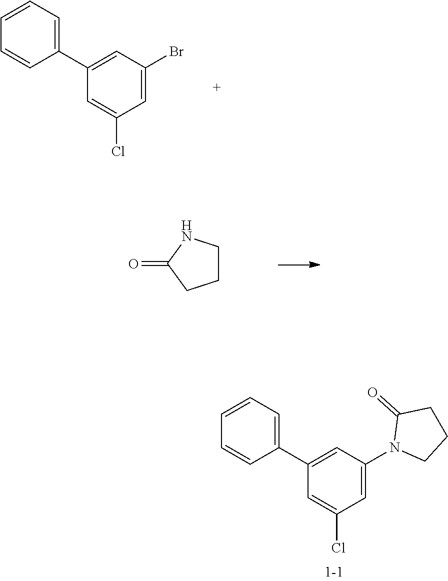 [SMILES: Clc1cc(Br)cc(-c2ccccc2)c1 ; O=C1CCCN1 ; O=C1CCCN1c1cc(Cl)cc(-c2ccccc2)c1]

1-Bromo-3-dichlorobenzene (10.0 g, 0.037 mol), 2-pyrrolidinone (3.8 g, 0.050 mol), Cs₂CO₃ (17.1 g, 0.052 mol), Pd(dba)₂ (1.1 g, 0.002 mol), Xant-Phos (3.9 g, 0.007 mol), and dioxane were added and reacted while stirring under reflux for 16 hours. After completion of the reaction, the reaction mixture was extracted, concentrated, and then columned to obtain 8.7 g of Intermediate 1-1 (yield 85.7%).

### (2) Preparation Example 2: Synthesis of Intermediate 1-2

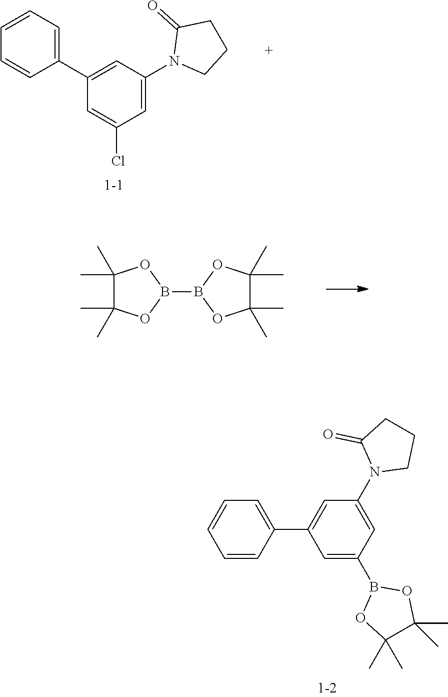

Intermediate 1-1 (10.0 g, 0.037 mol), Bis(pinacolato)diboron (11.2 g, 0.044 mol), KOAc (10.8 g, 0.110 mol), Pd(dppf)Cl₂ (1.4 g, 0.002 mol), and XPhos (5.3 g, 0.011 mol) were added with dioxane 200 mL and reacted while stirring at 100° C. for 12 hours. After completion of the reaction, the reaction mixture was extracted, concentrated, and then column and recrystallized to obtain 9.8 g of Intermediate 1-2 (yield 73.3%).

### (3) Preparation Example 3. Synthesis of Compound 1

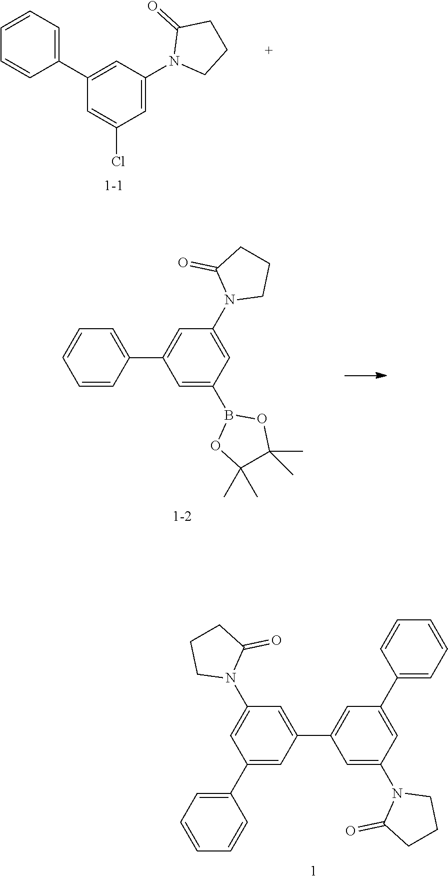 [SMILES: O=C1CCCN1c1cc(Cl)cc(-c2ccccc2)c1 ; CC1(C)OB(c2cc(-c3ccccc3)cc(N3CCCC3=O)c2)OC1(C)C ; O=C1CCCN1c1cc(-c2ccccc2)cc(-c2cc(-c3ccccc3)cc(N3CCCC3=O)c2)c1]

Intermediate 1-1 (10.0 g, 0.037 mol), Intermediate 1-2 (16.0 g, 0.044 mol), K₂CO₃ (15.3 g, 0.110 mol), Pd(OAc)₂ (2.1 g, 0.002 mol), X-Phos (1.8 g, 0.004 mol), THF 200 mL, and H₂O 50 mL were added and reacted while stirring at 70° C. for 6 hours. After completion of the reaction, the reaction mixture was extracted, concentrated, and then column and recrystallized to obtain 13.1 g of Compound 1 (yield 75.3%).

LC/MS: m/z=472[(M)⁺]

### Synthesis Example 2: Synthesis of Compound 17

### (1) Preparation Example 1: Synthesis of Intermediate 17-1

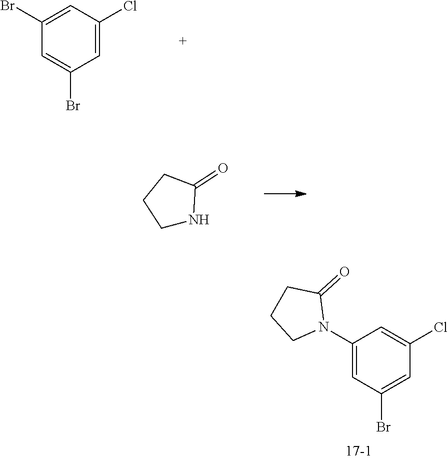 [SMILES: Clc1cc(Br)cc(Br)c1 ; O=C1CCCN1 ; O=C1CCCN1c1cc(Cl)cc(Br)c1]

1,3-dibromo-5-chlorobenzene (10.0 g, 0.037 mol), 2-pyrrolidinone (3.8 g, 0.044 mol), Cs₂CO₃ (16.9 g, 0.052 mol), Pd(dba)₂ (1.1 g, 0.002 mol), Xant-Phos (3.9 g, 0.007 mol), and dioxane were added and reacted while stirring under reflux for 16 hours. After completion of the reaction, the reaction mixture was extracted, concentrated, and then columned to obtain 6.3 g of Intermediate 17-1 (yield 62.0%).

### (2) Preparation Example 2: Synthesis of Intermediate 17-2

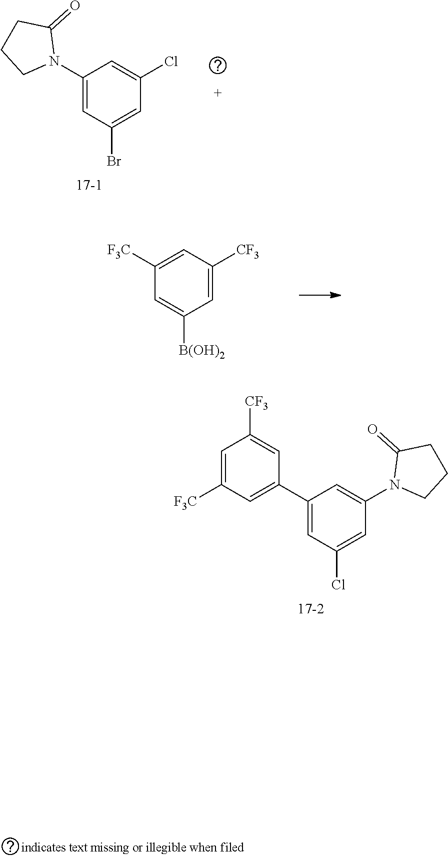 [SMILES: O=C1CCCN1c1cc(Cl)cc(Br)c1 ; OB(O)c1cc(C(F)(F)F)cc(C(F)(F)F)c1 ; O=C1CCCN1c1cc(Cl)cc(-c2cc(C(F)(F)F)cc(C(F)(F)F)c2)c1]

Intermediate 17-1 (10.0 g, 0.036 mol), 3,5-Bis(trifluoromethyl)phenylboronic acid (11.3 g, 0.044 mol), K₂CO₃ (15.1 g, 0.109 mol), and Pd(PPh₃)₄ (0.8 g, 0.0007 mol) were added with toluene 200 mL, ethanol 50 mL, and H₂O 50 mL and reacted while stirring at 80° C. for 6 hours. After completion of the reaction, the reaction mixture was extracted, concentrated, and then columned to obtain 10.5 g of Intermediate 17-2 (yield 70.7%).
(3) Preparation Example 3: Synthesis of Intermediate 17-3

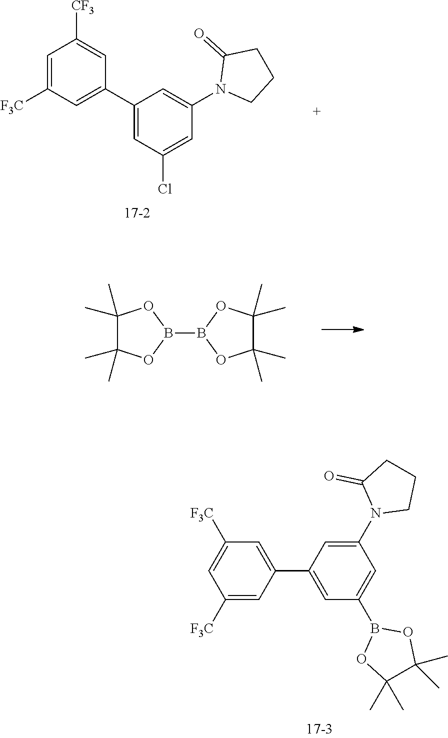

Intermediate 17-2 (10.0 g, 0.025 mol), Bis(pinacolato)diboron (16.9 g, 0.063 mol), KOAc (4.8 g, 0.049 mol), Pd(dppf)Cl₂ (0.5 g, 0.0007 mol), and XPhos (0.4 g, 0.0009 mol) were added with dioxane 200 mL and reacted while stirring at 100° C. for 12 hours. After completion of the reaction, the reaction mixture was extracted, concentrated, and then column and recrystallized to obtain 13.9 g of Intermediate 17-3 (yield 71.5%).

### (4) Preparation Example 4: Synthesis of Compound 17

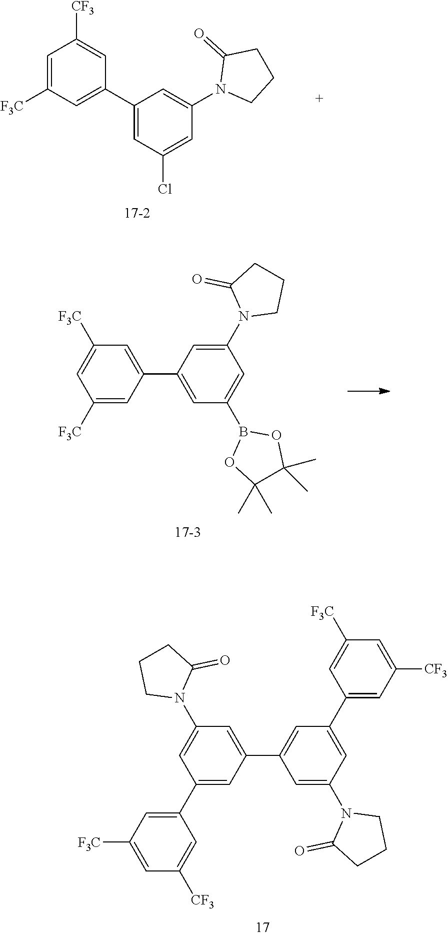 [SMILES: O=C1CCCN1c1cc(Cl)cc(-c2cc(C(F)(F)F)cc(C(F)(F)F)c2)c1 ; CC1(C)OB(c2cc(-c3cc(C(F)(F)F)cc(C(F)(F)F)c3)cc(N3CCCC3=O)c2)OC1(C)C ; O=C1CCCN1c1cc(-c2cc(-c3cc(C(F)(F)F)cc(C(F)(F)F)c3)cc(N3CCCC3=O)c2)cc(-c2cc(C(F)(F)F)cc(C(F)(F)F)c2)c1]

Intermediate 17-2 (10.0 g, 0.025 mol), Intermediate 17-3 (14.7 g, 0.029 mol), K₂CO₃ (10.2 g, 0.074 mol), Pd(OAc)₂ (1.4 g, 0.001 mol), X-Phos (1.2 g, 0.003 mol), THF 200 mL, and H₂O 50 mL were added and reacted while stirring at 70° C. for 6 hours. After completion of the reaction, the reaction mixture was extracted, concentrated, and then column and recrystallized to obtain 12.2 g of Compound 17 (yield 66.8%).

LC/MS: m/z=744[(M)⁺]

### Synthesis Example 3: Synthesis of Compound 59

### (1) Preparation Example 1: Synthesis of Intermediate 59-1

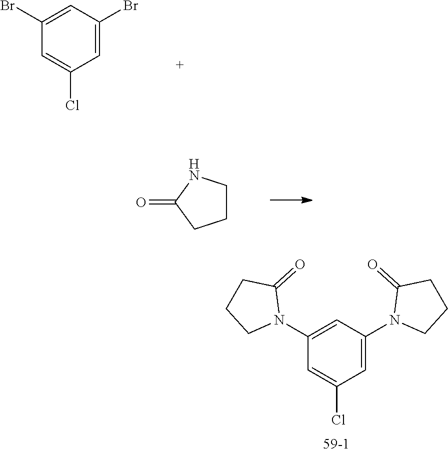 [SMILES: Clc1cc(Br)cc(Br)c1 ; O=C1CCCN1 ; O=C1CCCN1c1cc(Cl)cc(N2CCCC2=O)c1]

1,3-Dibromo-5-chlorobenzene (10.0 g, 0.037 mol), 2-Pyrrolidinone (7.6 g, 0.089 mol), Cs₂CO₃ (33.7 g, 0.104 mol), Pd(dba)₂ (2.1 g, 0.004 mol), Xant-Phos (15.4 g, 0.027 mol), and dioxane were added and reacted while stirring under reflux for 16 hours. After completion of the reaction, the reaction mixture was extracted, concentrated, and then columned to obtain 8.4 g of Intermediate 59-1 (yield 81.5%).

### (2) Preparation Example 2: Synthesis of Intermediate 59-2

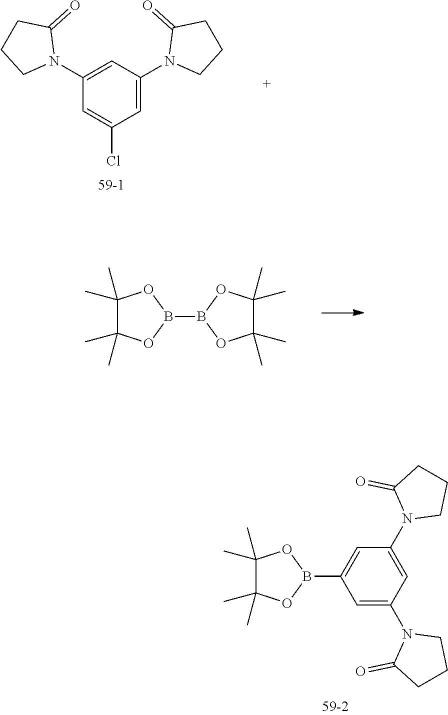

Intermediate 59-1 (10.0 g, 0.036 mol), Bis(pinacolato)diboron (10.9 g, 0.043 mol), KOAc (10.6 g, 0.108 mol), Pd(dppf)Cl₂ (1.3 g, 0.002 mol), and XPhos (5.1 g, 0.010 mol) were added with dioxane 200 mL and reacted while stirring at 100° C. for 12 hours. After completion of the reaction, the reaction mixture was extracted, concentrated, and then columned to obtain 9.3 g of Intermediate 59-2 (yield 70.0%).

### (3) Preparation Example 3. Synthesis of Intermediate 59-3

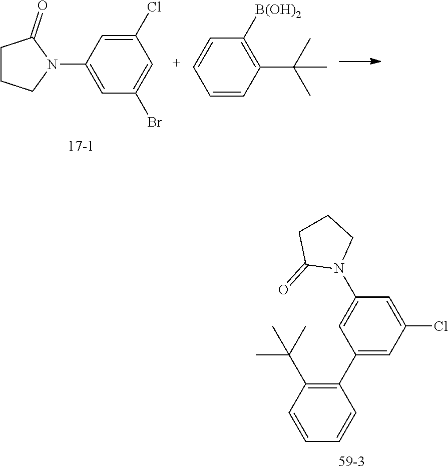

Intermediate 17-1 (10.0 g, 0.036 mol), (2-Tert-butylphenyl)boronic acid (7.8 g, 0.044 mol), K₂CO₃ (15.1 g, 0.109 mol), and Pd(PPh₃)₄ (0.8 g, 0.0008 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 8.3 g of Intermediate 59-3 (yield 69.5%).

### (4) Preparation Example 4: Synthesis of Compound 59

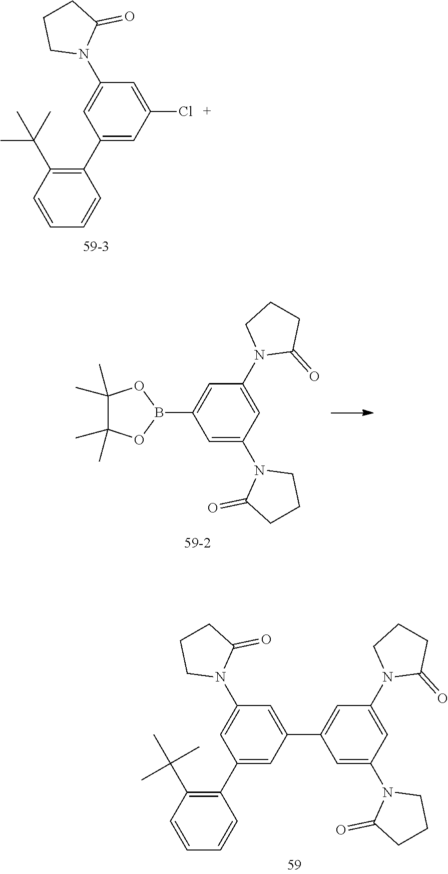

Intermediate 59-3 (10.0 g, 0.031 mol), Intermediate 59-2 (13.6 g, 0.037 mol), K₂CO₃ (12.7 g, 0.092 mol), Pd(OAC)₂ (1.8 g, 0.002 mol), X-Phos (1.5 g, 0.003 mol), THF 200 mL, and H₂O 50 mL were added and reacted while stirring at 70° C. for 6 hours. After completion of the reaction, the reaction mixture was extracted, concentrated, and then column and recrystallized to obtain 16.5 g of Compound 59 (yield 72.7%).

LC/MS: m/z=535 [(M)⁺]

### Synthesis Example 4: Synthesis of Compound 68

### (1) Preparation Example 1: Synthesis of Intermediate 68-1

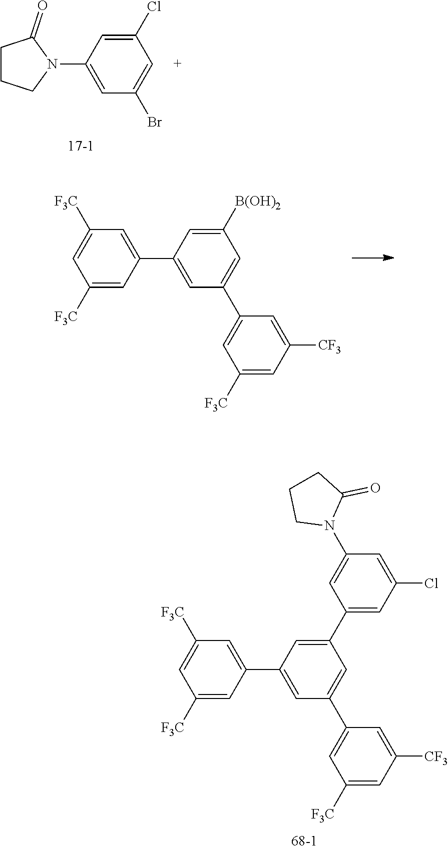

Intermediate 17-1 (10.0 g, 0.036 mol), [3,5-Bis[3,5-bis(trifluoromethyl)phenyl]phenyl]boronic acid (23.9 g, 0.044 mol), K₂CO₃ (15.1 g, 0.109 mol), and Pd(PPh₃)₄ (0.8 g, 0.0007 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 15.8 g of Intermediate 68-1 (yield 62.3%).

### (2) Preparation Example 2: Synthesis of Compound 68

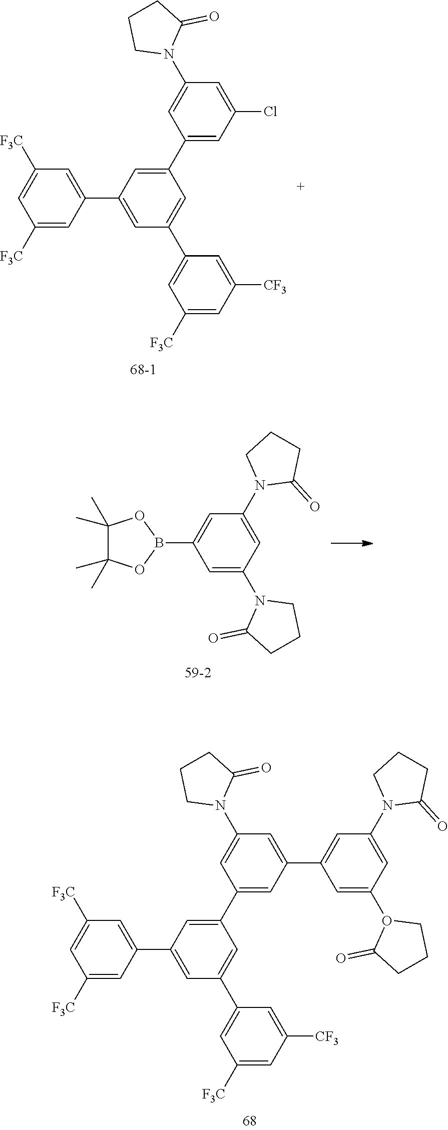

Intermediate 68-1 (10.0 g, 0.014 mol), Intermediate 59-2 (6.4 g, 0.017 mol), K₂CO₃ (6.0 g, 0.043 mol), Pd(OAc)₂ (0.8 g, 0.0007 mol), X-Phos (0.7 g, 0.001 mol), THF 200 mL, and H₂O 50 mL were added and reacted while stirring at 70° C. for 6 hours. After completion of the reaction, the reaction mixture was extracted, concentrated, and then column and recrystallized to obtain 8.2 g of Compound 68 (yield 63.1%).

LC/MS: m/z=903[(M)⁺]

### Synthesis Example 5: Synthesis of Compound 79

### (1) Preparation Example 1: Synthesis of Intermediate 79-1

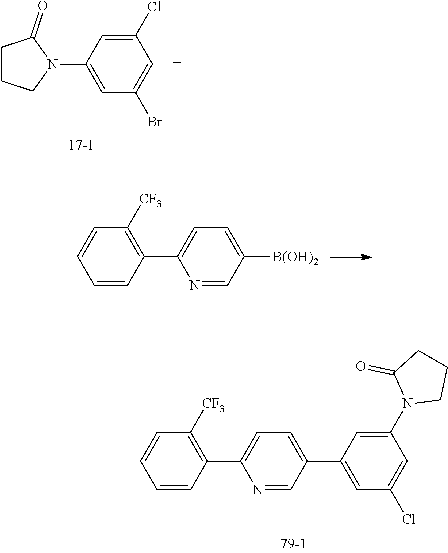

Intermediate 17-1 (10.0 g, 0.036 mol), 2-(2-Trifluoromethylphenyl)pyridine-5-boronic acid (11.7 g, 0.044 mol), K₂CO₃ (15.1 g, 0.109 mol), and Pd(PPh₃)₄ (0.8 g, 0.0007 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 8.9 g of Intermediate 79-1 (yield 58.6%).

### (2) Preparation Example 2: Synthesis of Compound 79

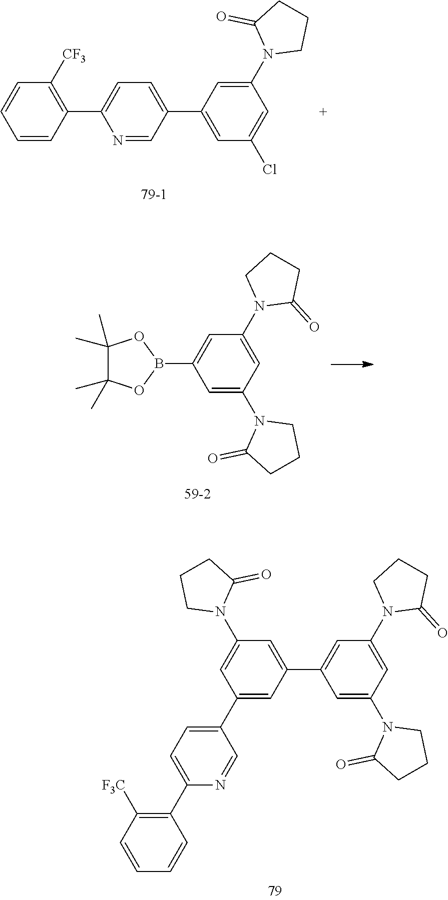 [SMILES: O=C1CCCN1c1cc(Cl)cc(-c2ccc(-c3ccccc3C(F)(F)F)nc2)c1 ; CC1(C)OB(c2cc(N3CCCC3=O)cc(N3CCCC3=O)c2)OC1(C)C ; O=C1CCCN1c1cc(-c2ccc(-c3ccccc3C(F)(F)F)nc2)cc(-c2cc(N3CCCC3=O)cc(N3CCCC3=O)c2)c1]

Intermediate 79-1 (10.0 g, 0.024 mol), Intermediate 59-2 (10.7 g, 0.029 mol), K₂CO₃ (10.0 g, 0.072 mol), Pd(OAc)₂ (1.4 g, 0.001 mol), X-Phos (1.1 g, 0.002 mol), THF 200 mL, and H₂O 50 mL were added and reacted while stirring at 70° C. for 6 hours. After completion of the reaction, the reaction mixture was extracted, concentrated, and then column and recrystallized to obtain 9.1 g of Compound 79 (yield 60.7%).

LC/MS: m/z=624[(M)⁺]

### Synthesis Example 6: Synthesis of Compound 86

### (1) Preparation Example 1: Synthesis of Compound 86

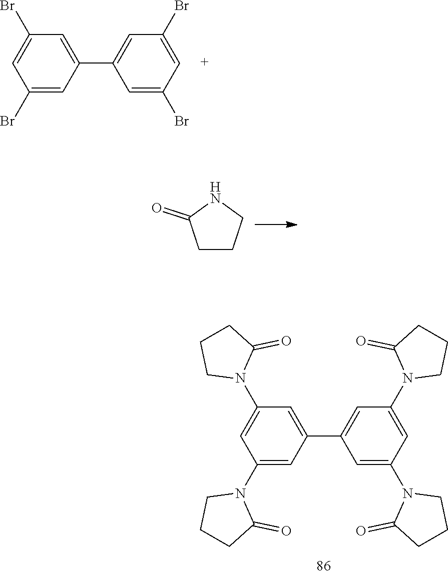

3,3′,5,5′-Tetrabromobiphenyl (10.0 g, 0.021 mol), 2-Pyrrolidinone (8.7 g, 0.102 mol), Cs₂CO₃ (38.8 g, 0.119 mol), Pd(dba)₂ (2.5 g, 0.004 mol), Xant-Phos (35.5 g, 0.061 mol), and dioxane were added and reacted while stirring under reflux for 16 hours. After completion of the reaction, the reaction mixture was extracted, concentrated, and then column and recrystallized to obtain 8.3 g of Compound 86 (yield 80.1%).

LC/MS: m/z=486[(M)⁺]

### Synthesis Example 7: Synthesis of Compound 109

### (1) Preparation Example 1: Synthesis of Intermediate 109-1

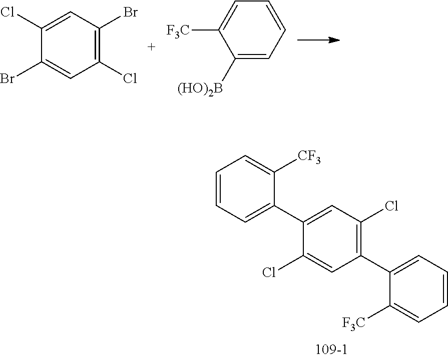

1,4-Dibromo-2,5-dichlorobenzene (10.0 g, 0.033 mol), (2-Tert-butylphenyl)boronic acid (15.0 g, 0.079 mol), K₂CO₃ (27.2 g, 0.197 mol), and Pd(PPh₃)₄ (0.8 g, 0.0007 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 8.8 g of Intermediate 109-1 (yield 61.6%).

### (2) Preparation Example 2: Synthesis of Compound 109

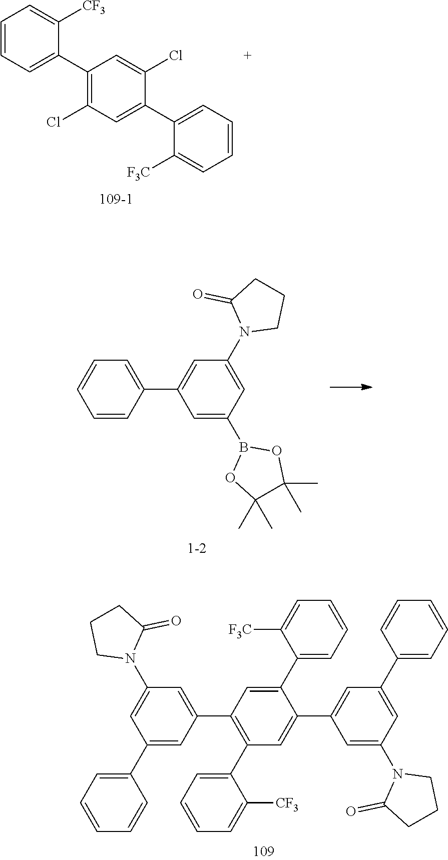 [SMILES: FC(F)(F)c1ccccc1-c1cc(Cl)c(-c2ccccc2C(F)(F)F)cc1Cl ; CC1(C)OB(c2cc(-c3ccccc3)cc(N3CCCC3=O)c2)OC1(C)C ; O=C1CCCN1c1cc(-c2ccccc2)cc(-c2cc(-c3ccccc3C(F)(F)F)c(-c3cc(-c4ccccc4)cc(N4CCCC4=O)c3)cc2-c2ccccc2C(F)(F)F)c1]

Intermediate 109-1 (10.0 g, 0.023 mol), Intermediate 1-2 (20.0 g, 0.055 mol), K₂CO₃ (19.1 g, 0.138 mol), Pd(OAc)₂ (2.7 g, 0.002 mol), X-Phos (1.6 g, 0.003 mol), THF 200 mL, and H₂O 50 mL were added and reacted while stirring at 70° C. for 6 hours. After completion of the reaction, the reaction mixture was extracted, concentrated, and then column and recrystallized to obtain 13.3 g of Compound 109 (yield 69.2%).

LC/MS: m/z=836[(M)^{m}]

### Synthesis Example 8: Synthesis of Compound 124

### (1) Preparation Example 1: Synthesis of Compound 124

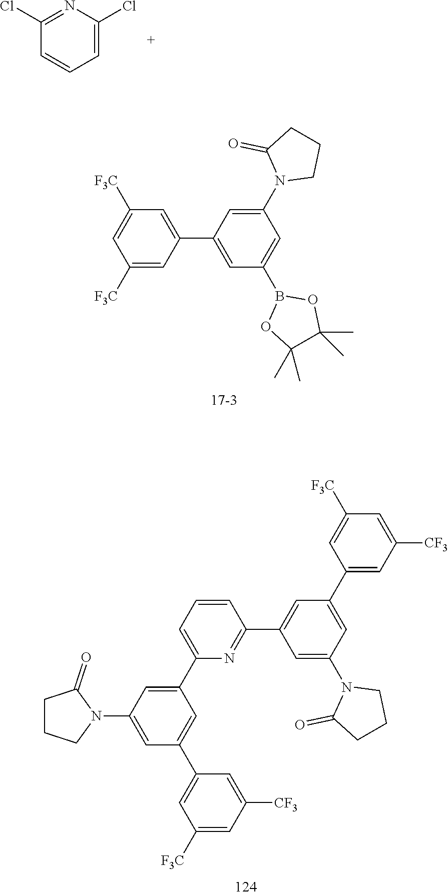

2.6-Dibromopyridine (10.0 g, 0.068 mol), Intermediate 17-3 (81.0 g, 0.162 mol), K₂CO₃ (56.0 g, 0.405 mol), and Pd(PPh₃)₄ (1.6 g, 0.001 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 40.8 g of Compound 124 (yield 73.5%).

LC/MS: m/z=821[(M)⁺]

### Synthesis Example 9: Synthesis of Compound 153

### (1) Preparation Example 1: Synthesis of Compound 153

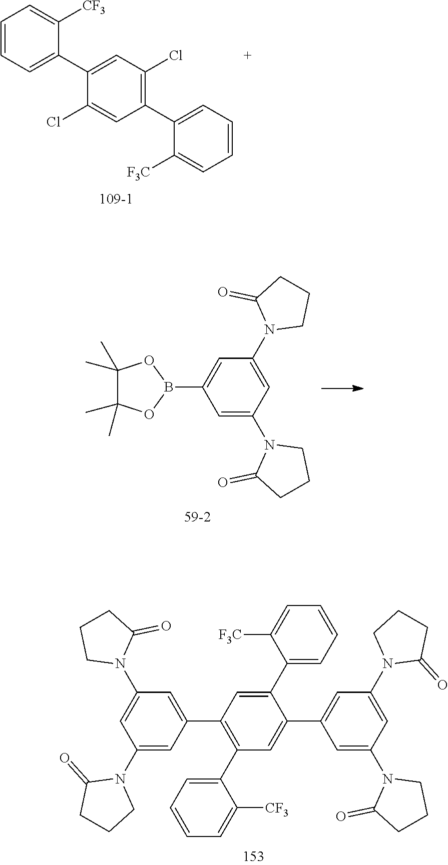 [SMILES: FC(F)(F)c1ccccc1-c1cc(Cl)c(-c2ccccc2C(F)(F)F)cc1Cl ; CC1(C)OB(c2cc(N3CCCC3=O)cc(N3CCCC3=O)c2)OC1(C)C ; O=C1CCCN1c1cc(-c2cc(-c3ccccc3C(F)(F)F)c(-c3cc(N4CCCC4=O)cc(N4CCCC4=O)c3)cc2-c2ccccc2C(F)(F)F)cc(N2CCCC2=O)c1]

Intermediate 109-1 (10.0 g, 0.023 mol), Intermediate 59-2 (20.4 g, 0.055 mol), K₂CO₃ (19.1 g, 0.138 mol), Pd(OAc)₂ (2.7 g, 0.002 mol), X-Phos (1.6 g, 0.003 mol), THF 200 mL, and H₂O 50 mL were added and reacted while stirring at 70° C. for 6 hours. After completion of the reaction, the reaction mixture was extracted, concentrated, and then column and recrystallized to obtain 12.4 g of Compound 153 (yield 63.4%).

LC/MS: m/z=850[(M)⁺]

### Synthesis Example 10: Synthesis of Compound 155

### (1) Preparation Example 1: Synthesis of Intermediate 155-1

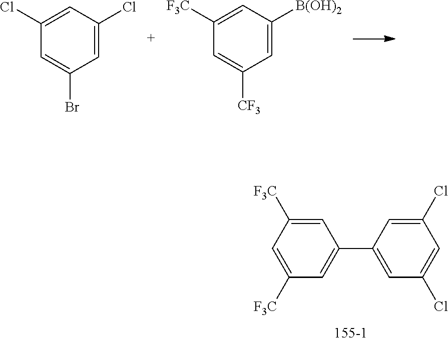

1-Bromo-3,5-dichlorobenzene (10.0 g, 0.044 mol), 3,5-Bis(trifluoromethyl)phenylboronic acid (13.7 g, 0.053 mol), K₂CO₃ (18.4 g, 0.133 mol), and Pd(PPh₃)₄ (1.0 g, 0.0009 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 10.7 g of Intermediate 155-1 (yield 67.3%).

### (2) Preparation Example 2: Synthesis of Compound 155

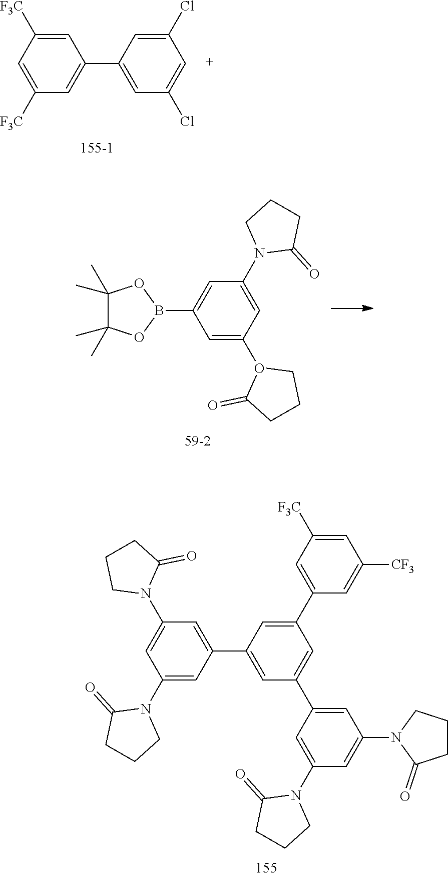

Intermediate 155-1 (10.0 g, 0.028 mol), Intermediate 59-2 (24.8 g, 0.067 mol), K₂CO₃ (23.1 g, 0.167 mol), Pd(OAc)₂ (3.2 g, 0.003 mol), X-Phos (2.0 g, 0.004 mol), THF 200 mL, and H₂O 50 mL were added and reacted while stirring at 70° C. for 6 hours. After completion of the reaction, the reaction mixture was extracted, concentrated, and then column and recrystallized to obtain 13.3 g of Compound 155 (yield 61.6%).

LC/MS: m/z=774[(M)⁺]

### Synthesis Example 11: Synthesis of Compound 164

### (1) Preparation Example 1: Synthesis of Intermediate 164-1

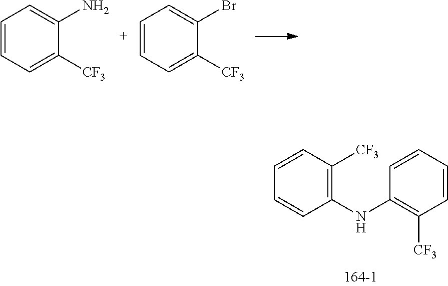

2-(Trifluoromethyl)aniline (10.0 g, 0.062 mol), 1-Bromo-2-(trifluoromethyl)benzene (21.0 g, 0.093 mol), NaOtBu (17.9 g, 0.186 mol), Pd(dba)₂ (1.4 g, 2.5 mmol), and t-Bu₃P (1.0 g, 5.0 mmol) were added with Toluene 150 mL and reacted while stirring at 70° C. for 4 hours. After completion of the reaction, the reaction mixture was extracted, concentrated, and then columned to obtain 12.8 g of Intermediate 164-1 (yield 67.6%).

### (2) Preparation Example 2: Synthesis of Intermediate 164-2

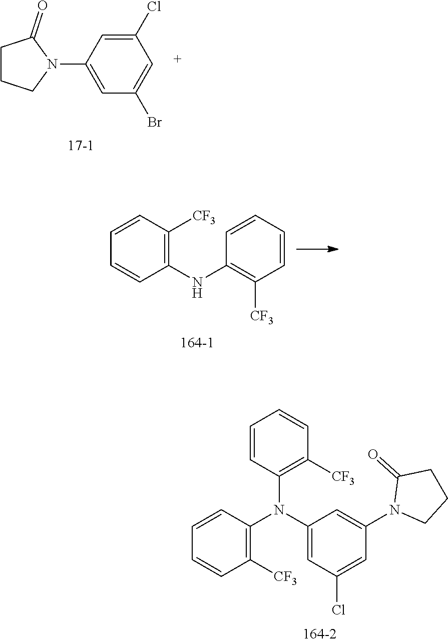

Intermediate 17-1 (10.0 g, 0.036 mol), Intermediate 164-1 (16.7 g, 0.055 mol), NaOtBu (10.5 g, 0.109 mol), Pd(dba)₂ (0.8 g, 1.5 mmol), and t-Bu₃P (0.6 g, 2.9 mmol) were added with Toluene 150 mL and reacted while stirring at 70° C. for 4 hours. After completion of the reaction, the reaction mixture was extracted, concentrated, and then columned to obtain 10.6 g of Intermediate 164-2 (yield 58.3%).

### (3) Preparation Example 3: Synthesis of Intermediate 164-3

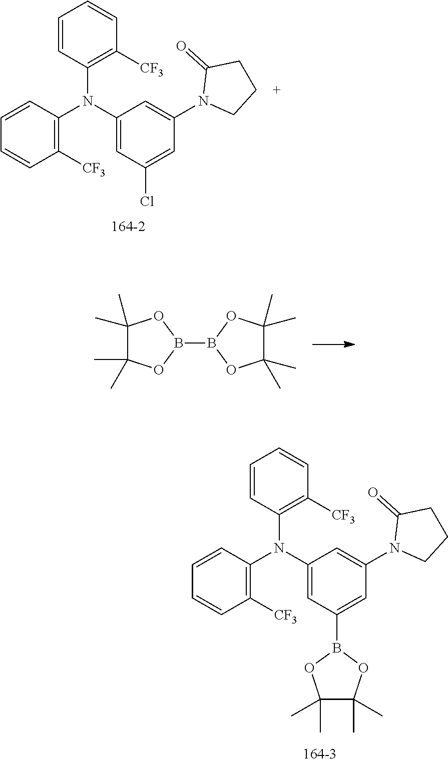

Intermediate 164-2 (10.0 g, 0.020 mol), Bis(pinacolato)diboron (6.1 g, 0.024 mol), KOAc (5.9 g, 0.060 mol), Pd(dppf)Cl₂ (0.7 g, 0.001 mol), and XPhos (2.9 g, 0.0006 mol) were added with dioxane 200 mL and reacted while stirring at 100° C. for 12 hours. After completion of the reaction, the reaction mixture was extracted, concentrated, and then column and recrystallized to obtain 8.3 g of Intermediate 164-3 (yield 70.1%).

### (4) Preparation Example 4: Synthesis of Compound 164

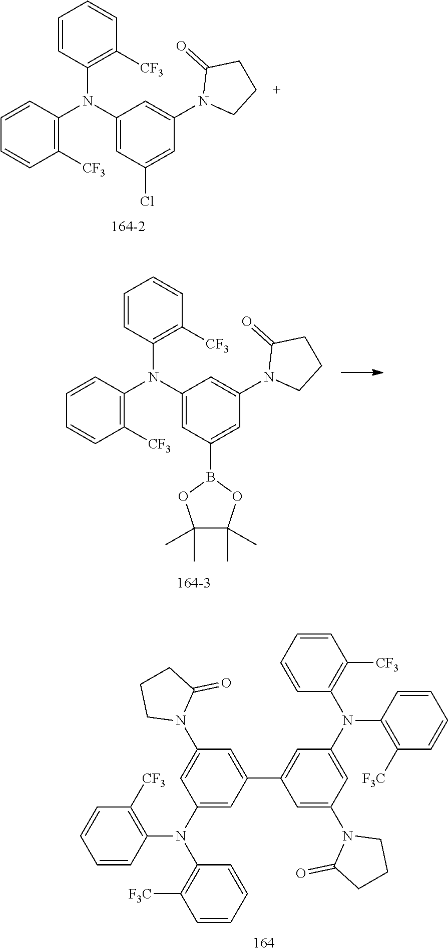 [SMILES: O=C1CCCN1c1cc(Cl)cc(N(c2ccccc2C(F)(F)F)c2ccccc2C(F)(F)F)c1 ; CC1(C)OB(c2cc(N3CCCC3=O)cc(N(c3ccccc3C(F)(F)F)c3ccccc3C(F)(F)F)c2)OC1(C)C ; O=C1CCCN1c1cc(-c2cc(N3CCCC3=O)cc(N(c3ccccc3C(F)(F)F)c3ccccc3C(F)(F)F)c2)cc(N(c2ccccc2C(F)(F)F)c2ccccc2C(F)(F)F)c1]

Intermediate 164-2 (10.0 g, 0.020 mol), Intermediate 164-3 (14.2 g, 0.024 mol), K₂CO₃ (8.3 g, 0.060 mol), Pd(OAc)₂ (1.2 g, 0.001 mol), X-Phos (0.9 g, 0.002 mol), TIFF 200 mL, and H₂O 50 mL were added and reacted while stirring at 70° C. for 6 hours. After completion of the reaction, the reaction mixture was extracted, concentrated, and then column and recrystallized to obtain 12.5 g of Compound 164 (yield 67.3%).

LC/MS: m/z=926[(M)⁺]

### Synthesis Example 12: Synthesis of Compound 230

### (1) Preparation Example 1: Synthesis of Intermediate 230-1

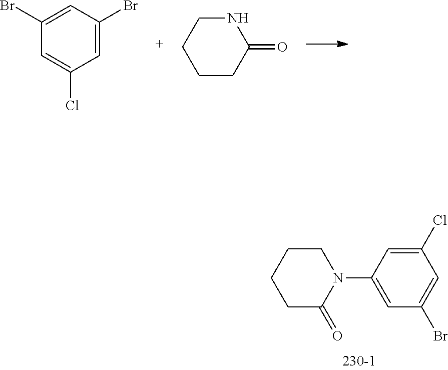 [SMILES: Clc1cc(Br)cc(Br)c1 ; O=C1CCCCN1 ; O=C1CCCCN1c1cc(Cl)cc(Br)c1]

1,3-Dibromo-5-chlorobenzene (10.0 g, 0.037 mol), 2-Piperidinone (4.4 g, 0.044 mol), Cs₂CO₃ (16.9 g, 0.052 mol), Pd(dba)₂ (1.1 g, 0.002 mol), Xant-Phos (3.9 g, 0.007 mol), and dioxane were added and reacted while stirring under reflux for 16 hours. After completion of the reaction, the reaction mixture was extracted, concentrated, and then columned to obtain 6.5 g of Intermediate 230-1 (yield 60.9%).

### (2) Preparation Example 2: Synthesis of Intermediate 230-2

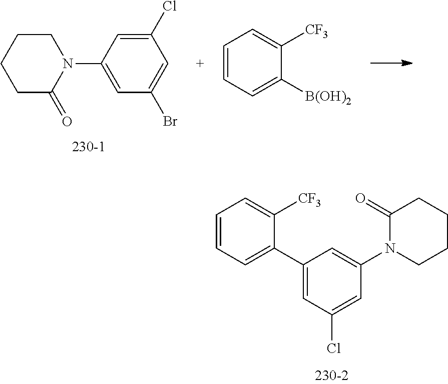 [SMILES: O=C1CCCCN1c1cc(Cl)cc(Br)c1 ; OB(O)c1ccccc1C(F)(F)F ; O=C1CCCCN1c1cc(Cl)cc(-c2ccccc2C(F)(F)F)c1]

Intermediate 230-1 (10.0 g, 0.035 mol), (2-Tert-butylphenyl)boronic acid (7.9 g, 0.042 mol), K₂CO₃ (14.4 g, 0.104 mol), and Pd(PPh₃)₄ (0.8 g, 0.0007 mol) were added with toluene 200 mL, ethanol 50 mL, and H₂O 50 mL and reacted while stirring at 80° C. for 6 hours. After completion of the reaction, the reaction mixture was extracted, concentrated, and then columned to obtain 8.3 g of Intermediate 230-2 (yield 67.7%).

### (3) Preparation Example 3: Synthesis of Intermediate 230-3

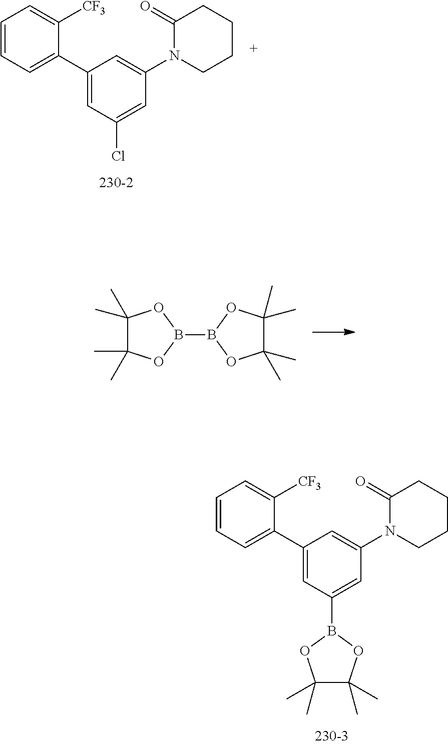

Intermediate 230-2 (10.0 g, 0.028 mol), Bis(pinacolato)diboron (8.6 g, 0.034 mol), KOAc (8.3 g, 0.085 mol), Pd(dppf)Cl₂ (1.0 g, 0.001 mol), and XPhos (4.0 g, 0.009 mol) were added with dioxane 200 mL and reacted while stirring at 100° C. for 12 hours. After completion of the reaction, the reaction mixture was extracted, concentrated, and then column and recrystallized to obtain 9.1 g of Intermediate 230-3 (yield 72.3%).

### (4) Preparation Example 4: Synthesis of Compound 230

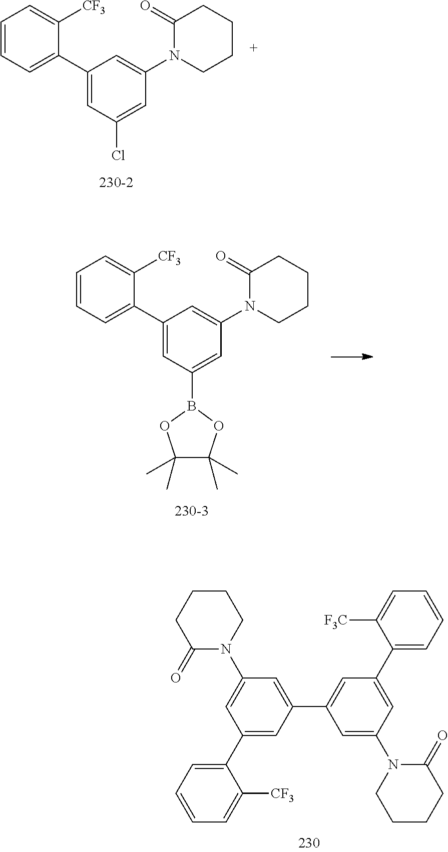 [SMILES: O=C1CCCCN1c1cc(Cl)cc(-c2ccccc2C(F)(F)F)c1 ; CC1(C)OB(c2cc(-c3ccccc3C(F)(F)F)cc(N3CCCCC3=O)c2)OC1(C)C ; O=C1CCCCN1c1cc(-c2cc(-c3ccccc3C(F)(F)F)cc(N3CCCCC3=O)c2)cc(-c2ccccc2C(F)(F)F)c1]

Intermediate 230-2 (10.0 g, 0.028 mol), Intermediate 230-3 (15.1 g, 0.040 mol), K₂CO₃ (11.7 g, 0.085 mol), Pd(OAc)₂ (1.6 g, 0.001 mol), X-Phos (1.3 g, 0.003 mol), THF 200 mL, and H₂O 50 mL were added and reacted while stirring at 70° C. for 6 hours. After completion of the reaction, the reaction mixture was extracted, concentrated, and then column and recrystallized to obtain 12.8 g of Compound 230 (yield 71.1%).

LC/MS: m/z=636[(M)⁺]

### Synthesis Example 13: Synthesis of Compound 241

### (1) Preparation Example 1: Synthesis of Intermediate 241-1

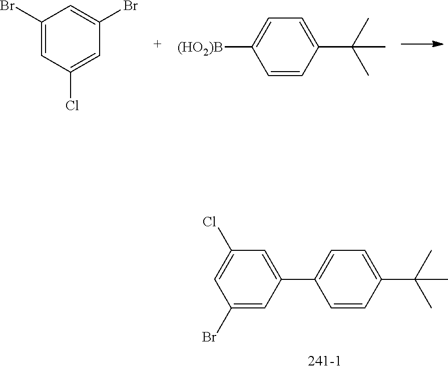

1,3-Dibromo-5-chlorobenzene (10.0 g, 0.037 mol), 4-tert-Butylphenylboronic acid (7.9 g, 0.044 mol), K₂CO₃ (15.3 g, 0.111 mol), and Pd(PPh₃)₄ (0.9 g, 0.0007 mol) were added with toluene 200 mL, ethanol 50 mL, and H₂O 50 mL and reacted while stirring at 80° C. for 6 hours. After completion of the reaction, the reaction mixture was extracted, concentrated, and then columned to obtain 7.5 g of Intermediate 241-1 (yield 62.7%).

### (2) Preparation Example 2: Synthesis of Intermediate 241-2

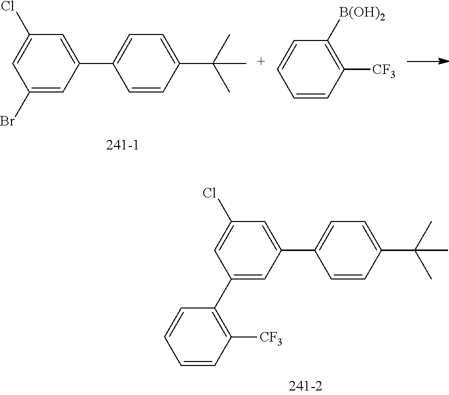

Intermediate 241-1 (10.0 g, 0.031 mol), (2-Tert-butylphenyl)boronic acid (7.0 g, 0.037 mol), K₂CO₃ (12.8 g, 0.093 mol), and Pd(PPh₃)₄ (0.7 g, 0.0006 mol) were added with toluene 200 mL, ethanol 50 mL, and H₂O 50 mL and reacted while stirring at 80° C. for 6 hours. After completion of the reaction, the reaction mixture was extracted, concentrated, and then columned to obtain 8.4 g of Intermediate 241-2 (yield 69.9%).

### (3) Preparation Example 3: Synthesis of Intermediate 241-3

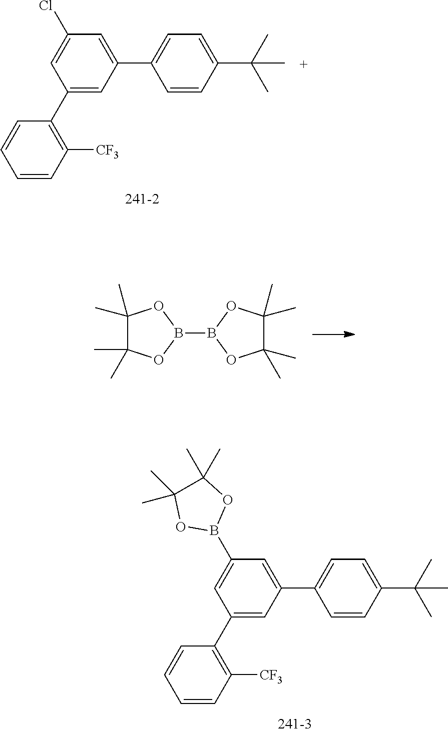

Intermediate 241-2 (10.0 g, 0.026 mol), Bis(pinacolato)diboron (7.8 g, 0.031 mol), KOAc (7.6 g, 0.077 mol), Pd(dppf)Cl₂ (0.9 g, 0.001 mol), and XPhos (1.1 g, 0.002 mol) were added with dioxane 200 mL and reacted while stirring at 100° C. for 12 hours. After completion of the reaction, the reaction mixture was extracted, concentrated, and then columned to obtain 8.9 g of Intermediate 241-3 (yield 71.9%).

### (4) Preparation Example 4: Synthesis of Intermediate 241-4

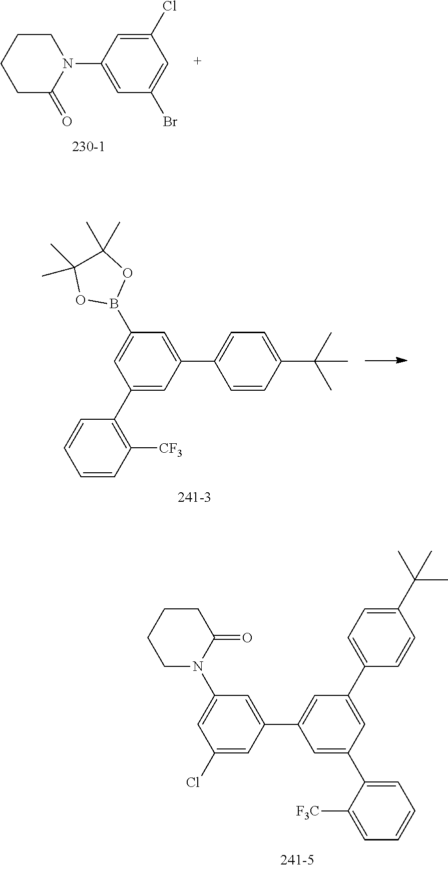 [SMILES: O=C1CCCCN1c1cc(Cl)cc(Br)c1 ; CC(C)(C)c1ccc(-c2cc(B3OC(C)(C)C(C)(C)O3)cc(-c3ccccc3C(F)(F)F)c2)cc1 ; CC(C)(C)c1ccc(-c2cc(-c3cc(Cl)cc(N4CCCCC4=O)c3)cc(-c3ccccc3C(F)(F)F)c2)cc1]

Intermediate 230-1 (10.0 g, 0.035 mol), Intermediate 241-3 (20.0 g, 0.042 mol), K₂CO₃ (14.4 g, 0.104 mol), and Pd(PPh₃)₄ (0.8 g, 0.0007 mol) were added with toluene 200 mL, ethanol 50 mL, and H₂O 50 mL and reacted while stirring at 80° C. for 6 hours. After completion of the reaction, the reaction mixture was extracted, concentrated, and then columned to obtain 13.1 g of Intermediate 241-4 (yield 67.3%).

### (5) Preparation Example 5: Synthesis of Intermediate 241-5

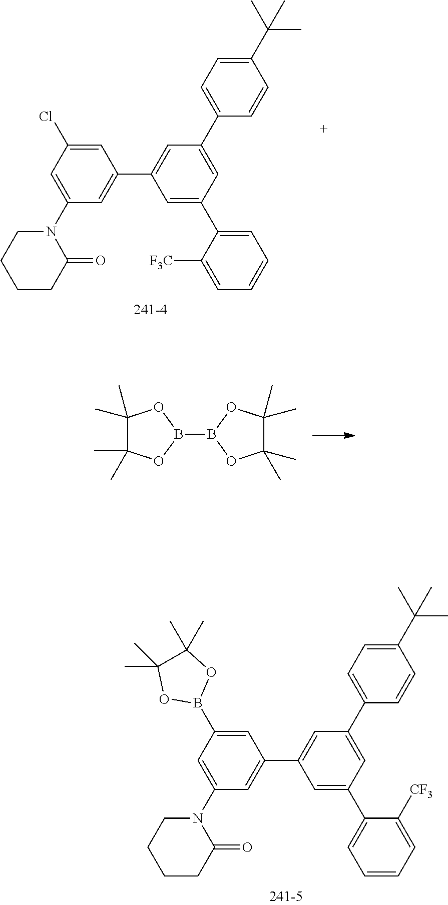

Intermediate 241-4 (10.0 g, 0.018 mol), Bis(pinacolato)diboron (5.4 g, 0.021 mol), KOAc (5.2 g, 0.053 mol), Pd(dppf)Cl₂ (0.7 g, 0.001 mol), and XPhos (0.8 g, 0.002 mol) were added with dioxane 200 mL and reacted while stirring at 100° C. for 12 hours. After completion of the reaction, the reaction mixture was extracted, concentrated, and then column and recrystallized to obtain 8.3 g of Intermediate 241-5 (yield 71.4%).

### (6) Preparation Example 6: Synthesis of Compound 241

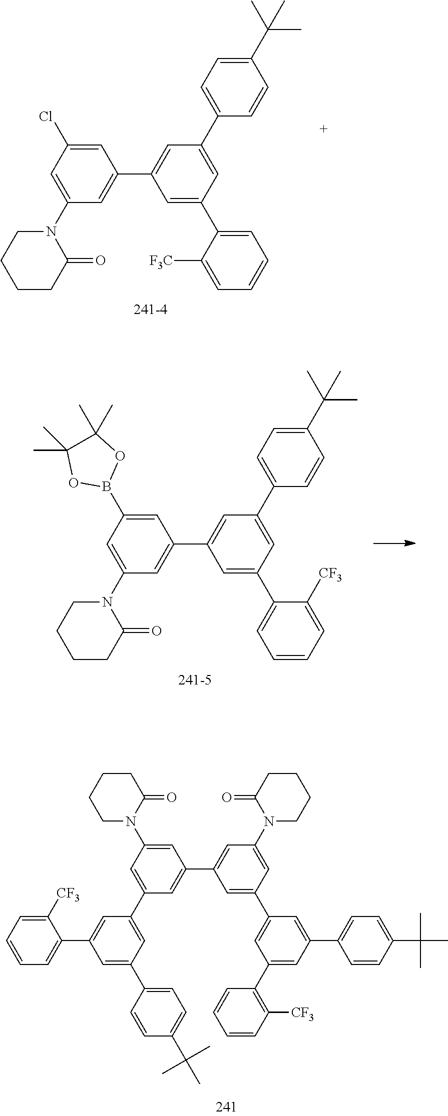

Intermediate 241-4 (10.0 g, 0.018 mol), Intermediate 241-5 (14.0 g, 0.021 mol), K₂CO₃ (7.4 g, 0.053 mol), Pd(OAc)₂ (1.0 g, 0.001 mol), X-Phos (0.8 g, 0.002 mol), THF 200 mL, and H₂O 50 mL were added and reacted while stirring at 70° C. for 6 hours. After completion of the reaction, the reaction mixture was extracted, concentrated, and then column and recrystallized to obtain 12.1 g of Compound 241 (yield 64.6%).

LC/MS: m/z=1053[(M)⁺]

### Synthesis Example 14: Synthesis of Compound 270

### (1) Preparation Example 1: Synthesis of Intermediate 270-1

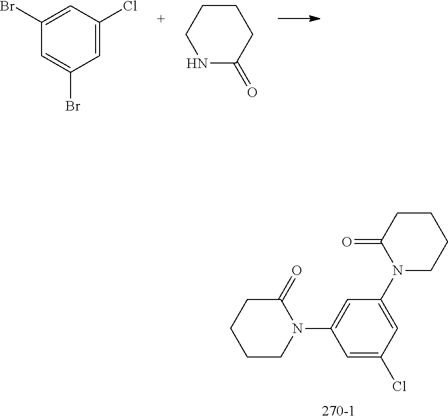 [SMILES: Clc1cc(Br)cc(Br)c1 ; O=C1CCCCN1 ; O=C1CCCCN1c1cc(Cl)cc(N2CCCCC2=O)c1]

1,3-Dibromo-5-chlorobenzene (10.0 g, 0.037 mol), 2-Piperidinone (8.8 g, 0.089 mol), Cs₂CO₃ (33.7 g, 0.104 mol), Pd(dba)₂ (2.1 g, 0.004 mol), Xant-Phos (15.4 g, 0.027 mol), and dioxane were added and reacted while stirring under reflux for 16 hours. After completion of the reaction, the reaction mixture was extracted, concentrated, and then columned to obtain 9.8 g of Intermediate 270-1 (yield 86.4%).

### (2) Preparation Example 2: Synthesis of Intermediate 270-2

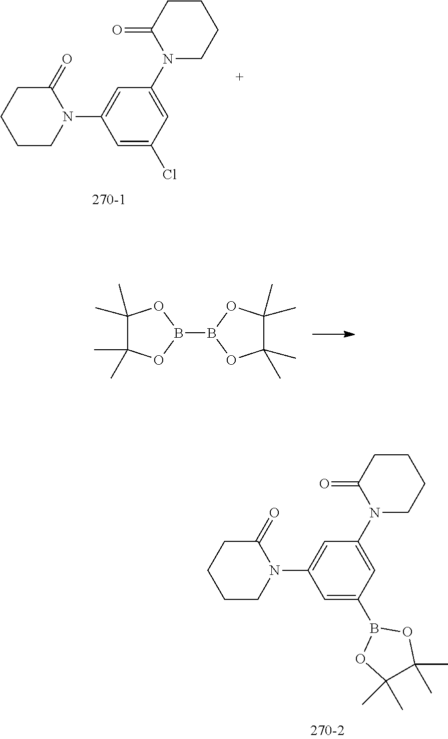

Intermediate 270-1 (10.0 g, 0.033 mol), Bis(pinacolato)diboron (9.9 g, 0.039 mol), KOAc (9.6 g, 0.098 mol), Pd(dppf)Cl₂ (1.2 g, 0.002 mol), and XPhos (4.7 g, 0.001 mol) were added with dioxane 200 mL and reacted while stirring at 100° C. for 12 hours. After completion of the reaction, the reaction mixture was extracted, concentrated, and then columned to obtain 9.5 g of Intermediate 270-2 (yield 73.23).

### (3) Preparation Example 3: Synthesis of Compound 270-3

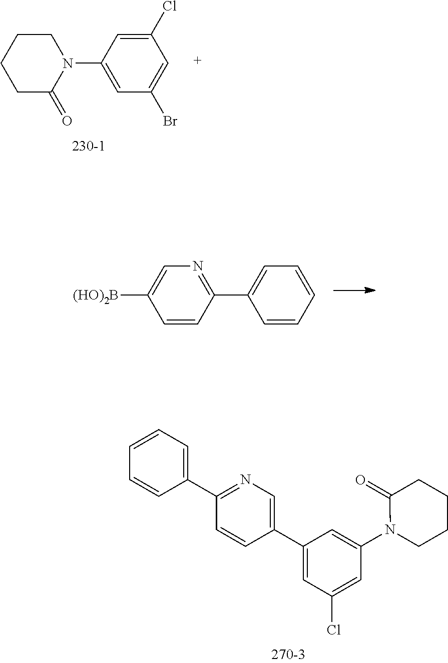 [SMILES: O=C1CCCCN1c1cc(Cl)cc(Br)c1 ; OB(O)c1ccc(-c2ccccc2)nc1 ; O=C1CCCCN1c1cc(Cl)cc(-c2ccc(-c3ccccc3)nc2)c1]

Intermediate 230-1 (10.0 g, 0.035 mol), B-(6-Phenyl-3-pyridinyl)boronic acid (8.3 g, 0.042 mol), K₂CO₃ (14.4 g, 0.104 mol), and Pd(PPh₃)₄ (0.8 g, 0.0007 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 8.5 g of Intermediate 270-3 (yield 67.6%).

### (4) Preparation Example 4: Synthesis of Compound 270

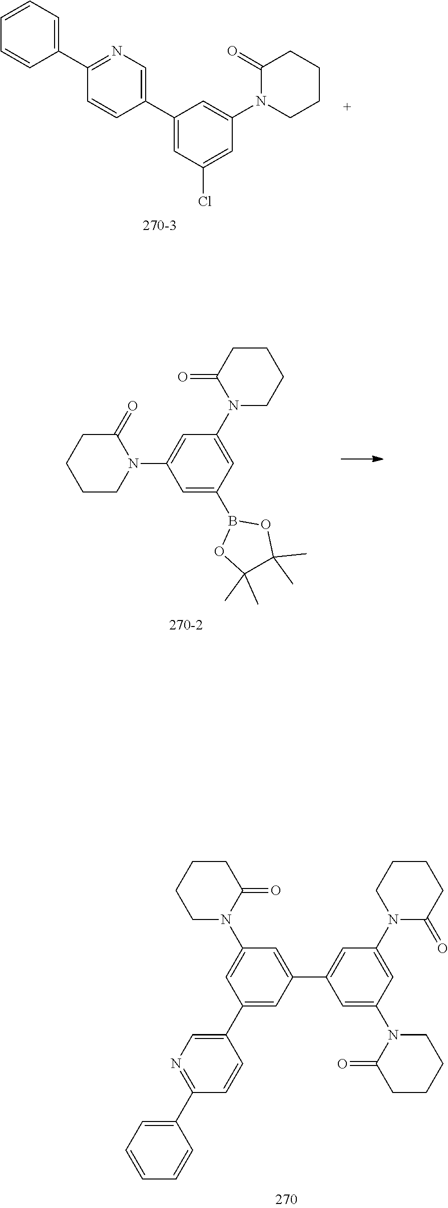 [SMILES: O=C1CCCCN1c1cc(Cl)cc(-c2ccc(-c3ccccc3)nc2)c1 ; CC1(C)OB(c2cc(N3CCCCC3=O)cc(N3CCCCC3=O)c2)OC1(C)C ; O=C1CCCCN1c1cc(-c2ccc(-c3ccccc3)nc2)cc(-c2cc(N3CCCCC3=O)cc(N3CCCCC3=O)c2)c1]

Intermediate 270-3 (10.0 g, 0.028 mol), Intermediate 270-2 (13.2 g, 0.033 mol), K₂CO₃ (11.4 g, 0.083 mol), Pd(OAc)₂ (1.6 g, 0.001 mol), X-Phos (1.3 g, 0.003 mol), THF 200 mL, and H₂O 50 mL were added and reacted while stirring at 70° C. for 6 hours. After completion of the reaction, the reaction mixture was extracted, concentrated, and then column and recrystallized to obtain 12.1 g of Compound 270 (yield 73.3%).

LC/MS: m/z=598[(M)⁺]

### Synthesis Example 15: Synthesis of Compound 288

### (1) Preparation Example 1: Synthesis of Intermediate 288-1

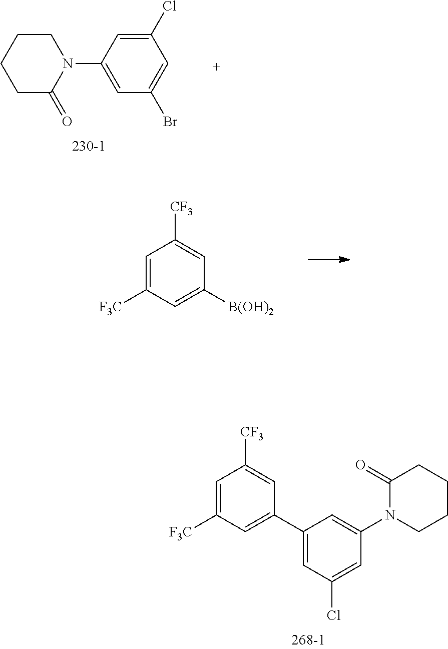 [SMILES: O=C1CCCCN1c1cc(Cl)cc(Br)c1 ; OB(O)c1cc(C(F)(F)F)cc(C(F)(F)F)c1 ; O=C1CCCCN1c1cc(Cl)cc(-c2cc(C(F)(F)F)cc(C(F)(F)F)c2)c1]

Intermediate 230-1 (10.0 g, 0.035 mol), 3,5-Bis(trifluoromethyl)phenylboronic acid (10.7 g, 0.042 mol), K₂CO₃ (14.4 g, 0.104 mol), and Pd(PPh₃)₄ (0.8 g, 0.0007 mol) were added with toluene 200 mL, ethanol 50 mL, and H₂O 50 mL and reacted while stirring at 80° C. for 6 hours. After completion of the reaction, the reaction mixture was extracted, concentrated, and then columned to obtain 9.8 g of Intermediate 288-1 (yield 67.1%).

### (2) Preparation Example 2: Synthesis of Intermediate 288-2

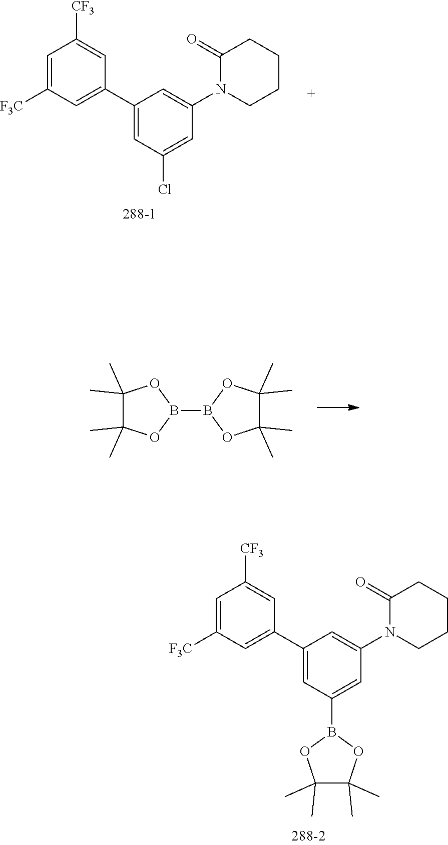

Intermediate 288-1 (10.0 g, 0.024 mol), Bis(pinacolato)diboron (7.2 g, 0.029 mol), KOAc (7.0 g, 0.071 mol), Pd(dppf)Cl₂ (0.9 g, 0.001 mol), and XPhos (3.4 g, 0.007 mol) were added with dioxane 200 mL and reacted while stirring at 100° C. for 12 hours. After completion of the reaction, the reaction mixture was extracted, concentrated, and then column and recrystallized to obtain 8.7 g of Intermediate 288-2 (yield 71.5%).

### (3) Preparation Example 3: Synthesis of Compound 288

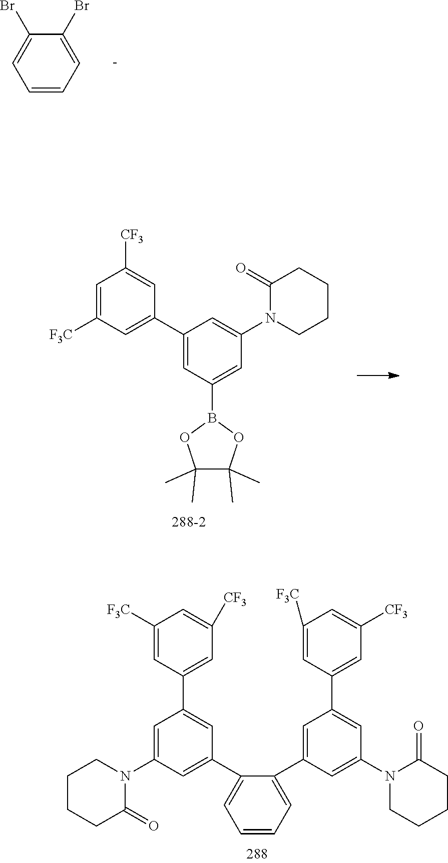 [SMILES: Brc1ccccc1Br ; CC1(C)OB(c2cc(-c3cc(C(F)(F)F)cc(C(F)(F)F)c3)cc(N3CCCCC3=O)c2)OC1(C)C ; O=C1CCCCN1c1cc(-c2cc(C(F)(F)F)cc(C(F)(F)F)c2)cc(-c2ccccc2-c2cc(-c3cc(C(F)(F)F)cc(C(F)(F)F)c3)cc(N3CCCCC3=O)c2)c1]

1.2-Dibromopyridine (10.0 g, 0.042 mol), Intermediate 288-2 (52.2 g, 0.102 mol), K₂CO₃ (35.2 g, 0.254 mol), and Pd(PPh₃)₄ (1.0 g, 0.0008 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 20.5 g of Compound 288 (yield 57.0%).

LC/MS: m/z=848[(M)⁺]

### Synthesis Example 16: Synthesis of Compound 340

### (1) Preparation Example 1: Synthesis of Intermediate 340-1

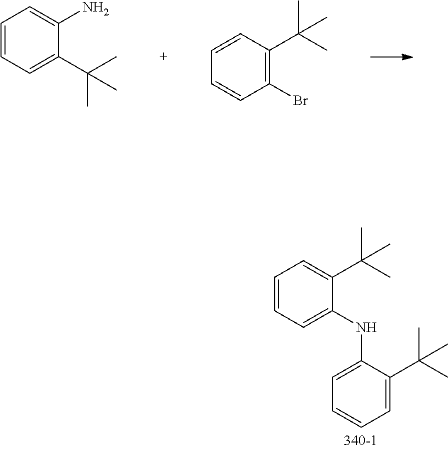 [SMILES: CC(C)(C)c1ccccc1N ; CC(C)(C)c1ccccc1Br ; CC(C)(C)c1ccccc1Nc1ccccc1C(C)(C)C]

2-tert-Butylaniline (10.0 g, 0.067 mol), 1-Bromo-2-(1,1-dimethylethyl)benzene (21.4 g, 0.101 mol), NaOtBu (19.3 g, 0.201 mol), Pd(dba)₂ (1.5 g, 2.7 mmol), and t-Bu₃P (1.1 g, 5.4 mmol) were added with Toluene 150 mL and reacted while stirring at 70° C. for 4 hours. After completion of the reaction, the reaction mixture was extracted, concentrated, and then columned to obtain 9.8 g of Intermediate 340-1 (yield 52.0%).

### (2) Preparation Example 2: Synthesis of Intermediate 340-2

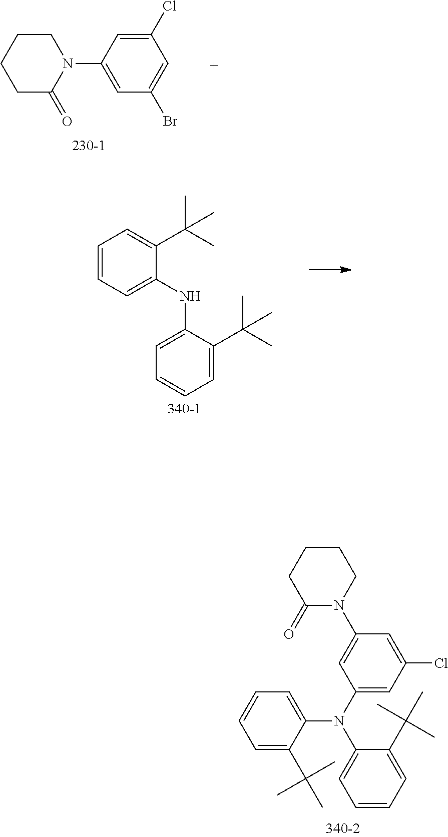 [SMILES: O=C1CCCCN1c1cc(Cl)cc(Br)c1 ; CC(C)(C)c1ccccc1Nc1ccccc1C(C)(C)C ; CC(C)(C)c1ccccc1N(c1cc(Cl)cc(N2CCCCC2=O)c1)c1ccccc1C(C)(C)C]

Intermediate 230-1 (10.0 g, 0.035 mol), Intermediate 340-1 (14.6 g, 0.052 mol), NaOtBu (10.0 g, 0.104 mol), Pd(dba)₂ (0.8 g, 1.4 mmol), and t-Bu₃P (0.6 g, 2.8 mmol) were added with Toluene 150 mL and reacted while stirring at 70° C. for 4 hours. After completion of the reaction, the reaction mixture was extracted, concentrated, and then columned to obtain 10.3 g of Intermediate 340-2 (yield 60.8%).

### (3) Preparation Example 3: Synthesis of Intermediate 340-3

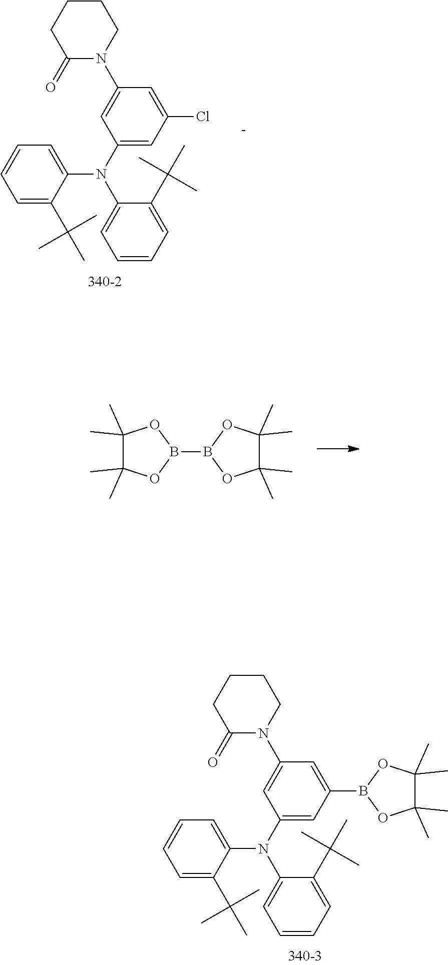

Intermediate 340-2 (10.0 g, 0.020 mol), Bis(pinacolato)diboron (6.2 g, 0.025 mol), KOAc (6.0 g, 0.061 mol), Pd(dppf)Cl₂ (0.8 g, 0.001 mol), and XPhos (2.9 g, 0.0006 mol) were added with dioxane 200 mL and reacted while stirring at 100° C. for 12 hours. After completion of the reaction, the reaction mixture was extracted, concentrated, and then column and recrystallized to obtain 8.6 g of Intermediate 340-3 (yield 72.4%).

### (4) Preparation Example 4: Synthesis of Compound 340

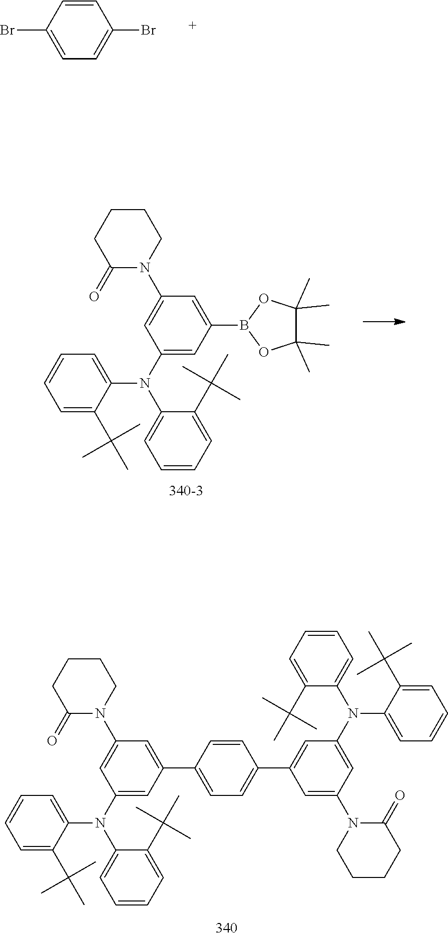 [SMILES: Brc1ccc(Br)cc1 ; CC(C)(C)c1ccccc1N(c1cc(B2OC(C)(C)C(C)(C)O2)cc(N2CCCCC2=O)c1)c1ccccc1C(C)(C)C ; CC(C)(C)c1ccccc1N(c1cc(-c2ccc(-c3cc(N4CCCCC4=O)cc(N(c4ccccc4C(C)(C)C)c4ccccc4C(C)(C)C)c3)cc2)cc(N2CCCCC2=O)c1)c1ccccc1C(C)(C)C]

1.4-Dibromopyridine (10.0 g, 0.042 mol), Intermediate 340-3 (59.1 g, 0.102 mol), K₂CO₃ (35.2 g, 0.254 mol), and Pd(PPh₃)₄ (1.0 g, 0.0008 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 26.1 g of Compound 340 (yield 62.6%).

LC/MS: m/z=983[(M)⁺]

### Synthesis Example 17: Synthesis of Compound 369

### (1) Preparation Example 1: Synthesis of Intermediate 369-1

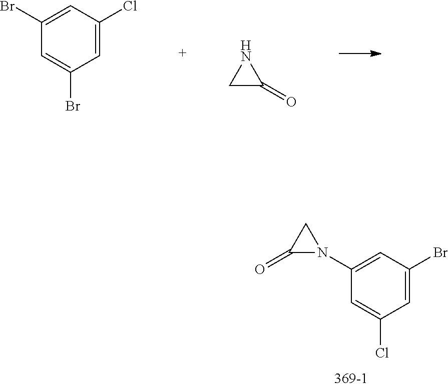

1,3-Dibromo-5-chlorobenzene (10.0 g, 0.037 mol), 2-Azetidinone (2.5 g, 0.044 mol), Cs₂CO₃ (16.9 g, 0.052 mol), Pd(dba)₂ (1.1 g, 0.002 mol), Xant-Phos (3.9 g, 0.007 mol), and dioxane were added and reacted while stirring under reflux for 16 hours. After completion of the reaction, the reaction mixture was extracted, concentrated, and then columned to obtain 5.4 g of Intermediate 369-1 (yield 59.2%).

### (2) Preparation Example 2: Synthesis of Intermediate 369-2

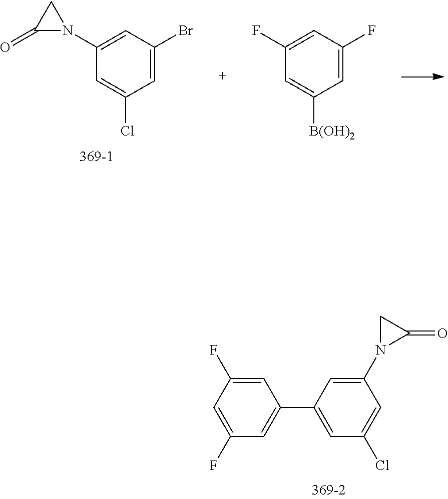 [SMILES: O=C1CN1c1cc(Cl)cc(Br)c1 ; OB(O)c1cc(F)cc(F)c1 ; O=C1CN1c1cc(Cl)cc(-c2cc(F)cc(F)c2)c1]

Intermediate 369-1 (10.0 g, 0.041 mol), 3,5-Difluorophenylboronic acid (7.7 g, 0.049 mol), K₂CO₃ (16.8 g, 0.122 mol), and Pd(PPh₃)₄ (0.9 g, 0.0008 mol) were added with toluene 200 mL, ethanol 50 mL, and H₂O 50 mL and reacted while stirring at 80° C. for 6 hours. After completion of the reaction, the reaction mixture was extracted, concentrated, and then columned to obtain 7.2 g of Intermediate 369-2 (yield 63.5%).

### (3) Preparation Example 3: Synthesis of Intermediate 369-3

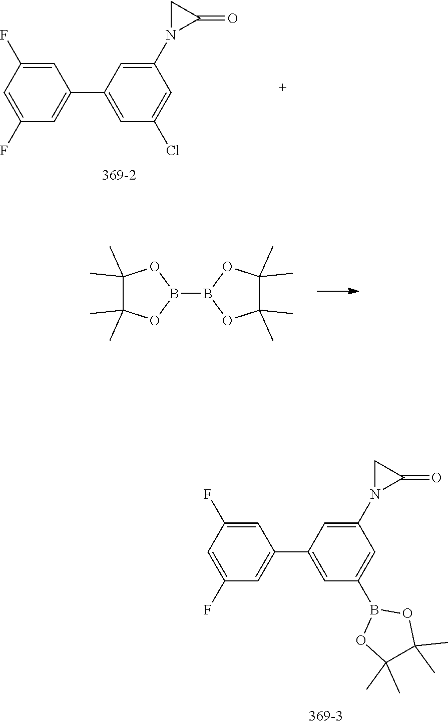

Intermediate 369-2 (10.0 g, 0.036 mol), Bis(pinacolato)diboron (10.9 g, 0.043 mol), KOAc (10.5 g, 0.107 mol), Pd(dppf)Cl₂ (1.3 g, 0.002 mol), and XPhos (5.1 g, 0.011 mol) were added with dioxane 200 mL and reacted while stirring at 100° C. for 12 hours. After completion of the reaction, the reaction mixture was extracted, concentrated, and then column and recrystallized to obtain 9.8 g of Intermediate 369-3 (yield 73.8%).

### (4) Preparation Example 4: Synthesis of Compound 369

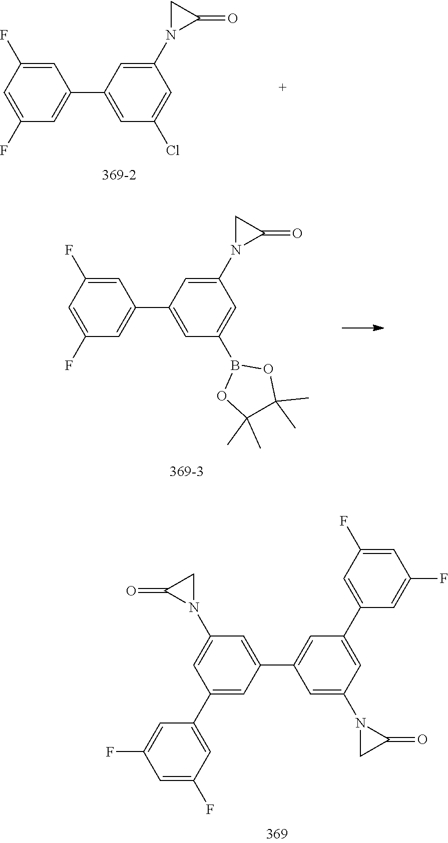 [SMILES: O=C1CN1c1cc(Cl)cc(-c2cc(F)cc(F)c2)c1 ; CC1(C)OB(c2cc(-c3cc(F)cc(F)c3)cc(N3CC3=O)c2)OC1(C)C ; O=C1CN1c1cc(-c2cc(F)cc(F)c2)cc(-c2cc(-c3cc(F)cc(F)c3)cc(N3CC3=O)c2)c1]

Intermediate 369-2 (10.0 g, 0.036 mol), Intermediate 369-3 (15.9 g, 0.043 mol), K₂CO₃ (14.9 g, 0.108 mol), Pd(OAC)₂ (2.1 g, 0.002 mol), X-Phos (1.7 g, 0.004 mol), THF 200 mL, and H₂O 50 mL were added and reacted while stirring at 70° C. for 6 hours. After completion of the reaction, the reaction mixture was extracted, concentrated, and then column and recrystallized to obtain 13.1 g of Compound 369 (yield 75.0%).

LC/MS: m/z=488[(M)⁺]

### Synthesis Example 18: Synthesis of Compound 421

### (1) Preparation Example 1: Synthesis of Intermediate 421-1

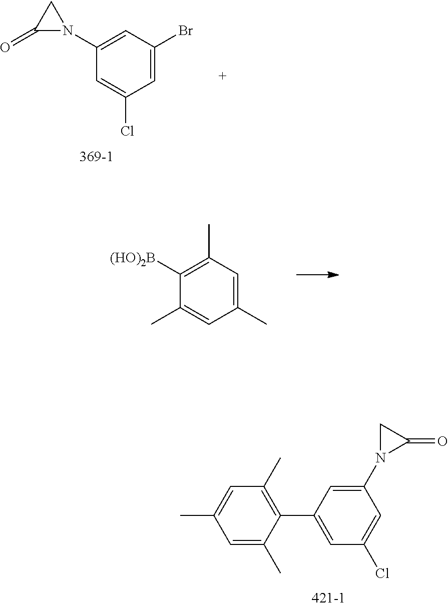

Intermediate 369-1 (10.0 g, 0.041 mol), 2,4,6-Trimethylphenylboronic acid (8.0 g, 0.049 mol), K₂CO₃ (16.8 g, 0.122 mol), and Pd(PPh₃)₄ (0.9 g, 0.0008 mol) were added with toluene 200 mL, ethanol 50 mL, and H₂O 50 mL and reacted while stirring at 80° C. for 6 hours. After completion of the reaction, the reaction mixture was extracted, concentrated, and then columned to obtain 7.7 g of Intermediate 421-1 (yield 66.4%).

### (2) Preparation Example 2: Synthesis of Intermediate 421-2

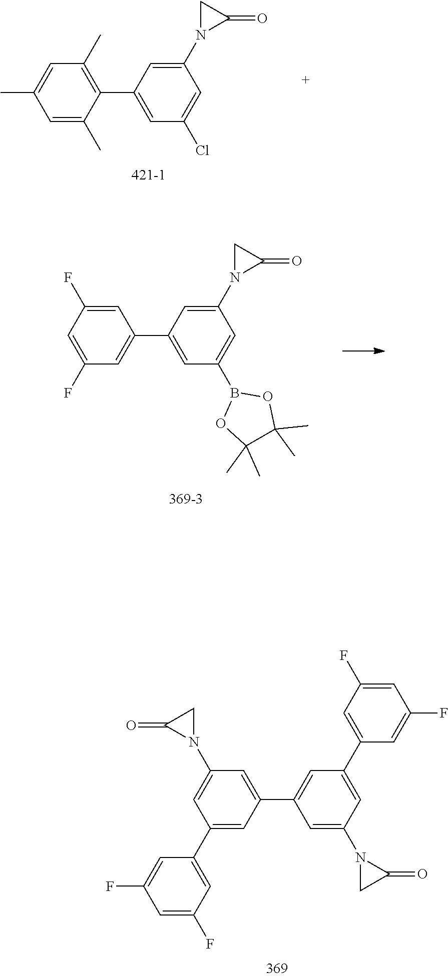 [SMILES: Cc1cc(C)c(-c2cc(Cl)cc(N3CC3=O)c2)c(C)c1 ; CC1(C)OB(c2cc(-c3cc(F)cc(F)c3)cc(N3CC3=O)c2)OC1(C)C ; O=C1CN1c1cc(-c2cc(F)cc(F)c2)cc(-c2cc(-c3cc(F)cc(F)c3)cc(N3CC3=O)c2)c1]

Intermediate 421-1 (10.0 g, 0.035 mol), Bis(pinacolato)diboron (10.7 g, 0.042 mol), KOAc (10.3 g, 0.105 mol), Pd(dppf)Cl₂ (1.3 g, 0.002 mol), and XPhos (5.0 g, 0.011 mol) were added with dioxane 200 mL and reacted while stirring at 100° C. for 12 hours. After completion of the reaction, the reaction mixture was extracted, concentrated, and then columned to obtain 9.9 g of Intermediate 421-2 (yield 75.0%).

### (3) Preparation Example 3: Synthesis of Intermediate 421-3

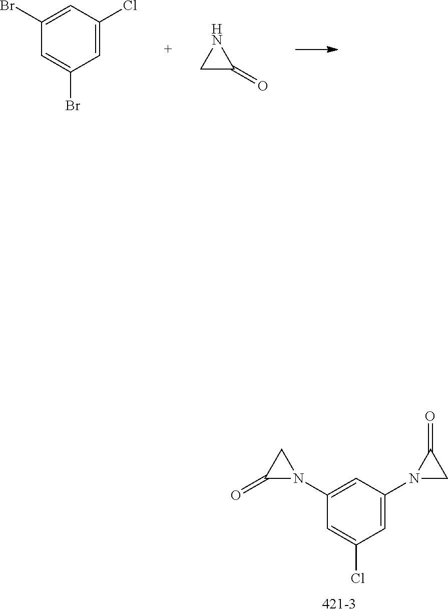

1,3-Dibromo-5-chlorobenzene (10.0 g, 0.037 mol), 2-Azetidinone (5.1 g, 0.089 mol), Cs₂CO₃ (33.7 g, 0.104 mol), Pd(dba)₂ (2.1 g, 0.004 mol), Xant-Phos (15.4 g, 0.027 mol), and dioxane were added and reacted while stirring under reflux for 16 hours. After completion of the reaction, the reaction mixture was extracted, concentrated, and then columned to obtain 6.8 g of Intermediate 421-3 (yield 82.6%).

### (4) Preparation Example 4: Synthesis of Intermediate 421-4

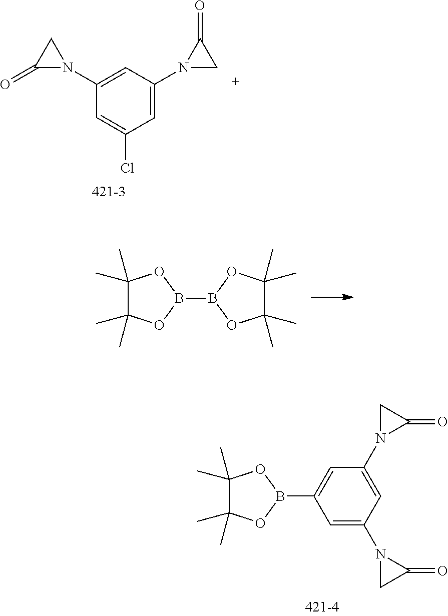

Intermediate 421-3 (10.0 g, 0.045 mol), Bis(pinacolato)diboron (13.7 g, 0.054 mol), KOAc (13.2 g, 0.135 mol), Pd(dppf)Cl₂ (1.6 g, 0.003 mol), and XPhos (6.4 g, 0.0005 mol) were added with dioxane 200 mL and reacted while stirring at 100° C. for 12 hours. After completion of the reaction, the reaction mixture was extracted, concentrated, and then columned to obtain 10.1 g of Intermediate 421-4 (yield 71.6%).

### (5) Preparation Example 5: Synthesis of Intermediate 421-5

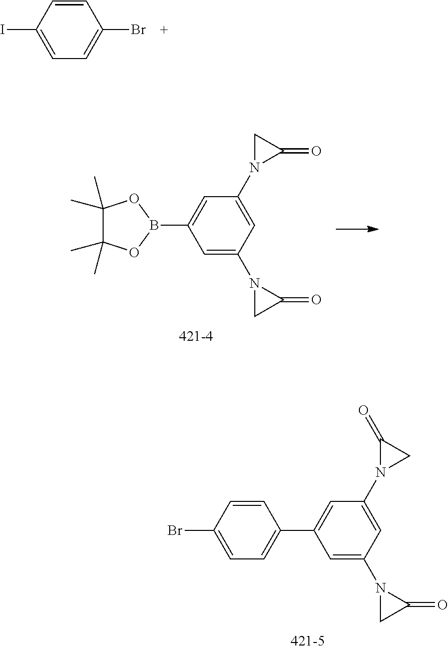 [SMILES: Brc1ccc(I)cc1 ; CC1(C)OB(c2cc(N3CC3=O)cc(N3CC3=O)c2)OC1(C)C ; O=C1CN1c1cc(-c2ccc(Br)cc2)cc(N2CC2=O)c1]

1-Bromo-4-iodobenzene (10.0 g, 0.035 mol), intermediate 421-4 (13.3 g, 0.042 mol), K₂CO₃ (14.7 g, 0.106 mol), and Pd(PPh₃)₄ (0.8 g, 0.0007 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 7.5 g of Intermediate 421-5 (yield 61.8%).

### (6) Preparation Example 6: Synthesis of Compound 421

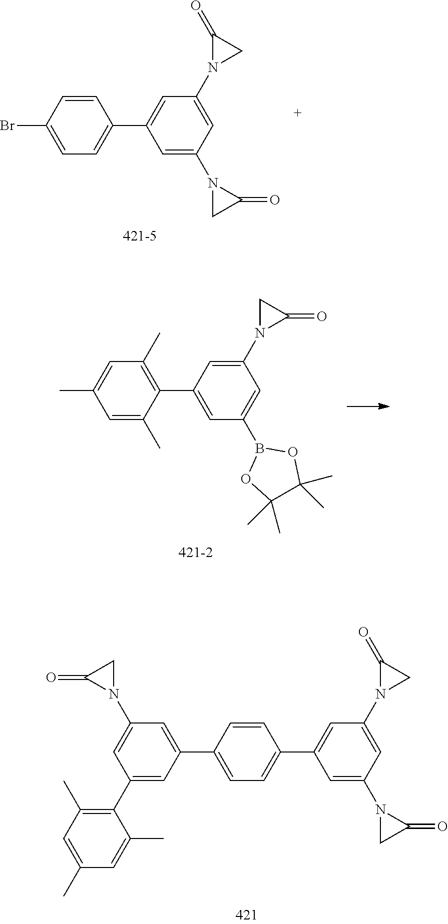

Intermediate 421-5 (10.0 g, 0.029 mol), Intermediate 421-2 (13.2 g, 0.035 mol), K₂CO₃ (12.1 g, 0.087 mol), and Pd(PPh₃)₄ (0.7 g, 0.0006 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 10.7 g of Compound 421 (yield 71.5%).

LC/MS: m/z=513[(M)⁺]

### Synthesis Example 19: Synthesis of Compound 524

### (1) Preparation Example 1: Synthesis of Intermediate 524-1

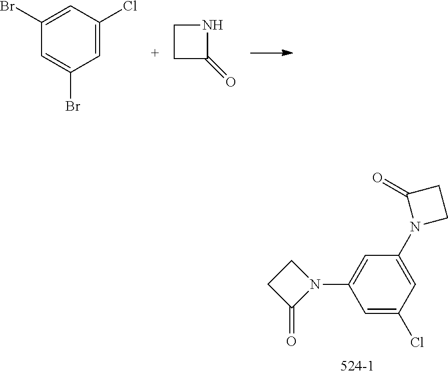 [SMILES: Clc1cc(Br)cc(Br)c1 ; O=C1CCN1 ; O=C1CCN1c1cc(Cl)cc(N2CCC2=O)c1]

1,3-Dibromo-5-chlorobenzene (10.0 g, 0.037 mol, 2-Azetidinone (6.3 g, 0.089 mol), Cs₂CO₃ (33.7 g, 0.104 mol), Pd(dba) (2.1 g, 0.004 mol), Xant-Phos (15.4 g, 0.027 mol), and dioxane were added and reacted while stirring under reflux for 16 hours. After completion of the reaction, the reaction mixture was extracted, concentrated, and then columned to obtain 8.1 g of Intermediate 524-1 (yield 87.4%).

### (2) Preparation Example 2: Synthesis of Intermediate 524-2

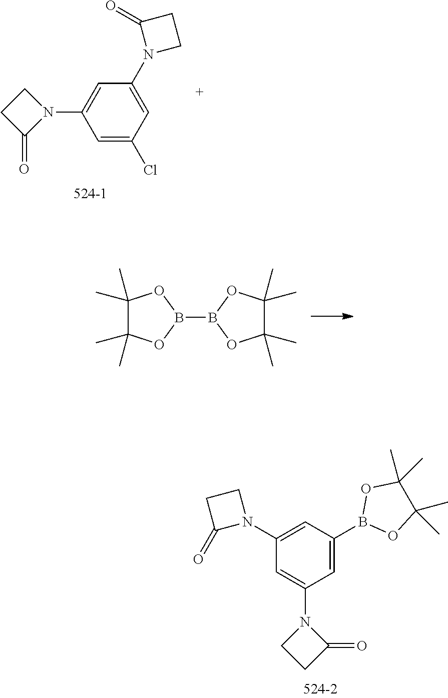

Intermediate 524-1 (10.0 g, 0.040 mol), Bis(pinacolato)diboron (12.2 g, 0.048 mol), KOAc (11.7 g, 0.120 mol), Pd(dppf)Cl₂ (1.5 g, 0.002 mol), and XPhos (5.7 g, 0.012 mol) were added with dioxane 200 mL and reacted while stirring at 100° C. for 12 hours. After completion of the reaction, the reaction mixture was extracted, concentrated, and then columned to obtain 9.7 g of Intermediate 524-2 (yield 71.1%).

### (3) Preparation Example 3: Synthesis of Intermediate 524-3

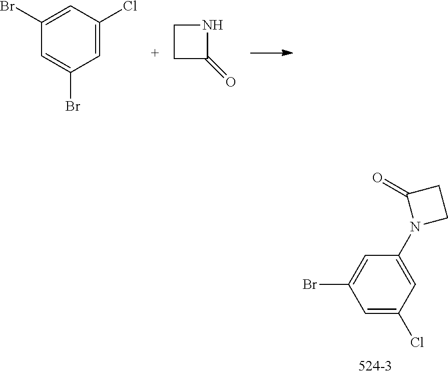 [SMILES: Clc1cc(Br)cc(Br)c1 ; O=C1CCN1 ; O=C1CCN1c1cc(Cl)cc(Br)c1]

1,3-Dibromo-5-chlorobenzene (10.0 g, 0.037 mol), 2-Azetidinone (3.2 g, 0.044 mol), Cs₂CO₃ (16.9 g, 0.052 mol), Pd(dba)₂ (1.1 g, 0.002 mol), Xant-Phos (3.9 g, 0.007 mol), and dioxane were added and reacted while stirring under reflux for 16 hours. After completion of the reaction, the reaction mixture was extracted, concentrated, and then columned to obtain 6.3 g of Intermediate 524-3 (yield 65.4%).

### (4) Preparation Example 4: Synthesis of Intermediate 524-4

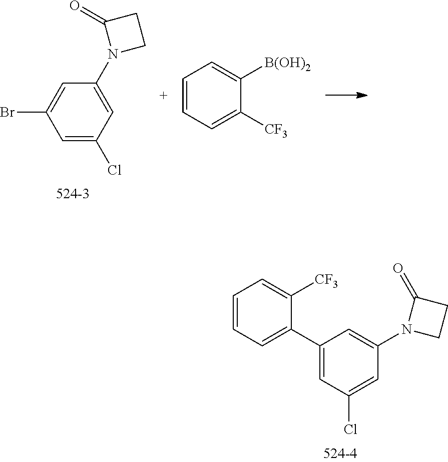 [SMILES: O=C1CCN1c1cc(Cl)cc(Br)c1 ; OB(O)c1ccccc1C(F)(F)F ; O=C1CCN1c1cc(Cl)cc(-c2ccccc2C(F)(F)F)c1]

Intermediate 524-3 (10.0 g, 0.038 mol), 2-(Trifluoromethyl)phenylboronic acid (8.8 g, 0.046 mol), K₂CO₃ (15.9 g, 0.115 mol), and Pd(PPh₃)₄ (0.9 g, 0.001 mol) were added with toluene 200 mL, ethanol 50 mL, and H₂O 50 mL and reacted while stirring at 80° C. for 6 hours. After completion of the reaction, the reaction mixture was extracted, concentrated, and then columned to obtain 8.2 g of Intermediate 524-4 (yield 65.6%).

### (5) Preparation Example 5: Synthesis of Intermediate 524-5

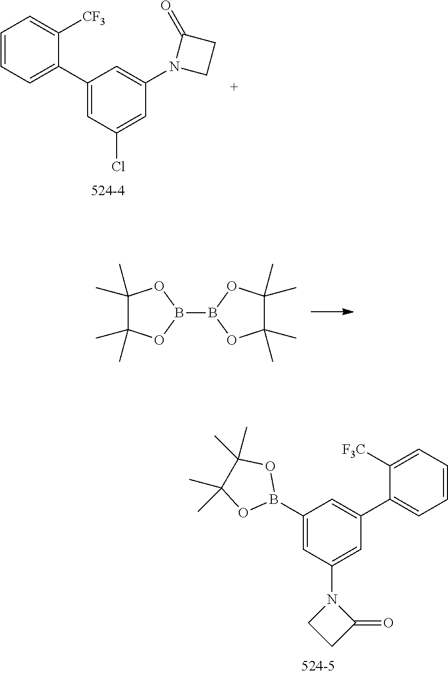

Intermediate 524-4 (10.0 g, 0.031 mol), Bis(pinacolato)diboron (9.4 g, 0.037 mol), KOAc (9.0 g, 0.092 mol), Pd(dppf)Cl₂ (1.1 g, 0.002 mol), and XPhos (4.4 g, 0.009 mol) were added with dioxane 200 mL and reacted while stirring at 100° C. for 12 hours. After completion of the reaction, the reaction mixture was extracted, concentrated, and then columned to obtain 8.9 g of Intermediate 524-5 (yield 69.5%).

### (6) Preparation Example 6: Synthesis of Intermediate 524-6

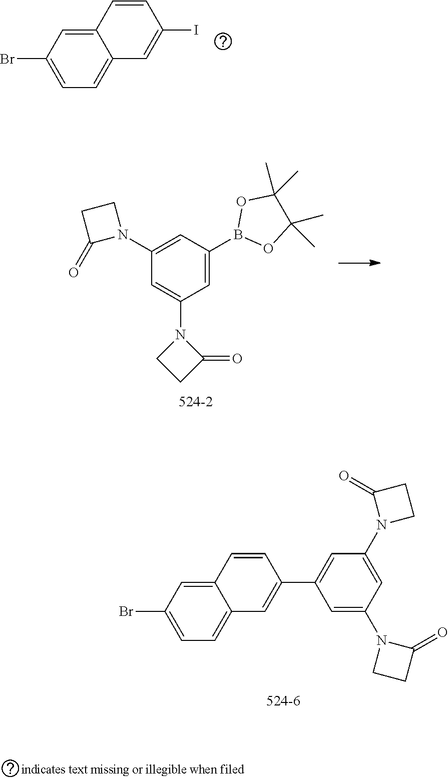 [SMILES: Brc1ccc2cc(I)ccc2c1 ; CC1(C)OB(c2cc(N3CCC3=O)cc(N3CCC3=O)c2)OC1(C)C ; O=C1CCN1c1cc(-c2ccc3cc(Br)ccc3c2)cc(N2CCC2=O)c1]

2-Bromo-6-iodobenzene (10.0 g, 0.030 mol), Intermediate 524-2 (12.3 g, 0.036 mol), K₂CO₃ (12.5 g, 0.090 mol), and Pd(PPh₃)₄ (0.7 g, 0.001 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 7.8 g of Intermediate 524-6 (yield 61.7%).

### (7) Preparation Example 7: Synthesis of Compound 524

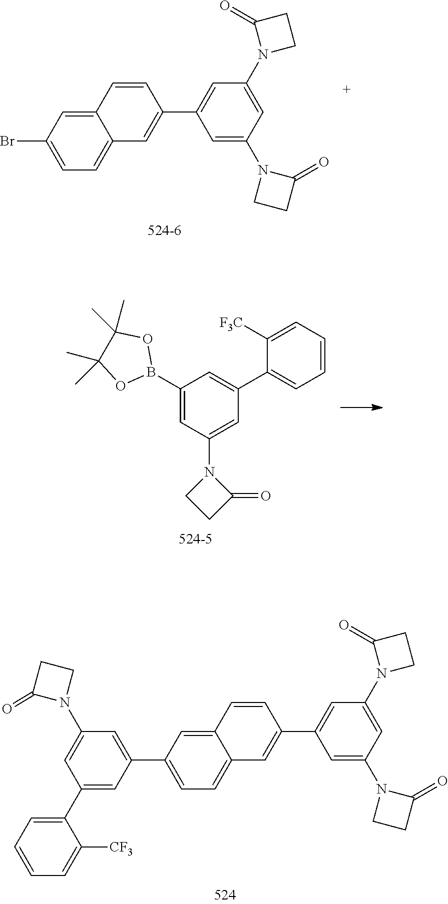 [SMILES: O=C1CCN1c1cc(-c2ccc3cc(Br)ccc3c2)cc(N2CCC2=O)c1 ; CC1(C)OB(c2cc(-c3ccccc3C(F)(F)F)cc(N3CCC3=O)c2)OC1(C)C ; O=C1CCN1c1cc(-c2ccc3cc(-c4cc(N5CCC5=O)cc(N5CCC5=O)c4)ccc3c2)cc(-c2ccccc2C(F)(F)F)c1]

Intermediate 524-6 (10.0 g, 0.024 mol), Intermediate 524-5 (11.9 g, 0.029 mol), K₂CO₃ (9.8 g, 0.071 mol), and Pd(PPh₃)₄ (0.6 g, 0.0005 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 11.2 g of Compound 524 (yield 74.7%).

LC/MS: m/z=631[(M)⁺]

### Synthesis Example 20: Synthesis of Compound 560

### (1) Preparation Example 1: Synthesis of Intermediate 560-1

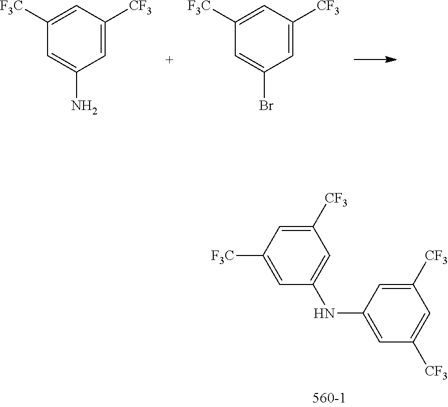 [SMILES: Nc1cc(C(F)(F)F)cc(C(F)(F)F)c1 ; FC(F)(F)c1cc(Br)cc(C(F)(F)F)c1 ; FC(F)(F)c1cc(Nc2cc(C(F)(F)F)cc(C(F)(F)F)c2)cc(C(F)(F)F)c1]

3,5-Bis(trifluoromethyl)benzenamine (10.0 g, 0.044 mol), 1-Bromo-3,5-bis(trifluoromethyl)benzene (19.5 g, 0.066 mol), NaOtBu (12.6 g, 0.131 mol), Pd(dba)₂ (1.0 g, 1.7 mmol), and t-Bu₃P (0.7 g, 3.5 mmol) were added with Toluene 150 mL and reacted while stirring at 70° C. for 4 hours. After completion of the reaction, the reaction mixture was extracted, concentrated, and then columned to obtain 10.5 g of Intermediate 560-1 (yield 54.5%).

### (2) Preparation Example 2: Synthesis of Intermediate 560-2

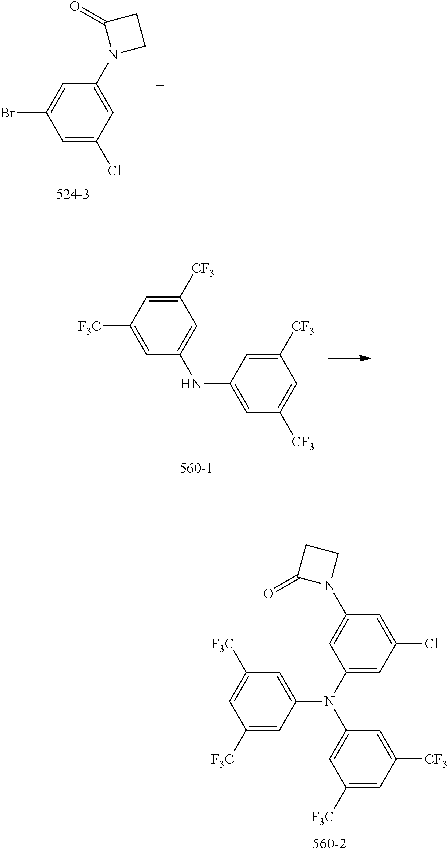 [SMILES: O=C1CCN1c1cc(Cl)cc(Br)c1 ; FC(F)(F)c1cc(Nc2cc(C(F)(F)F)cc(C(F)(F)F)c2)cc(C(F)(F)F)c1 ; O=C1CCN1c1cc(Cl)cc(N(c2cc(C(F)(F)F)cc(C(F)(F)F)c2)c2cc(C(F)(F)F)cc(C(F)(F)F)c2)c1]

Intermediate 524-3 (10.0 g, 0.038 mol), Intermediate 560-1 (25.4 g, 0.058 mol), NaOtBu (11.1 g, 0.115 mol), Pd(dba)₂ (0.9 g, 1.5 mmol), and t-Bu₃P (0.6 g, 3.1 mmol) were added with Toluene 150 mL and reacted while stirring at 70° C. for 4 hours. After completion of the reaction, the reaction mixture was extracted, concentrated, and then columned to obtain 13.1 g of Intermediate 560-2 (yield 55.0%).

### (3) Preparation Example 3: Synthesis of Intermediate 560-3

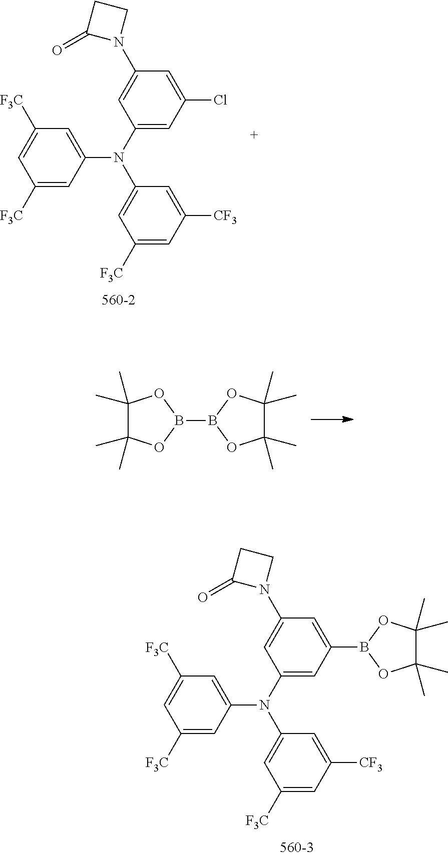

Intermediate 560-2 (10.0 g, 0.016 mol), Bis(pinacolato)diboron (4.9 g, 0.019 mol), KOAc (4.7 g, 0.048 mol), Pd(dppf)Cl₂ (0.6 g, 0.001 mol), and XPhos (2.3 g, 0.005 mol) were added with dioxane 200 mL and reacted while stirring at 100° C. for 12 hours. After completion of the reaction, the reaction mixture was extracted, concentrated, and then columned to obtain 7.7 g of Intermediate 560-3 (yield 67.1%).

### (4) Preparation Example 4: Synthesis of Intermediate 560-4

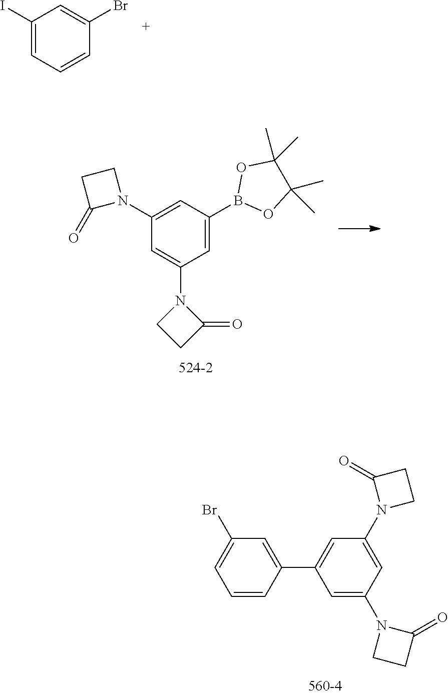 [SMILES: Brc1cccc(I)c1 ; CC1(C)OB(c2cc(N3CCC3=O)cc(N3CCC3=O)c2)OC1(C)C ; O=C1CCN1c1cc(-c2cccc(Br)c2)cc(N2CCC2=O)c1]

1-Chloro-3-iodobenzene (10.0 g, 0.035 mol), intermediate 524-2 (14.5 g, 0.042 mol), K₂CO₃ (14.7 g, 0.106 mol), and Pd(PPh₃)₄ (0.8 g, 0.0008 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 8.3 g of Intermediate 560-4 (yield 63.3%).

### (5) Preparation Example 5: Synthesis of Compound 560

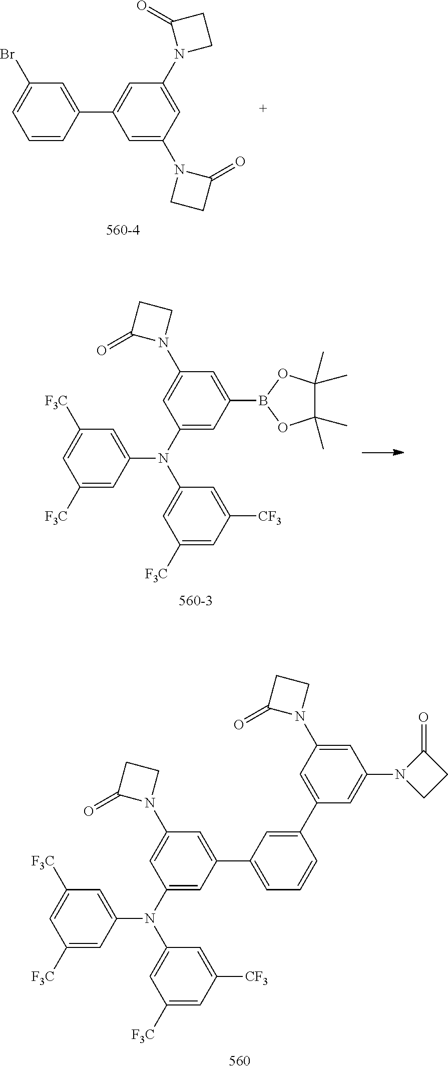 [SMILES: O=C1CCN1c1cc(-c2cccc(Br)c2)cc(N2CCC2=O)c1 ; CC1(C)OB(c2cc(N3CCC3=O)cc(N(c3cc(C(F)(F)F)cc(C(F)(F)F)c3)c3cc(C(F)(F)F)cc(C(F)(F)F)c3)c2)OC1(C)C ; O=C1CCN1c1cc(-c2cccc(-c3cc(N4CCC4=O)cc(N(c4cc(C(F)(F)F)cc(C(F)(F)F)c4)c4cc(C(F)(F)F)cc(C(F)(F)F)c4)c3)c2)cc(N2CCC2=O)c1]

Intermediate 560-4 (10.0 g, 0.027 mol), Intermediate 560-3 (23.0 g, 0.032 mol), K₂CO₃ (11.2 g, 0.081 mol), and Pd(PPh₃)₄ (0.6 g, 0.001 mol) were added with toluene 200 mL, ethanol 50 mL, and H₂O 50 mL and reacted while stirring at 80° C. for 6 hours. After completion of the reaction, the reaction mixture was extracted, concentrated, and then column and recrystallized to obtain 16.3 g of Compound 560 (yield 69.0%).

LC/MS: m/z=876[(M)⁺]

### Device Example (CPL)

In Example according to the present invention, an Ag-containing ITO glass substrate having dimensions of 25 mm×25 mm×0.7 mm was used as an anode, patterned to have a light emitting area of 2 mm×2 mm, followed by cleaning. After the patterned ITO substrate was mounted in a vacuum chamber, organic materials and metals were deposited in the following structure on the substrate at a process pressure of 1×10⁻⁶ torr or more.

### Device Examples 1 to 101

The compounds implemented according to the present invention was employed in a light efficiency improving layer to fabricate an organic light-emitting device having the following device structure, and then the luminescent and driving characteristics according to the compound implemented according to the present invention were measured.

Ag:ITO/hole injecting layer (HAT-CN, 5 nm)/hole transport layer (α-NPB, 100 nm)/electron blocking layer (TCTA, 10 nm)/light emitting layer (20 nm)/electron transport layer (201:Liq, 30 nm)/LiF (1 nm)/Mg:Ag (15 nm)/light efficiency improving layer (70 nm)

On a glass substrate, HAT-CN was formed into a 5 nm-thick film to form a hole injecting layer on an Ag-containing ITO transparent electrode. Thereafter, α-NPB was formed into a 100 nm-thick film to form a hole transport layer, and then TCTA was formed into a 10 nm-thick film to form an electron blocking layer. Thereafter, a 20 nm-thick light emitting layer was formed by co-depositing BH1 as a host compound and BD1 as a dopant compound, a 30 nm-thick electron transport layer (doped with Liq 50% of the following [201] compound) was deposited, and then LiF was formed into a 1 nm-thick film to form an electron injecting layer, and Mg:Ag at a ratio of 1:9 was formed into a 15 nm-thick film to form a cathode. Thereafter, a light efficiency improving layer (capping layer) was formed into a 70 nm-thick film using the compound implemented in the present invention to fabricate an organic light-emitting device.

### Device Comparative Example 1

An organic light-emitting device for Device Comparative Example 1 was fabricated in the same manner, except that the light efficiency improving layer in Example was not provided

### Device Comparative Example 2

An organic light-emitting device for Device Comparative Example 2 was fabricated in the same manner, except that Alq₃ was used instead of the compound of the present invention as the light efficiency improving layer compound in Example.

### Experimental Example 1: Luminescent Properties of Device Examples 1 to 101

The driving voltages, current efficiencies, and color coordinates of the organic light-emitting devices fabricated in Examples and Comparative Examples above were measured using a source meter (Model 237, Keithley) and a luminance meter (PR-650, Photo Research). The result values at 1,000 nits were shown in Table 1 below.

TABLE 1

| | Light efficiency | | | | |
|---|---|---|---|---|---|
| Example | improving layer | V | cd/A | CIEx | CIEy |
| 1 | Formula 1 | 3.58 | 8.92 | 0.1417 | 0.0504 |
| 2 | Formula 2 | 3.55 | 8.68 | 0.1390 | 0.0473 |
| 3 | Formula 3 | 3.38 | 8.85 | 0.1424 | 0.0505 |
| 4 | Formula 4 | 3.51 | 8.69 | 0.1439 | 0.0467 |
| 5 | Formula 5 | 3.44 | 8.84 | 0.1452 | 0.0455 |
| 6 | Formula 6 | 3.75 | 8.42 | 0.1405 | 0.0445 |
| 7 | Formula 7 | 3.54 | 8.78 | 0.1414 | 0.0490 |
| 8 | Formula 8 | 3.66 | 8.45 | 0.1426 | 0.0455 |
| 9 | Formula 9 | 3.40 | 9.01 | 0.1411 | 0.0556 |
| 10 | Formula 12 | 3.55 | 8.75 | 0.1437 | 0.0491 |
| 11 | Formula 13 | 3.69 | 8.34 | 0.1420 | 0.0465 |
| 12 | Formula 14 | 3.45 | 8.82 | 0.1374 | 0.0495 |
| 13 | Formula 15 | 3.31 | 9.01 | 0.1414 | 0.0548 |
| 14 | Formula 17 | 3.37 | 8.80 | 0.1397 | 0.0555 |
| 15 | Formula 18 | 3.70 | 8.32 | 0.1434 | 0.0431 |
| 16 | Formula 25 | 3.51 | 8.78 | 0.1427 | 0.0488 |
| 17 | Formula 26 | 3.35 | 9.04 | 0.1415 | 0.0533 |
| 18 | Formula 27 | 3.52 | 8.75 | 0.1386 | 0.0485 |
| 19 | Formula 28 | 3.41 | 8.79 | 0.1428 | 0.0482 |
| 20 | Formula 30 | 3.35 | 9.06 | 0.1434 | 0.0475 |
| 21 | Formula 31 | 3.70 | 8.44 | 0.1412 | 0.0440 |
| 22 | Formula 38 | 3.62 | 8.52 | 0.1416 | 0.0435 |
| 23 | Formula 40 | 3.37 | 8.98 | 0.1385 | 0.0539 |
| 24 | Formula 41 | 3.39 | 8.92 | 0.1447 | 0.0474 |
| 25 | Formula 47 | 3.70 | 8.75 | 0.1423 | 0.0483 |
| 26 | Formula 53 | 3.38 | 8.87 | 0.1431 | 0.0485 |
| 27 | Formula 59 | 3.51 | 8.62 | 0.1393 | 0.0496 |
| 28 | Formula 60 | 3.66 | 8.44 | 0.1424 | 0.0481 |
| 29 | Formula 63 | 3.33 | 9.01 | 0.1416 | 0.0515 |
| 30 | Formula 68 | 3.38 | 8.97 | 0.1382 | 0.0578 |
| 31 | Formula 71 | 3.55 | 8.64 | 0.1431 | 0.0475 |
| 32 | Formula 79 | 3.63 | 8.35 | 0.1441 | 0.0432 |
| 33 | Formula 82 | 3.46 | 8.40 | 0.1424 | 0.0468 |
| 34 | Formula 86 | 3.42 | 8.68 | 0.1380 | 0.0530 |
| 35 | Formula 93 | 3.64 | 8.26 | 0.1408 | 0.0518 |
| 36 | Formula 98 | 3.47 | 8.42 | 0.1427 | 0.0449 |
| 37 | Formula 109 | 3.34 | 8.87 | 0.1431 | 0.0464 |
| 38 | Formula 111 | 3.80 | 8.82 | 0.1399 | 0.0483 |
| 39 | Formula 123 | 3.69 | 9.06 | 0.1442 | 0.0568 |
| 40 | Formula 124 | 3.28 | 8.76 | 0.1395 | 0.0568 |
| 41 | Formula 130 | 3.64 | 8.22 | 0.1413 | 0.0513 |
| 42 | Formula 135 | 3.68 | 8.35 | 0.1404 | 0.0500 |
| 43 | Formula 138 | 3.33 | 8.80 | 0.1421 | 0.0486 |
| 44 | Formula 141 | 3.45 | 8.57 | 0.1387 | 0.0502 |
| 45 | Formula 152 | 3.34 | 8.80 | 0.1441 | 0.0443 |
| 46 | Formula 153 | 3.38 | 8.59 | 0.1451 | 0.0461 |
| 47 | Formula 155 | 3.42 | 8.94 | 0.1416 | 0.0532 |
| 48 | Formula 160 | 3.34 | 8.95 | 0.1411 | 0.0563 |
| 49 | Formula 164 | 3.45 | 8.50 | 0.1423 | 0.0492 |
| 50 | Formula 179 | 3.26 | 8.71 | 0.1402 | 0.0536 |
| 51 | Formula 183 | 3.54 | 8.64 | 0.1424 | 0.0517 |
| 52 | Formula 192 | 3.32 | 8.78 | 0.1405 | 0.0563 |
| 53 | Formula 197 | 3.59 | 8.35 | 0.1401 | 0.0510 |
| 54 | Formula 201 | 3.46 | 8.60 | 0.1395 | 0.0493 |
| 55 | Formula 203 | 3.57 | 8.34 | 0.1412 | 0.0468 |
| 56 | Formula 213 | 3.26 | 8.72 | 0.1415 | 0.0564 |
| 57 | Formula 218 | 3.58 | 8.89 | 0.1423 | 0.0491 |
| 58 | Formula 223 | 3.34 | 8.78 | 0.1446 | 0.0454 |
| 59 | Formula 230 | 3.61 | 8.35 | 0.1410 | 0.0443 |
| 60 | Formula 238 | 3.34 | 8.84 | 0.1415 | 0.0563 |
| 61 | Formula 243 | 3.80 | 8.91 | 0.1424 | 0.0499 |
| 62 | Formula 254 | 3.75 | 8.42 | 0.1440 | 0.0465 |
| 63 | Formula 258 | 3.46 | 8.74 | 0.1414 | 0.0508 |
| 64 | Formula 267 | 3.43 | 8.80 | 0.1454 | 0.0455 |
| 65 | Formula 270 | 3.58 | 8.52 | 0.1411 | 0.0511 |
| 66 | Formula 277 | 3.72 | 8.86 | 0.1386 | 0.0517 |
| 67 | Formula 280 | 3.59 | 8.94 | 0.1418 | 0.0511 |
| 68 | Formula 282 | 3.87 | 8.54 | 0.1421 | 0.0459 |
| 69 | Formula 287 | 3.69 | 8.82 | 0.1380 | 0.0492 |
| 70 | Formula 288 | 3.93 | 8.50 | 0.1395 | 0.0517 |
| 71 | Formula 294 | 3.58 | 9.15 | 0.1405 | 0.0570 |
| 72 | Formula 298 | 3.69 | 8.74 | 0.1383 | 0.0501 |
| 73 | Formula 301 | 3.63 | 8.89 | 0.1400 | 0.0568 |
| 74 | Formula 311 | 3.71 | 8.74 | 0.1385 | 0.0509 |
| 75 | Formula 313 | 3.59 | 9.02 | 0.1418 | 0.0501 |
| 76 | Formula 320 | 3.74 | 8.83 | 0.1384 | 0.0497 |
| 77 | Formula 333 | 3.65 | 8.94 | 0.1397 | 0.0574 |
| 78 | Formula 340 | 3.74 | 9.17 | 0.1411 | 0.0510 |
| 79 | Formula 351 | 3.76 | 8.84 | 0.1405 | 0.0517 |
| 80 | Formula 369 | 3.70 | 9.16 | 0.1381 | 0.0543 |
| 81 | Formula 371 | 3.77 | 8.97 | 0.1404 | 0.0498 |
| 82 | Formula 391 | 3.70 | 9.01 | 0.1395 | 0.0574 |
| 83 | Formula 401 | 3.64 | 9.27 | 0.1381 | 0.0530 |
| 84 | Formula 403 | 4.03 | 8.64 | 0.1430 | 0.0449 |
| 85 | Formula 418 | 3.70 | 9.18 | 0.1382 | 0.0563 |
| 86 | Formula 421 | 3.59 | 9.04 | 0.1407 | 0.0562 |
| 87 | Formula 480 | 3.91 | 8.52 | 0.1420 | 0.0472 |
| 88 | Formula 495 | 3.67 | 8.85 | 0.1406 | 0.0518 |
| 89 | Formula 500 | 3.63 | 8.94 | 0.1415 | 0.0503 |
| 90 | Formula 501 | 3.79 | 8.66 | 0.1407 | 0.0521 |
| 91 | Formula 509 | 3.72 | 8.82 | 0.1421 | 0.0482 |
| 92 | Formula 511 | 3.89 | 8.44 | 0.1430 | 0.0479 |
| 93 | Formula 521 | 3.76 | 8.60 | 0.1408 | 0.0511 |
| 94 | Formula 524 | 3.49 | 9.17 | 0.1397 | 0.0530 |
| 95 | Formula 533 | 3.92 | 8.51 | 0.1410 | 0.0503 |
| 96 | Formula 540 | 3.49 | 9.26 | 0.1427 | 0.0487 |
| 97 | Formula 549 | 3.60 | 9.22 | 0.1439 | 0.0463 |
| 98 | Formula 553 | 3.99 | 8.68 | 0.1449 | 0.0464 |
| 99 | Formula 560 | 3.67 | 9.21 | 0.1381 | 0.0567 |
| 100 | Formula 563 | 3.78 | 8.80 | 0.1399 | 0.0493 |
| 101 | Formula 566 | 3.70 | 9.07 | 0.1415 | 0.0568 |
| Comparative | Not used | 4.68 | 7.03 | 0.1502 | 0.1412 |
| Example 1 | | | | | |
| Comparative | Alq₃ | 4.33 | 7.84 | 0.1471 | 0.0583 |
| Example 2 | | | | | |

As described in the results shown in Table 1, it was confirmed that in the case of the organic light-emitting device in which the compound according to the present invention was applied to the light efficiency improving layer provided in the device, the driving voltage was reduced and the current efficiency was improved compared to a conventional device without a light efficiency improving layer and a conventional device employed with a compound (Alq₃) used as the light efficiency improving layer (Comparative Examples 1 and 2).

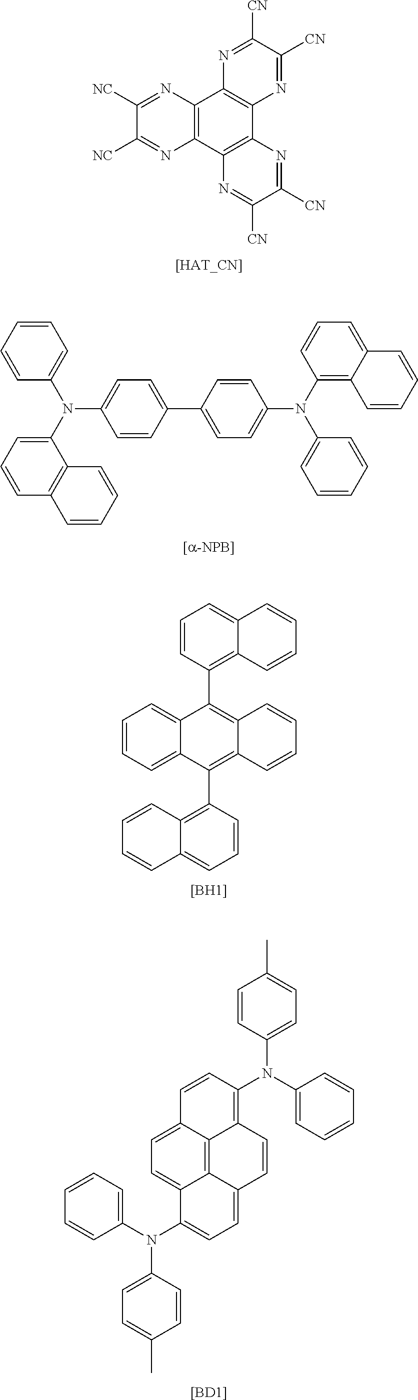

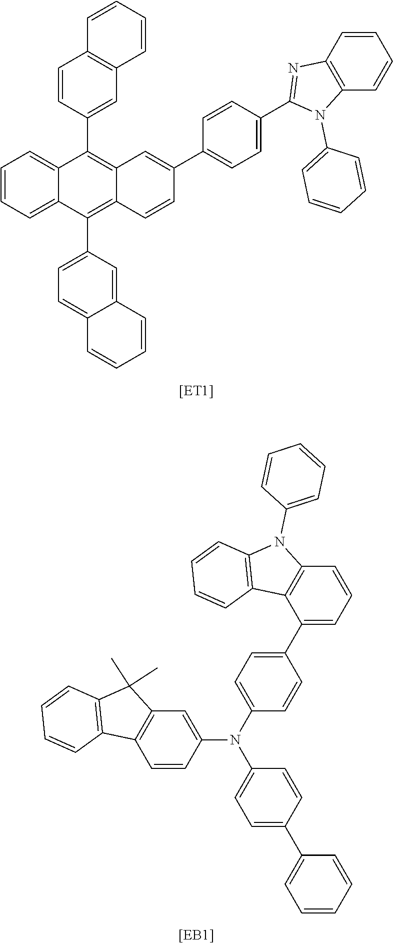 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CC1(C)c2ccccc2-c2ccc(N(c3ccc(-c4ccccc4)cc3)c3ccc(-c4cccc5c4c4ccccc4n5-c4ccccc4)cc3)cc21]

### INDUSTRIAL APPLICABILITY

According to the present invention, an organic compound is employed as a material for a light efficiency improving layer provided in an organic light-emitting device to achieve low-voltage driving and improved luminescent properties such as excellent luminous efficiency, color purity, etc. by increasing the light efficiency of the organic light-emitting device, and thus the present invention can be effectively used industrially in various lighting and display devices.

1. A compound represented by Formula I below: 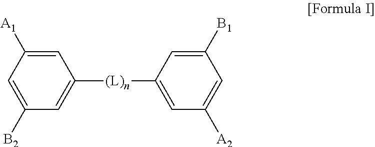
In Formula I above,
L is a single bond or any one selected from a substituted or unsubstituted arylene group having 6 to 30 carbon atoms and a substituted or unsubstituted heteroarylene group having 3 to 30 carbon atoms,
n is an integer of 0 to 2, and when the n is 2, a plurality of L are the same as or different from each other,
A₁ and A₂ are the same as or different from each other, and each independently selected from structures shown in Structural Formula 1 below (‘*’ indicates a linking site), 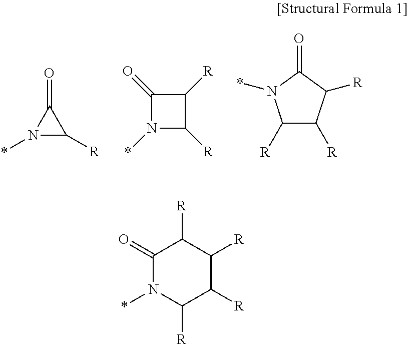
in Structural Formula 1 above,
in each structure, R is each independently any one selected from hydrogen, deuterium, a cyano group, a halogen group, a substituted or unsubstituted alkyl group having 1 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted cycloalkyl group having 3 to 20 carbon atoms, a substituted or unsubstituted aryl group having 6 to 30 carbon atoms, and a substituted or unsubstituted heteroaryl group having 2 to 30 carbon atoms, and
in each structure of Structural Formula 1, a plurality of R are the same as or different from each other,
B₁ and B₂ are the same as or different from each other, and each independently any one selected from hydrogen, deuterium, a cyano group, a halogen group, a substituted or unsubstituted alkyl group having 1 to 20 carbon atoms, a substituted or unsubstituted alkoxy group having 1 to 20 carbon atoms, a substituted or unsubstituted halogenated alkyl group having 1 to 20 carbon atoms, a substituted or unsubstituted halogenated alkoxy group having 1 to 20 carbon atoms, a substituted or unsubstituted cycloalkyl group having 3 to 20 carbon atoms, a substituted or unsubstituted aryl group having 6 to 30 carbon atoms, a substituted or unsubstituted heteroaryl group having 2 to 30 carbon atoms, Structural Formula 1 above, and Structural Formula 2 below. 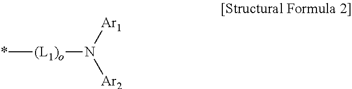
in Structural Formula 2 above,
L₁ is a single bond or any one selected from a substituted or unsubstituted arylene group having 6 to 30 carbon atoms and a substituted or unsubstituted heteroarylene group having 3 to 30 carbon atoms,
is an integer of 0 to 2, and when the o is 2, a plurality of L₁ are the same as or different from each other, and
Ar₁ and Ar₂ are the same as or different from each other, and each independently any one selected from a substituted or unsubstituted aryl group having 6 to 30 carbon atoms and a substituted or unsubstituted heteroaryl group having 2 to 30 carbon atoms.

2. The compound of claim 1, wherein the ‘substituted or unsubstituted’ in the definition of R, B₁, B₂, L, L₁, Ar₁ and Ar₂ above means substitution of R, B₁, B₂, L, L₁, Ar₁ and Ar₂ with one or two or more substituents selected from the group consisting of deuterium, a halogen group, a cyano group, a nitro group, a hydroxy group, a silyl group, an alkyl group, an amine group, a halogenated alkyl group, a deuterated alkyl group, a cycloalkyl group, a heterocycloalkyl group, an alkoxy group, a halogenated alkoxy group, a deuterated alkoxy group, an aryl group, a heteroaryl group, an alkylsilyl group and an arylsilyl group, substitution with a substituent to which two or more of the substituents are linked, or having no substituent.

3. The compound of claim 1, wherein Formula 1 above is selected from Compounds 1 to 573 below:

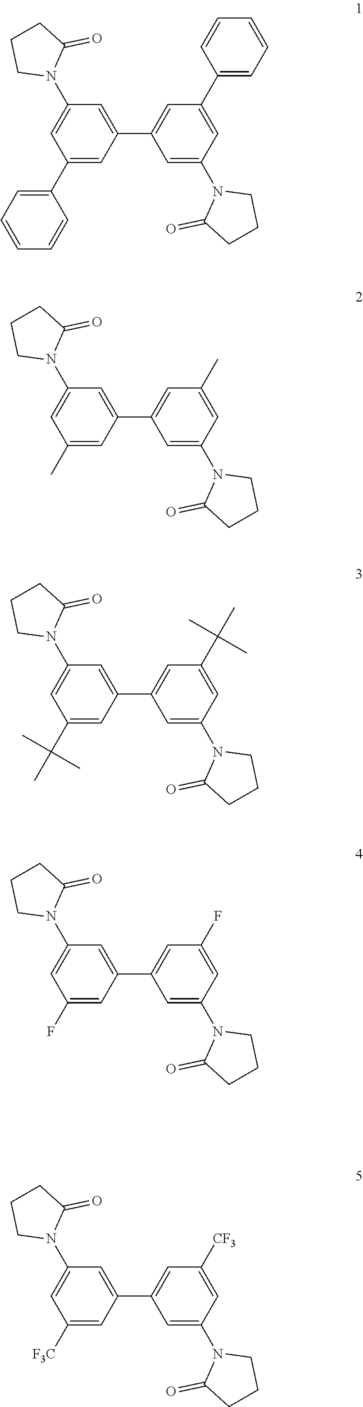 [SMILES: O=C1CCCN1c1cc(-c2ccccc2)cc(-c2cc(-c3ccccc3)cc(N3CCCC3=O)c2)c1 ; Cc1cc(-c2cc(C)cc(N3CCCC3=O)c2)cc(N2CCCC2=O)c1 ; CC(C)(C)c1cc(-c2cc(N3CCCC3=O)cc(C(C)(C)C)c2)cc(N2CCCC2=O)c1 ; O=C1CCCN1c1cc(F)cc(-c2cc(F)cc(N3CCCC3=O)c2)c1 ; O=C1CCCN1c1cc(-c2cc(N3CCCC3=O)cc(C(F)(F)F)c2)cc(C(F)(F)F)c1]

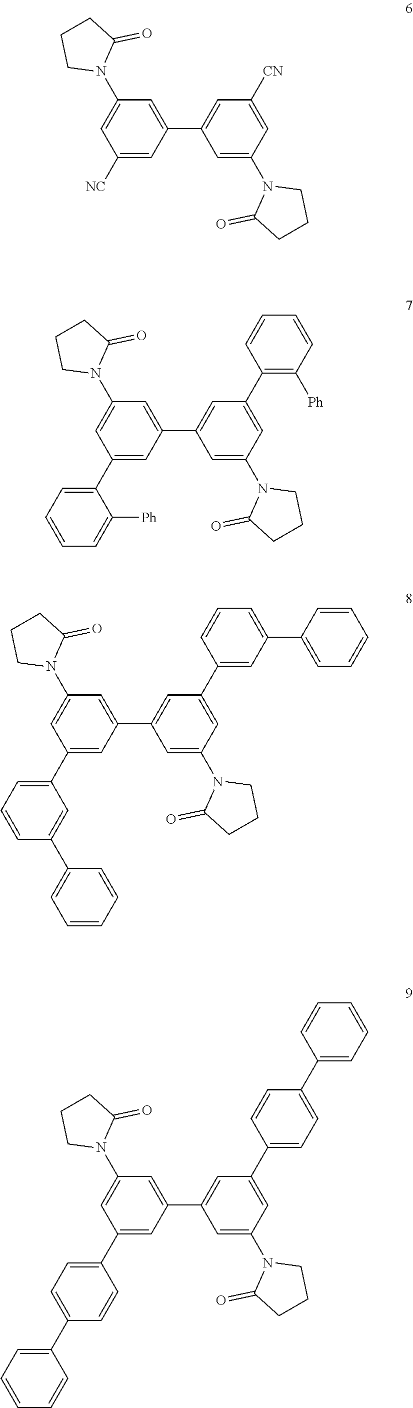 [SMILES: N#Cc1cc(-c2cc(C#N)cc(N3CCCC3=O)c2)cc(N2CCCC2=O)c1 ; O=C1CCCN1c1cc(-c2cc(-c3ccccc3-c3ccccc3)cc(N3CCCC3=O)c2)cc(-c2ccccc2-c2ccccc2)c1 ; O=C1CCCN1c1cc(-c2cccc(-c3ccccc3)c2)cc(-c2cc(-c3cccc(-c4ccccc4)c3)cc(N3CCCC3=O)c2)c1 ; O=C1CCCN1c1cc(-c2ccc(-c3ccccc3)cc2)cc(-c2cc(-c3ccc(-c4ccccc4)cc3)cc(N3CCCC3=O)c2)c1]

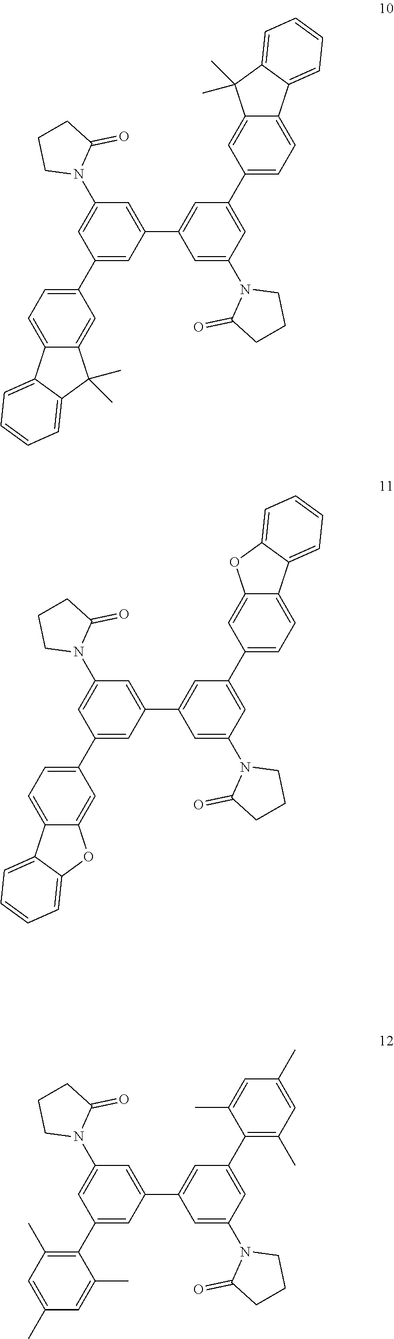 [SMILES: CC1(C)c2ccccc2-c2ccc(-c3cc(-c4cc(-c5ccc6c(c5)C(C)(C)c5ccccc5-6)cc(N5CCCC5=O)c4)cc(N4CCCC4=O)c3)cc21 ; O=C1CCCN1c1cc(-c2cc(-c3ccc4c(c3)oc3ccccc34)cc(N3CCCC3=O)c2)cc(-c2ccc3c(c2)oc2ccccc23)c1 ; Cc1cc(C)c(-c2cc(-c3cc(-c4c(C)cc(C)cc4C)cc(N4CCCC4=O)c3)cc(N3CCCC3=O)c2)c(C)c1]

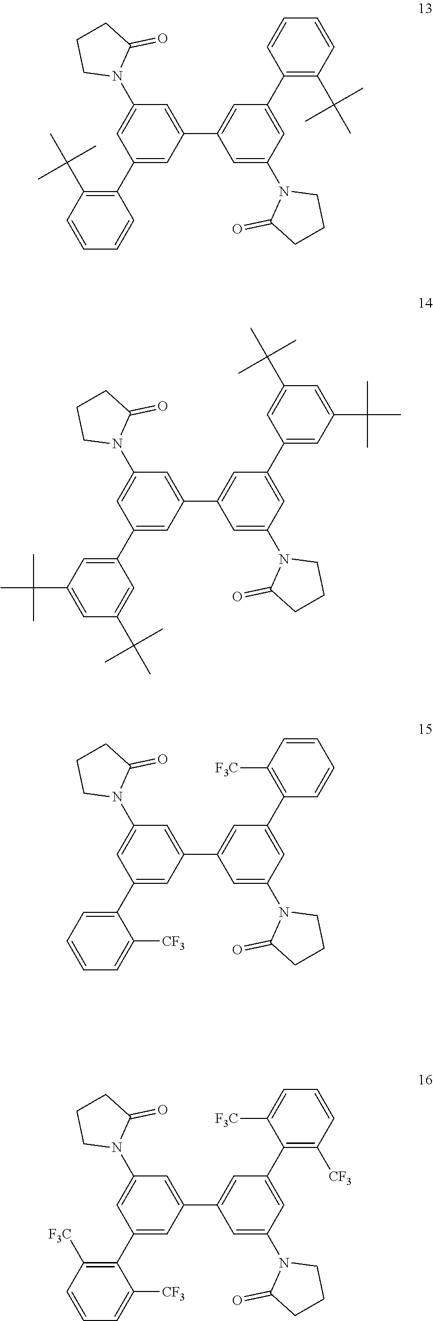

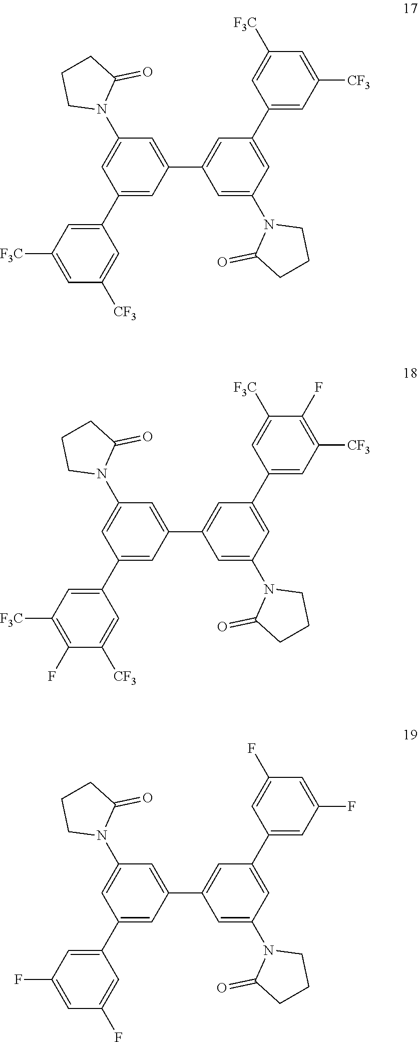 [SMILES: O=C1CCCN1c1cc(-c2cc(-c3cc(C(F)(F)F)cc(C(F)(F)F)c3)cc(N3CCCC3=O)c2)cc(-c2cc(C(F)(F)F)cc(C(F)(F)F)c2)c1 ; O=C1CCCN1c1cc(-c2cc(-c3cc(C(F)(F)F)c(F)c(C(F)(F)F)c3)cc(N3CCCC3=O)c2)cc(-c2cc(C(F)(F)F)c(F)c(C(F)(F)F)c2)c1 ; O=C1CCCN1c1cc(-c2cc(F)cc(F)c2)cc(-c2cc(-c3cc(F)cc(F)c3)cc(N3CCCC3=O)c2)c1]

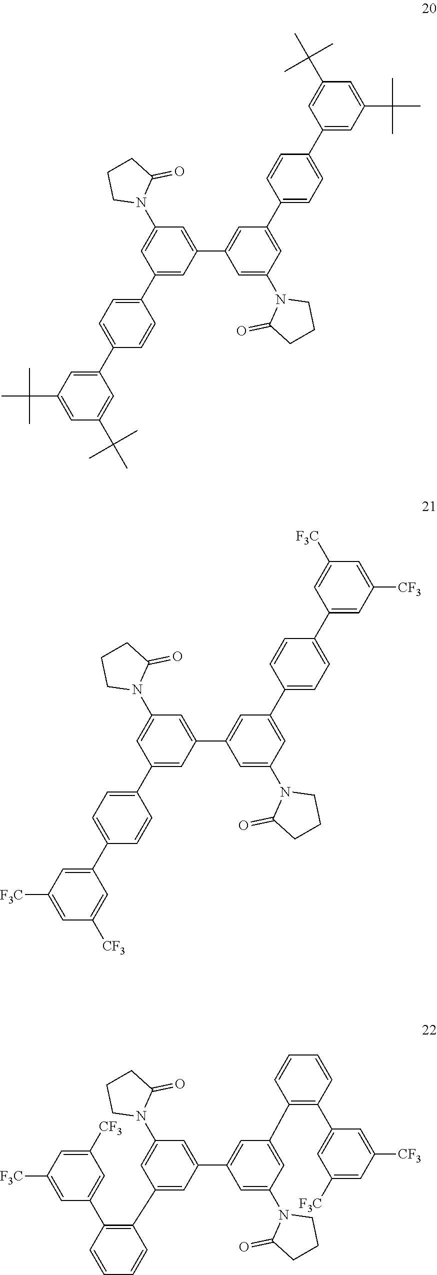 [SMILES: CC(C)(C)c1cc(-c2ccc(-c3cc(-c4cc(-c5ccc(-c6cc(C(C)(C)C)cc(C(C)(C)C)c6)cc5)cc(N5CCCC5=O)c4)cc(N4CCCC4=O)c3)cc2)cc(C(C)(C)C)c1 ; O=C1CCCN1c1cc(-c2ccc(-c3cc(C(F)(F)F)cc(C(F)(F)F)c3)cc2)cc(-c2cc(-c3ccc(-c4cc(C(F)(F)F)cc(C(F)(F)F)c4)cc3)cc(N3CCCC3=O)c2)c1 ; O=C1CCCN1c1cc(-c2cc(-c3ccccc3-c3cc(C(F)(F)F)cc(C(F)(F)F)c3)cc(N3CCCC3=O)c2)cc(-c2ccccc2-c2cc(C(F)(F)F)cc(C(F)(F)F)c2)c1]

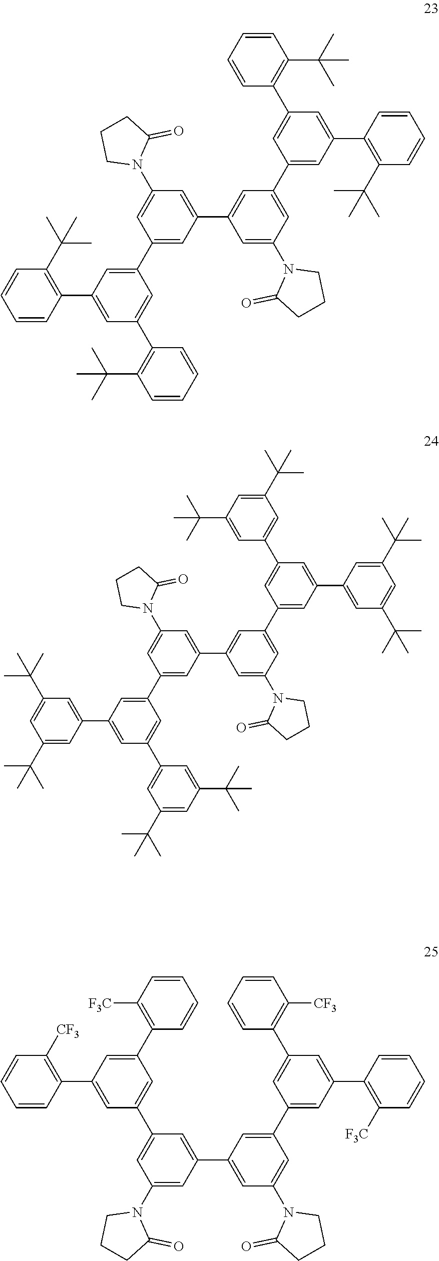 [SMILES: CC(C)(C)c1ccccc1-c1cc(-c2cc(-c3cc(-c4cc(-c5ccccc5C(C)(C)C)cc(-c5ccccc5C(C)(C)C)c4)cc(N4CCCC4=O)c3)cc(N3CCCC3=O)c2)cc(-c2ccccc2C(C)(C)C)c1 ; CC(C)(C)c1cc(-c2cc(-c3cc(-c4cc(-c5cc(-c6cc(C(C)(C)C)cc(C(C)(C)C)c6)cc(-c6cc(C(C)(C)C)cc(C(C)(C)C)c6)c5)cc(N5CCCC5=O)c4)cc(N4CCCC4=O)c3)cc(-c3cc(C(C)(C)C)cc(C(C)(C)C)c3)c2)cc(C(C)(C)C)c1 ; O=C1CCCN1c1cc(-c2cc(-c3ccccc3C(F)(F)F)cc(-c3ccccc3C(F)(F)F)c2)cc(-c2cc(-c3cc(-c4ccccc4C(F)(F)F)cc(-c4ccccc4C(F)(F)F)c3)cc(N3CCCC3=O)c2)c1]

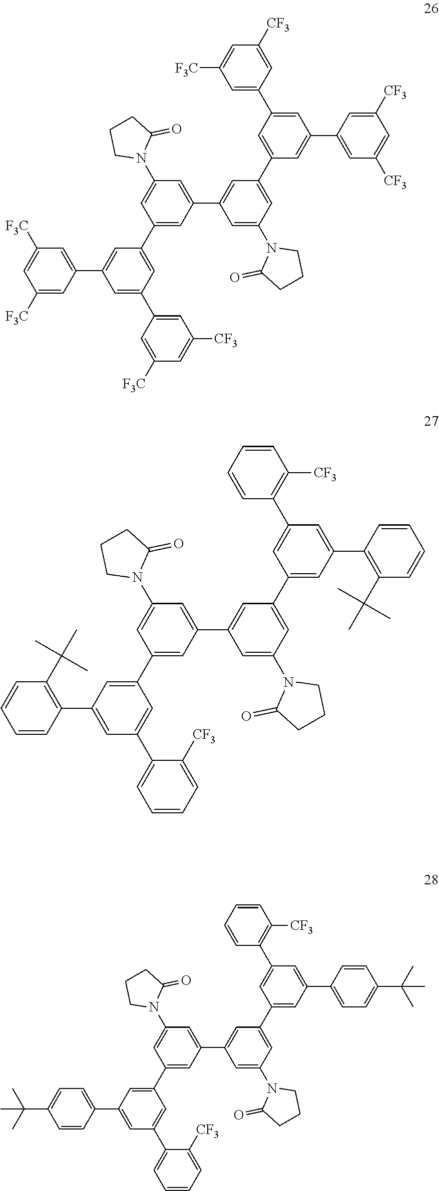 [SMILES: O=C1CCCN1c1cc(-c2cc(-c3cc(C(F)(F)F)cc(C(F)(F)F)c3)cc(-c3cc(C(F)(F)F)cc(C(F)(F)F)c3)c2)cc(-c2cc(-c3cc(-c4cc(C(F)(F)F)cc(C(F)(F)F)c4)cc(-c4cc(C(F)(F)F)cc(C(F)(F)F)c4)c3)cc(N3CCCC3=O)c2)c1 ; CC(C)(C)c1ccccc1-c1cc(-c2cc(-c3cc(-c4cc(-c5ccccc5C(C)(C)C)cc(-c5ccccc5C(F)(F)F)c4)cc(N4CCCC4=O)c3)cc(N3CCCC3=O)c2)cc(-c2ccccc2C(F)(F)F)c1 ; CC(C)(C)c1ccc(-c2cc(-c3cc(-c4cc(-c5cc(-c6ccc(C(C)(C)C)cc6)cc(-c6ccccc6C(F)(F)F)c5)cc(N5CCCC5=O)c4)cc(N4CCCC4=O)c3)cc(-c3ccccc3C(F)(F)F)c2)cc1]

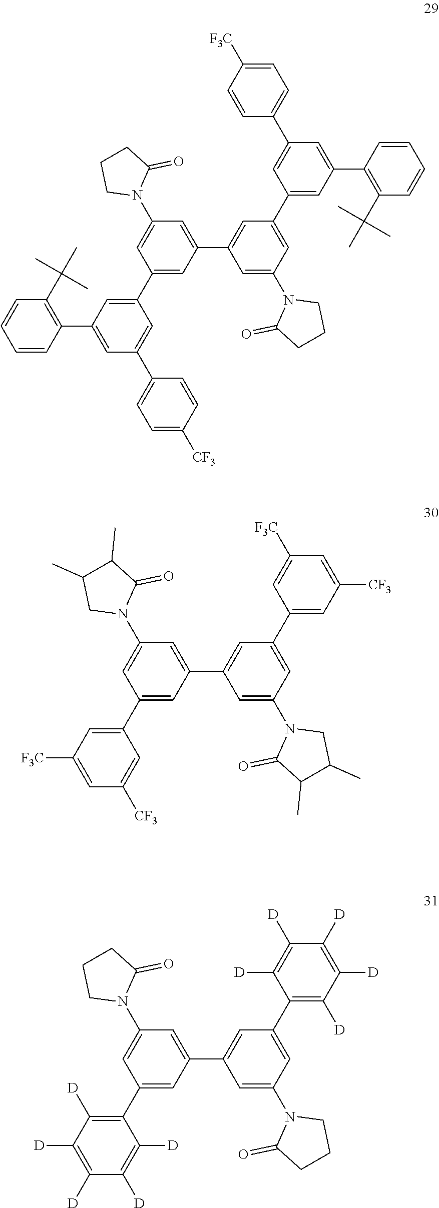 [SMILES: CC(C)(C)c1ccccc1-c1cc(-c2ccc(C(F)(F)F)cc2)cc(-c2cc(-c3cc(-c4cc(-c5ccc(C(F)(F)F)cc5)cc(-c5ccccc5C(C)(C)C)c4)cc(N4CCCC4=O)c3)cc(N3CCCC3=O)c2)c1 ; CC1CN(c2cc(-c3cc(-c4cc(C(F)(F)F)cc(C(F)(F)F)c4)cc(N4CC(C)C(C)C4=O)c3)cc(-c3cc(C(F)(F)F)cc(C(F)(F)F)c3)c2)C(=O)C1C ; [2H]c1c([2H])c([2H])c(-c2cc(-c3cc(-c4c([2H])c([2H])c([2H])c([2H])c4[2H])cc(N4CCCC4=O)c3)cc(N3CCCC3=O)c2)c([2H])c1[2H]]

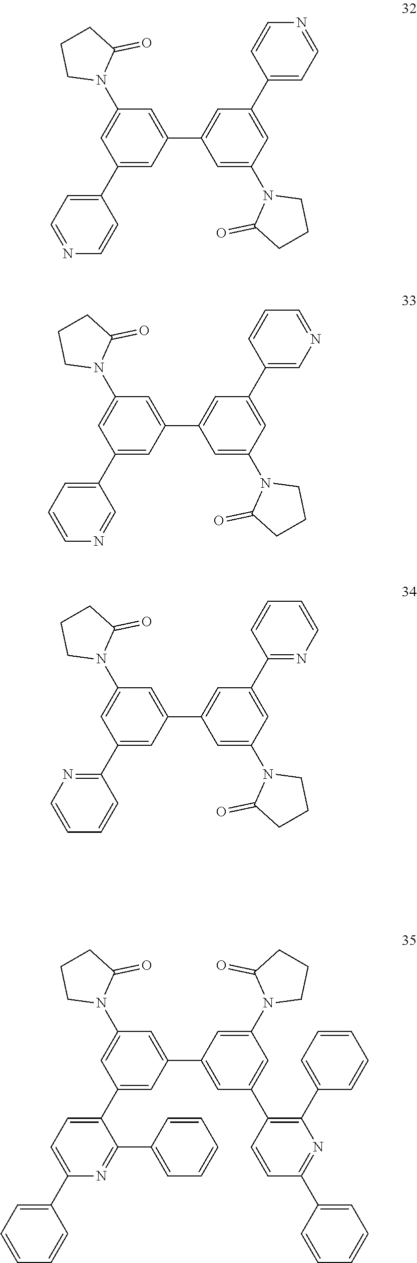 [SMILES: O=C1CCCN1c1cc(-c2ccncc2)cc(-c2cc(-c3ccncc3)cc(N3CCCC3=O)c2)c1 ; O=C1CCCN1c1cc(-c2cccnc2)cc(-c2cc(-c3cccnc3)cc(N3CCCC3=O)c2)c1 ; O=C1CCCN1c1cc(-c2cc(-c3ccccn3)cc(N3CCCC3=O)c2)cc(-c2ccccn2)c1 ; O=C1CCCN1c1cc(-c2cc(-c3ccc(-c4ccccc4)nc3-c3ccccc3)cc(N3CCCC3=O)c2)cc(-c2ccc(-c3ccccc3)nc2-c2ccccc2)c1]

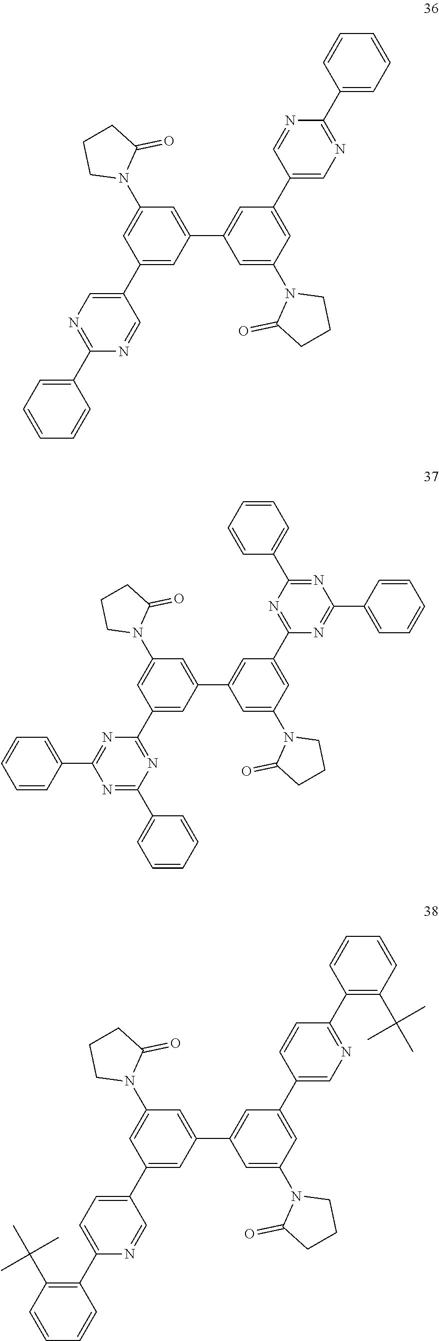 [SMILES: O=C1CCCN1c1cc(-c2cnc(-c3ccccc3)nc2)cc(-c2cc(-c3cnc(-c4ccccc4)nc3)cc(N3CCCC3=O)c2)c1 ; O=C1CCCN1c1cc(-c2cc(-c3nc(-c4ccccc4)nc(-c4ccccc4)n3)cc(N3CCCC3=O)c2)cc(-c2nc(-c3ccccc3)nc(-c3ccccc3)n2)c1 ; CC(C)(C)c1ccccc1-c1ccc(-c2cc(-c3cc(-c4ccc(-c5ccccc5C(C)(C)C)nc4)cc(N4CCCC4=O)c3)cc(N3CCCC3=O)c2)cn1]

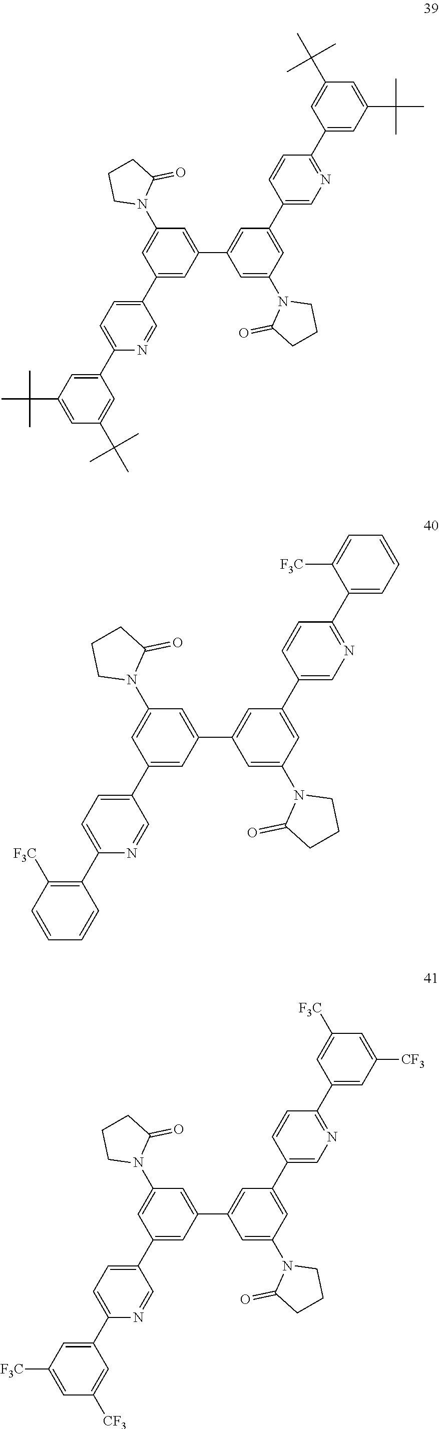 [SMILES: CC(C)(C)c1cc(-c2ccc(-c3cc(-c4cc(-c5ccc(-c6cc(C(C)(C)C)cc(C(C)(C)C)c6)nc5)cc(N5CCCC5=O)c4)cc(N4CCCC4=O)c3)cn2)cc(C(C)(C)C)c1 ; O=C1CCCN1c1cc(-c2ccc(-c3ccccc3C(F)(F)F)nc2)cc(-c2cc(-c3ccc(-c4ccccc4C(F)(F)F)nc3)cc(N3CCCC3=O)c2)c1 ; O=C1CCCN1c1cc(-c2ccc(-c3cc(C(F)(F)F)cc(C(F)(F)F)c3)nc2)cc(-c2cc(-c3ccc(-c4cc(C(F)(F)F)cc(C(F)(F)F)c4)nc3)cc(N3CCCC3=O)c2)c1]

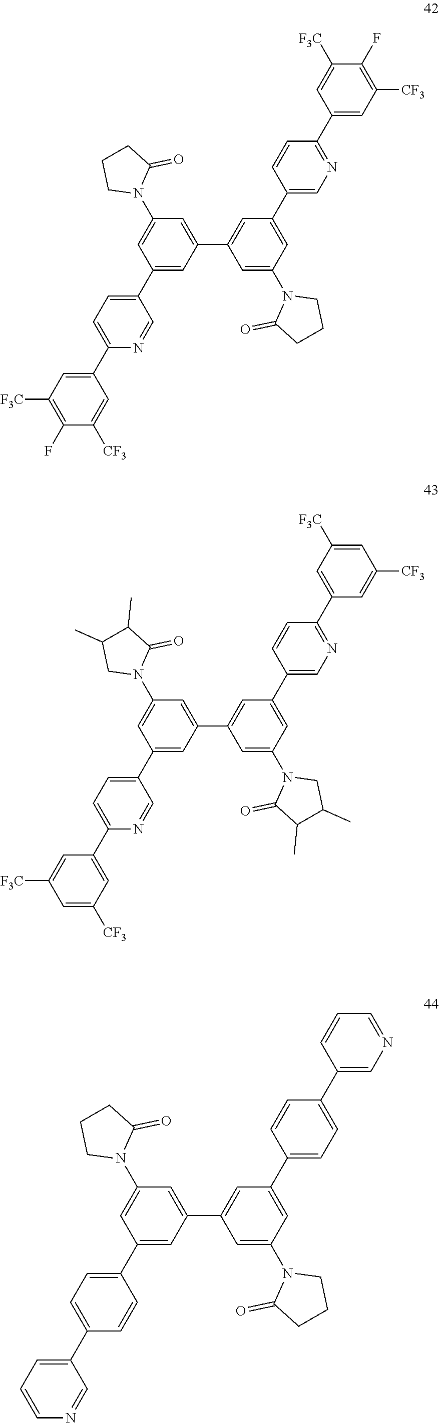 [SMILES: O=C1CCCN1c1cc(-c2ccc(-c3cc(C(F)(F)F)c(F)c(C(F)(F)F)c3)nc2)cc(-c2cc(-c3ccc(-c4cc(C(F)(F)F)c(F)c(C(F)(F)F)c4)nc3)cc(N3CCCC3=O)c2)c1 ; CC1CN(c2cc(-c3ccc(-c4cc(C(F)(F)F)cc(C(F)(F)F)c4)nc3)cc(-c3cc(-c4ccc(-c5cc(C(F)(F)F)cc(C(F)(F)F)c5)nc4)cc(N4CC(C)C(C)C4=O)c3)c2)C(=O)C1C ; O=C1CCCN1c1cc(-c2ccc(-c3cccnc3)cc2)cc(-c2cc(-c3ccc(-c4cccnc4)cc3)cc(N3CCCC3=O)c2)c1]

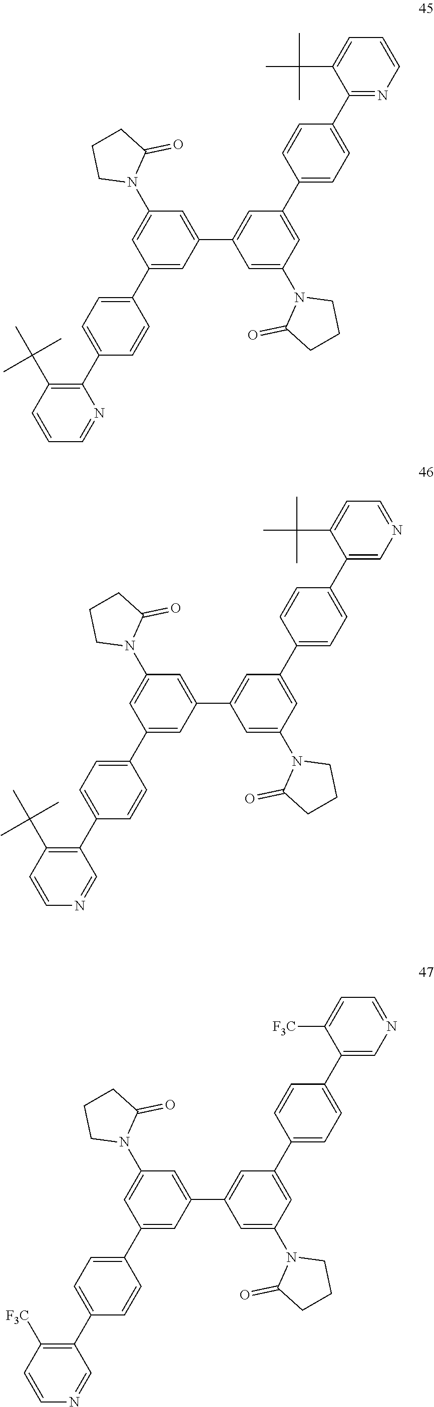 [SMILES: CC(C)(C)c1cccnc1-c1ccc(-c2cc(-c3cc(-c4ccc(-c5ncccc5C(C)(C)C)cc4)cc(N4CCCC4=O)c3)cc(N3CCCC3=O)c2)cc1 ; CC(C)(C)c1ccncc1-c1ccc(-c2cc(-c3cc(-c4ccc(-c5cnccc5C(C)(C)C)cc4)cc(N4CCCC4=O)c3)cc(N3CCCC3=O)c2)cc1 ; O=C1CCCN1c1cc(-c2ccc(-c3cnccc3C(F)(F)F)cc2)cc(-c2cc(-c3ccc(-c4cnccc4C(F)(F)F)cc3)cc(N3CCCC3=O)c2)c1]

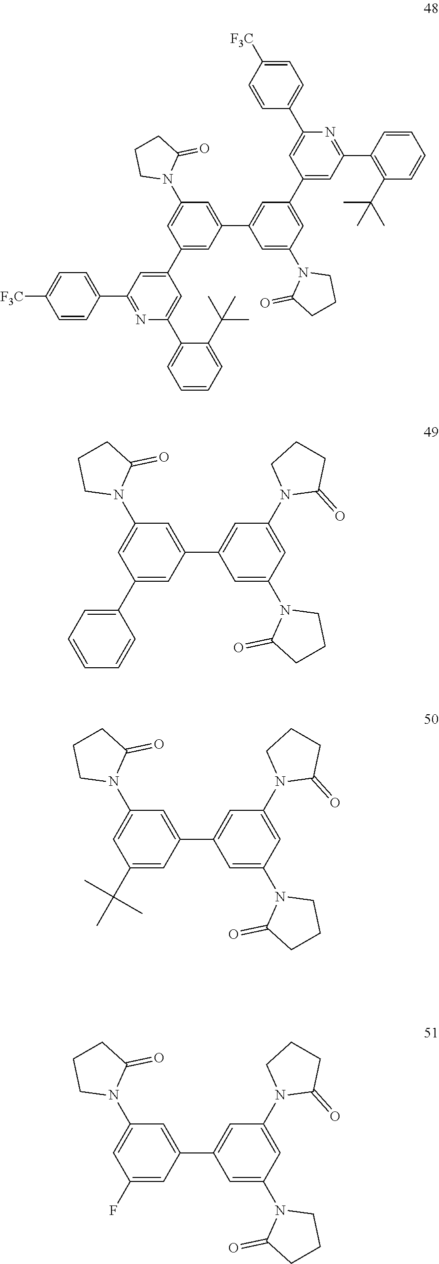 [SMILES: CC(C)(C)c1ccccc1-c1cc(-c2cc(-c3cc(-c4cc(-c5ccc(C(F)(F)F)cc5)nc(-c5ccccc5C(C)(C)C)c4)cc(N4CCCC4=O)c3)cc(N3CCCC3=O)c2)cc(-c2ccc(C(F)(F)F)cc2)n1 ; O=C1CCCN1c1cc(-c2ccccc2)cc(-c2cc(N3CCCC3=O)cc(N3CCCC3=O)c2)c1 ; CC(C)(C)c1cc(-c2cc(N3CCCC3=O)cc(N3CCCC3=O)c2)cc(N2CCCC2=O)c1 ; O=C1CCCN1c1cc(F)cc(-c2cc(N3CCCC3=O)cc(N3CCCC3=O)c2)c1]

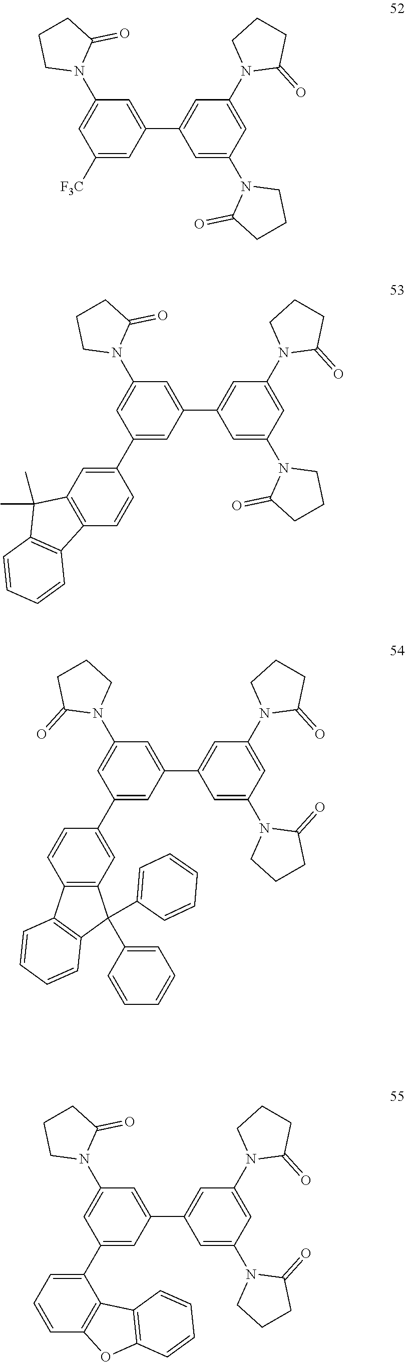 [SMILES: O=C1CCCN1c1cc(-c2cc(N3CCCC3=O)cc(C(F)(F)F)c2)cc(N2CCCC2=O)c1 ; CC1(C)c2ccccc2-c2ccc(-c3cc(-c4cc(N5CCCC5=O)cc(N5CCCC5=O)c4)cc(N4CCCC4=O)c3)cc21 ; O=C1CCCN1c1cc(-c2cc(N3CCCC3=O)cc(N3CCCC3=O)c2)cc(-c2ccc3c(c2)C(c2ccccc2)(c2ccccc2)c2ccccc2-3)c1 ; O=C1CCCN1c1cc(-c2cc(N3CCCC3=O)cc(N3CCCC3=O)c2)cc(-c2cccc3oc4ccccc4c23)c1]

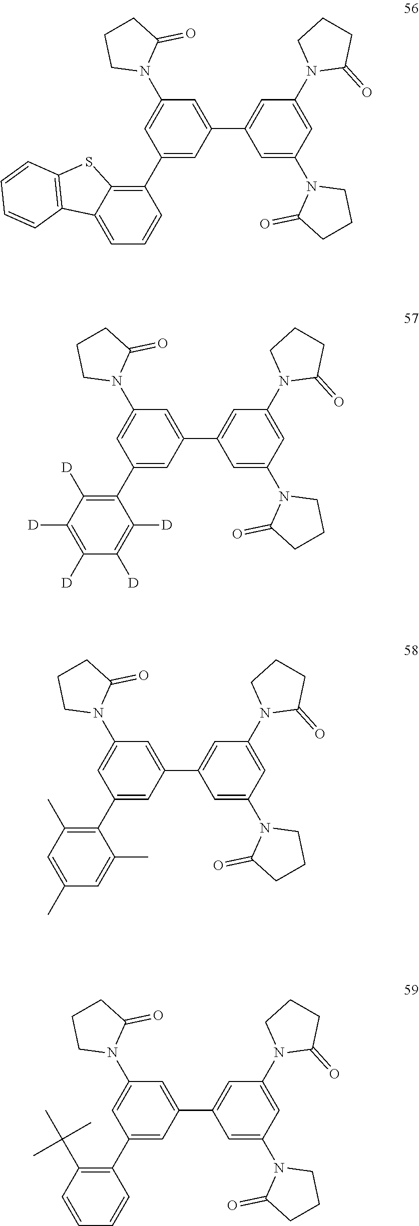 [SMILES: O=C1CCCN1c1cc(-c2cc(N3CCCC3=O)cc(N3CCCC3=O)c2)cc(-c2cccc3c2sc2ccccc23)c1 ; [2H]c1c([2H])c([2H])c(-c2cc(-c3cc(N4CCCC4=O)cc(N4CCCC4=O)c3)cc(N3CCCC3=O)c2)c([2H])c1[2H] ; Cc1cc(C)c(-c2cc(-c3cc(N4CCCC4=O)cc(N4CCCC4=O)c3)cc(N3CCCC3=O)c2)c(C)c1 ; CC(C)(C)c1ccccc1-c1cc(-c2cc(N3CCCC3=O)cc(N3CCCC3=O)c2)cc(N2CCCC2=O)c1]

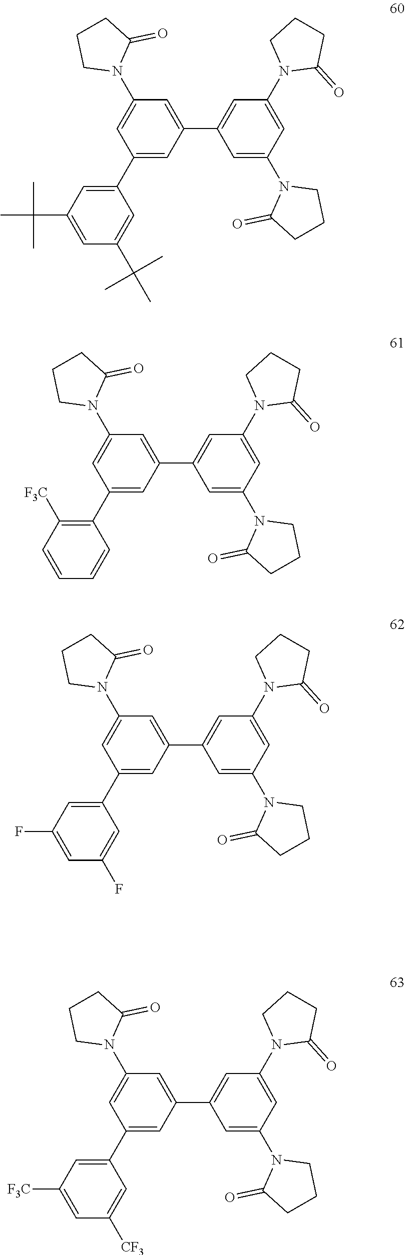 [SMILES: CC(C)(C)c1cc(-c2cc(-c3cc(N4CCCC4=O)cc(N4CCCC4=O)c3)cc(N3CCCC3=O)c2)cc(C(C)(C)C)c1 ; O=C1CCCN1c1cc(-c2cc(N3CCCC3=O)cc(N3CCCC3=O)c2)cc(-c2ccccc2C(F)(F)F)c1 ; O=C1CCCN1c1cc(-c2cc(F)cc(F)c2)cc(-c2cc(N3CCCC3=O)cc(N3CCCC3=O)c2)c1 ; O=C1CCCN1c1cc(-c2cc(N3CCCC3=O)cc(N3CCCC3=O)c2)cc(-c2cc(C(F)(F)F)cc(C(F)(F)F)c2)c1]

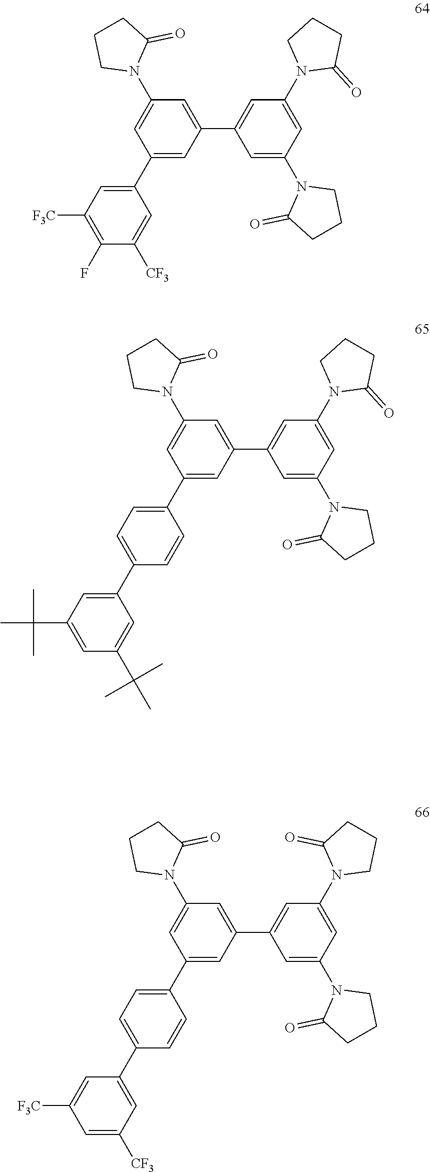 [SMILES: O=C1CCCN1c1cc(-c2cc(N3CCCC3=O)cc(N3CCCC3=O)c2)cc(-c2cc(C(F)(F)F)c(F)c(C(F)(F)F)c2)c1 ; CC(C)(C)c1cc(-c2ccc(-c3cc(-c4cc(N5CCCC5=O)cc(N5CCCC5=O)c4)cc(N4CCCC4=O)c3)cc2)cc(C(C)(C)C)c1 ; O=C1CCCN1c1cc(-c2ccc(-c3cc(C(F)(F)F)cc(C(F)(F)F)c3)cc2)cc(-c2cc(N3CCCC3=O)cc(N3CCCC3=O)c2)c1]

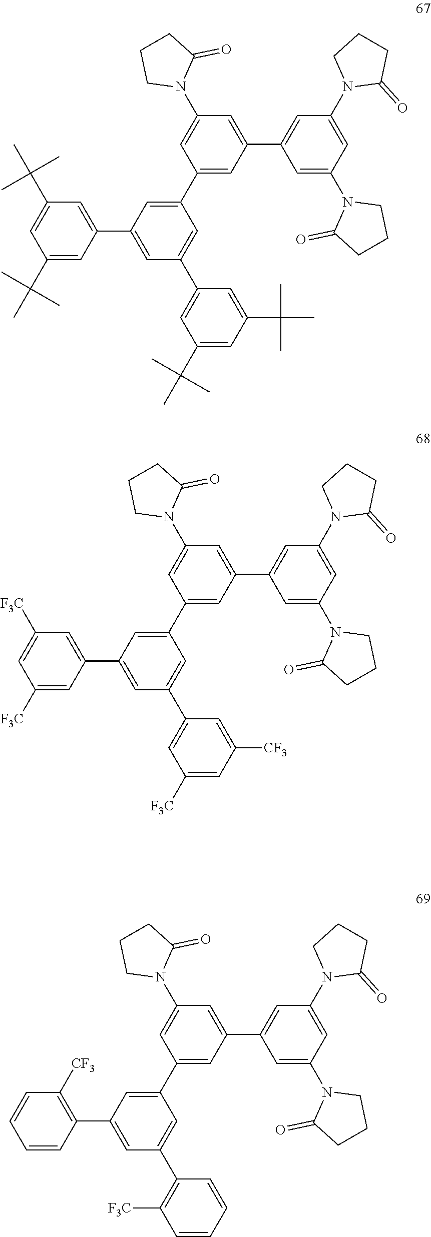 [SMILES: CC(C)(C)c1cc(-c2cc(-c3cc(-c4cc(N5CCCC5=O)cc(N5CCCC5=O)c4)cc(N4CCCC4=O)c3)cc(-c3cc(C(C)(C)C)cc(C(C)(C)C)c3)c2)cc(C(C)(C)C)c1 ; O=C1CCCN1c1cc(-c2cc(-c3cc(C(F)(F)F)cc(C(F)(F)F)c3)cc(-c3cc(C(F)(F)F)cc(C(F)(F)F)c3)c2)cc(-c2cc(N3CCCC3=O)cc(N3CCCC3=O)c2)c1 ; O=C1CCCN1c1cc(-c2cc(-c3ccccc3C(F)(F)F)cc(-c3ccccc3C(F)(F)F)c2)cc(-c2cc(N3CCCC3=O)cc(N3CCCC3=O)c2)c1]

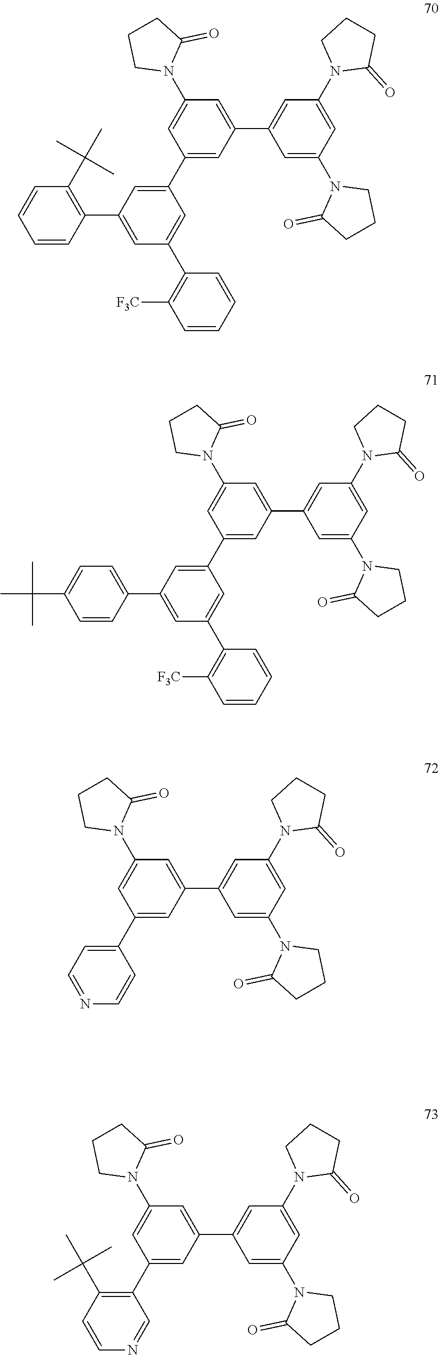 [SMILES: CC(C)(C)c1ccccc1-c1cc(-c2cc(-c3cc(N4CCCC4=O)cc(N4CCCC4=O)c3)cc(N3CCCC3=O)c2)cc(-c2ccccc2C(F)(F)F)c1 ; CC(C)(C)c1ccc(-c2cc(-c3cc(-c4cc(N5CCCC5=O)cc(N5CCCC5=O)c4)cc(N4CCCC4=O)c3)cc(-c3ccccc3C(F)(F)F)c2)cc1 ; O=C1CCCN1c1cc(-c2ccncc2)cc(-c2cc(N3CCCC3=O)cc(N3CCCC3=O)c2)c1 ; CC(C)(C)c1ccncc1-c1cc(-c2cc(N3CCCC3=O)cc(N3CCCC3=O)c2)cc(N2CCCC2=O)c1]

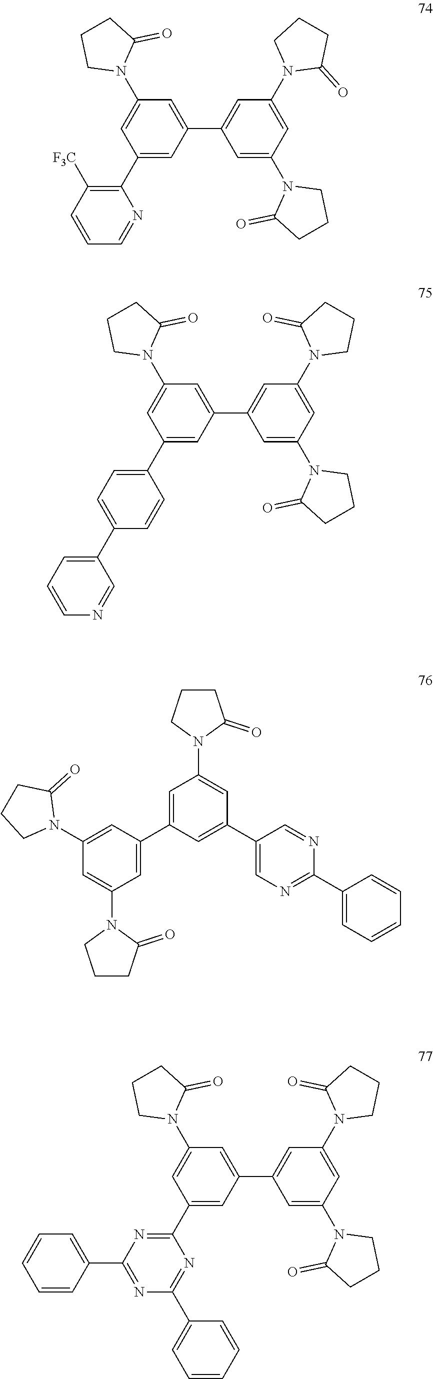 [SMILES: O=C1CCCN1c1cc(-c2cc(N3CCCC3=O)cc(N3CCCC3=O)c2)cc(-c2ncccc2C(F)(F)F)c1 ; O=C1CCCN1c1cc(-c2ccc(-c3cccnc3)cc2)cc(-c2cc(N3CCCC3=O)cc(N3CCCC3=O)c2)c1 ; O=C1CCCN1c1cc(-c2cnc(-c3ccccc3)nc2)cc(-c2cc(N3CCCC3=O)cc(N3CCCC3=O)c2)c1 ; O=C1CCCN1c1cc(-c2cc(N3CCCC3=O)cc(N3CCCC3=O)c2)cc(-c2nc(-c3ccccc3)nc(-c3ccccc3)n2)c1]

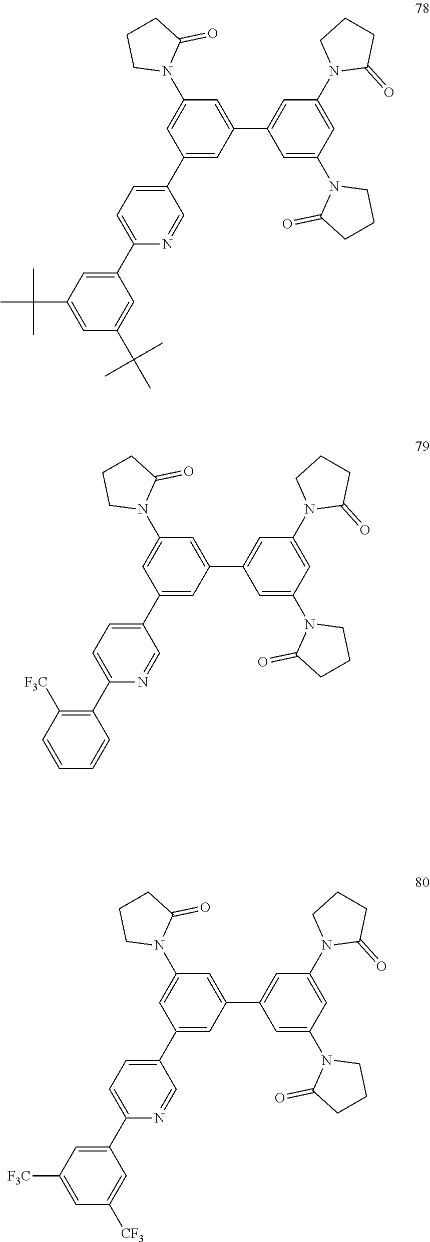 [SMILES: CC(C)(C)c1cc(-c2ccc(-c3cc(-c4cc(N5CCCC5=O)cc(N5CCCC5=O)c4)cc(N4CCCC4=O)c3)cn2)cc(C(C)(C)C)c1 ; O=C1CCCN1c1cc(-c2ccc(-c3ccccc3C(F)(F)F)nc2)cc(-c2cc(N3CCCC3=O)cc(N3CCCC3=O)c2)c1 ; O=C1CCCN1c1cc(-c2ccc(-c3cc(C(F)(F)F)cc(C(F)(F)F)c3)nc2)cc(-c2cc(N3CCCC3=O)cc(N3CCCC3=O)c2)c1]

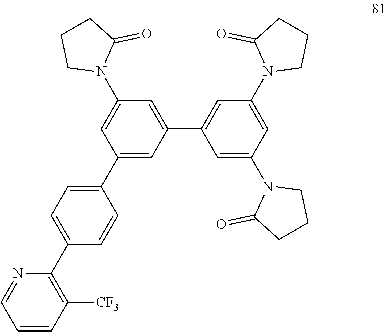 [SMILES: O=C1CCCN1c1cc(-c2ccc(-c3ncccc3C(F)(F)F)cc2)cc(-c2cc(N3CCCC3=O)cc(N3CCCC3=O)c2)c1]

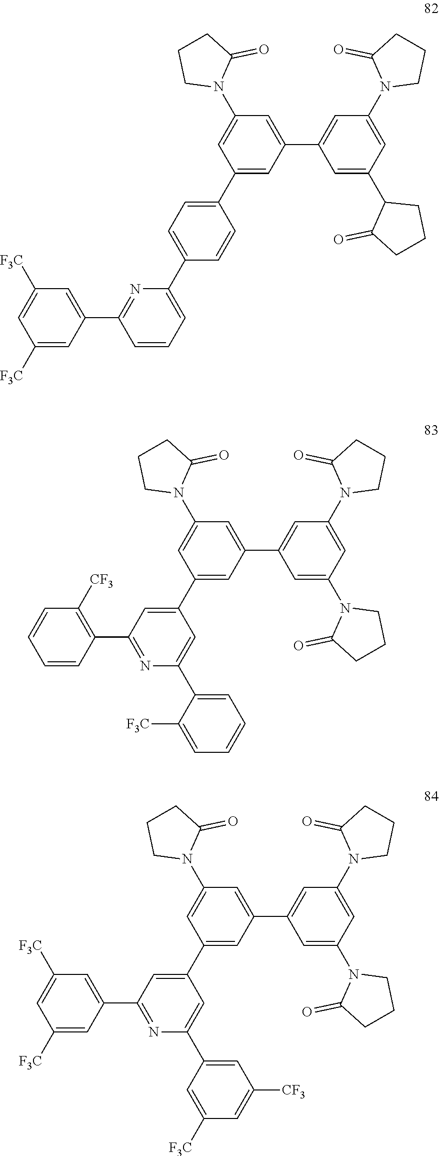 [SMILES: O=C1CCCC1c1cc(-c2cc(-c3ccc(-c4cccc(-c5cc(C(F)(F)F)cc(C(F)(F)F)c5)n4)cc3)cc(N3CCCC3=O)c2)cc(N2CCCC2=O)c1 ; O=C1CCCN1c1cc(-c2cc(N3CCCC3=O)cc(N3CCCC3=O)c2)cc(-c2cc(-c3ccccc3C(F)(F)F)nc(-c3ccccc3C(F)(F)F)c2)c1 ; O=C1CCCN1c1cc(-c2cc(N3CCCC3=O)cc(N3CCCC3=O)c2)cc(-c2cc(-c3cc(C(F)(F)F)cc(C(F)(F)F)c3)nc(-c3cc(C(F)(F)F)cc(C(F)(F)F)c3)c2)c1]

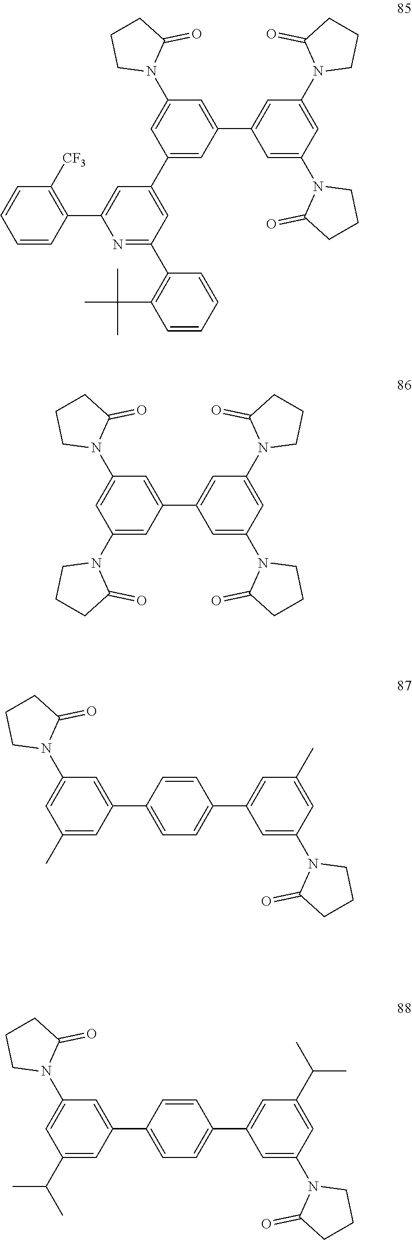 [SMILES: CC(C)(C)c1ccccc1-c1cc(-c2cc(-c3cc(N4CCCC4=O)cc(N4CCCC4=O)c3)cc(N3CCCC3=O)c2)cc(-c2ccccc2C(F)(F)F)n1 ; O=C1CCCN1c1cc(-c2cc(N3CCCC3=O)cc(N3CCCC3=O)c2)cc(N2CCCC2=O)c1 ; Cc1cc(-c2ccc(-c3cc(C)cc(N4CCCC4=O)c3)cc2)cc(N2CCCC2=O)c1 ; CC(C)c1cc(-c2ccc(-c3cc(C(C)C)cc(N4CCCC4=O)c3)cc2)cc(N2CCCC2=O)c1]

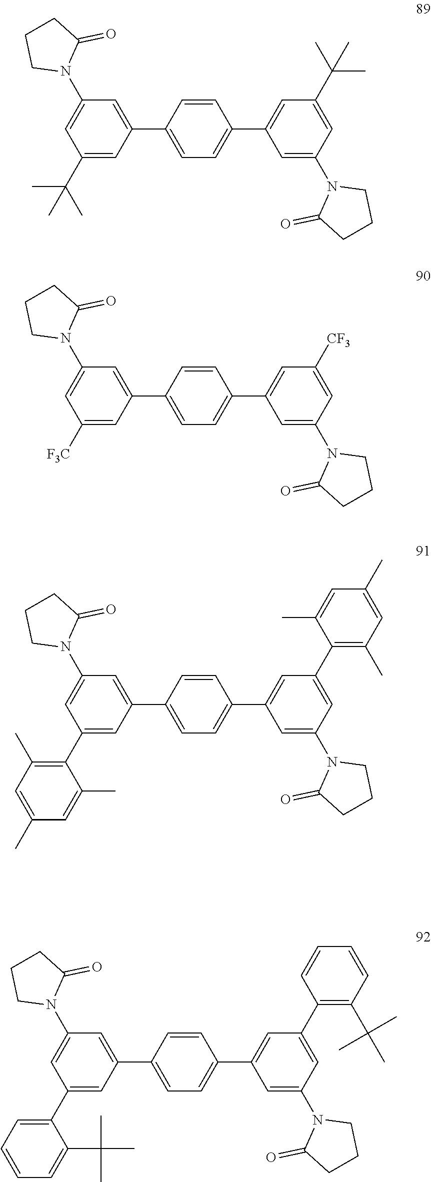 [SMILES: CC(C)(C)c1cc(-c2ccc(-c3cc(N4CCCC4=O)cc(C(C)(C)C)c3)cc2)cc(N2CCCC2=O)c1 ; O=C1CCCN1c1cc(-c2ccc(-c3cc(N4CCCC4=O)cc(C(F)(F)F)c3)cc2)cc(C(F)(F)F)c1 ; Cc1cc(C)c(-c2cc(-c3ccc(-c4cc(-c5c(C)cc(C)cc5C)cc(N5CCCC5=O)c4)cc3)cc(N3CCCC3=O)c2)c(C)c1 ; CC(C)(C)c1ccccc1-c1cc(-c2ccc(-c3cc(-c4ccccc4C(C)(C)C)cc(N4CCCC4=O)c3)cc2)cc(N2CCCC2=O)c1]

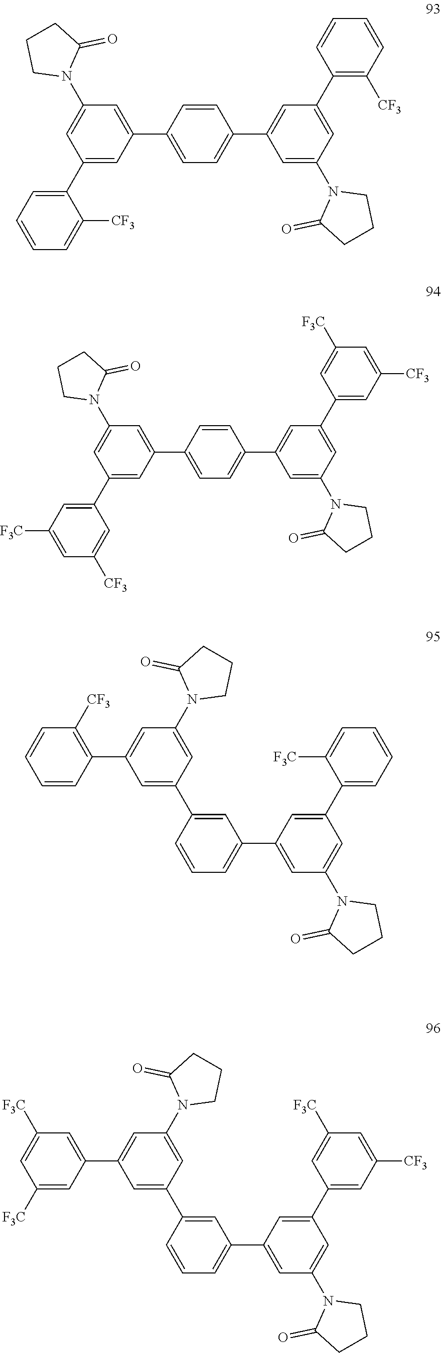 [SMILES: O=C1CCCN1c1cc(-c2ccc(-c3cc(-c4ccccc4C(F)(F)F)cc(N4CCCC4=O)c3)cc2)cc(-c2ccccc2C(F)(F)F)c1 ; O=C1CCCN1c1cc(-c2ccc(-c3cc(-c4cc(C(F)(F)F)cc(C(F)(F)F)c4)cc(N4CCCC4=O)c3)cc2)cc(-c2cc(C(F)(F)F)cc(C(F)(F)F)c2)c1 ; O=C1CCCN1c1cc(-c2cccc(-c3cc(-c4ccccc4C(F)(F)F)cc(N4CCCC4=O)c3)c2)cc(-c2ccccc2C(F)(F)F)c1 ; O=C1CCCN1c1cc(-c2cccc(-c3cc(-c4cc(C(F)(F)F)cc(C(F)(F)F)c4)cc(N4CCCC4=O)c3)c2)cc(-c2cc(C(F)(F)F)cc(C(F)(F)F)c2)c1]

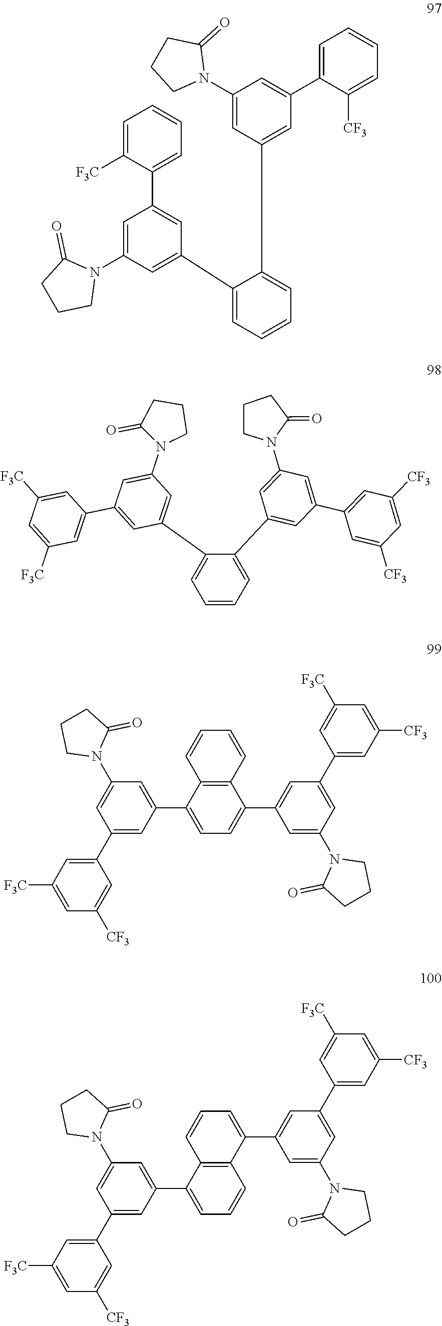 [SMILES: O=C1CCCN1c1cc(-c2ccccc2-c2cc(-c3ccccc3C(F)(F)F)cc(N3CCCC3=O)c2)cc(-c2ccccc2C(F)(F)F)c1 ; O=C1CCCN1c1cc(-c2cc(C(F)(F)F)cc(C(F)(F)F)c2)cc(-c2ccccc2-c2cc(-c3cc(C(F)(F)F)cc(C(F)(F)F)c3)cc(N3CCCC3=O)c2)c1 ; O=C1CCCN1c1cc(-c2cc(C(F)(F)F)cc(C(F)(F)F)c2)cc(-c2ccc(-c3cc(-c4cc(C(F)(F)F)cc(C(F)(F)F)c4)cc(N4CCCC4=O)c3)c3ccccc23)c1 ; O=C1CCCN1c1cc(-c2cc(C(F)(F)F)cc(C(F)(F)F)c2)cc(-c2cccc3c(-c4cc(-c5cc(C(F)(F)F)cc(C(F)(F)F)c5)cc(N5CCCC5=O)c4)cccc23)c1]

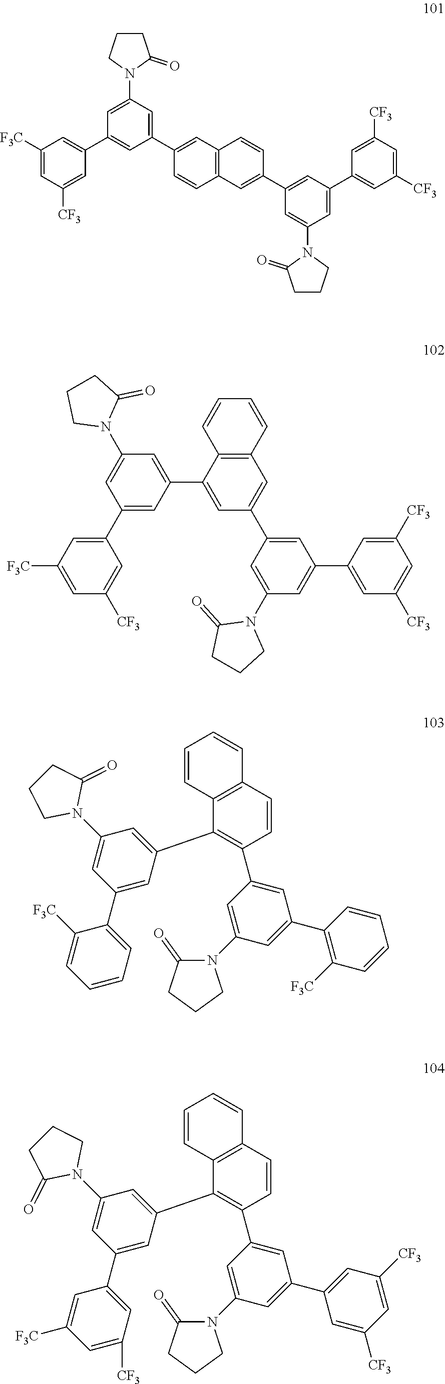 [SMILES: O=C1CCCN1c1cc(-c2cc(C(F)(F)F)cc(C(F)(F)F)c2)cc(-c2ccc3cc(-c4cc(-c5cc(C(F)(F)F)cc(C(F)(F)F)c5)cc(N5CCCC5=O)c4)ccc3c2)c1 ; O=C1CCCN1c1cc(-c2cc(C(F)(F)F)cc(C(F)(F)F)c2)cc(-c2cc(-c3cc(-c4cc(C(F)(F)F)cc(C(F)(F)F)c4)cc(N4CCCC4=O)c3)c3ccccc3c2)c1 ; O=C1CCCN1c1cc(-c2ccccc2C(F)(F)F)cc(-c2ccc3ccccc3c2-c2cc(-c3ccccc3C(F)(F)F)cc(N3CCCC3=O)c2)c1 ; O=C1CCCN1c1cc(-c2cc(C(F)(F)F)cc(C(F)(F)F)c2)cc(-c2ccc3ccccc3c2-c2cc(-c3cc(C(F)(F)F)cc(C(F)(F)F)c3)cc(N3CCCC3=O)c2)c1]

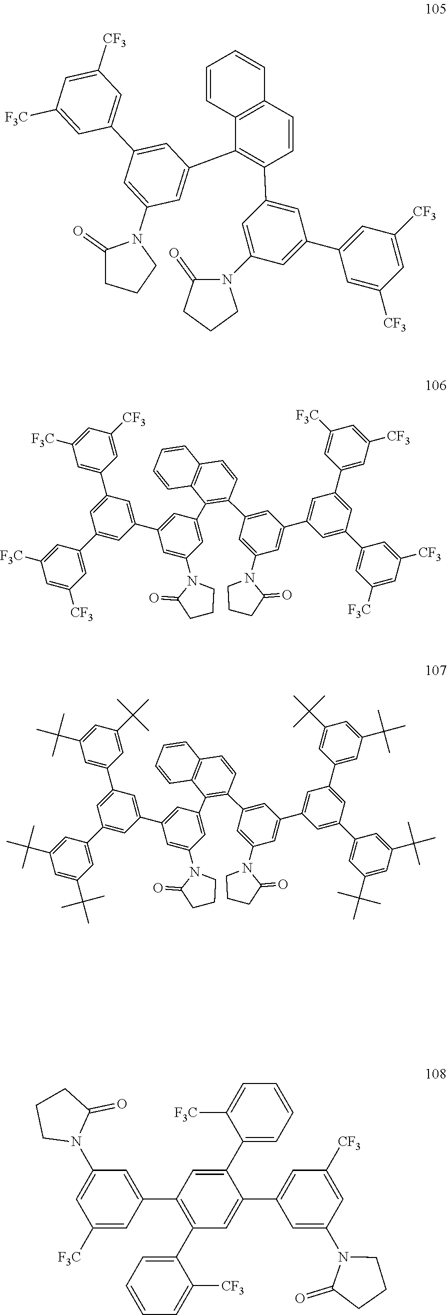

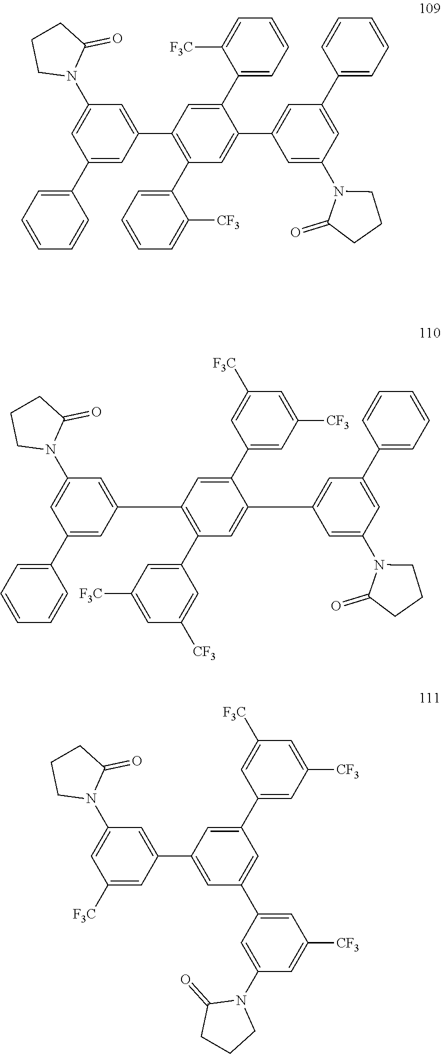 [SMILES: O=C1CCCN1c1cc(-c2ccccc2)cc(-c2cc(-c3ccccc3C(F)(F)F)c(-c3cc(-c4ccccc4)cc(N4CCCC4=O)c3)cc2-c2ccccc2C(F)(F)F)c1 ; O=C1CCCN1c1cc(-c2ccccc2)cc(-c2cc(-c3cc(C(F)(F)F)cc(C(F)(F)F)c3)c(-c3cc(-c4ccccc4)cc(N4CCCC4=O)c3)cc2-c2cc(C(F)(F)F)cc(C(F)(F)F)c2)c1 ; O=C1CCCN1c1cc(-c2cc(-c3cc(N4CCCC4=O)cc(C(F)(F)F)c3)cc(-c3cc(C(F)(F)F)cc(C(F)(F)F)c3)c2)cc(C(F)(F)F)c1]

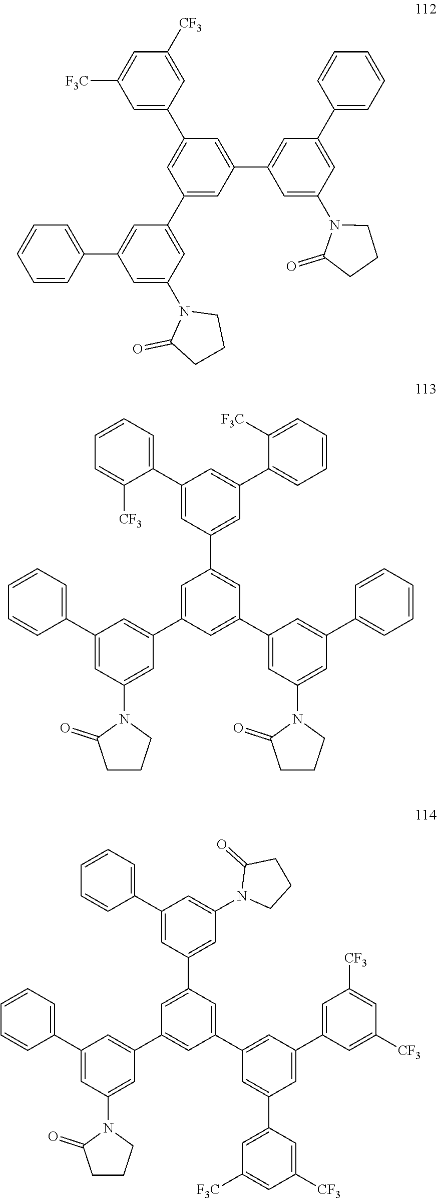 [SMILES: O=C1CCCN1c1cc(-c2ccccc2)cc(-c2cc(-c3cc(-c4ccccc4)cc(N4CCCC4=O)c3)cc(-c3cc(C(F)(F)F)cc(C(F)(F)F)c3)c2)c1 ; O=C1CCCN1c1cc(-c2ccccc2)cc(-c2cc(-c3cc(-c4ccccc4C(F)(F)F)cc(-c4ccccc4C(F)(F)F)c3)cc(-c3cc(-c4ccccc4)cc(N4CCCC4=O)c3)c2)c1 ; O=C1CCCN1c1cc(-c2ccccc2)cc(-c2cc(-c3cc(-c4cc(C(F)(F)F)cc(C(F)(F)F)c4)cc(-c4cc(C(F)(F)F)cc(C(F)(F)F)c4)c3)cc(-c3cc(-c4ccccc4)cc(N4CCCC4=O)c3)c2)c1]

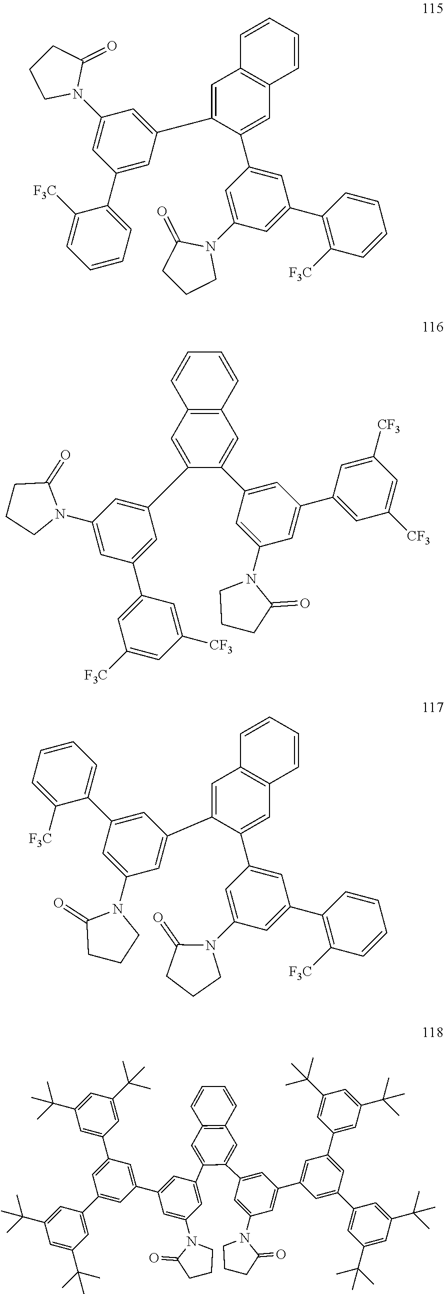

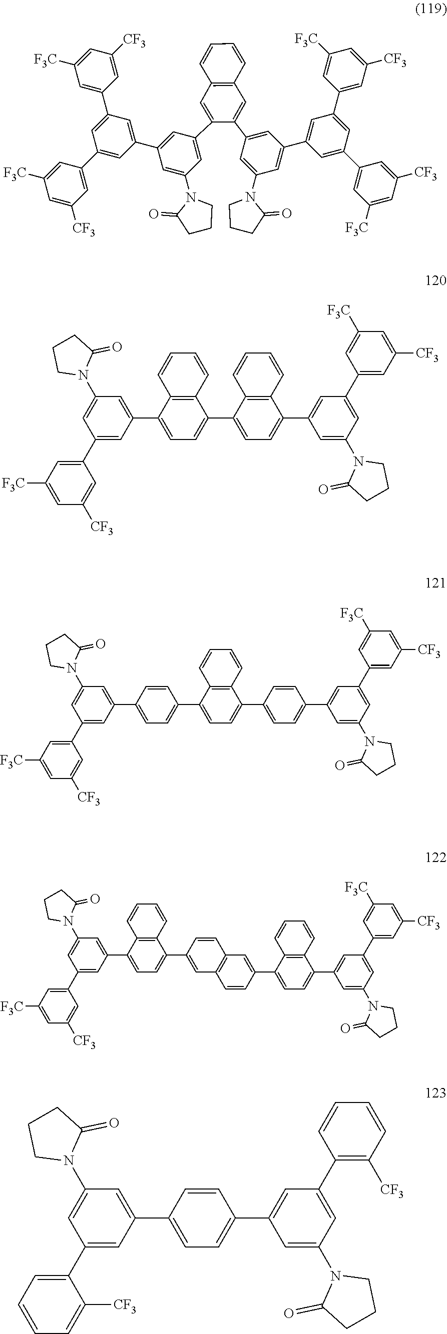

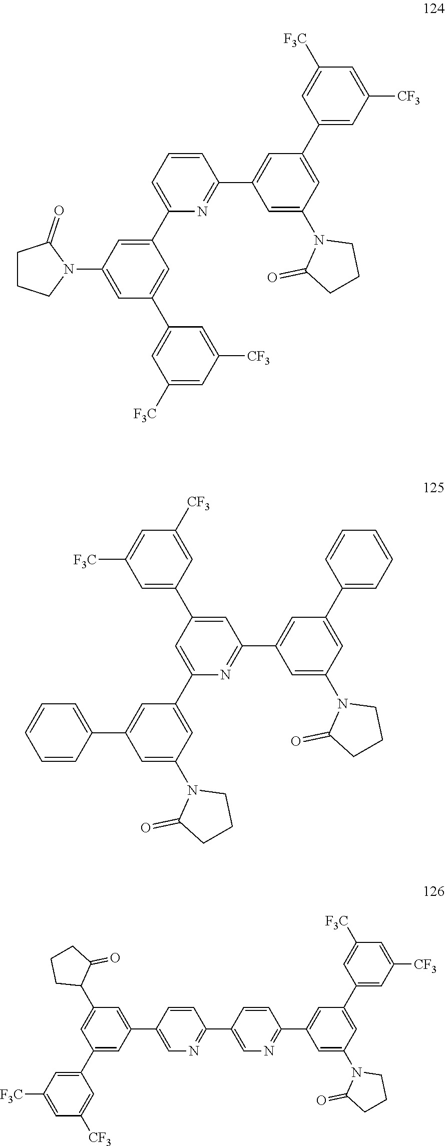 [SMILES: O=C1CCCN1c1cc(-c2cc(C(F)(F)F)cc(C(F)(F)F)c2)cc(-c2cccc(-c3cc(-c4cc(C(F)(F)F)cc(C(F)(F)F)c4)cc(N4CCCC4=O)c3)n2)c1 ; O=C1CCCN1c1cc(-c2ccccc2)cc(-c2cc(-c3cc(C(F)(F)F)cc(C(F)(F)F)c3)cc(-c3cc(-c4ccccc4)cc(N4CCCC4=O)c3)n2)c1 ; O=C1CCCC1c1cc(-c2ccc(-c3ccc(-c4cc(-c5cc(C(F)(F)F)cc(C(F)(F)F)c5)cc(N5CCCC5=O)c4)nc3)nc2)cc(-c2cc(C(F)(F)F)cc(C(F)(F)F)c2)c1]

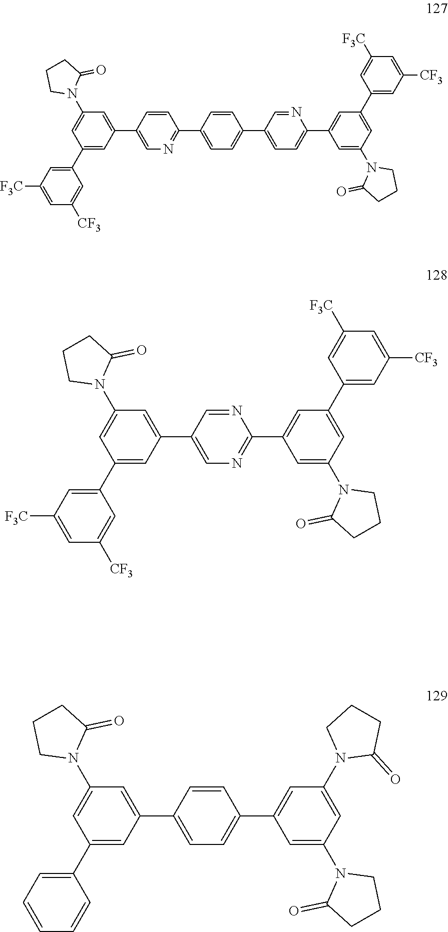 [SMILES: O=C1CCCN1c1cc(-c2ccc(-c3ccc(-c4ccc(-c5cc(-c6cc(C(F)(F)F)cc(C(F)(F)F)c6)cc(N6CCCC6=O)c5)nc4)cc3)nc2)cc(-c2cc(C(F)(F)F)cc(C(F)(F)F)c2)c1 ; O=C1CCCN1c1cc(-c2cnc(-c3cc(-c4cc(C(F)(F)F)cc(C(F)(F)F)c4)cc(N4CCCC4=O)c3)nc2)cc(-c2cc(C(F)(F)F)cc(C(F)(F)F)c2)c1 ; O=C1CCCN1c1cc(-c2ccccc2)cc(-c2ccc(-c3cc(N4CCCC4=O)cc(N4CCCC4=O)c3)cc2)c1]

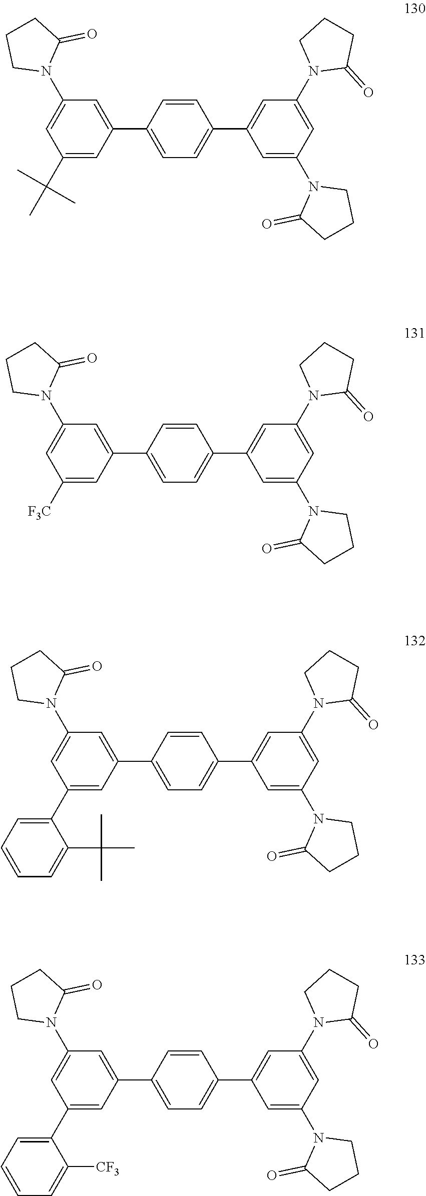 [SMILES: CC(C)(C)c1cc(-c2ccc(-c3cc(N4CCCC4=O)cc(N4CCCC4=O)c3)cc2)cc(N2CCCC2=O)c1 ; O=C1CCCN1c1cc(-c2ccc(-c3cc(N4CCCC4=O)cc(C(F)(F)F)c3)cc2)cc(N2CCCC2=O)c1 ; CC(C)(C)c1ccccc1-c1cc(-c2ccc(-c3cc(N4CCCC4=O)cc(N4CCCC4=O)c3)cc2)cc(N2CCCC2=O)c1 ; O=C1CCCN1c1cc(-c2ccc(-c3cc(N4CCCC4=O)cc(N4CCCC4=O)c3)cc2)cc(-c2ccccc2C(F)(F)F)c1]

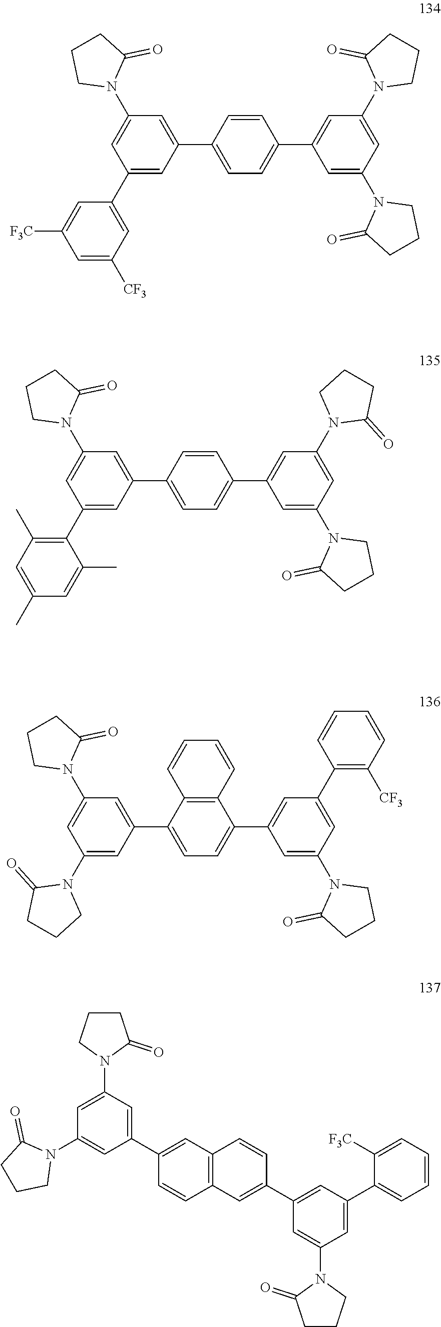 [SMILES: O=C1CCCN1c1cc(-c2ccc(-c3cc(N4CCCC4=O)cc(N4CCCC4=O)c3)cc2)cc(-c2cc(C(F)(F)F)cc(C(F)(F)F)c2)c1 ; Cc1cc(C)c(-c2cc(-c3ccc(-c4cc(N5CCCC5=O)cc(N5CCCC5=O)c4)cc3)cc(N3CCCC3=O)c2)c(C)c1 ; O=C1CCCN1c1cc(-c2ccccc2C(F)(F)F)cc(-c2ccc(-c3cc(N4CCCC4=O)cc(N4CCCC4=O)c3)c3ccccc23)c1 ; O=C1CCCN1c1cc(-c2ccc3cc(-c4cc(N5CCCC5=O)cc(N5CCCC5=O)c4)ccc3c2)cc(-c2ccccc2C(F)(F)F)c1]

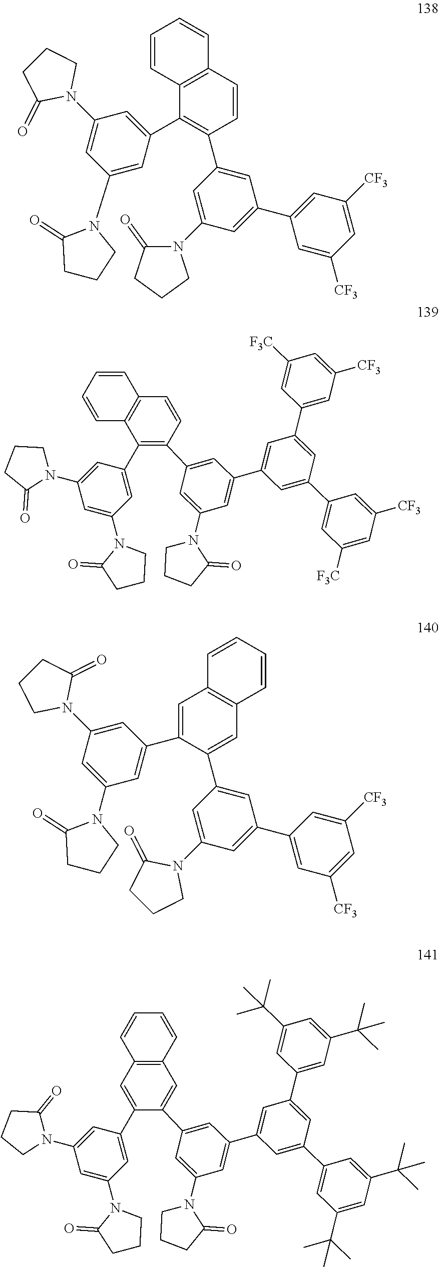 [SMILES: O=C1CCCN1c1cc(-c2cc(C(F)(F)F)cc(C(F)(F)F)c2)cc(-c2ccc3ccccc3c2-c2cc(N3CCCC3=O)cc(N3CCCC3=O)c2)c1 ; O=C1CCCN1c1cc(-c2cc(-c3cc(C(F)(F)F)cc(C(F)(F)F)c3)cc(-c3cc(C(F)(F)F)cc(C(F)(F)F)c3)c2)cc(-c2ccc3ccccc3c2-c2cc(N3CCCC3=O)cc(N3CCCC3=O)c2)c1 ; O=C1CCCN1c1cc(-c2cc(C(F)(F)F)cc(C(F)(F)F)c2)cc(-c2cc3ccccc3cc2-c2cc(N3CCCC3=O)cc(N3CCCC3=O)c2)c1 ; CC(C)(C)c1cc(-c2cc(-c3cc(-c4cc5ccccc5cc4-c4cc(N5CCCC5=O)cc(N5CCCC5=O)c4)cc(N4CCCC4=O)c3)cc(-c3cc(C(C)(C)C)cc(C(C)(C)C)c3)c2)cc(C(C)(C)C)c1]

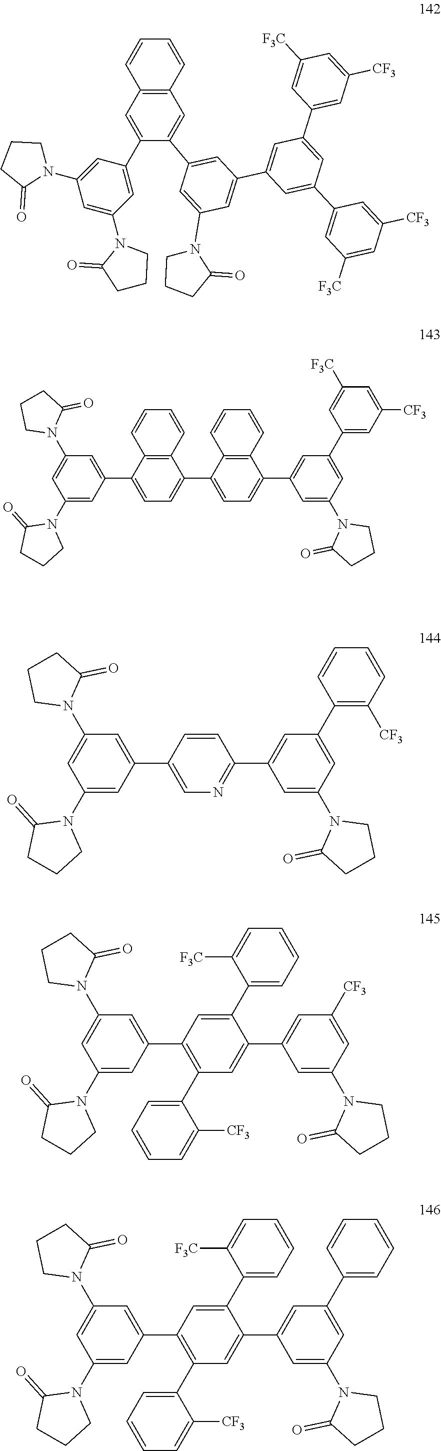

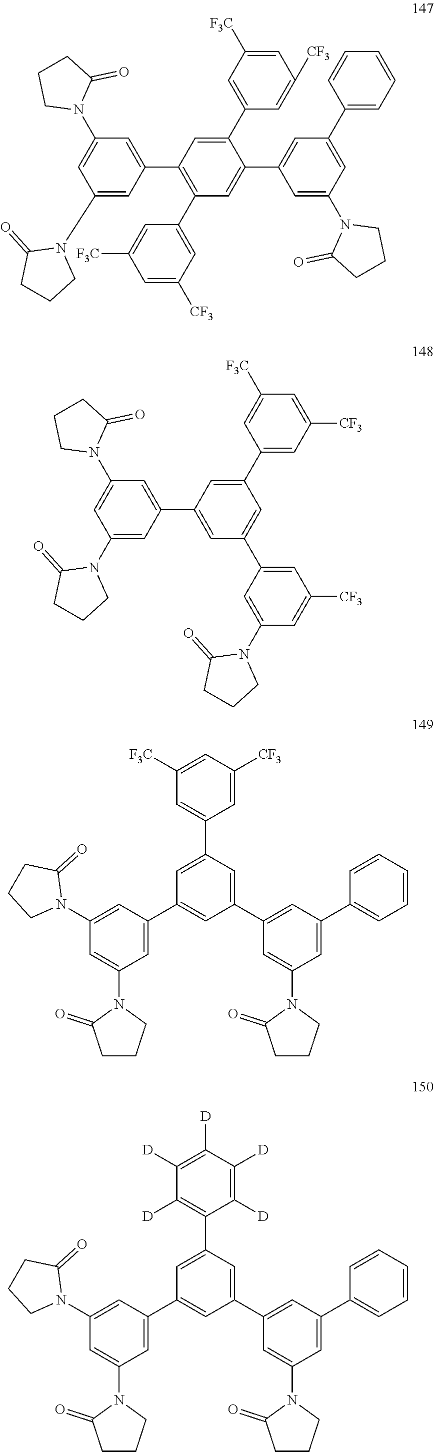 [SMILES: O=C1CCCN1c1cc(-c2ccccc2)cc(-c2cc(-c3cc(C(F)(F)F)cc(C(F)(F)F)c3)c(-c3cc(N4CCCC4=O)cc(N4CCCC4=O)c3)cc2-c2cc(C(F)(F)F)cc(C(F)(F)F)c2)c1 ; O=C1CCCN1c1cc(-c2cc(-c3cc(N4CCCC4=O)cc(C(F)(F)F)c3)cc(-c3cc(C(F)(F)F)cc(C(F)(F)F)c3)c2)cc(N2CCCC2=O)c1 ; O=C1CCCN1c1cc(-c2ccccc2)cc(-c2cc(-c3cc(N4CCCC4=O)cc(N4CCCC4=O)c3)cc(-c3cc(C(F)(F)F)cc(C(F)(F)F)c3)c2)c1 ; [2H]c1c([2H])c([2H])c(-c2cc(-c3cc(-c4ccccc4)cc(N4CCCC4=O)c3)cc(-c3cc(N4CCCC4=O)cc(N4CCCC4=O)c3)c2)c([2H])c1[2H]]

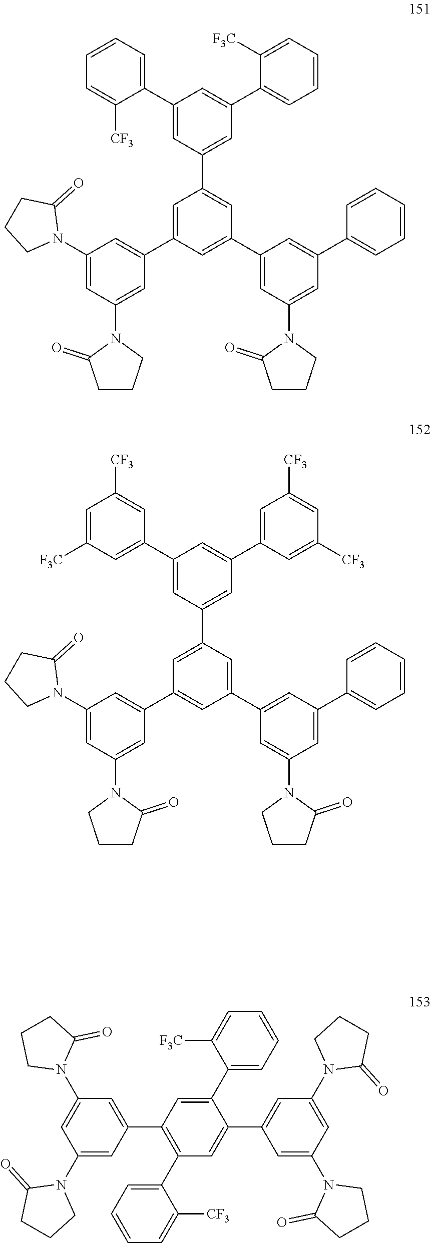 [SMILES: O=C1CCCN1c1cc(-c2ccccc2)cc(-c2cc(-c3cc(-c4ccccc4C(F)(F)F)cc(-c4ccccc4C(F)(F)F)c3)cc(-c3cc(N4CCCC4=O)cc(N4CCCC4=O)c3)c2)c1 ; O=C1CCCN1c1cc(-c2ccccc2)cc(-c2cc(-c3cc(-c4cc(C(F)(F)F)cc(C(F)(F)F)c4)cc(-c4cc(C(F)(F)F)cc(C(F)(F)F)c4)c3)cc(-c3cc(N4CCCC4=O)cc(N4CCCC4=O)c3)c2)c1 ; O=C1CCCN1c1cc(-c2cc(-c3ccccc3C(F)(F)F)c(-c3cc(N4CCCC4=O)cc(N4CCCC4=O)c3)cc2-c2ccccc2C(F)(F)F)cc(N2CCCC2=O)c1]

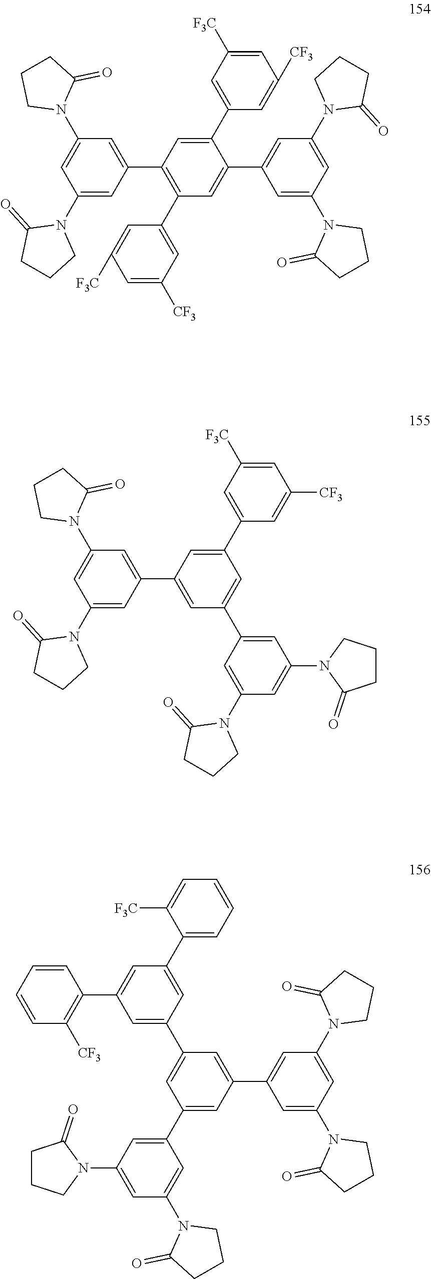 [SMILES: O=C1CCCN1c1cc(-c2cc(-c3cc(C(F)(F)F)cc(C(F)(F)F)c3)c(-c3cc(N4CCCC4=O)cc(N4CCCC4=O)c3)cc2-c2cc(C(F)(F)F)cc(C(F)(F)F)c2)cc(N2CCCC2=O)c1 ; O=C1CCCN1c1cc(-c2cc(-c3cc(N4CCCC4=O)cc(N4CCCC4=O)c3)cc(-c3cc(C(F)(F)F)cc(C(F)(F)F)c3)c2)cc(N2CCCC2=O)c1 ; O=C1CCCN1c1cc(-c2cc(-c3cc(-c4ccccc4C(F)(F)F)cc(-c4ccccc4C(F)(F)F)c3)cc(-c3cc(N4CCCC4=O)cc(N4CCCC4=O)c3)c2)cc(N2CCCC2=O)c1]

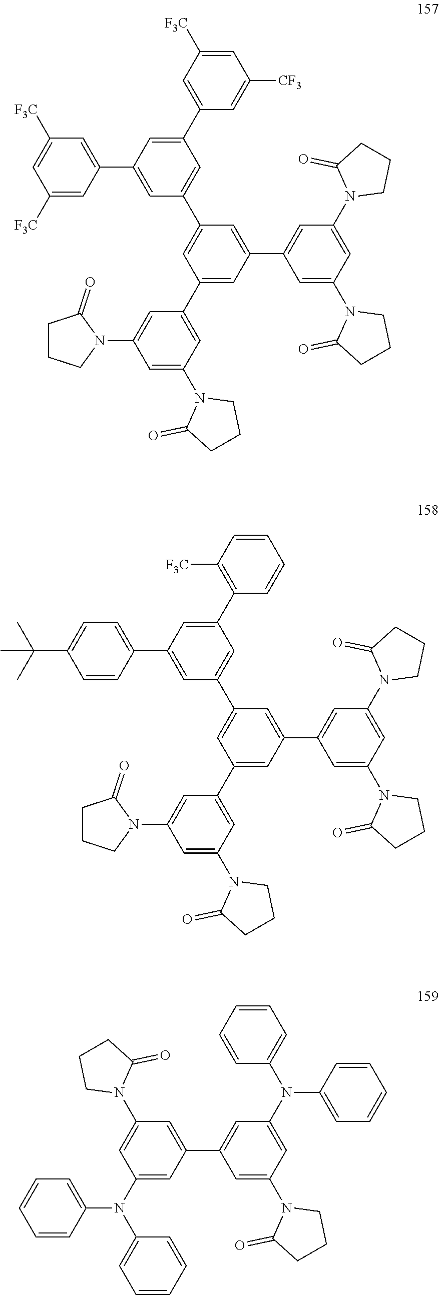 [SMILES: O=C1CCCN1c1cc(-c2cc(-c3cc(-c4cc(C(F)(F)F)cc(C(F)(F)F)c4)cc(-c4cc(C(F)(F)F)cc(C(F)(F)F)c4)c3)cc(-c3cc(N4CCCC4=O)cc(N4CCCC4=O)c3)c2)cc(N2CCCC2=O)c1 ; CC(C)(C)c1ccc(-c2cc(-c3cc(-c4cc(N5CCCC5=O)cc(N5CCCC5=O)c4)cc(-c4cc(N5CCCC5=O)cc(N5CCCC5=O)c4)c3)cc(-c3ccccc3C(F)(F)F)c2)cc1 ; O=C1CCCN1c1cc(-c2cc(N3CCCC3=O)cc(N(c3ccccc3)c3ccccc3)c2)cc(N(c2ccccc2)c2ccccc2)c1]

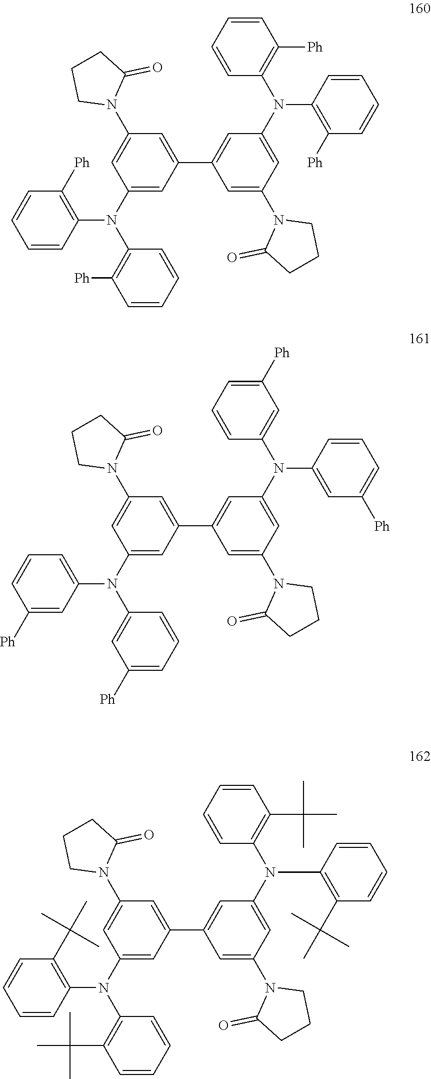 [SMILES: O=C1CCCN1c1cc(-c2cc(N3CCCC3=O)cc(N(c3ccccc3-c3ccccc3)c3ccccc3-c3ccccc3)c2)cc(N(c2ccccc2-c2ccccc2)c2ccccc2-c2ccccc2)c1 ; O=C1CCCN1c1cc(-c2cc(N3CCCC3=O)cc(N(c3cccc(-c4ccccc4)c3)c3cccc(-c4ccccc4)c3)c2)cc(N(c2cccc(-c3ccccc3)c2)c2cccc(-c3ccccc3)c2)c1 ; CC(C)(C)c1ccccc1N(c1cc(-c2cc(N3CCCC3=O)cc(N(c3ccccc3C(C)(C)C)c3ccccc3C(C)(C)C)c2)cc(N2CCCC2=O)c1)c1ccccc1C(C)(C)C]

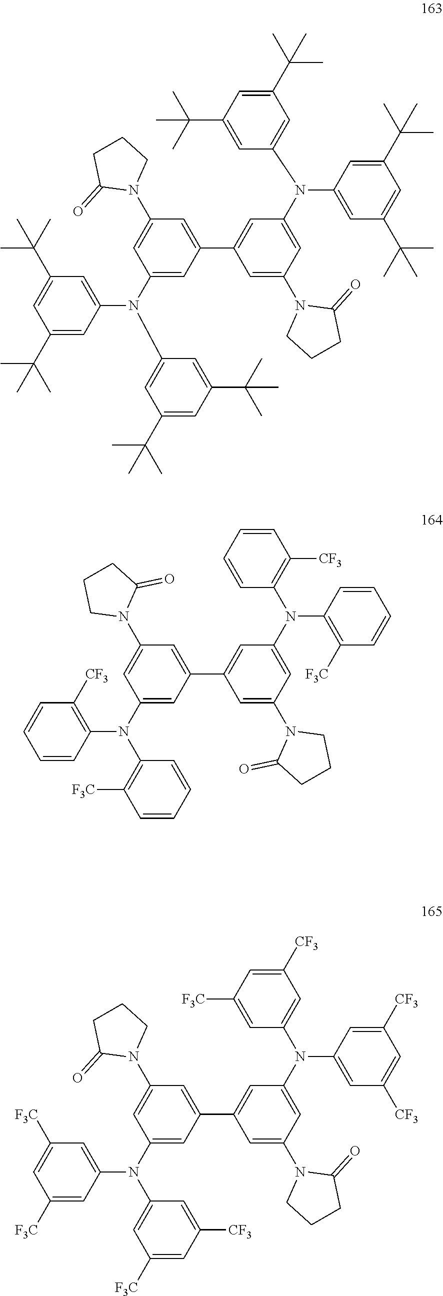 [SMILES: CC(C)(C)c1cc(N(c2cc(-c3cc(N4CCCC4=O)cc(N(c4cc(C(C)(C)C)cc(C(C)(C)C)c4)c4cc(C(C)(C)C)cc(C(C)(C)C)c4)c3)cc(N3CCCC3=O)c2)c2cc(C(C)(C)C)cc(C(C)(C)C)c2)cc(C(C)(C)C)c1 ; O=C1CCCN1c1cc(-c2cc(N3CCCC3=O)cc(N(c3ccccc3C(F)(F)F)c3ccccc3C(F)(F)F)c2)cc(N(c2ccccc2C(F)(F)F)c2ccccc2C(F)(F)F)c1 ; O=C1CCCN1c1cc(-c2cc(N3CCCC3=O)cc(N(c3cc(C(F)(F)F)cc(C(F)(F)F)c3)c3cc(C(F)(F)F)cc(C(F)(F)F)c3)c2)cc(N(c2cc(C(F)(F)F)cc(C(F)(F)F)c2)c2cc(C(F)(F)F)cc(C(F)(F)F)c2)c1]

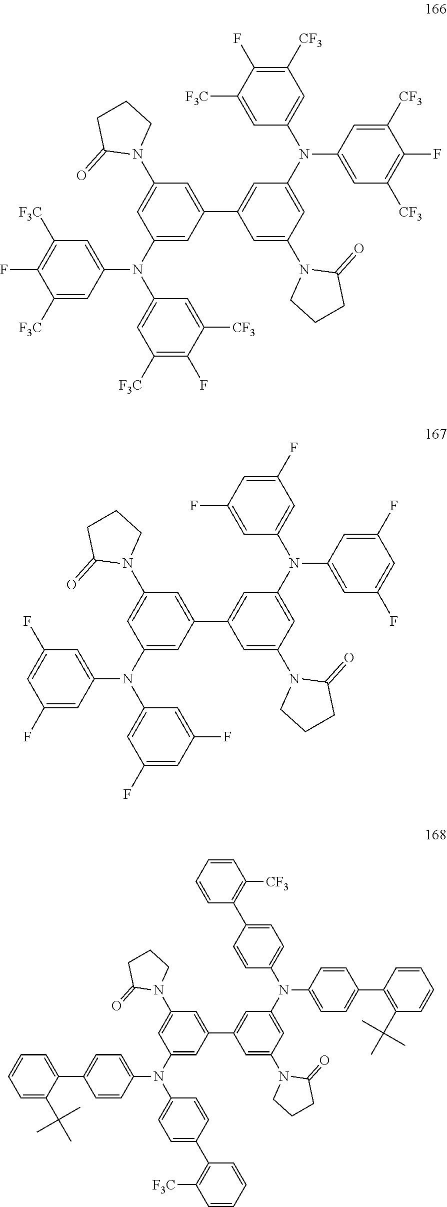 [SMILES: O=C1CCCN1c1cc(-c2cc(N3CCCC3=O)cc(N(c3cc(C(F)(F)F)c(F)c(C(F)(F)F)c3)c3cc(C(F)(F)F)c(F)c(C(F)(F)F)c3)c2)cc(N(c2cc(C(F)(F)F)c(F)c(C(F)(F)F)c2)c2cc(C(F)(F)F)c(F)c(C(F)(F)F)c2)c1 ; O=C1CCCN1c1cc(-c2cc(N3CCCC3=O)cc(N(c3cc(F)cc(F)c3)c3cc(F)cc(F)c3)c2)cc(N(c2cc(F)cc(F)c2)c2cc(F)cc(F)c2)c1 ; CC(C)(C)c1ccccc1-c1ccc(N(c2ccc(-c3ccccc3C(F)(F)F)cc2)c2cc(-c3cc(N4CCCC4=O)cc(N(c4ccc(-c5ccccc5C(C)(C)C)cc4)c4ccc(-c5ccccc5C(F)(F)F)cc4)c3)cc(N3CCCC3=O)c2)cc1]

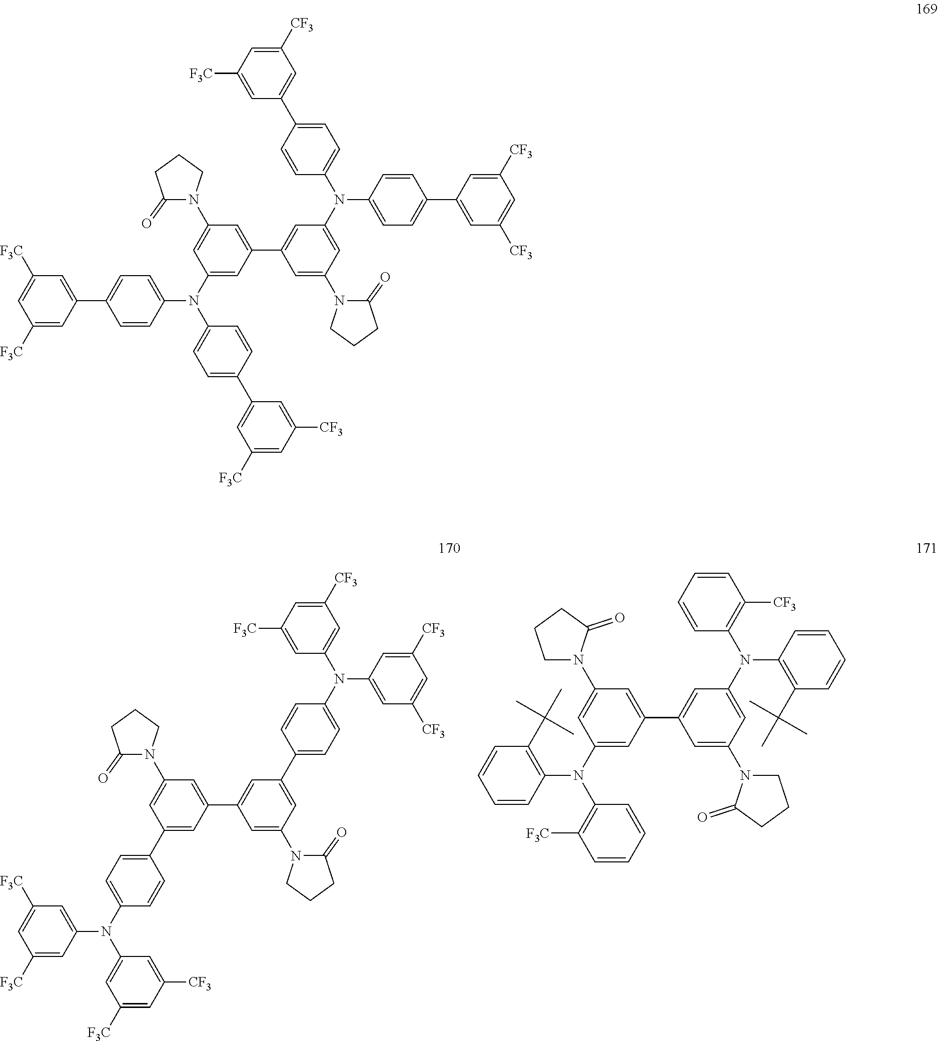

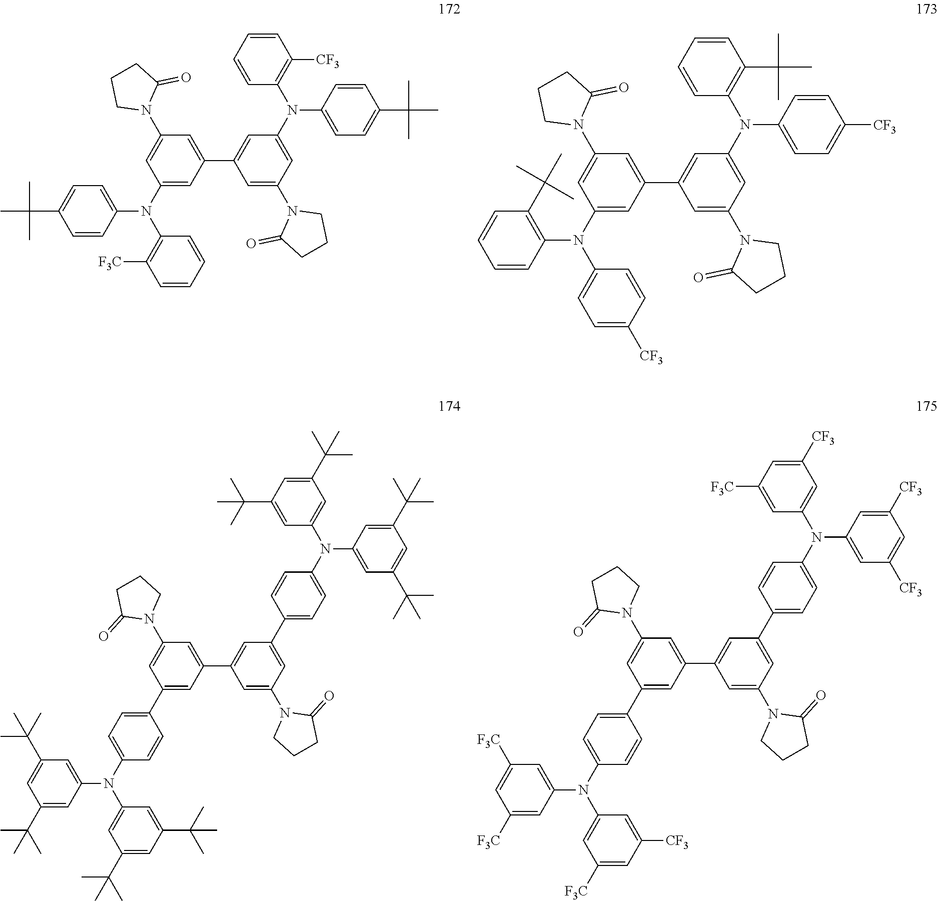

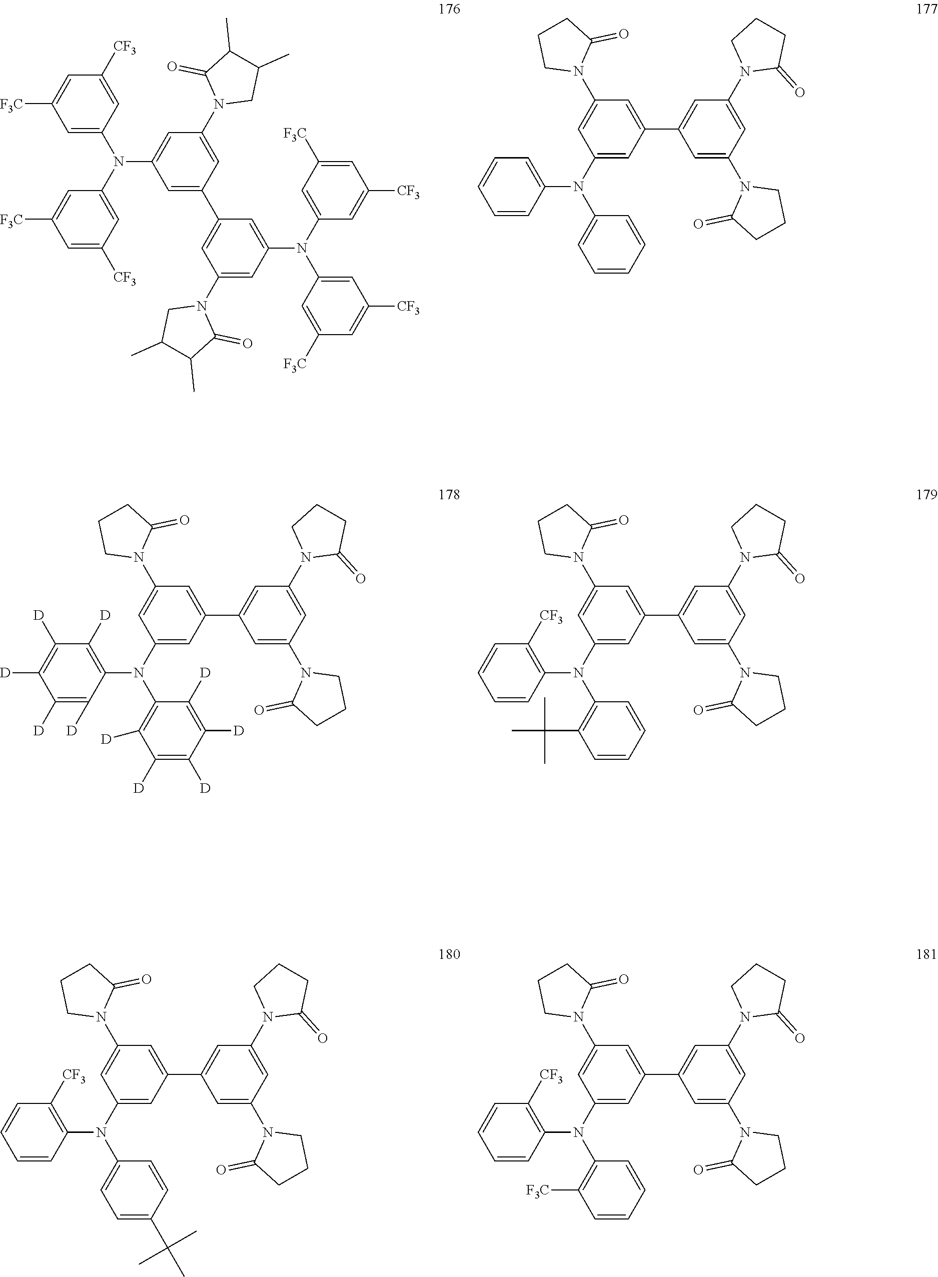

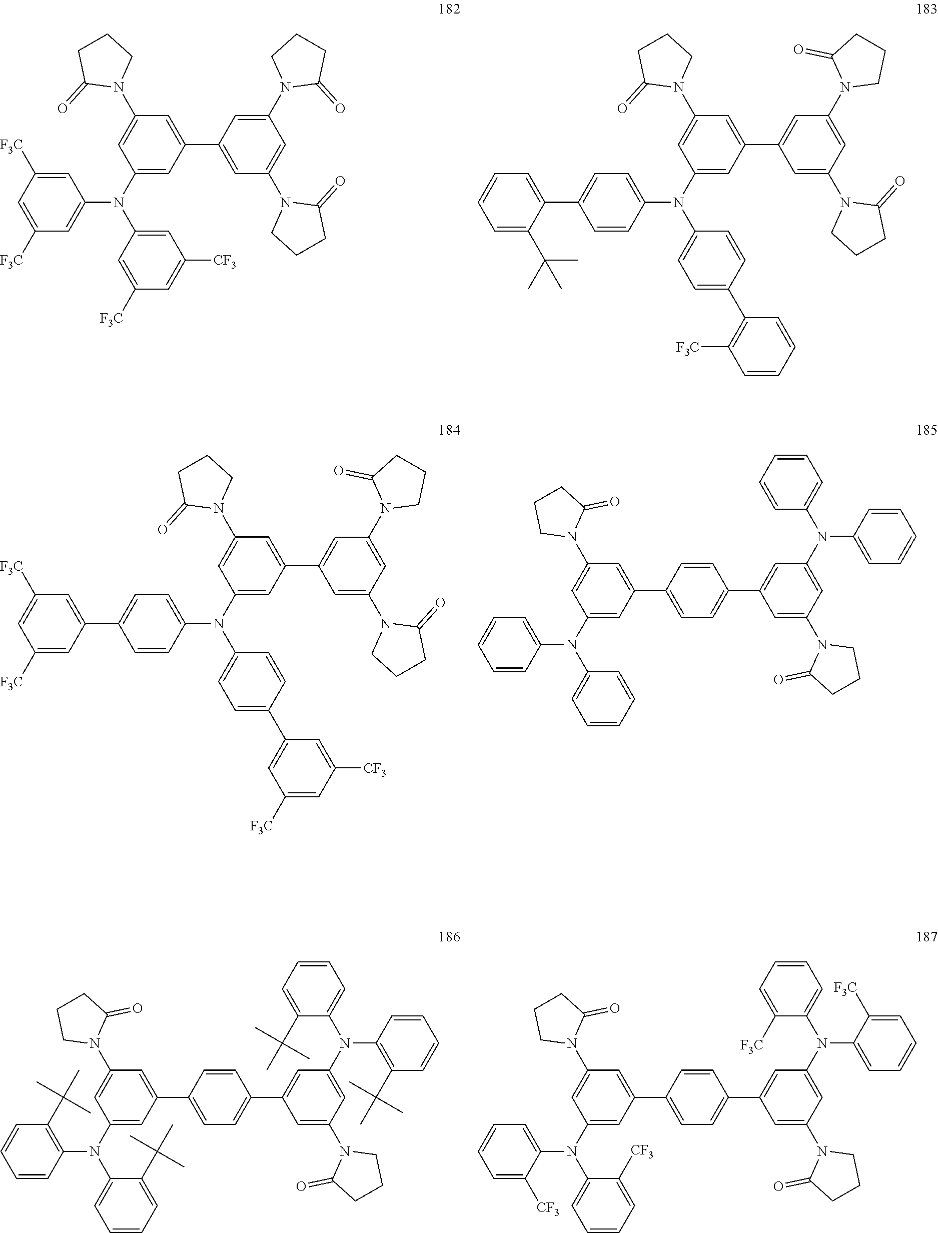

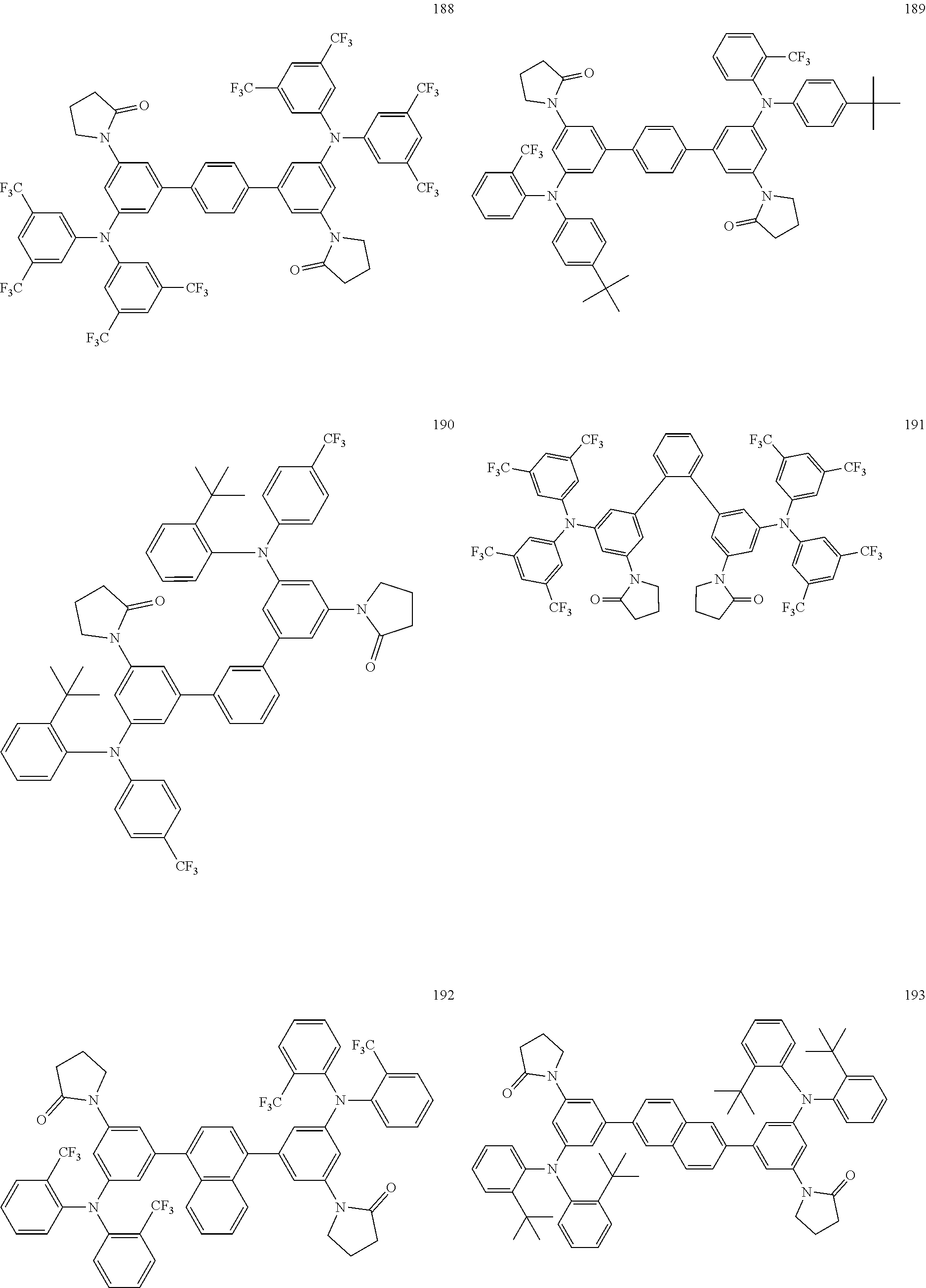

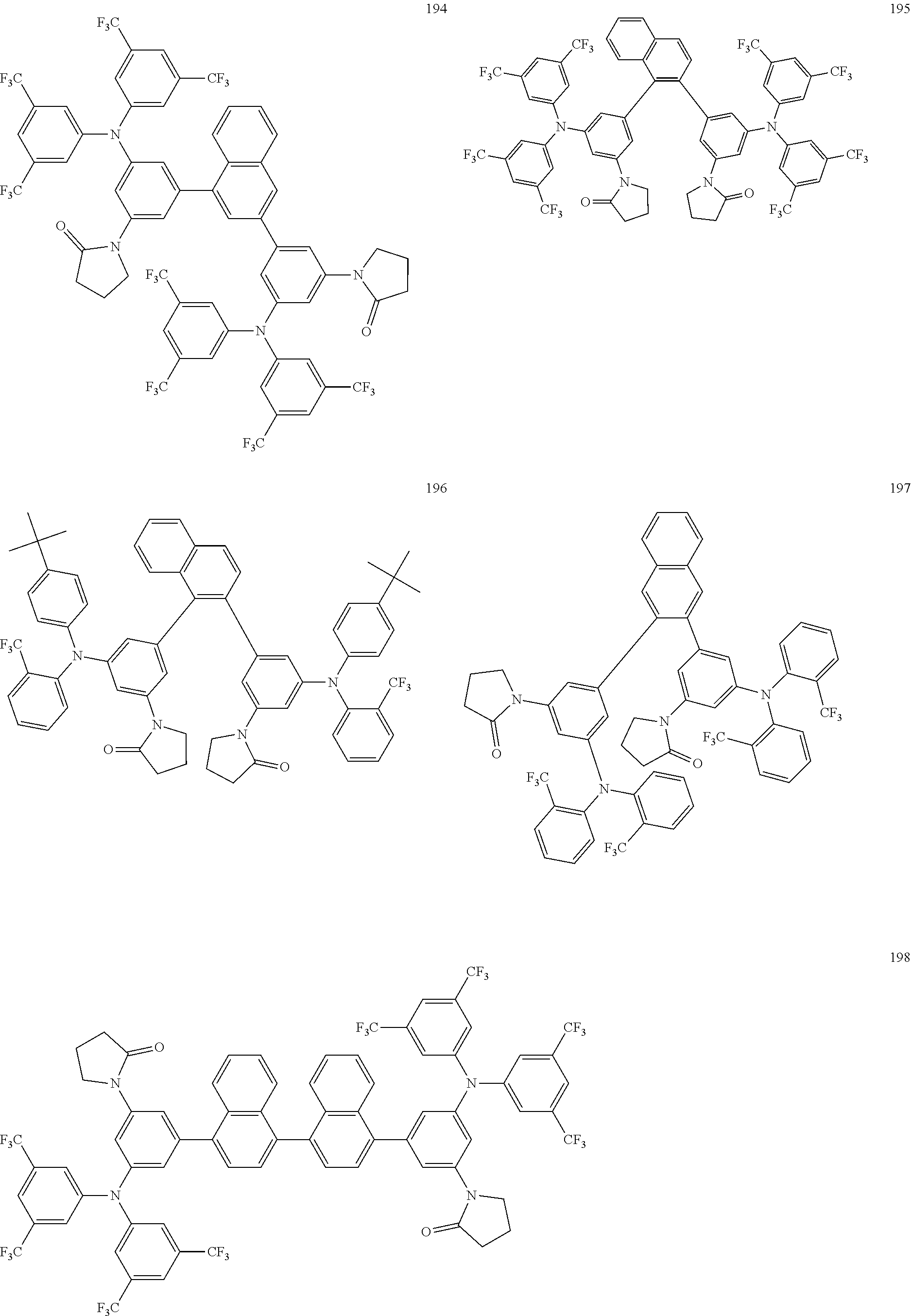

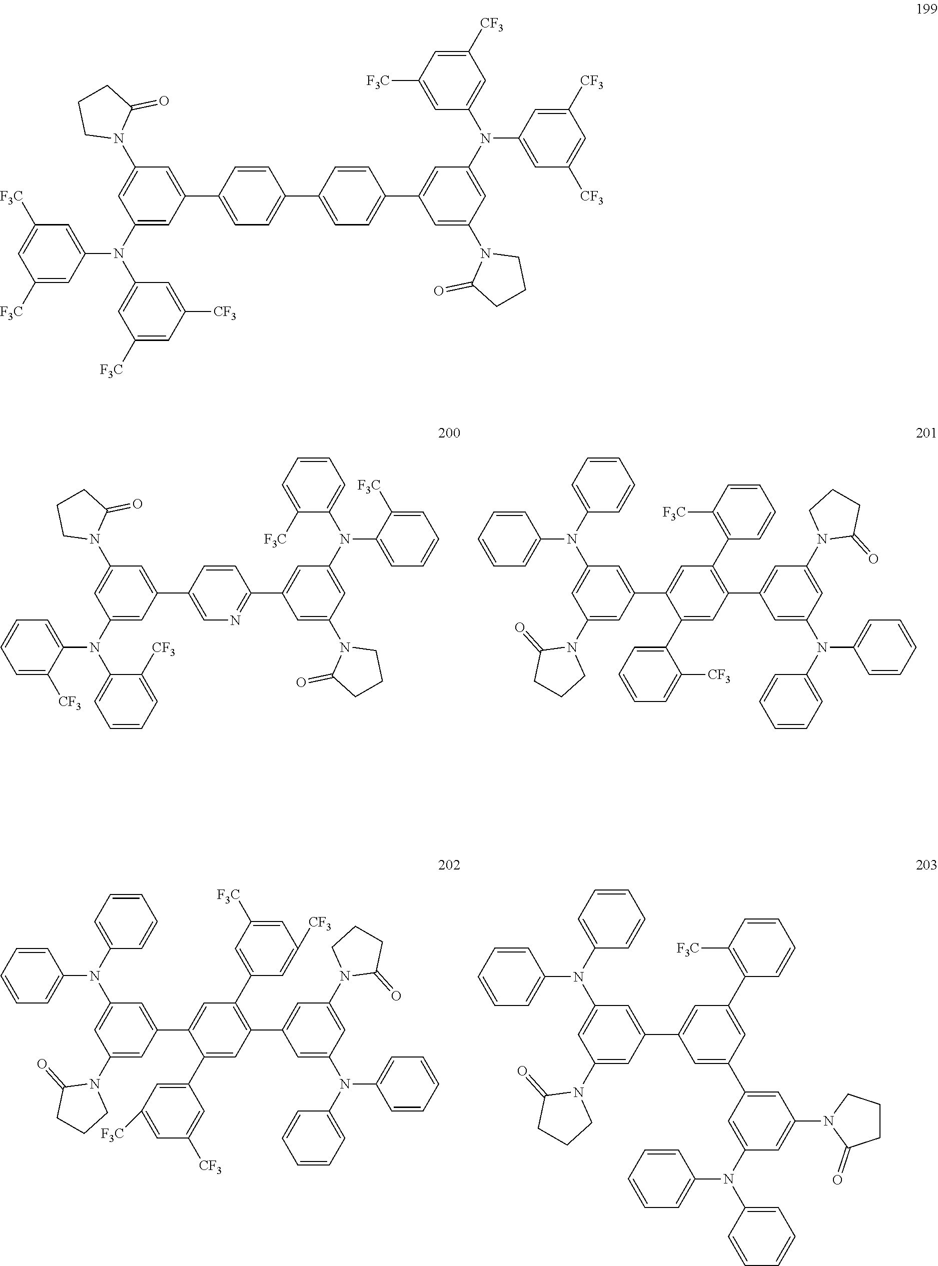

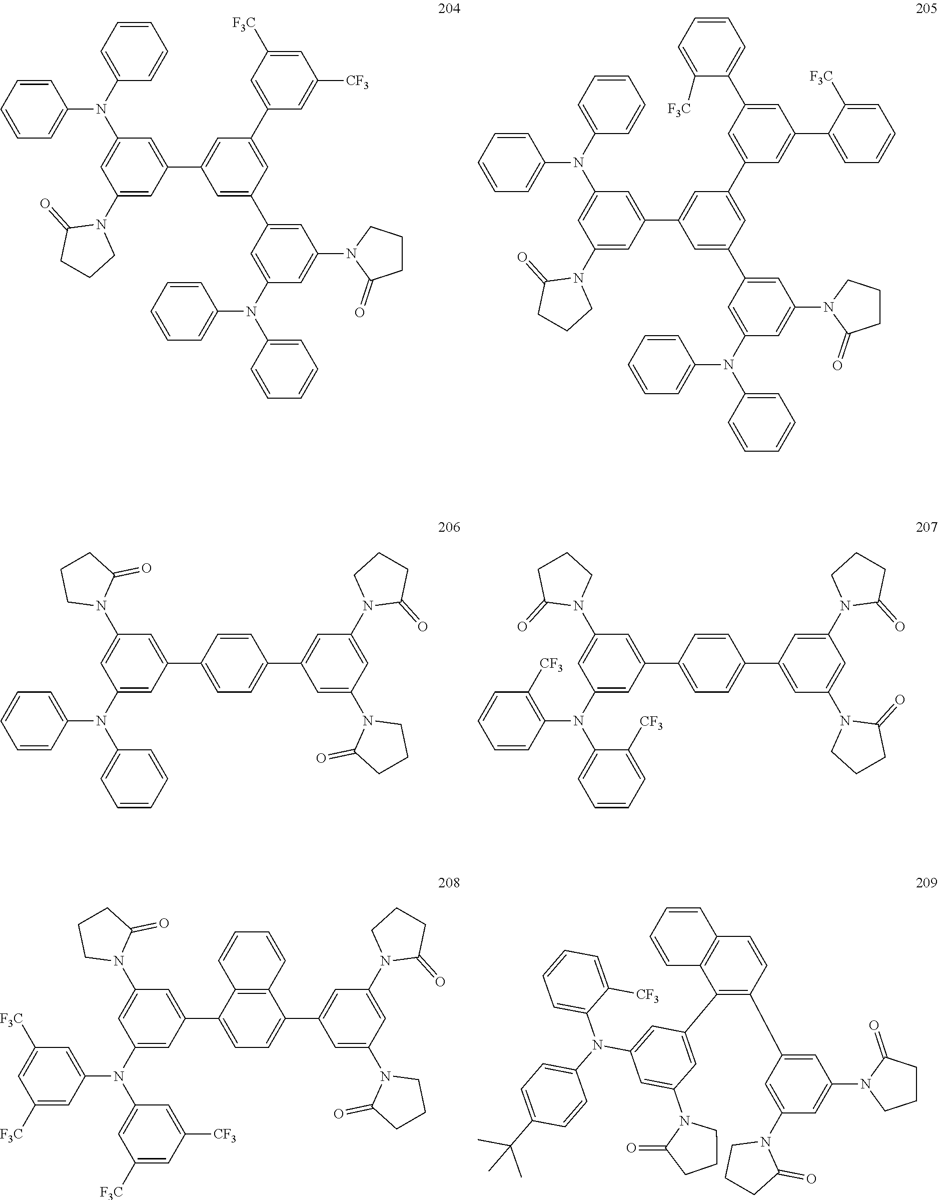

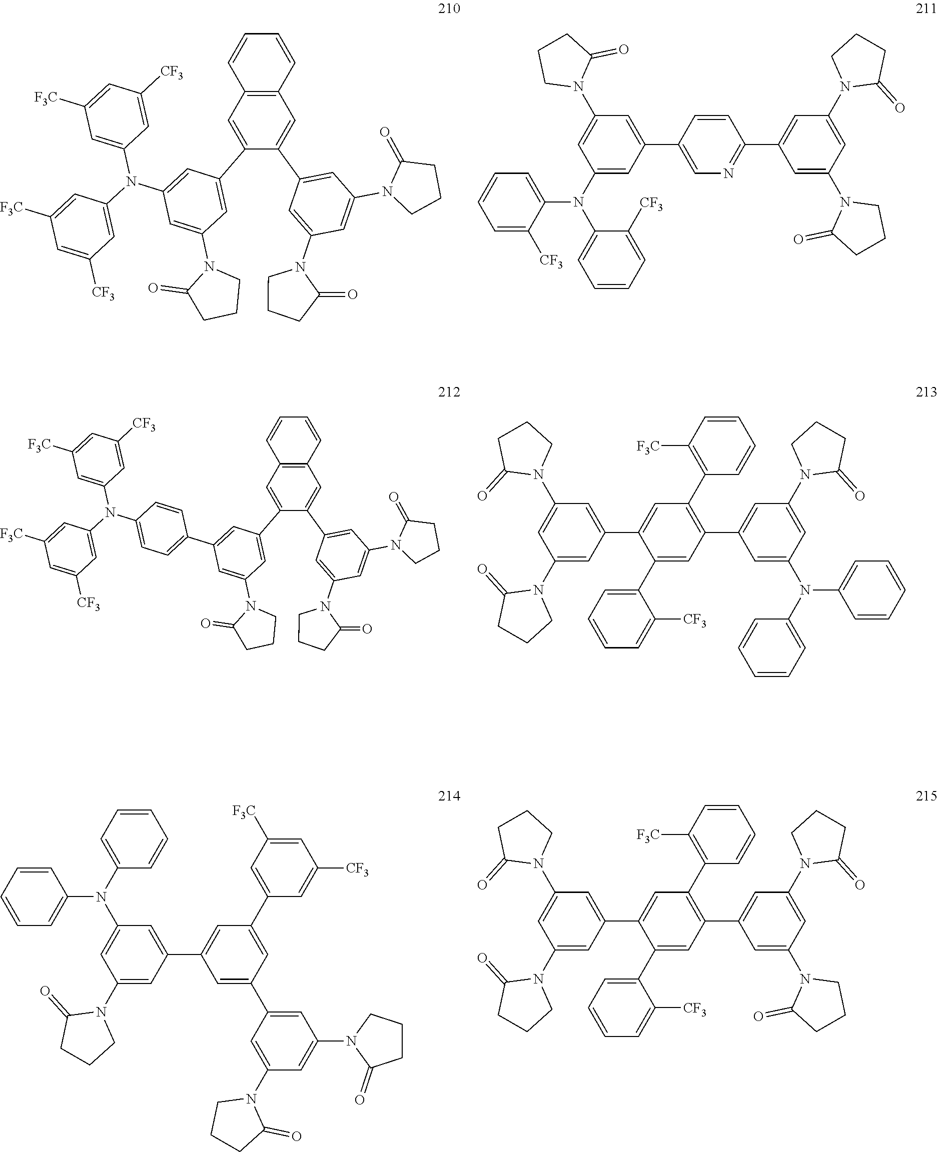

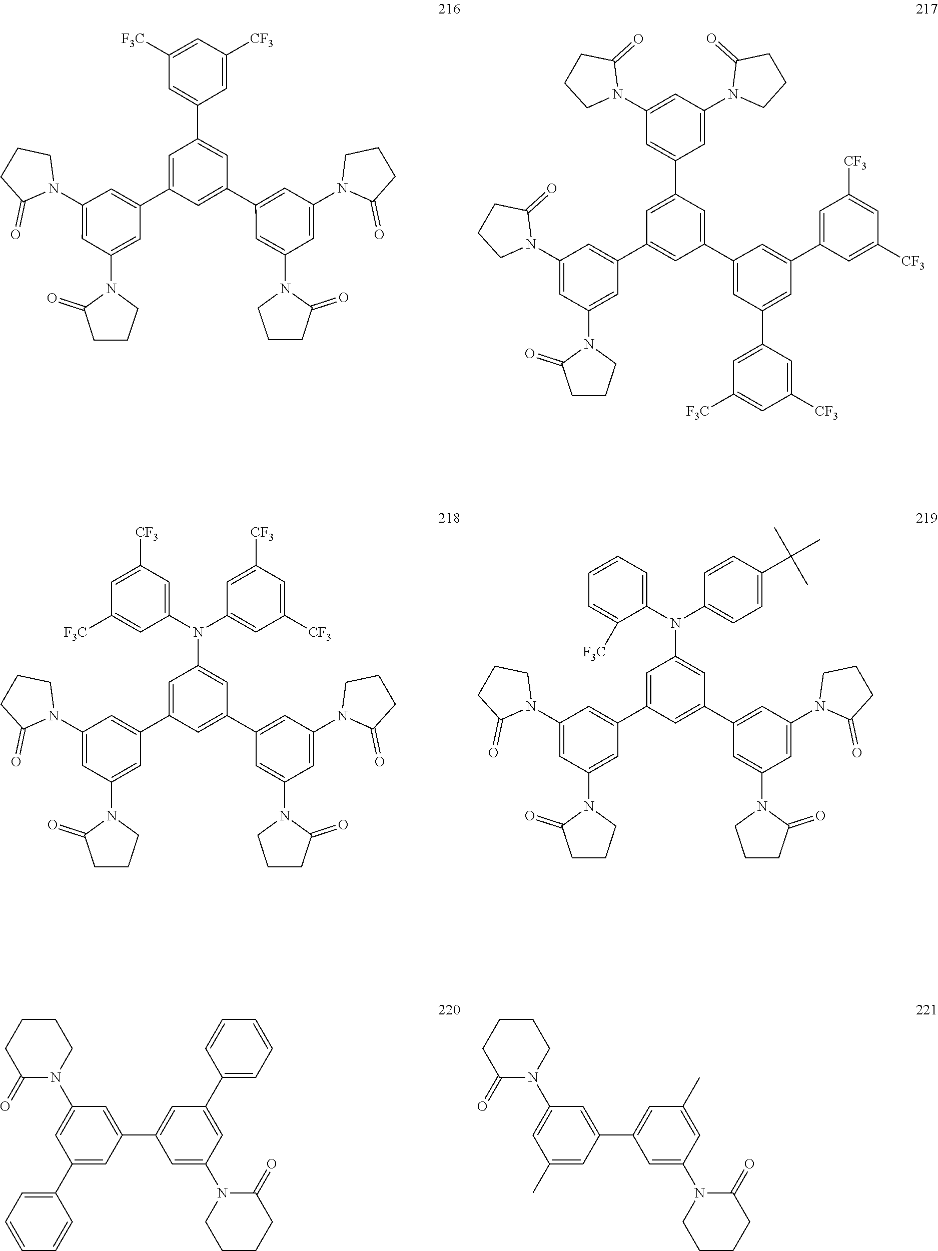

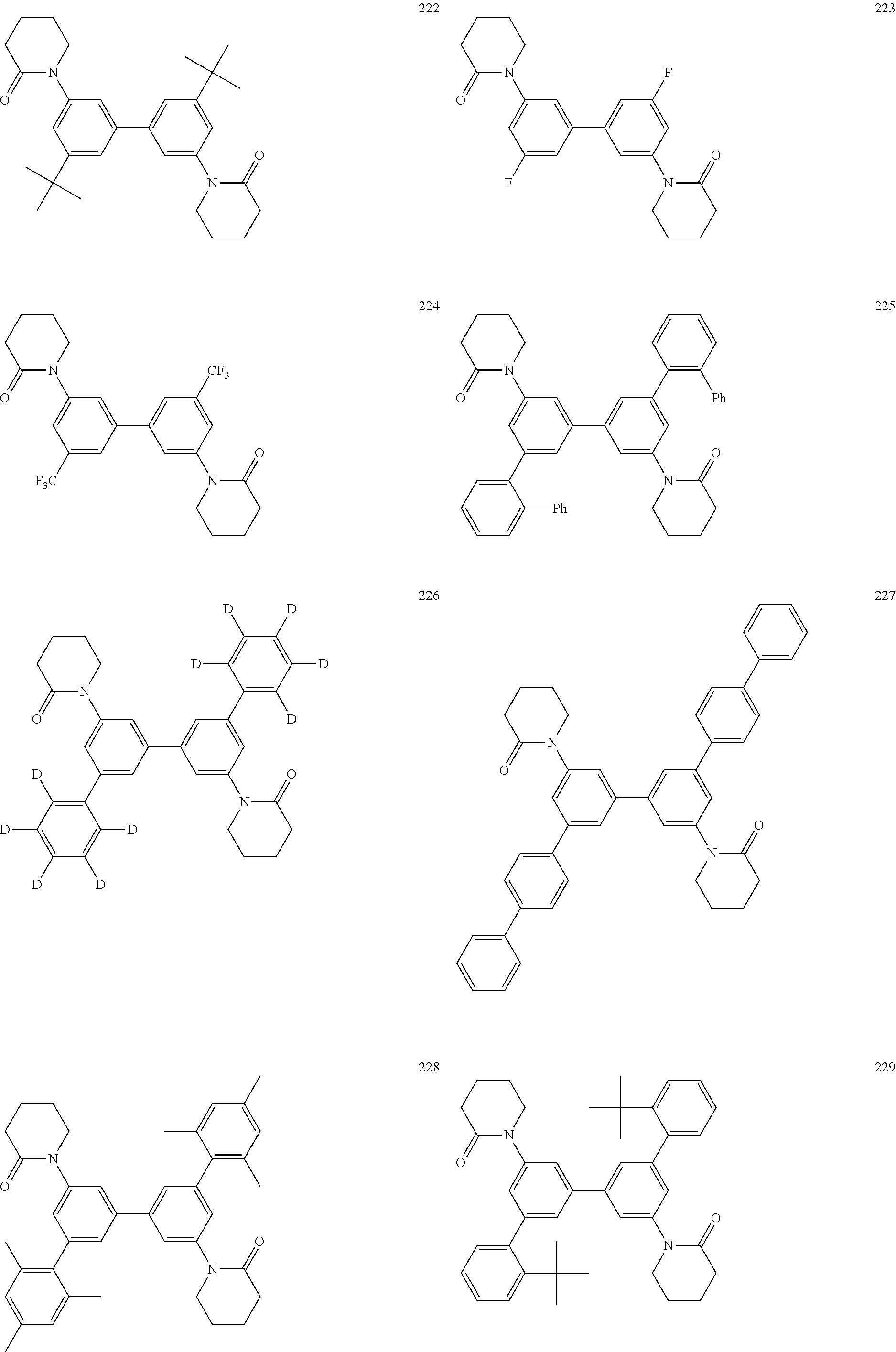

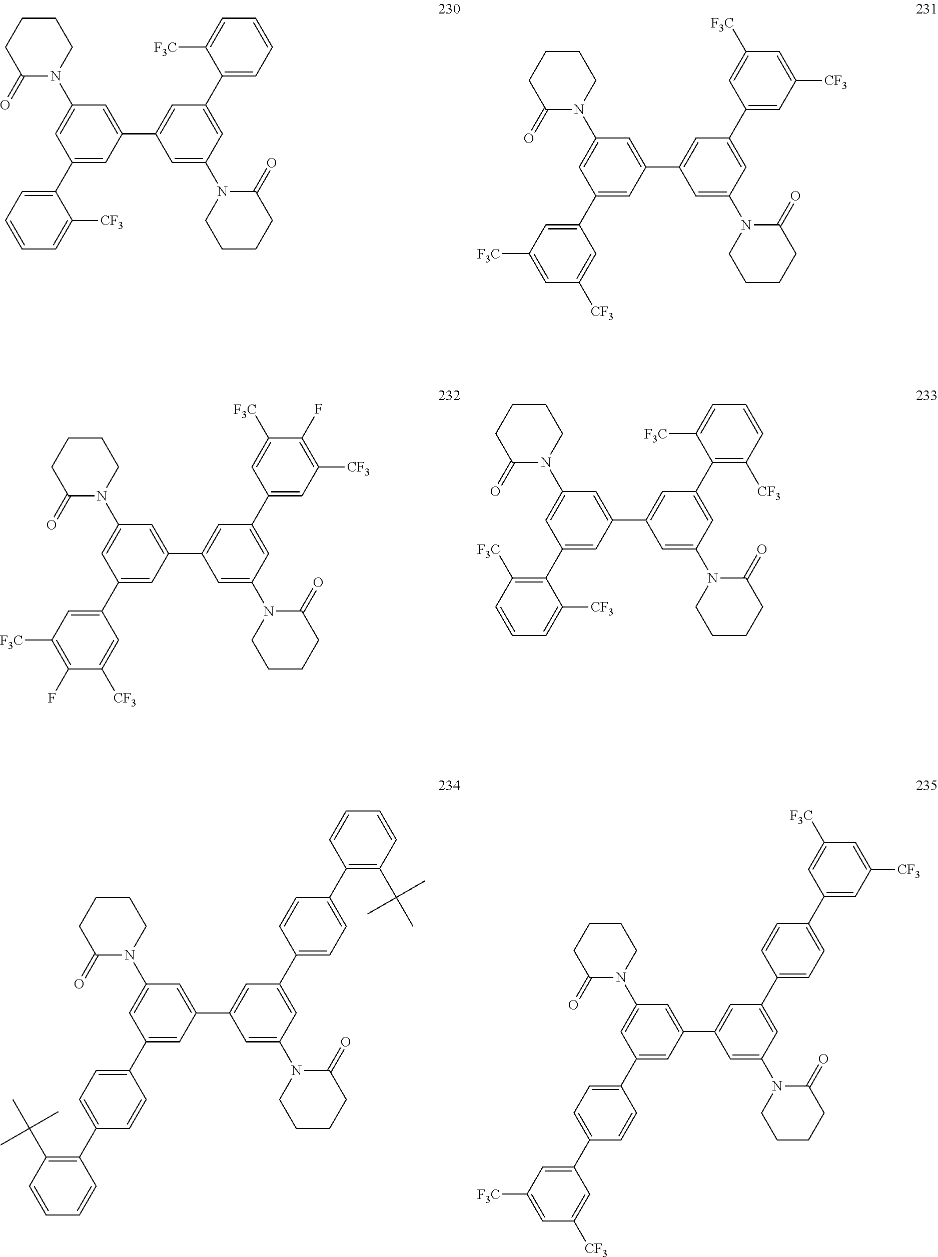

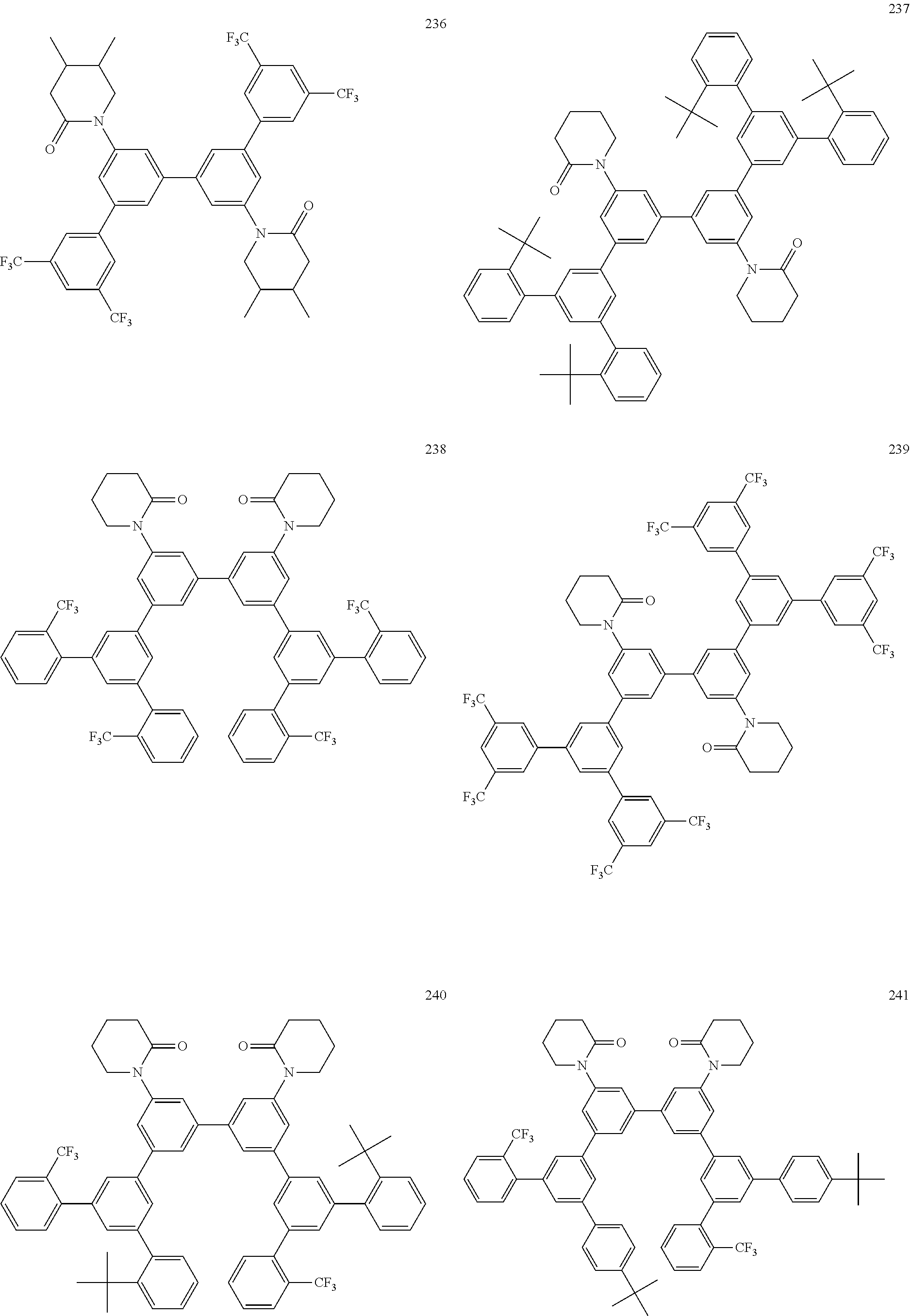

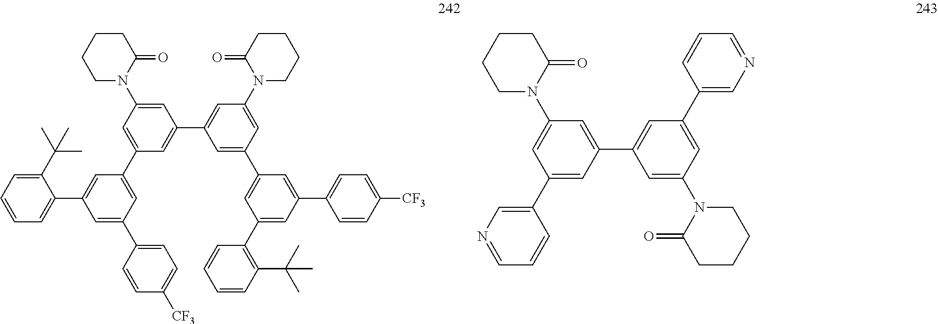 [SMILES: CC(C)(C)c1ccccc1-c1cc(-c2ccc(C(F)(F)F)cc2)cc(-c2cc(-c3cc(-c4cc(-c5ccc(C(F)(F)F)cc5)cc(-c5ccccc5C(C)(C)C)c4)cc(N4CCCCC4=O)c3)cc(N3CCCCC3=O)c2)c1 ; O=C1CCCCN1c1cc(-c2cccnc2)cc(-c2cc(-c3cccnc3)cc(N3CCCCC3=O)c2)c1]

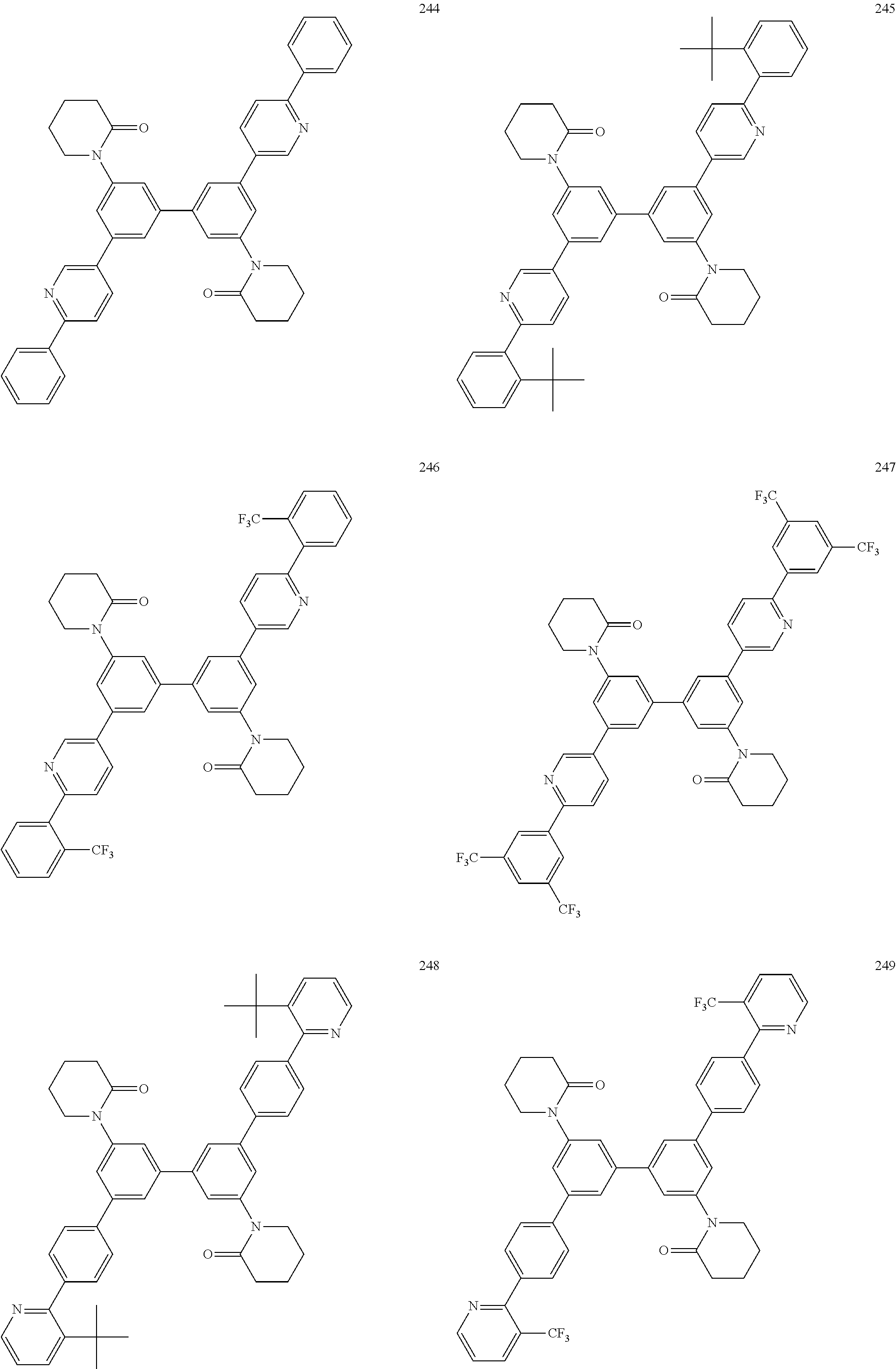

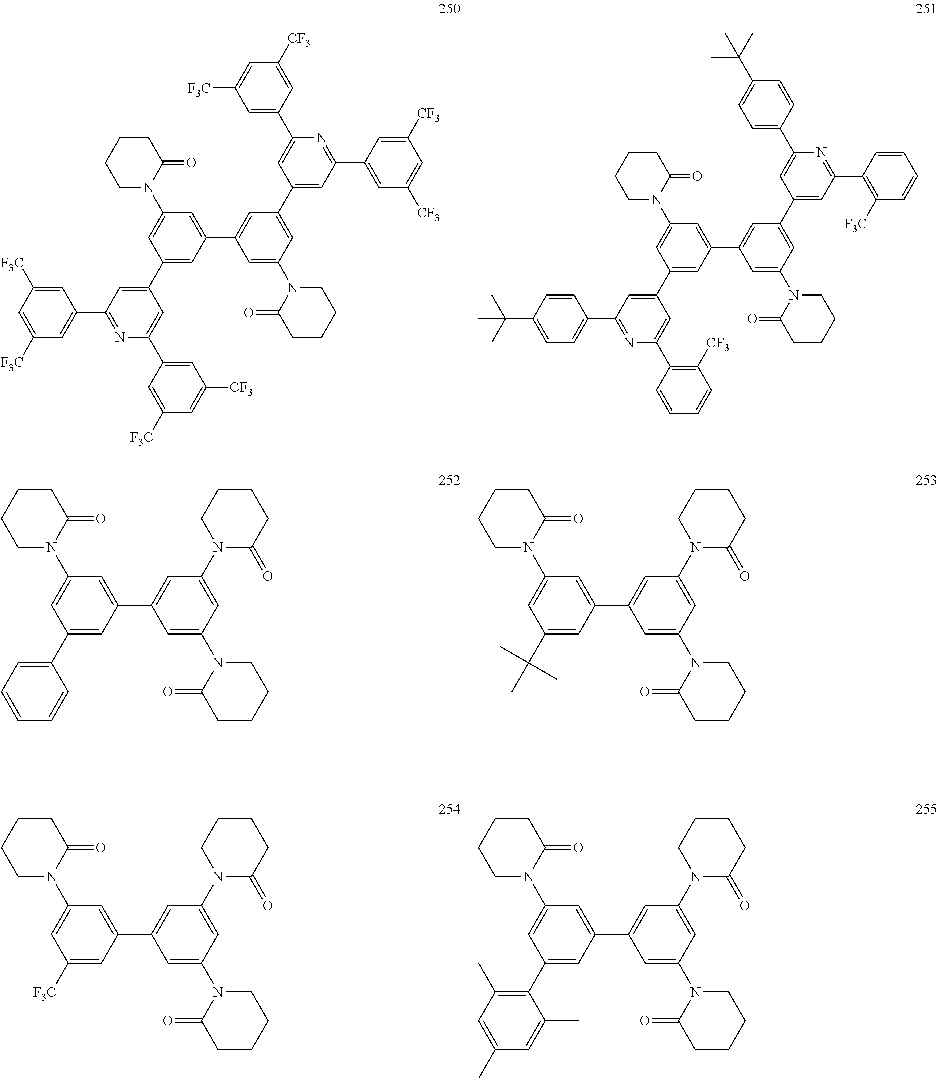

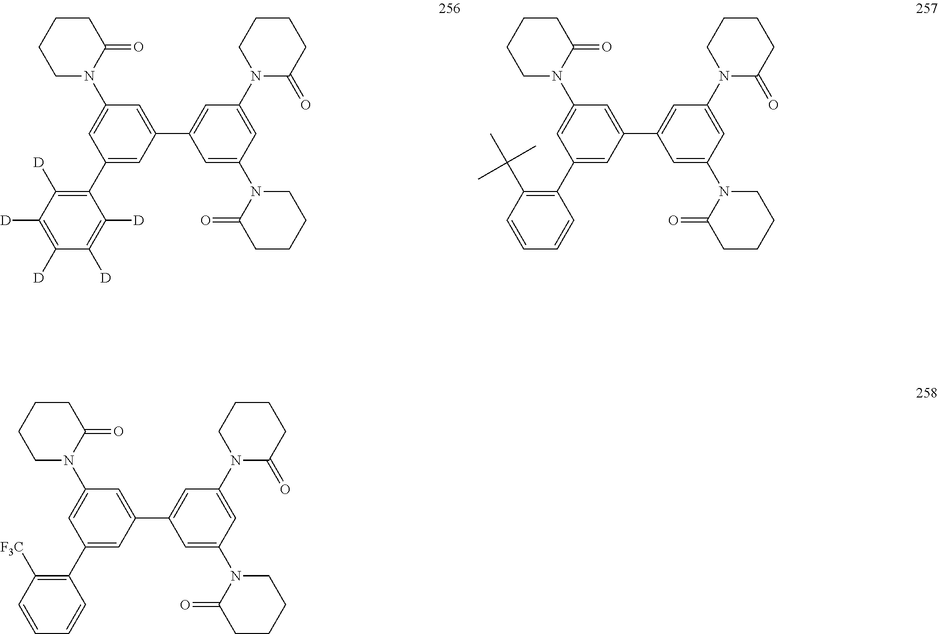 [SMILES: [2H]c1c([2H])c([2H])c(-c2cc(-c3cc(N4CCCCC4=O)cc(N4CCCCC4=O)c3)cc(N3CCCCC3=O)c2)c([2H])c1[2H] ; CC(C)(C)c1ccccc1-c1cc(-c2cc(N3CCCCC3=O)cc(N3CCCCC3=O)c2)cc(N2CCCCC2=O)c1 ; O=C1CCCCN1c1cc(-c2cc(N3CCCCC3=O)cc(N3CCCCC3=O)c2)cc(-c2ccccc2C(F)(F)F)c1]

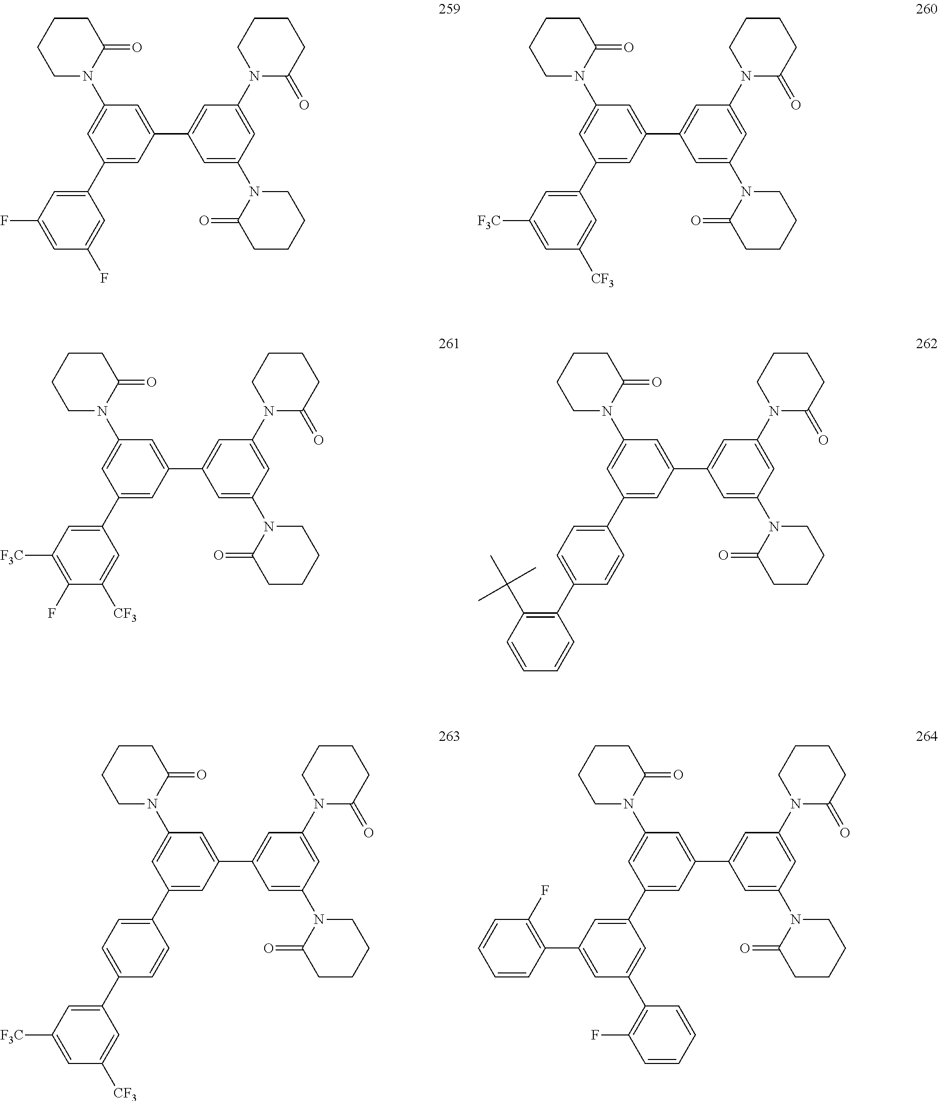

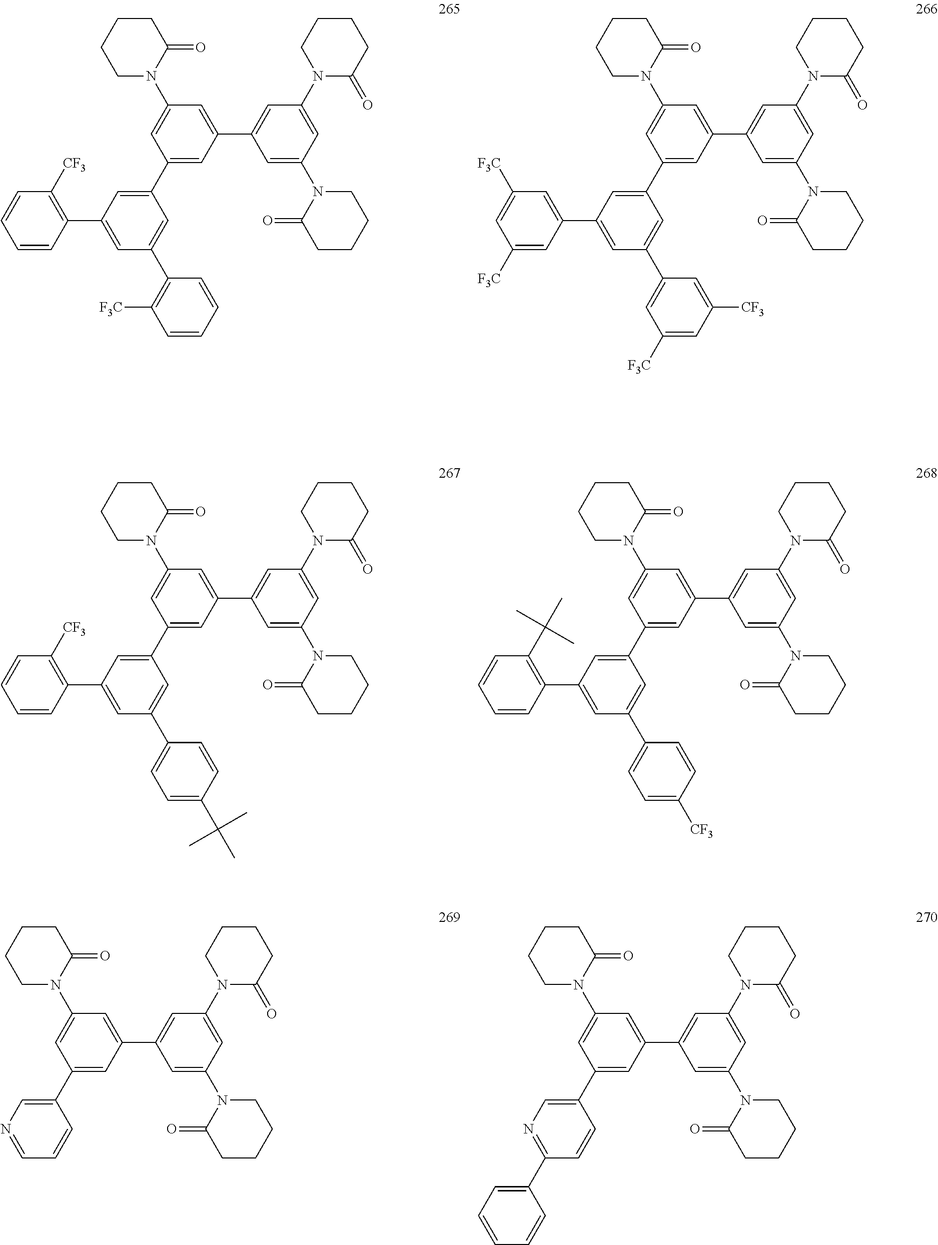

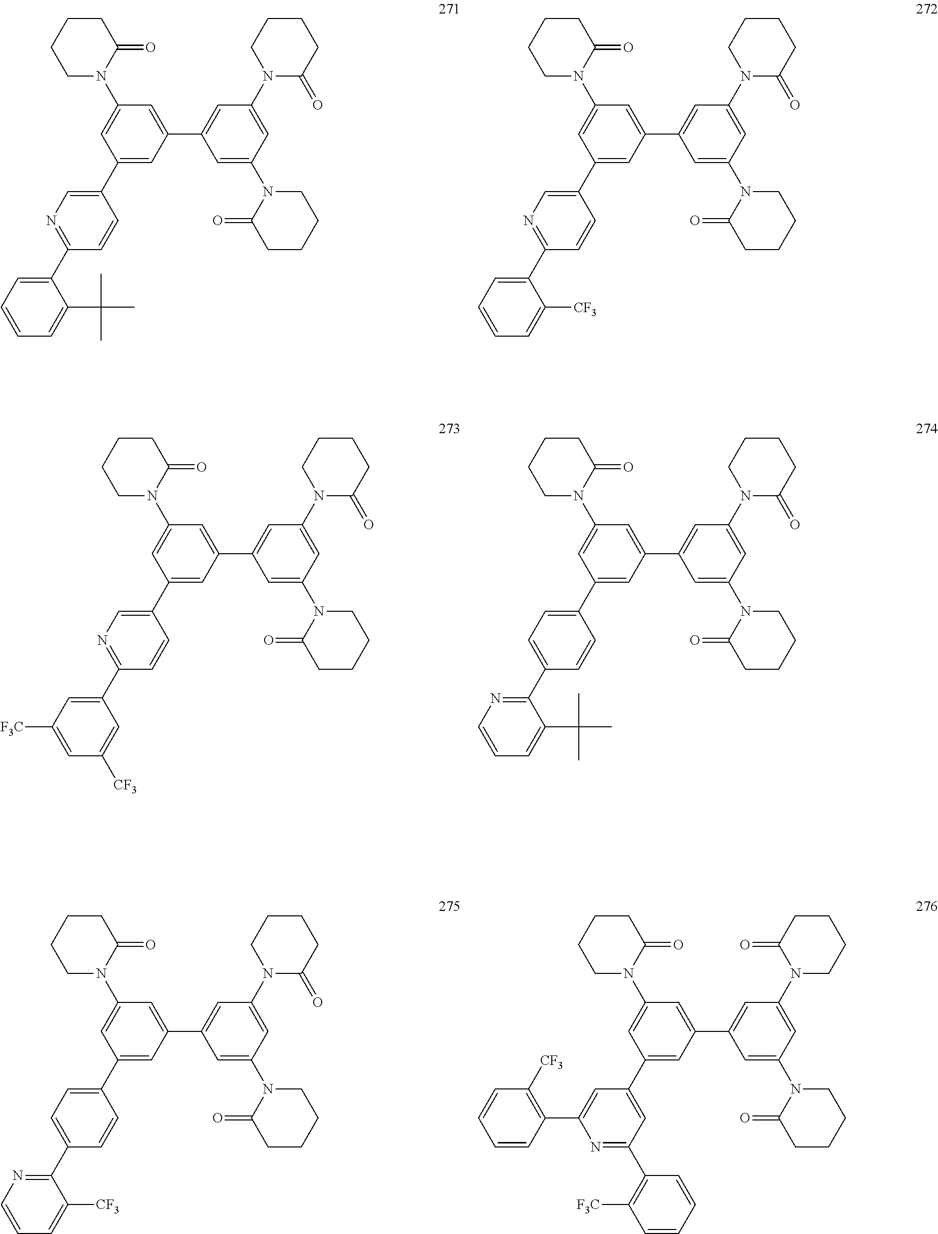

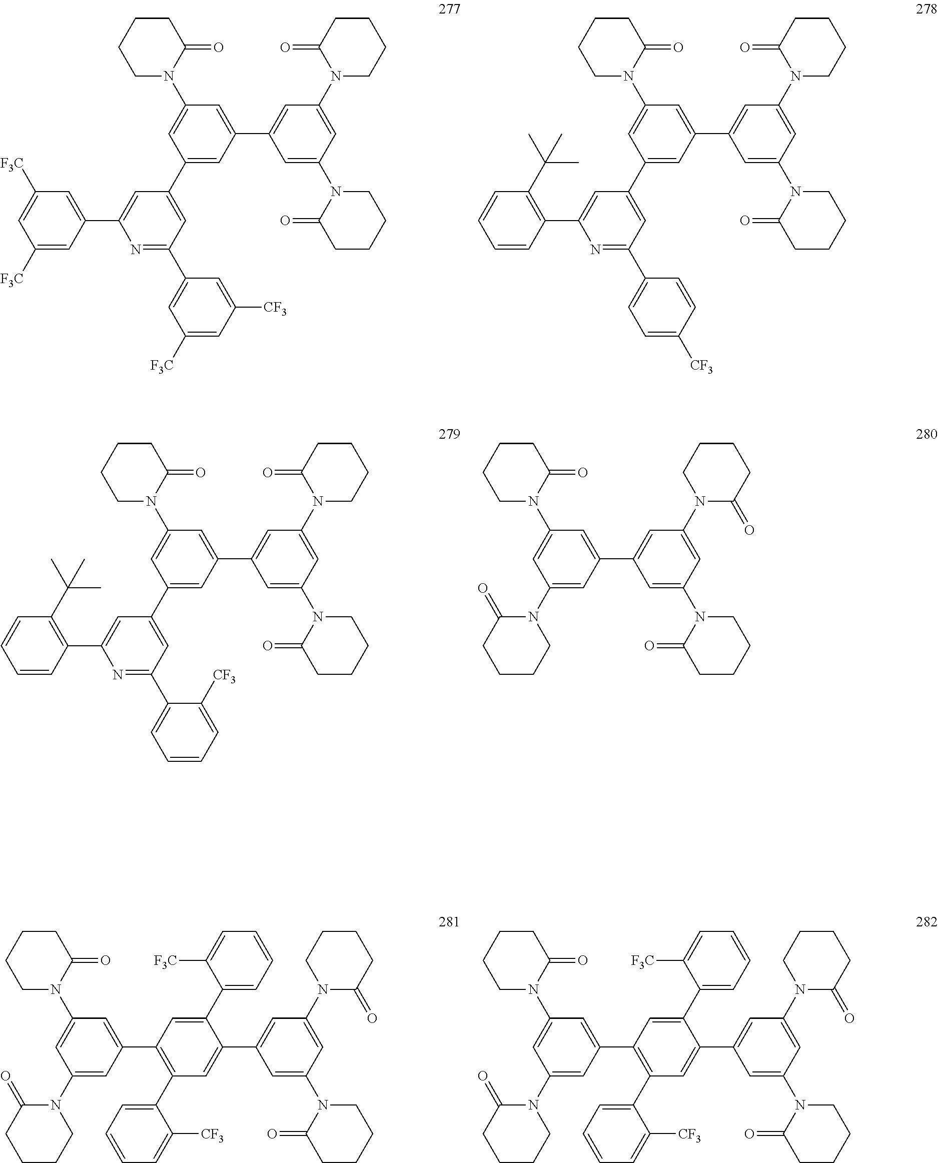

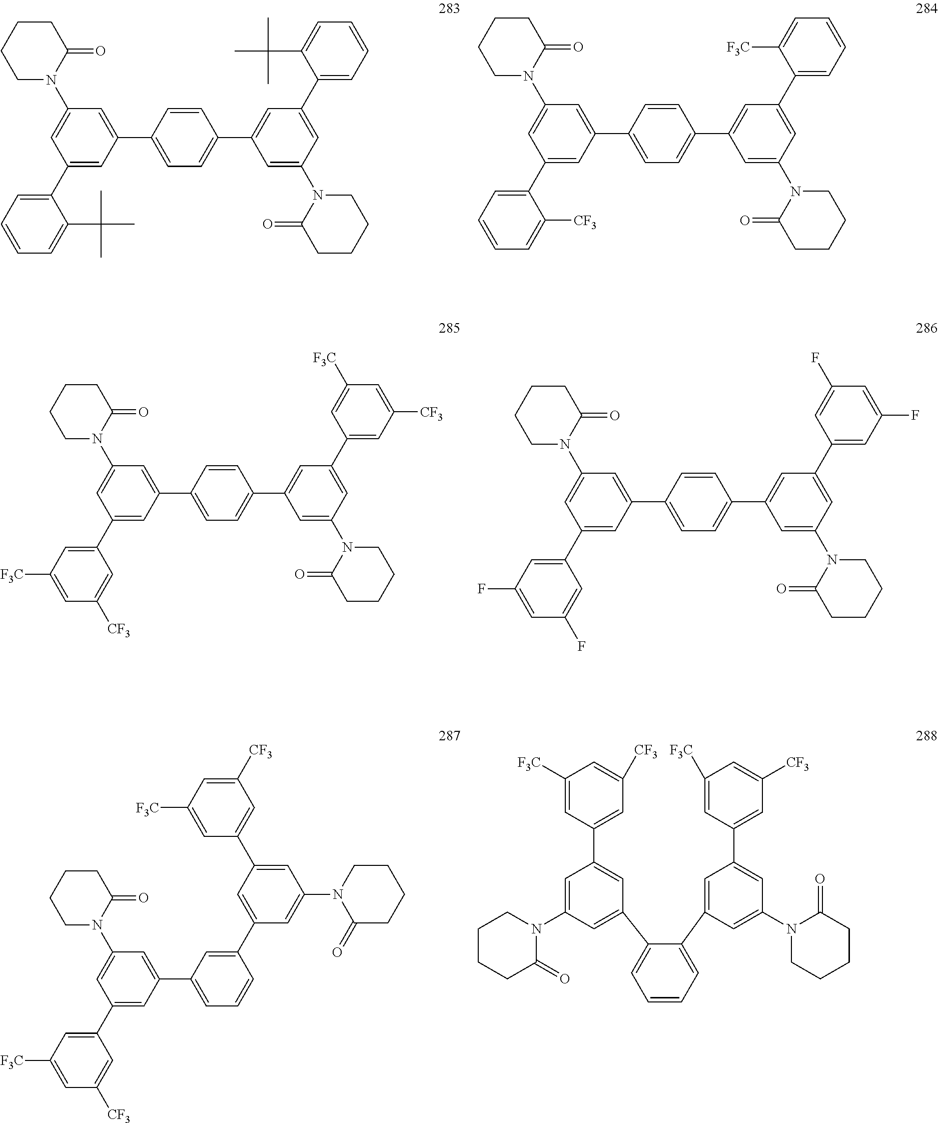

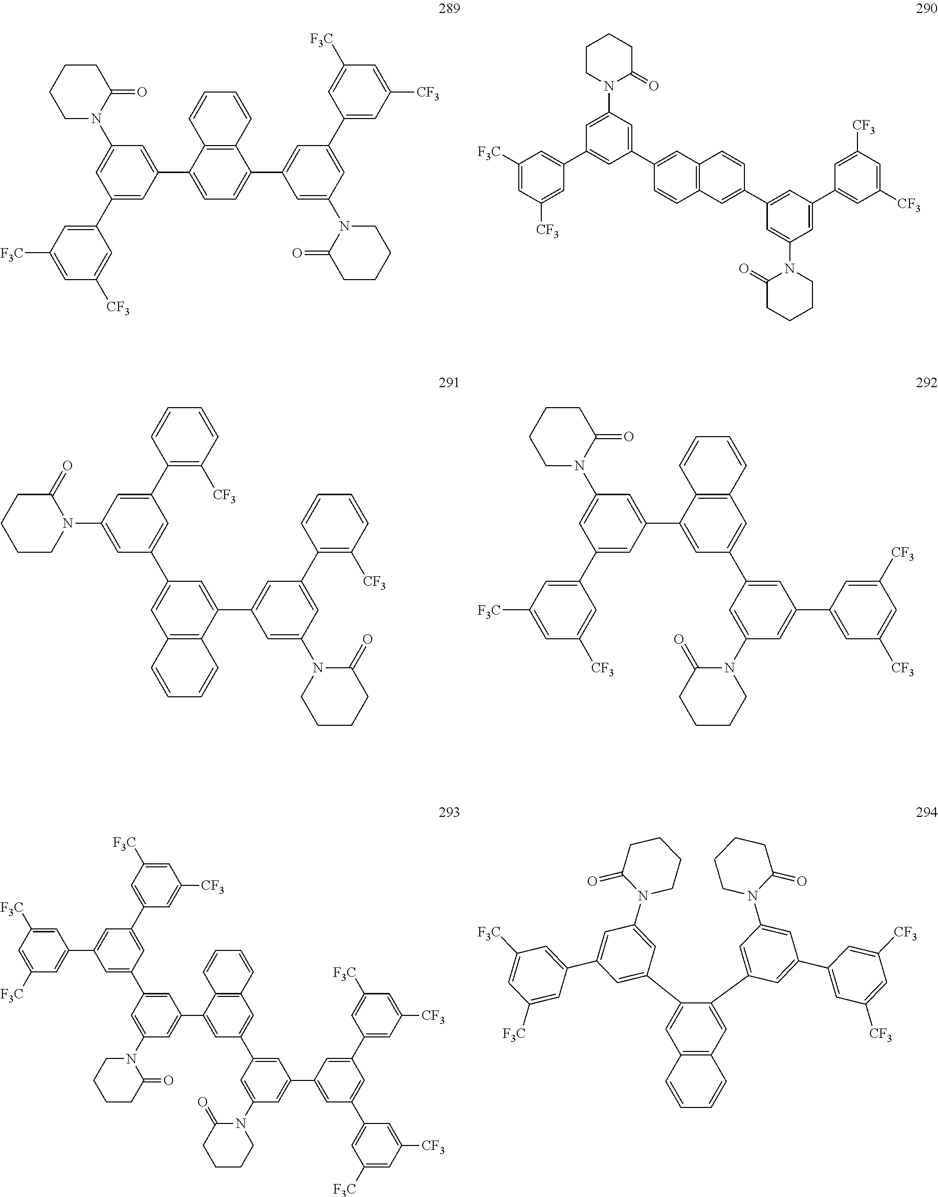

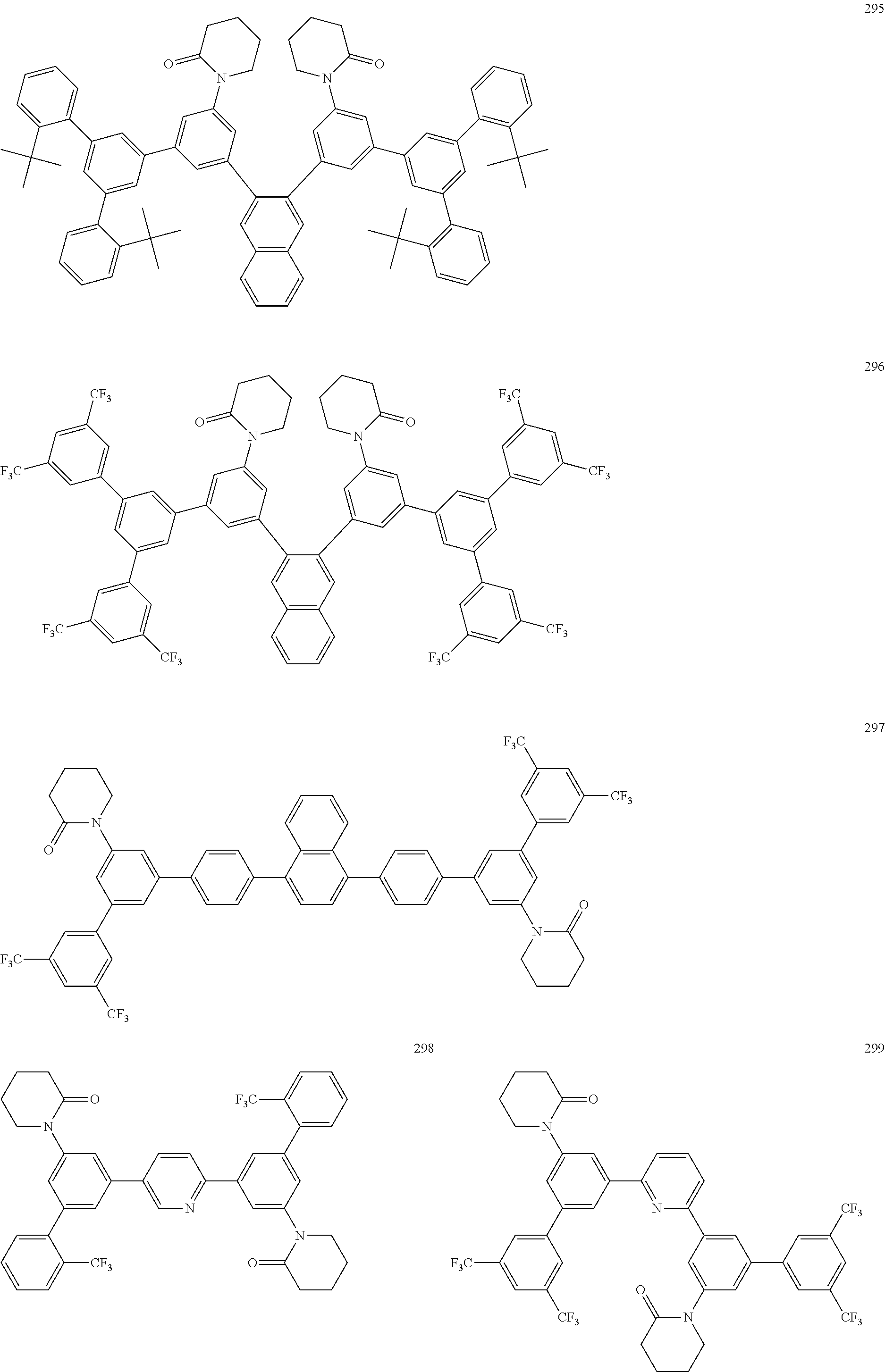

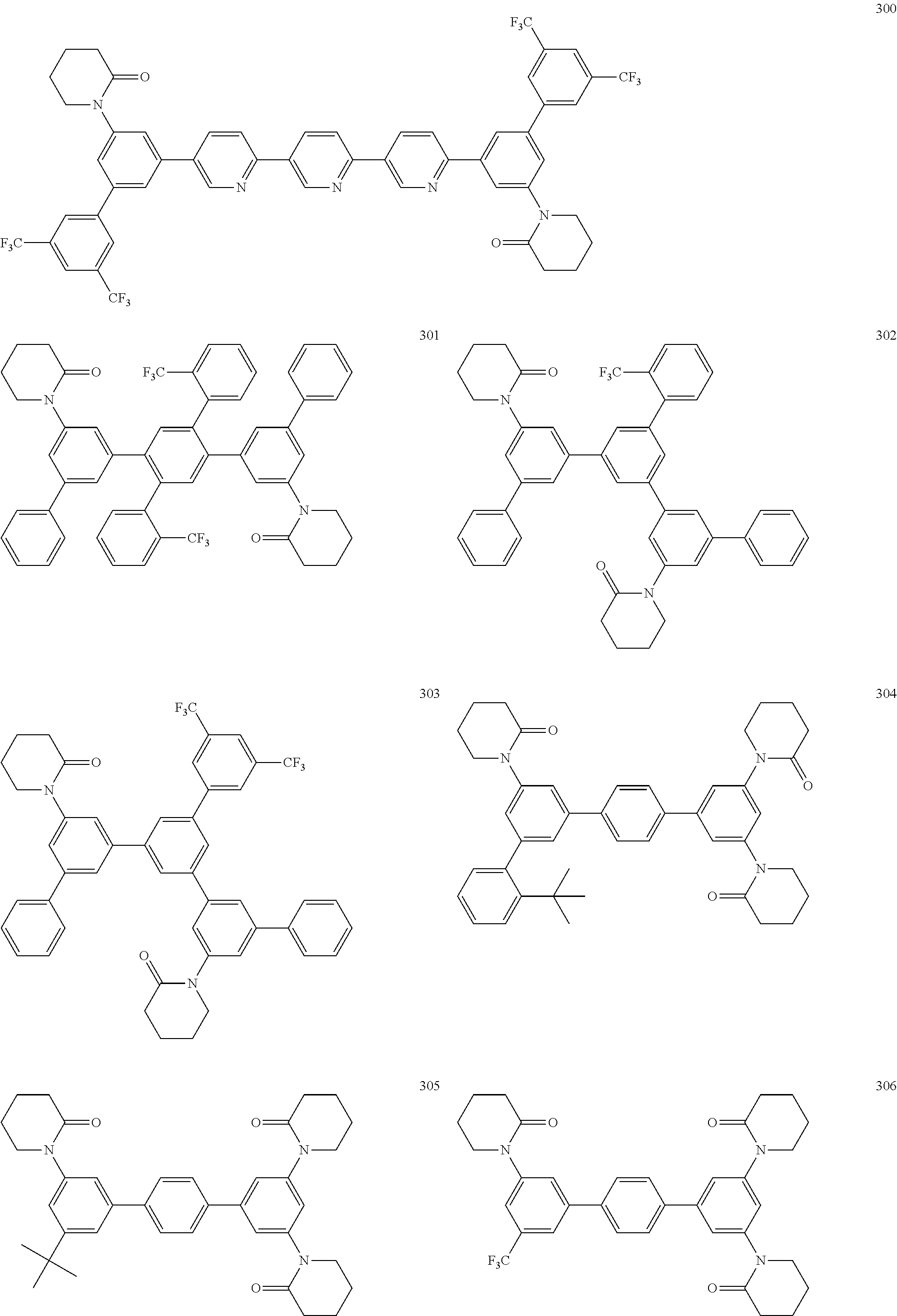

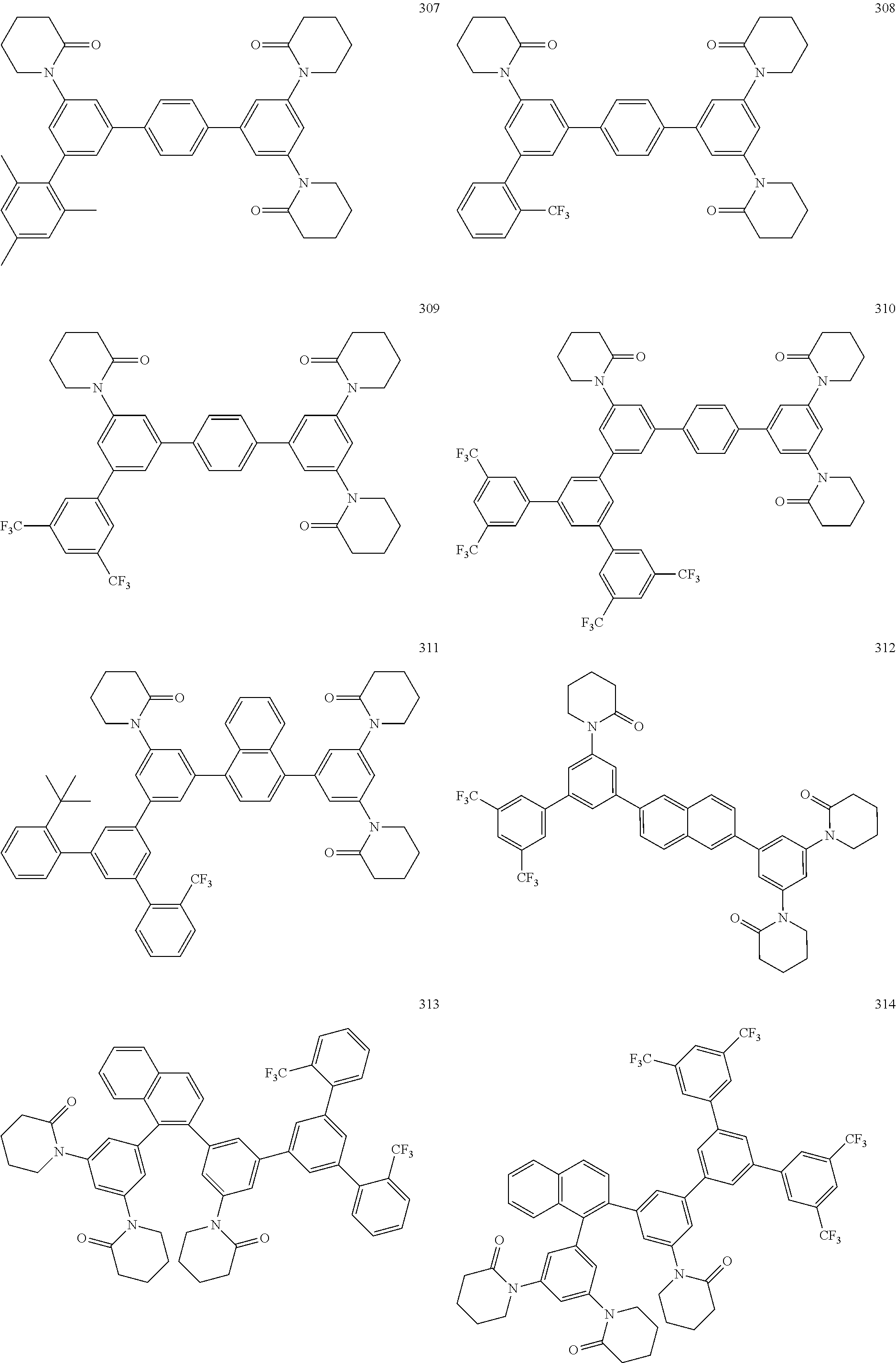

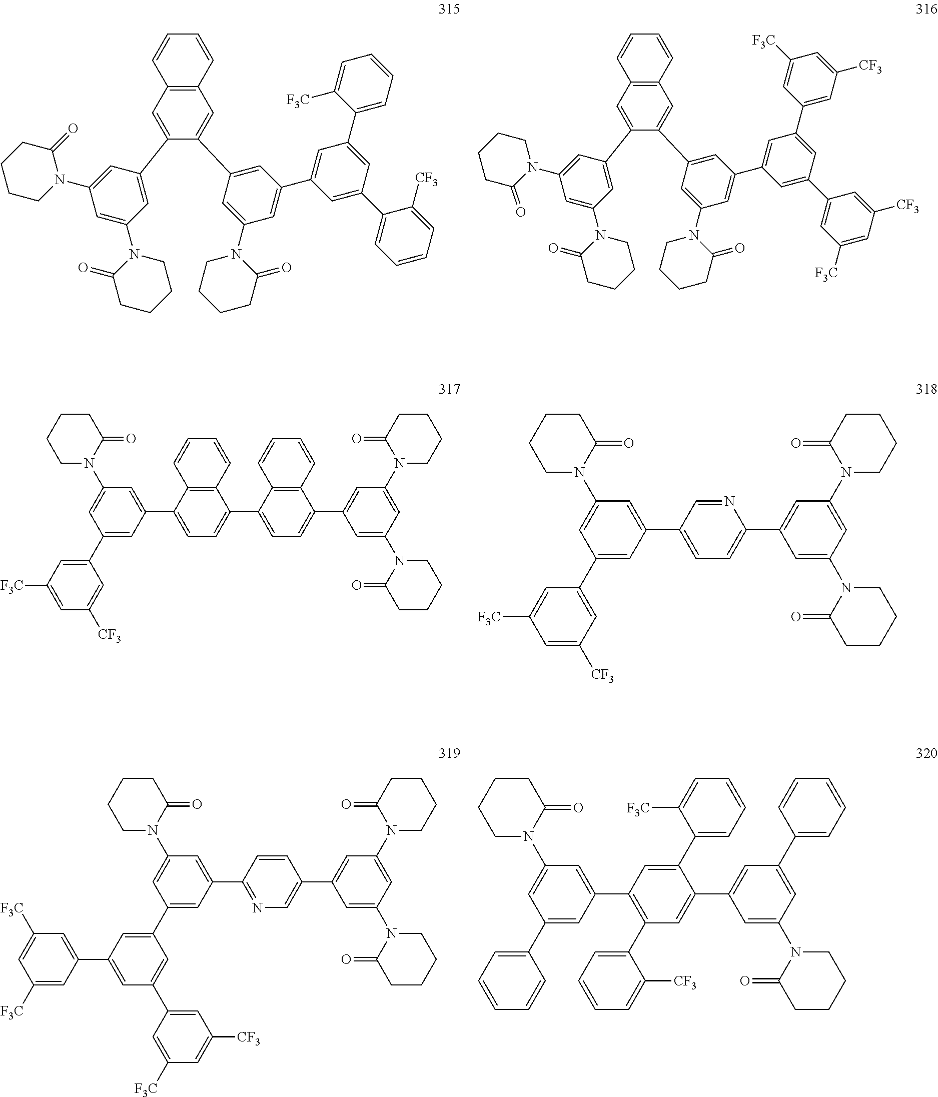

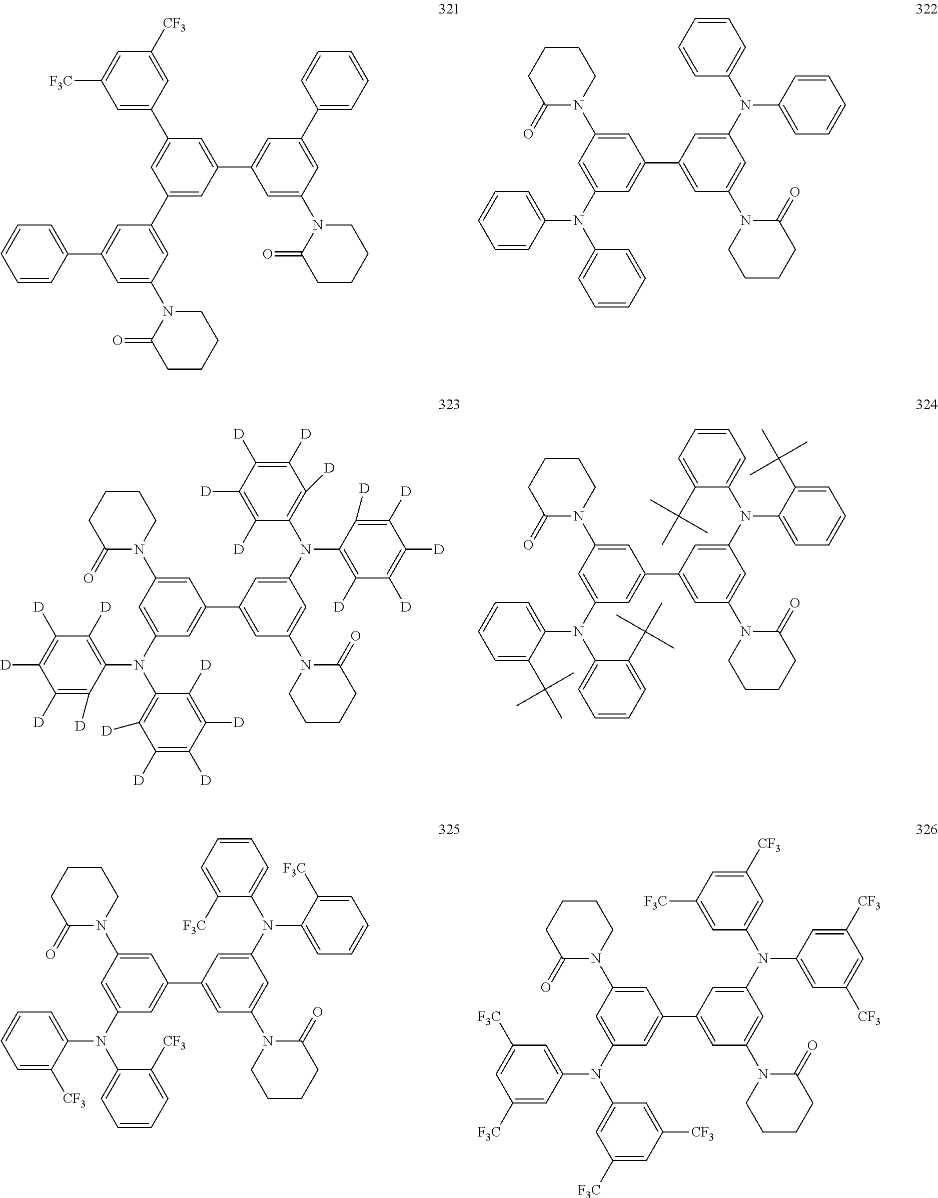

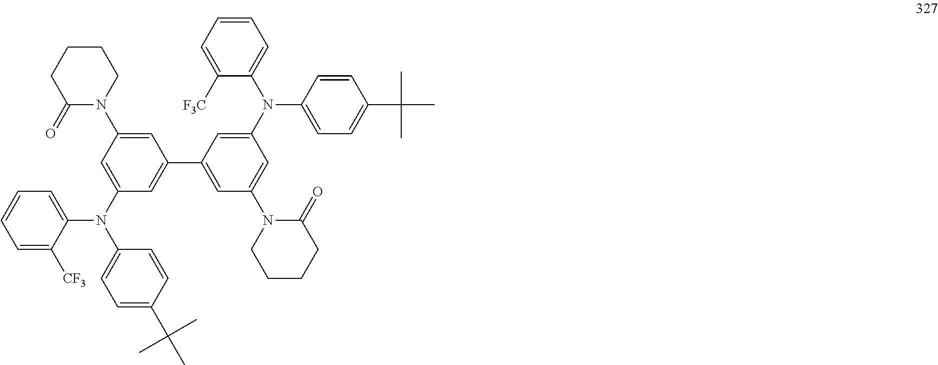 [SMILES: CC(C)(C)c1ccc(N(c2cc(-c3cc(N4CCCCC4=O)cc(N(c4ccc(C(C)(C)C)cc4)c4ccccc4C(F)(F)F)c3)cc(N3CCCCC3=O)c2)c2ccccc2C(F)(F)F)cc1]

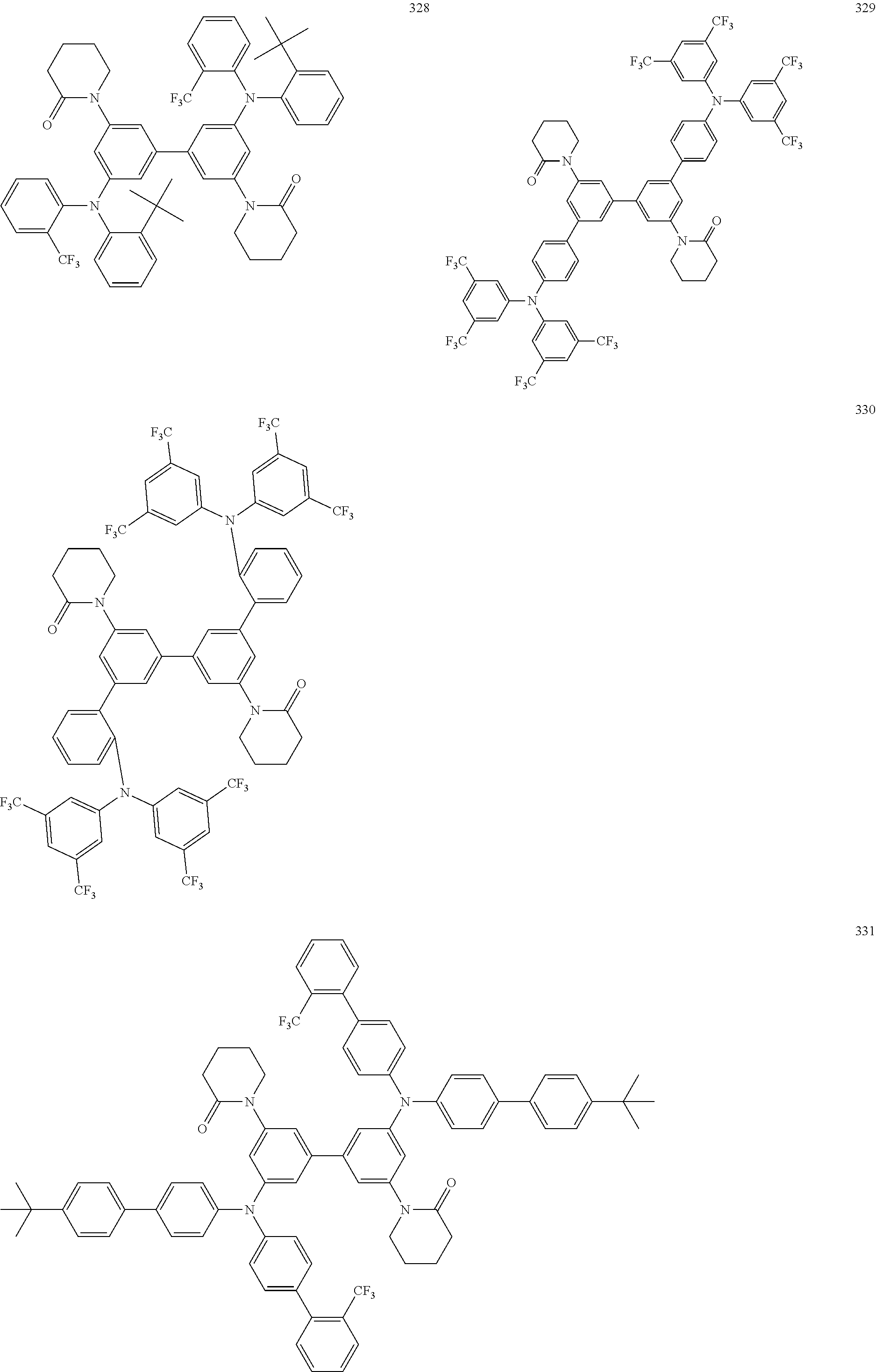

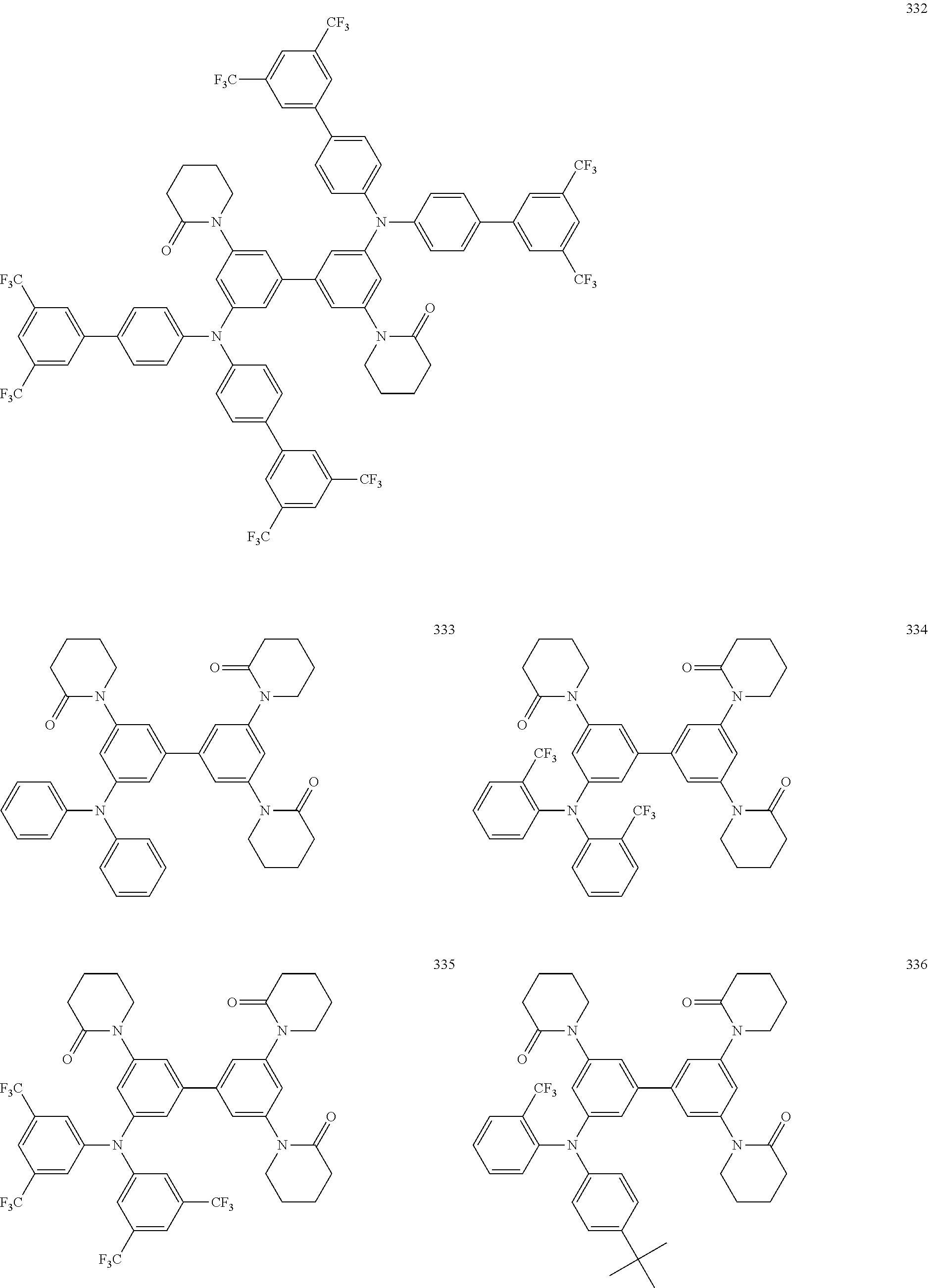

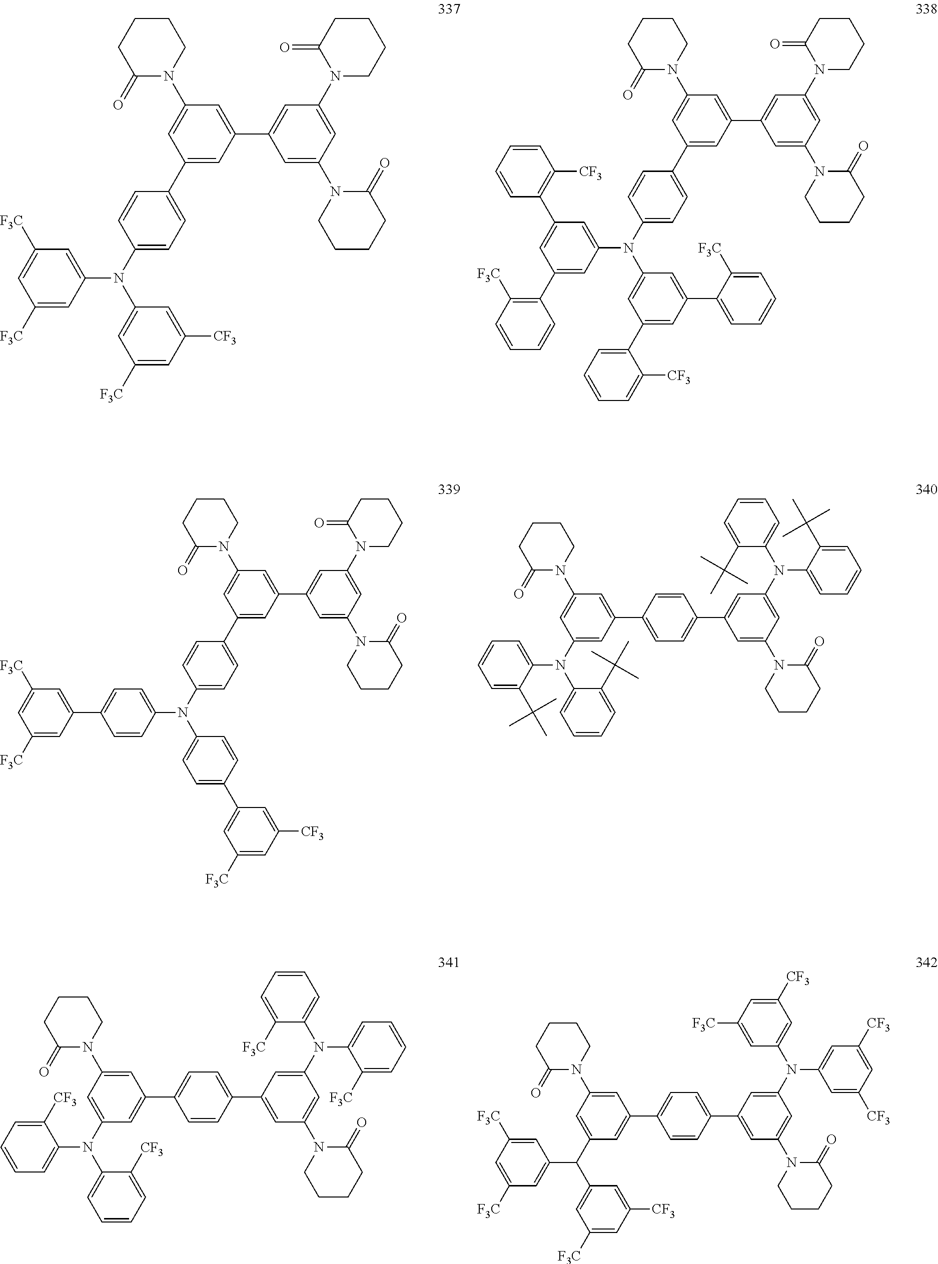

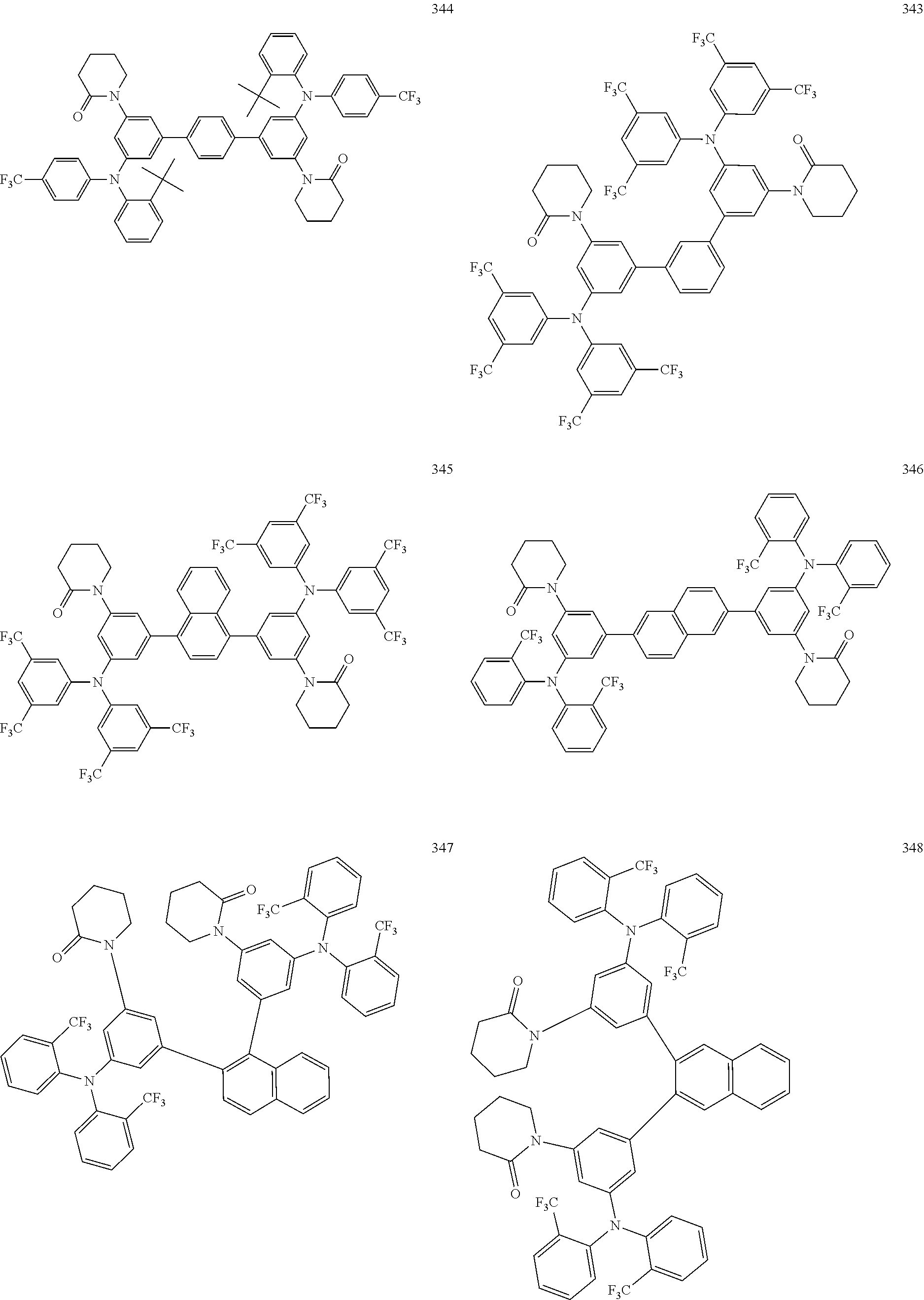

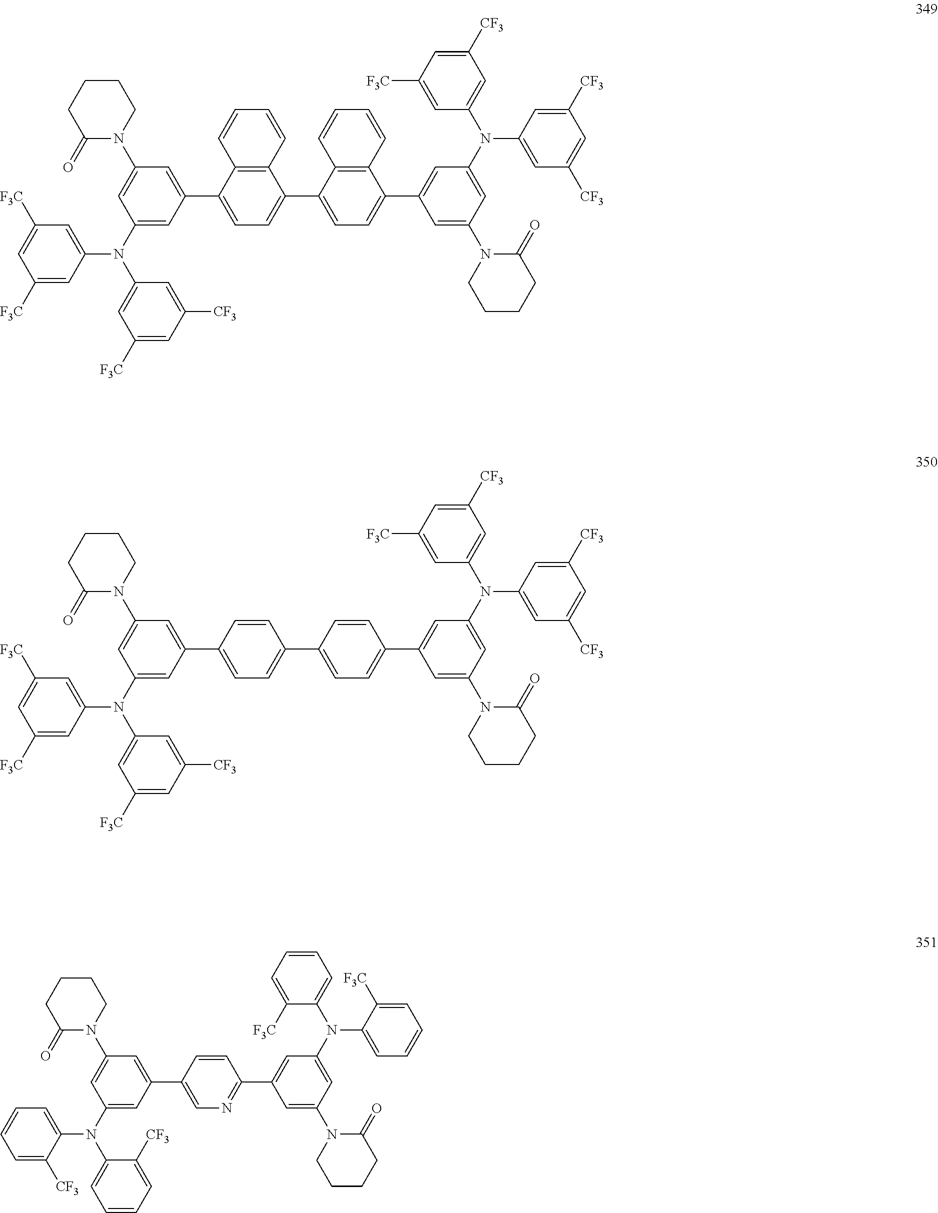

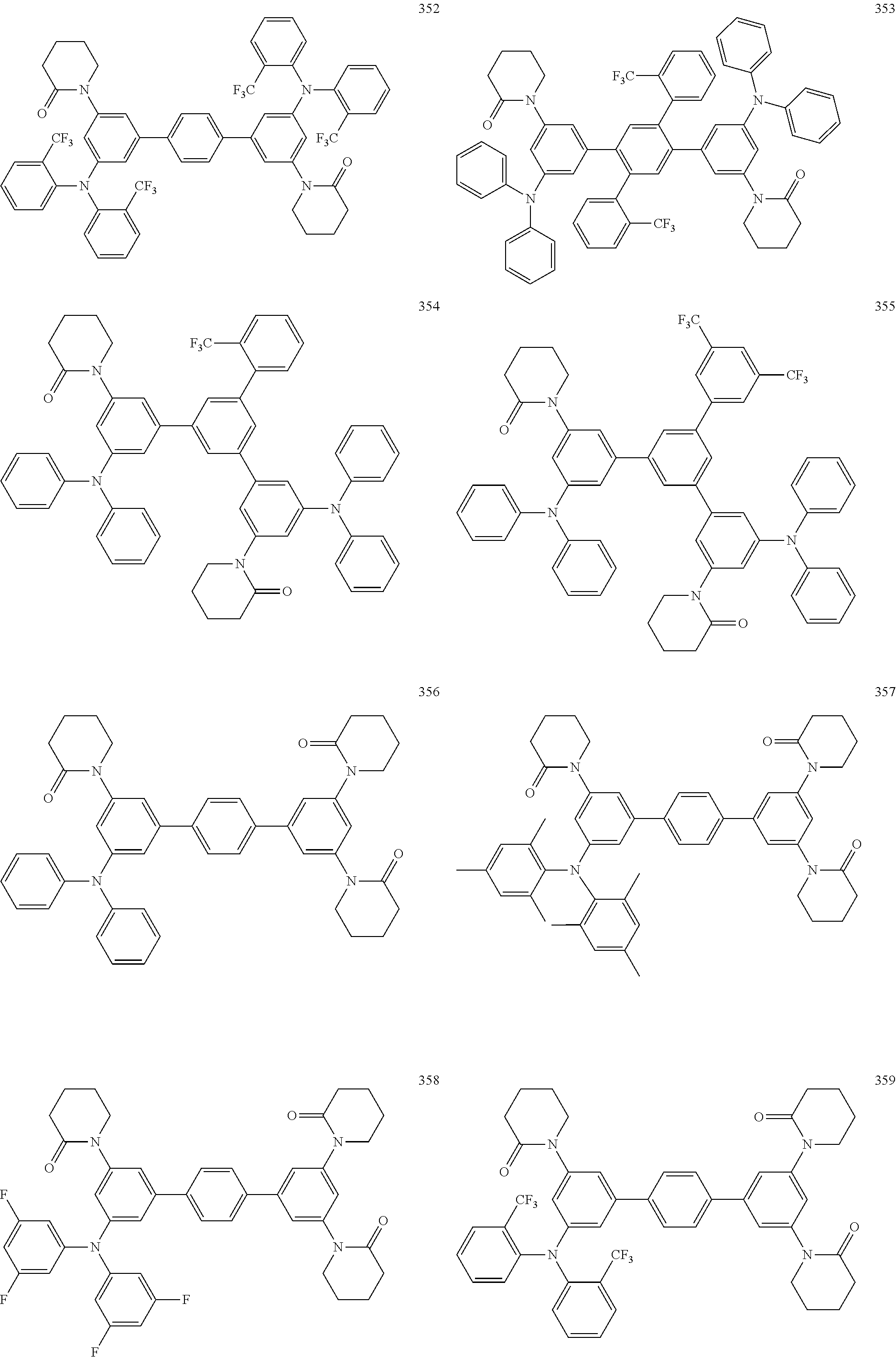

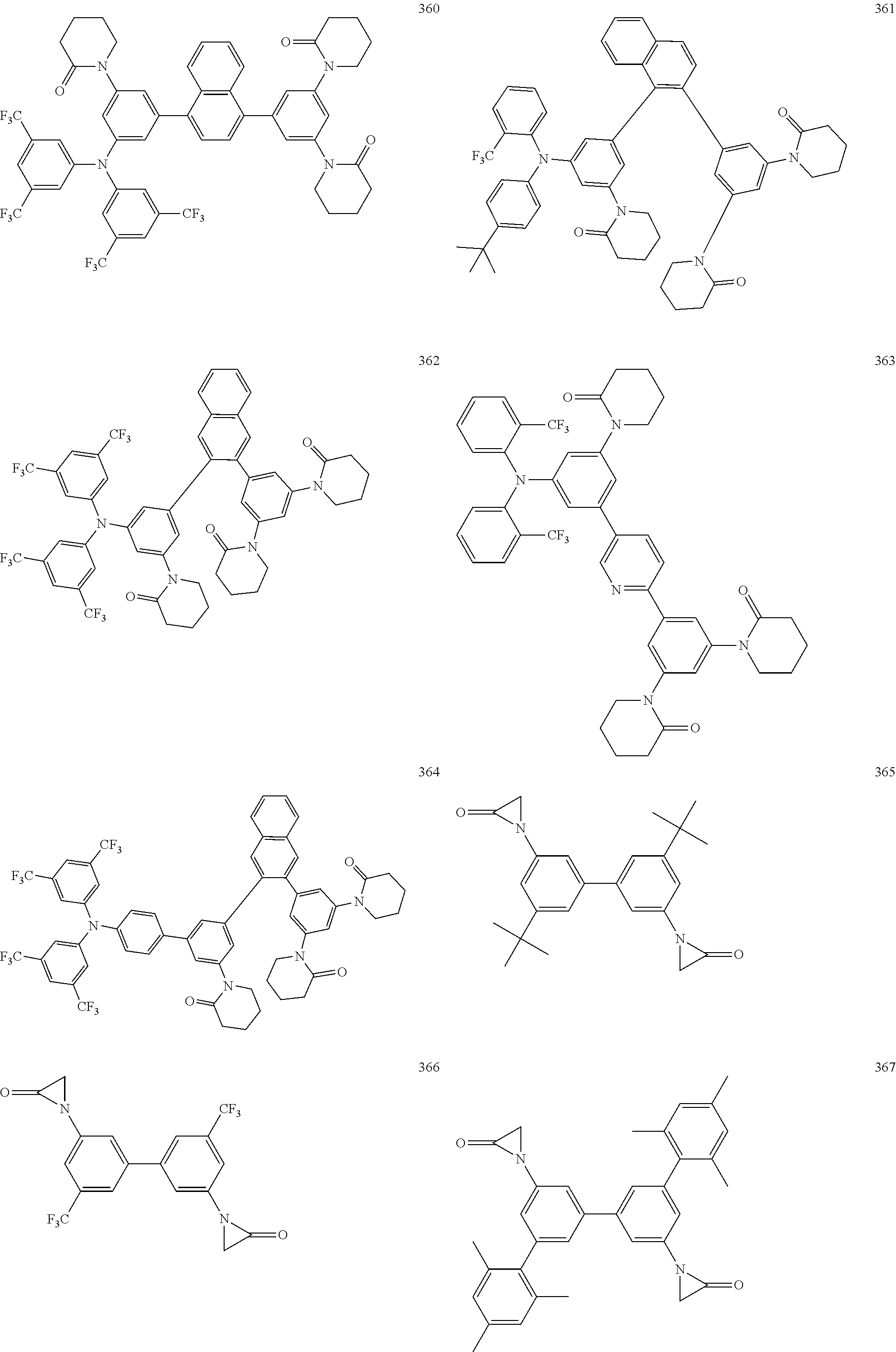

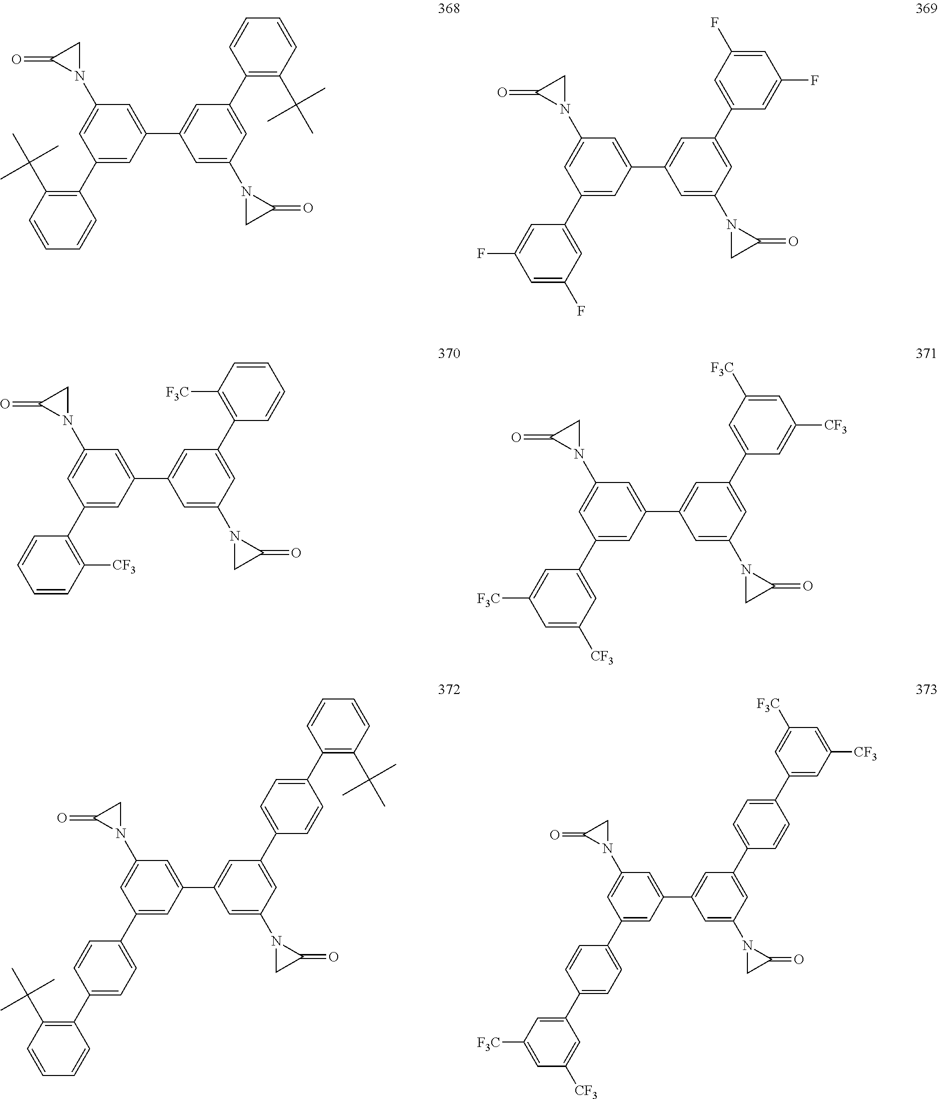

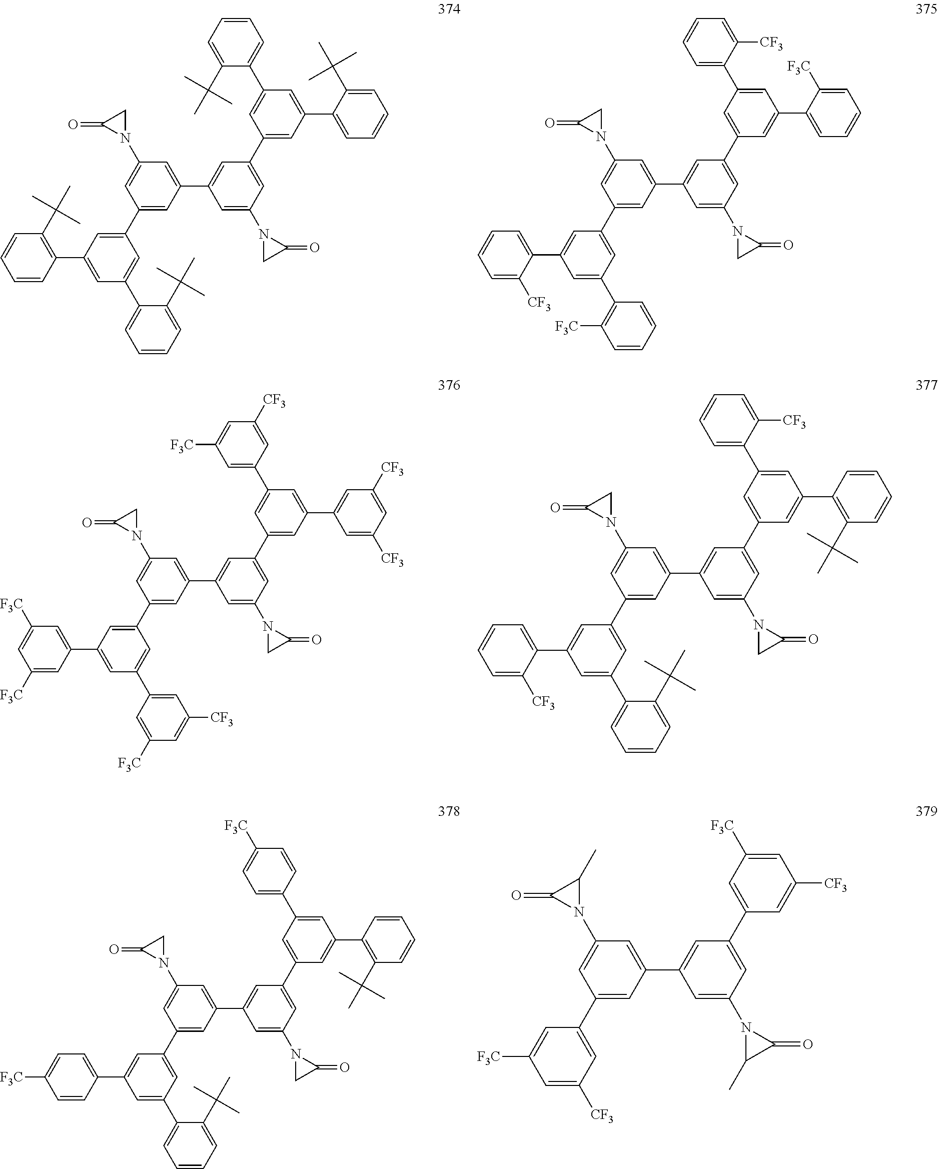

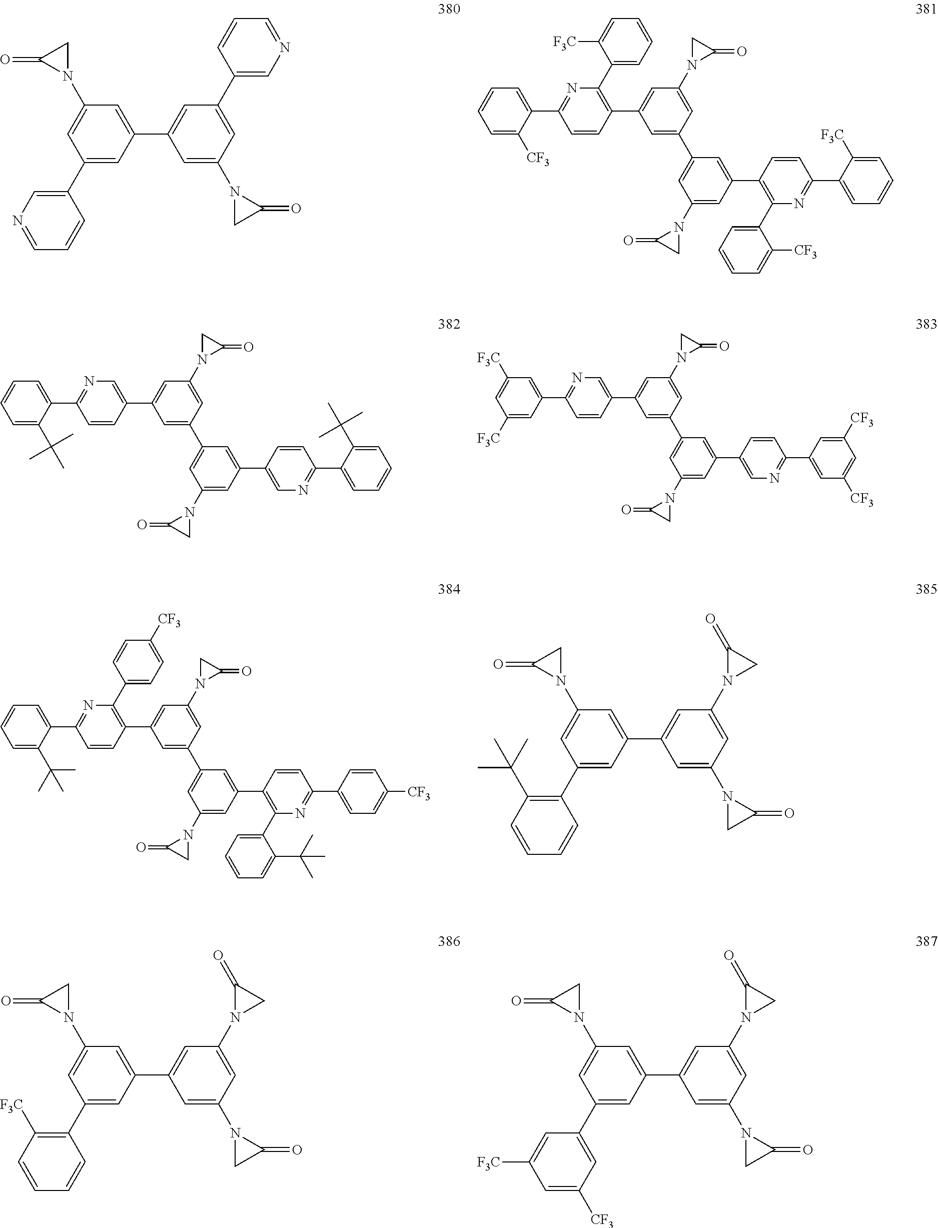

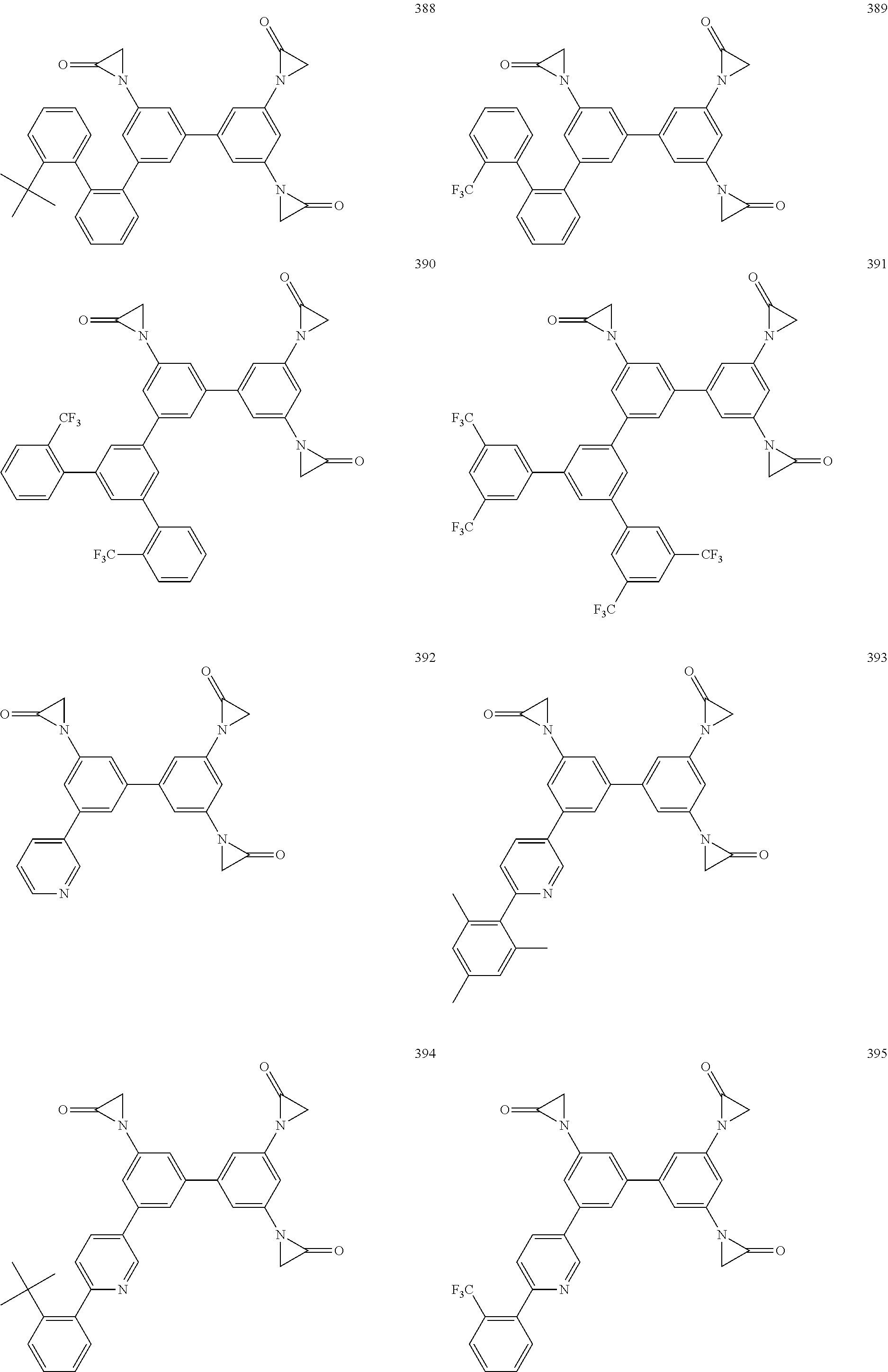

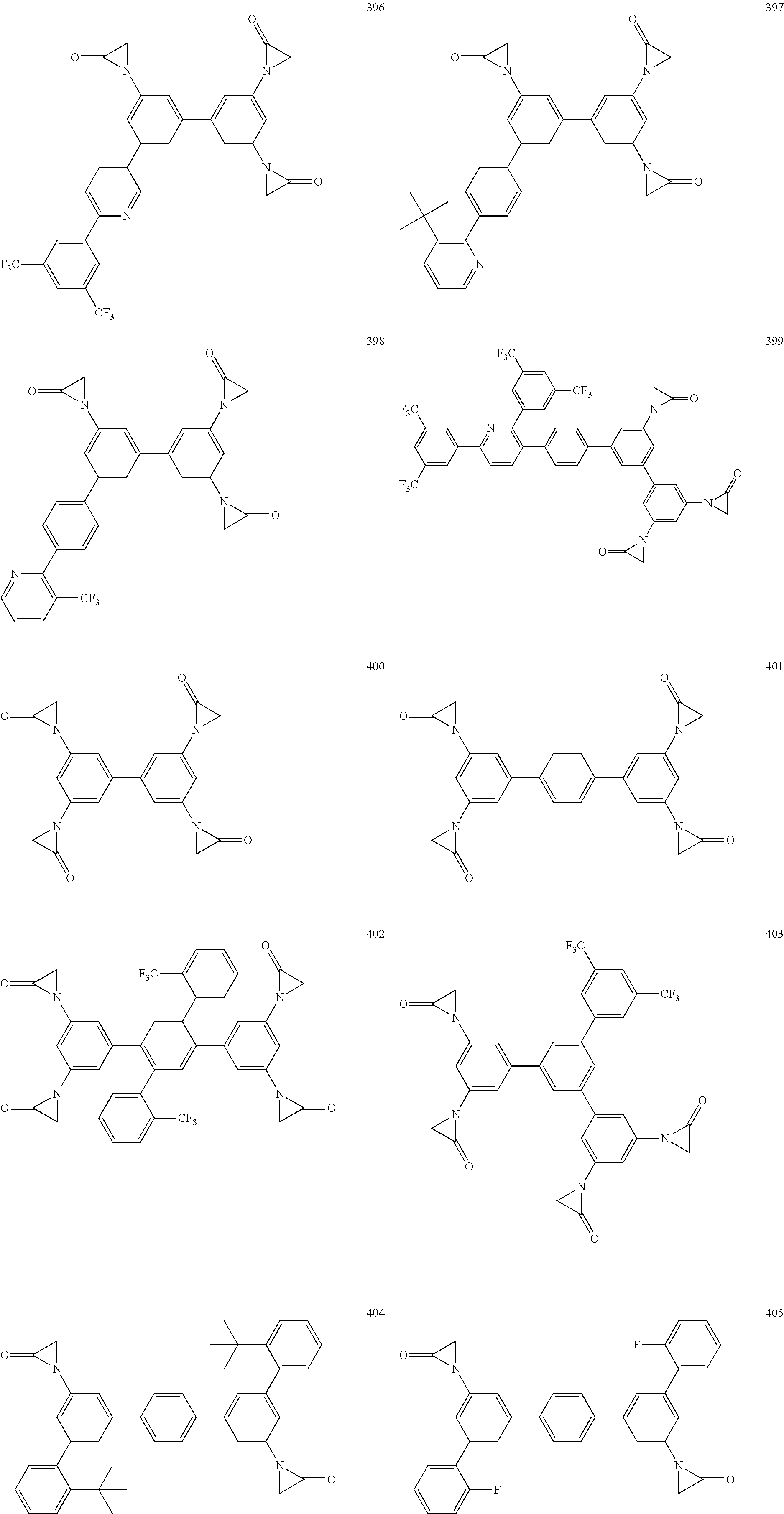

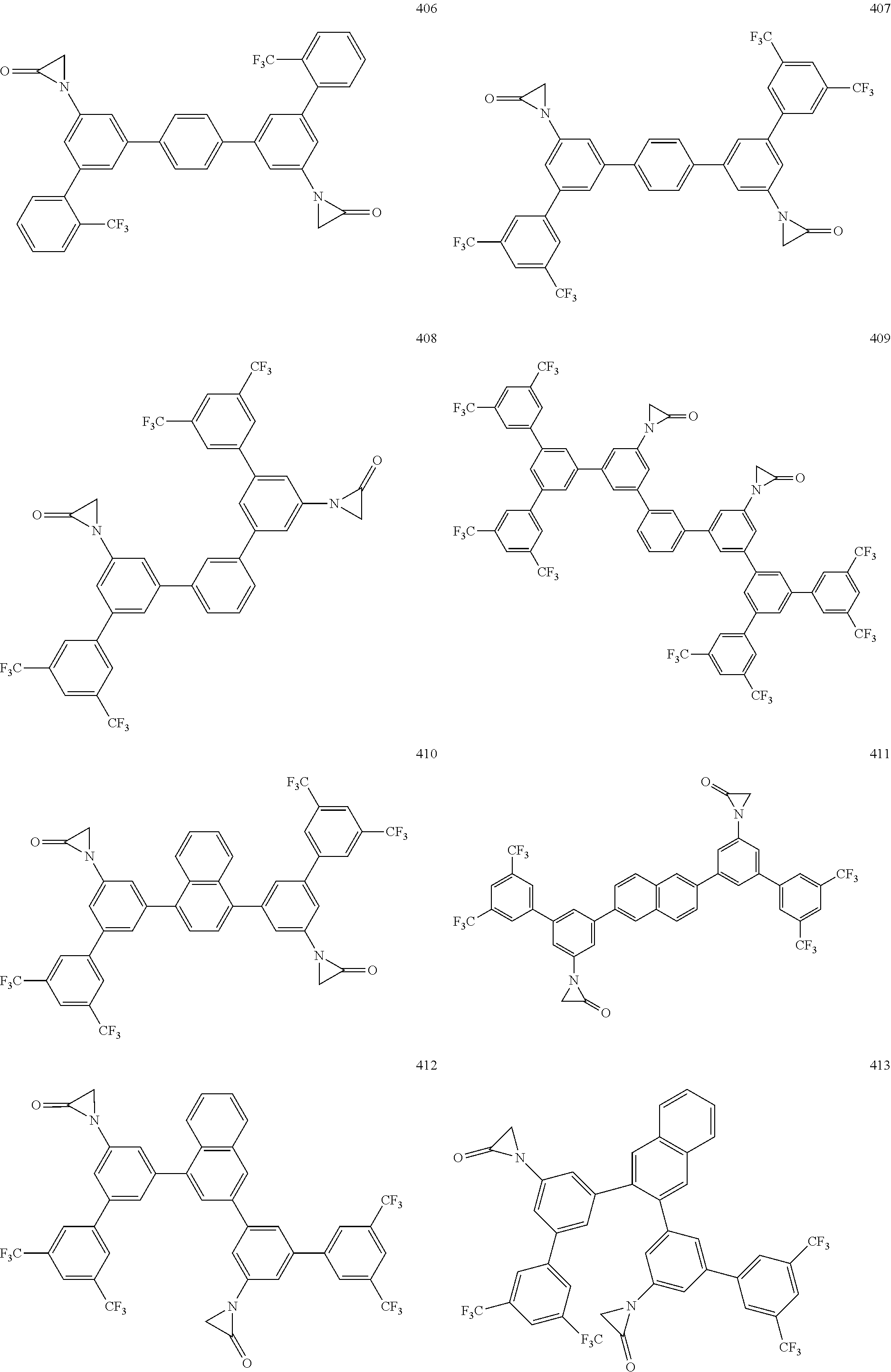

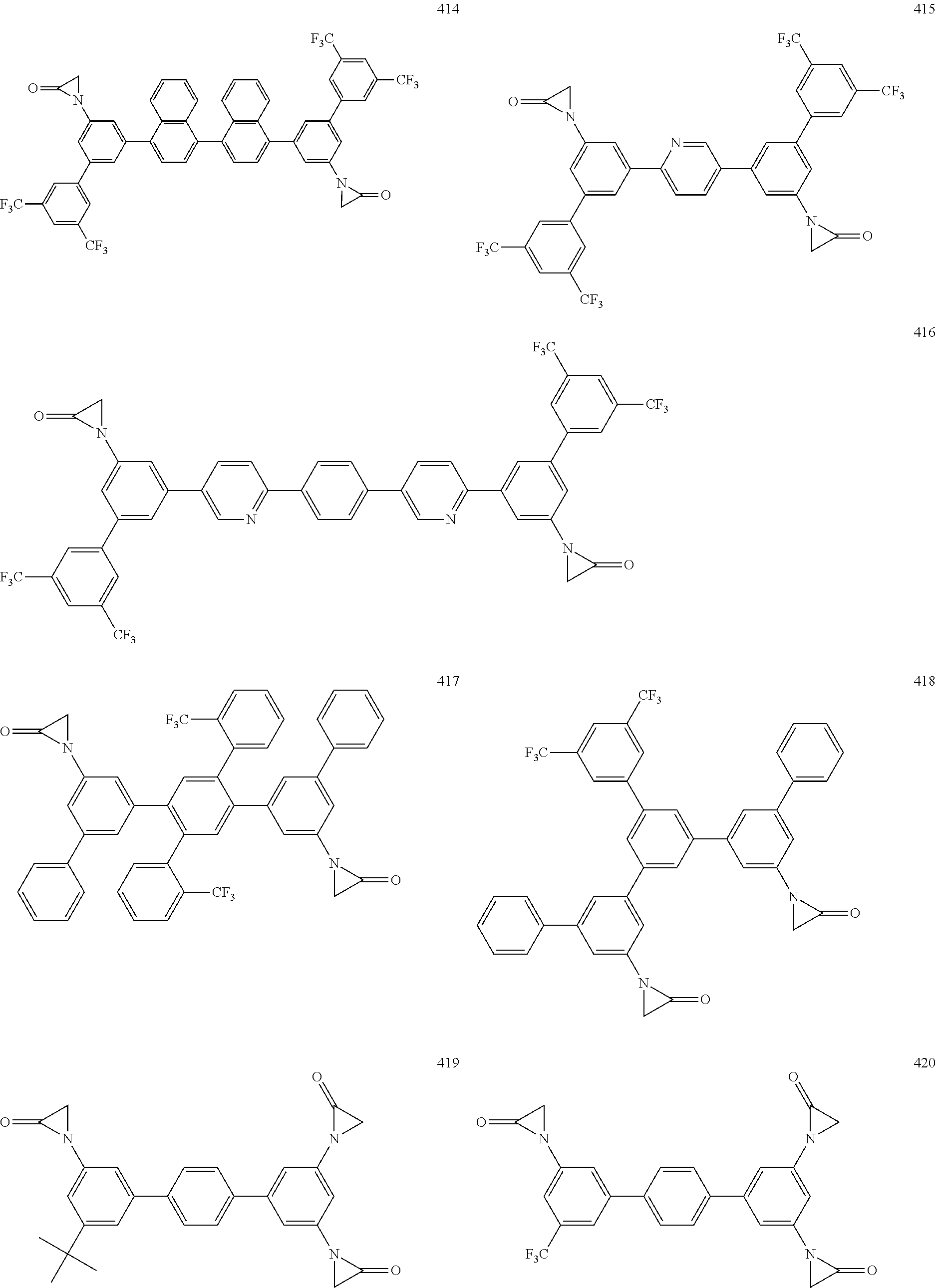

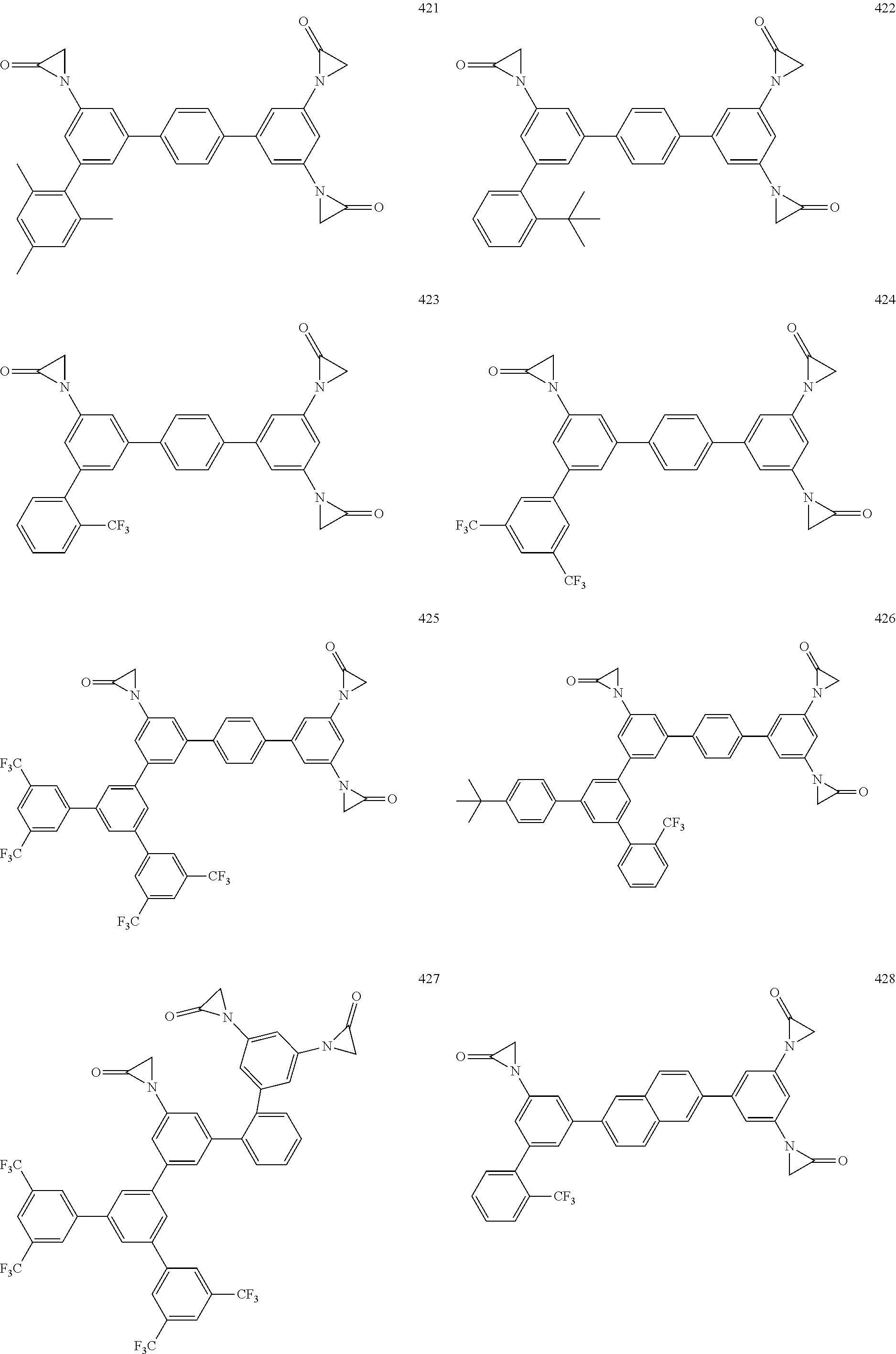

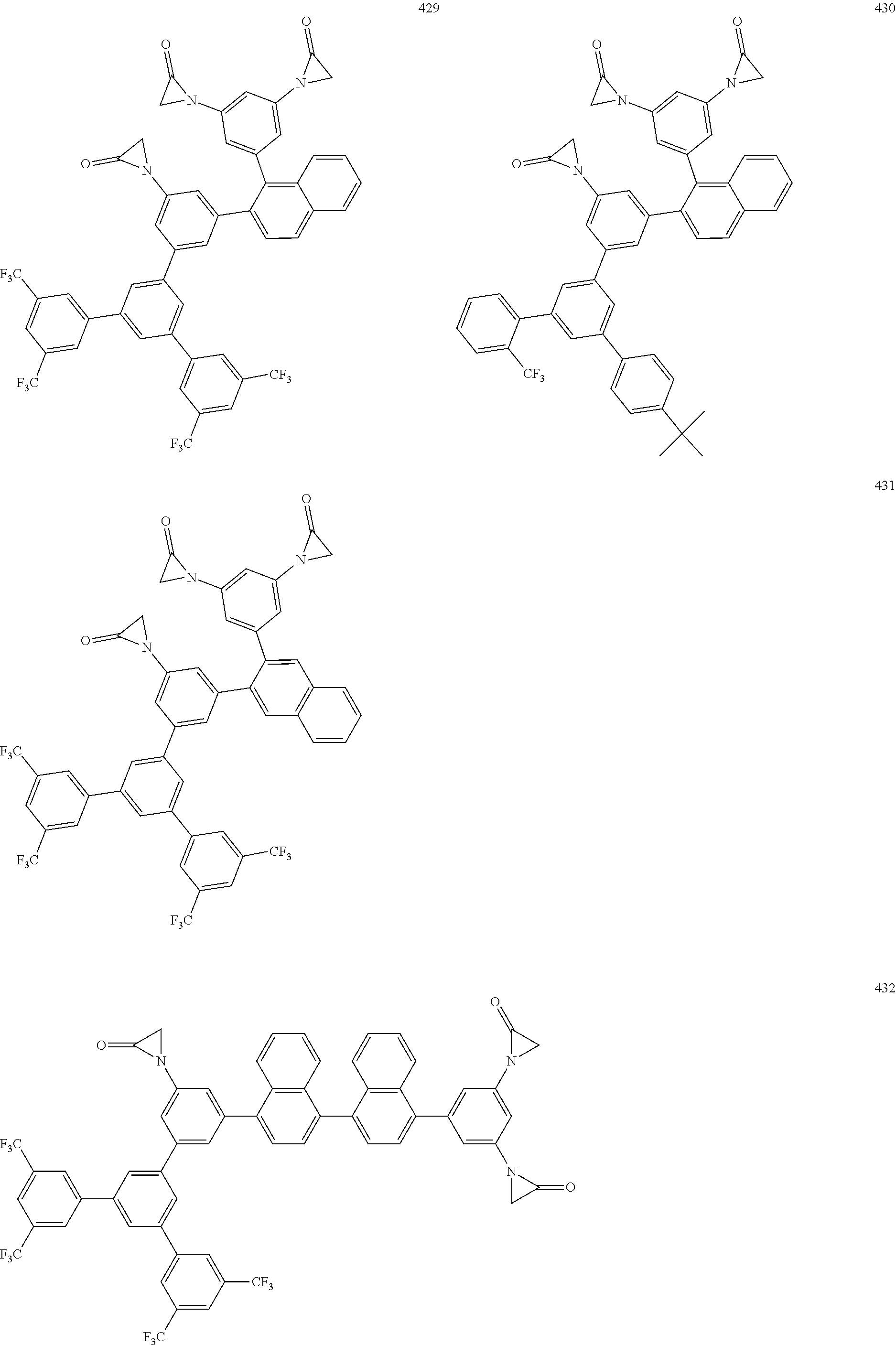

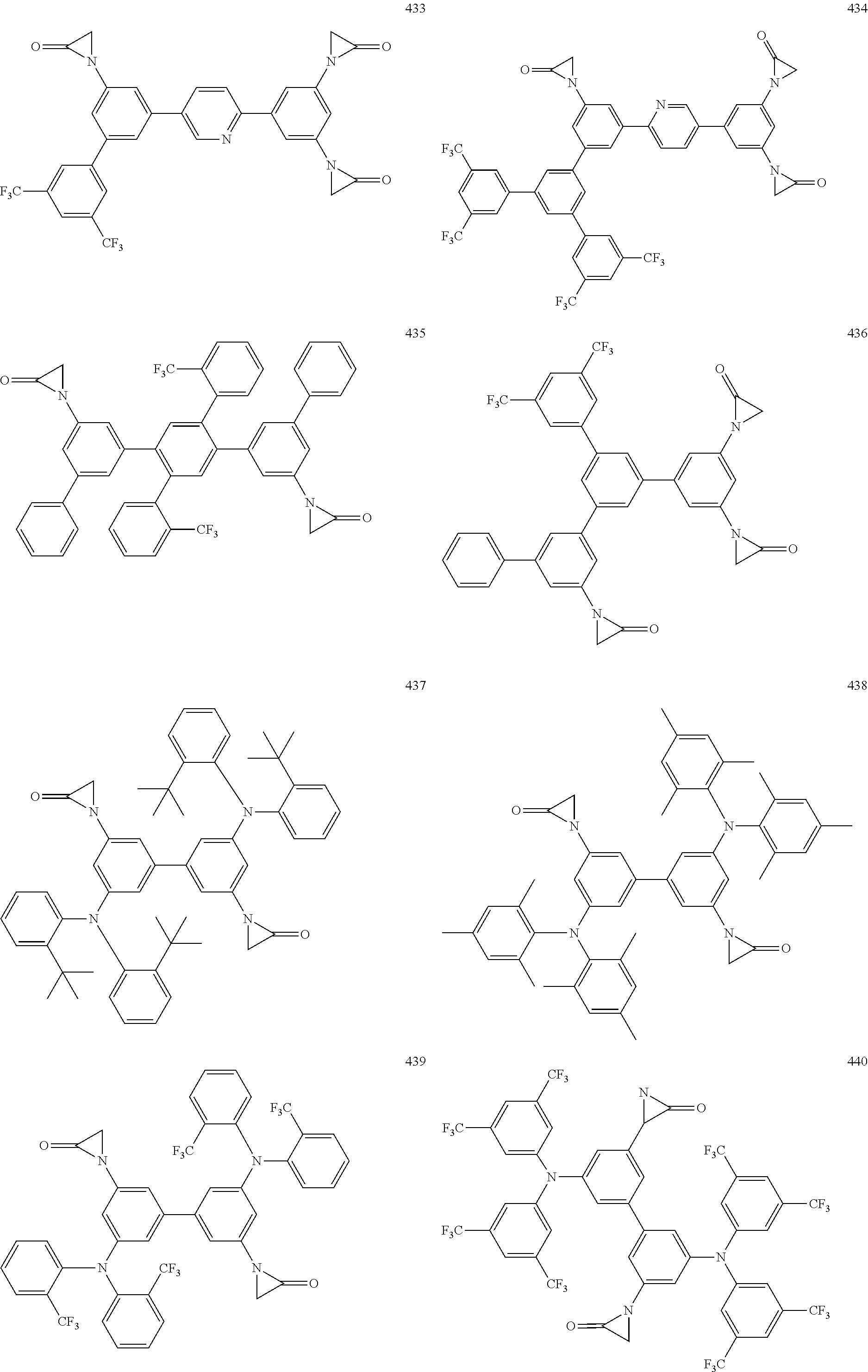

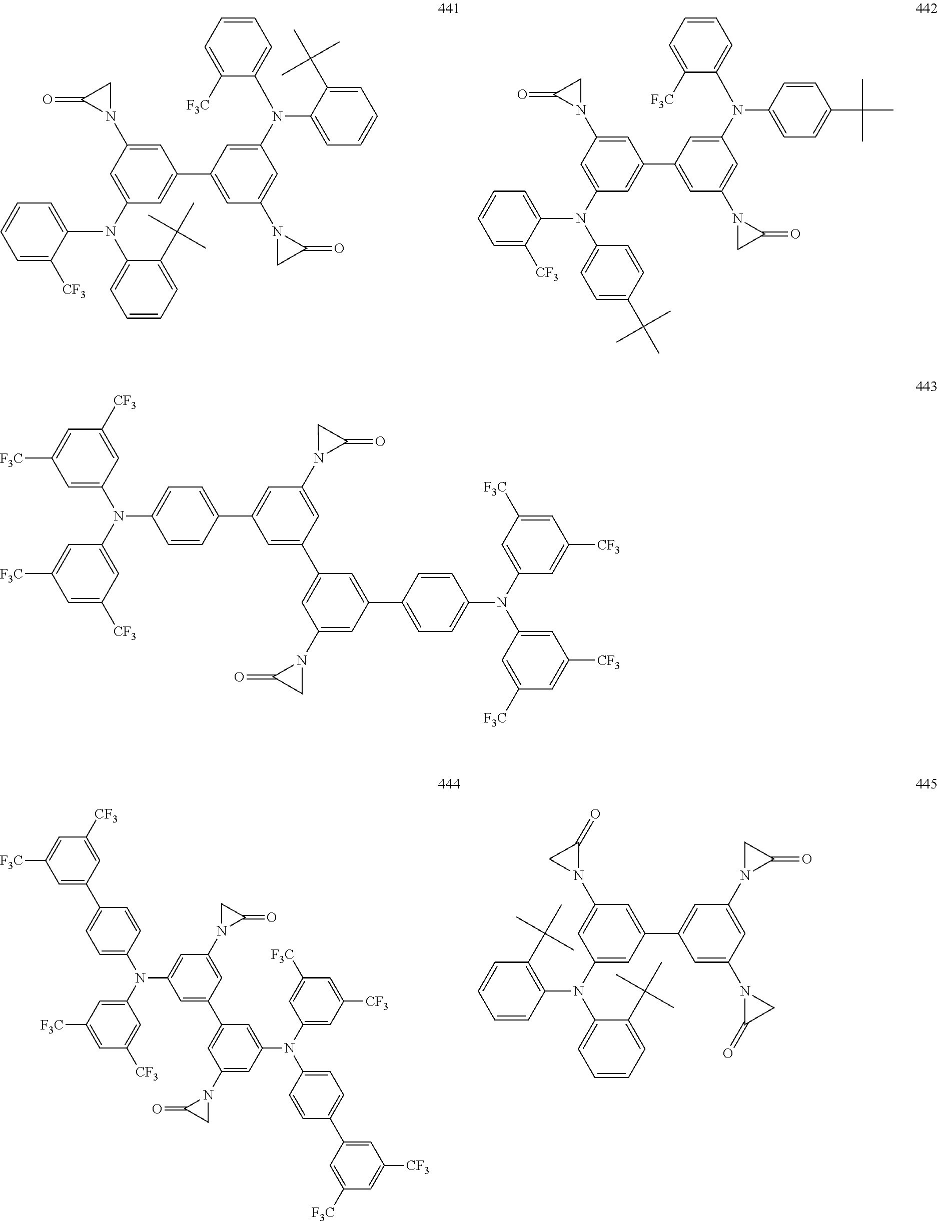

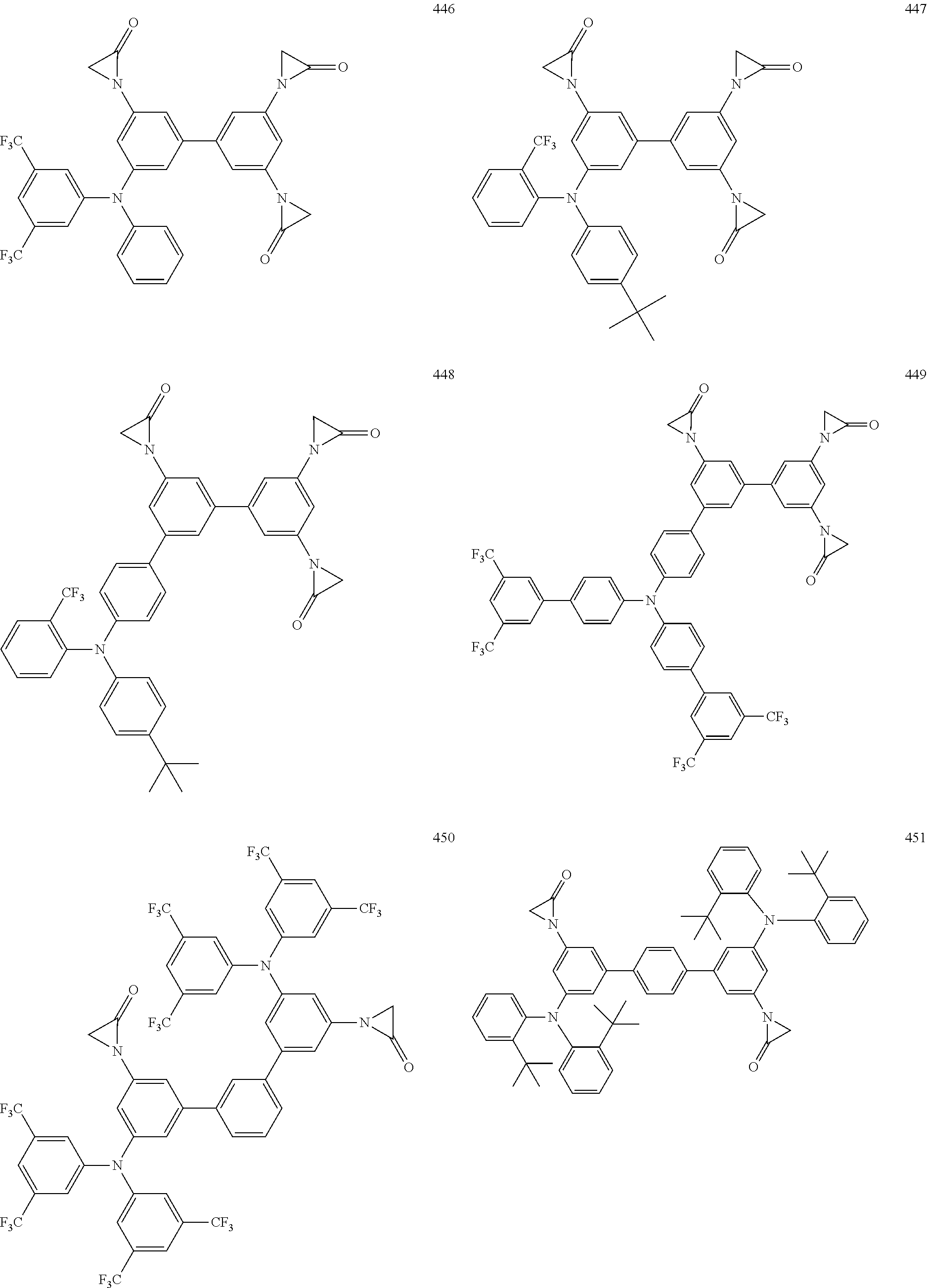

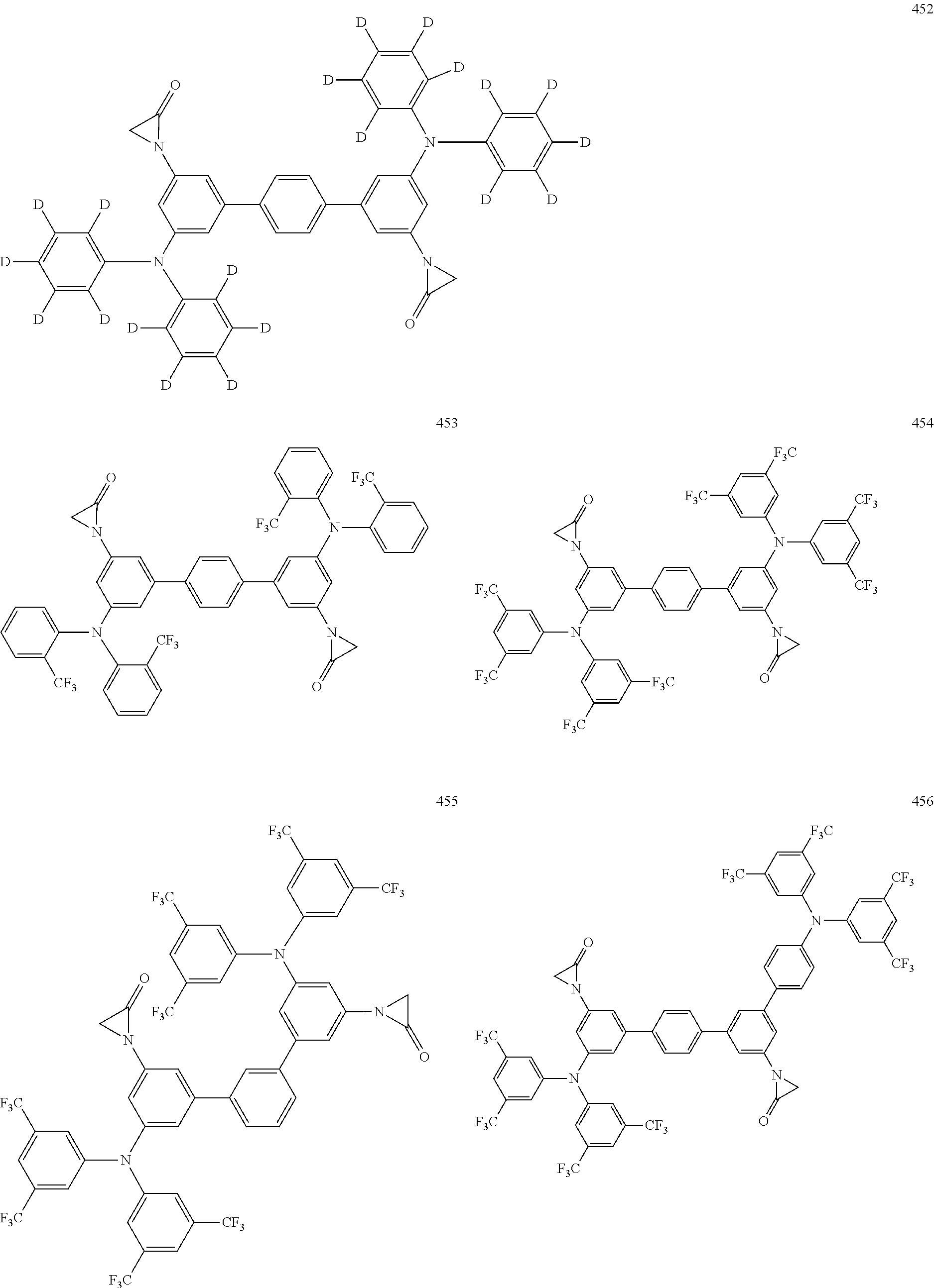

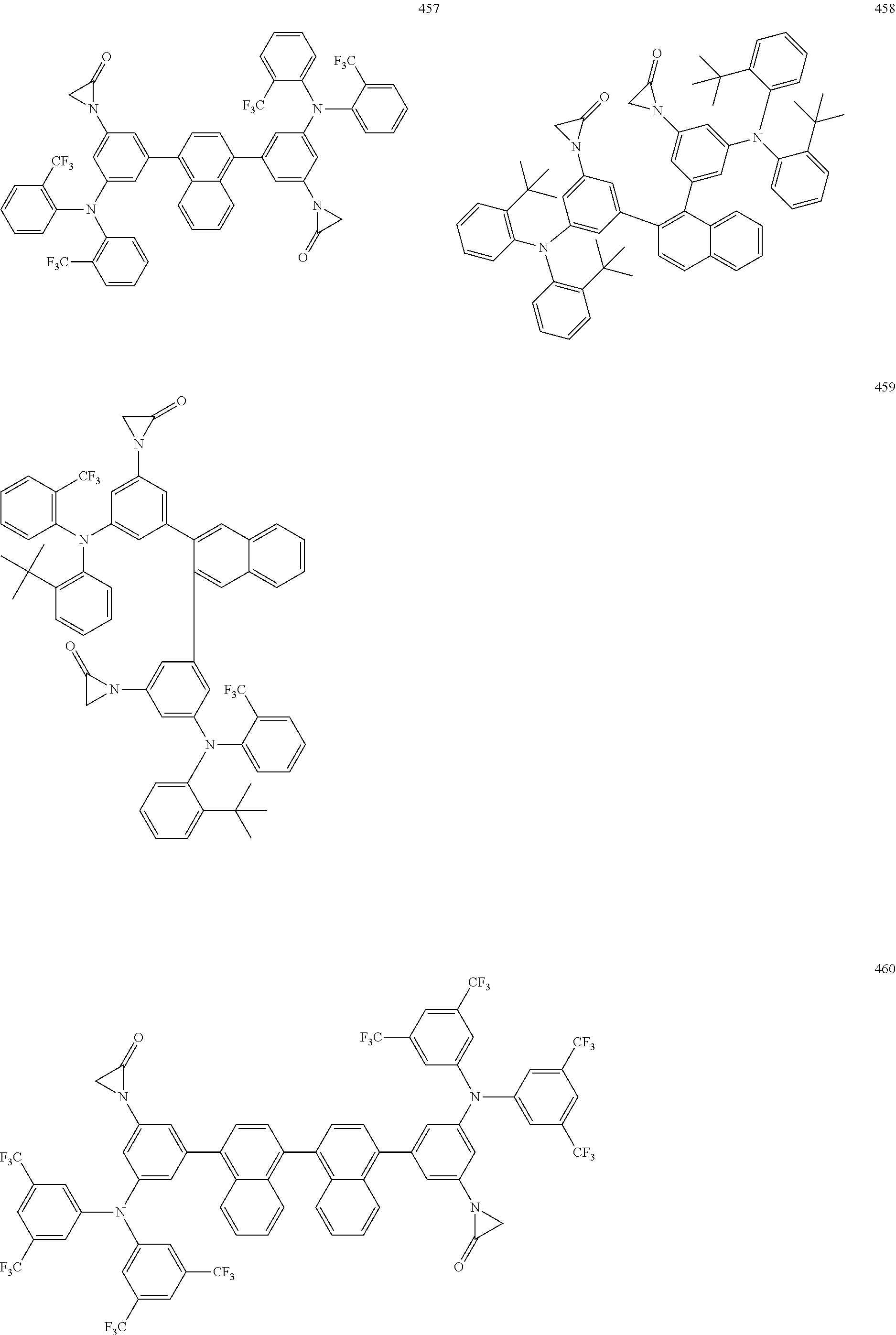

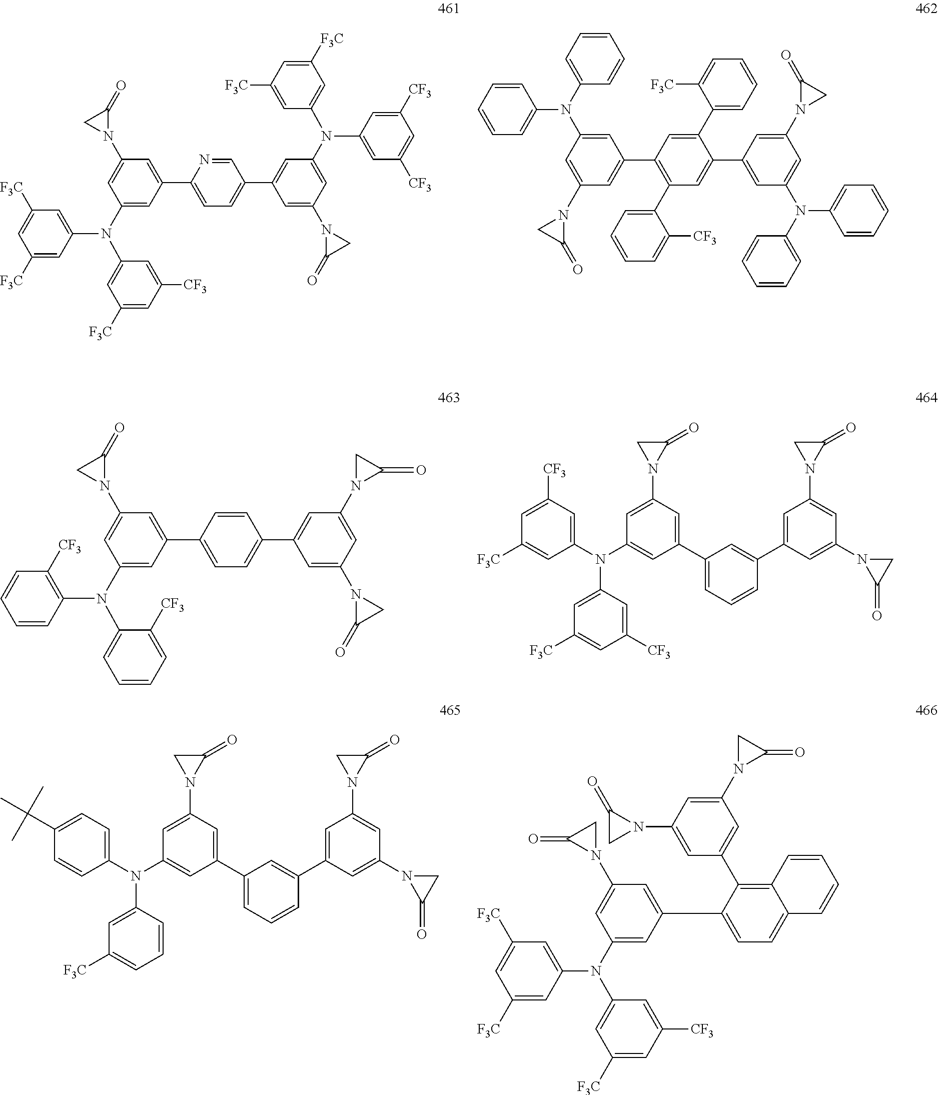

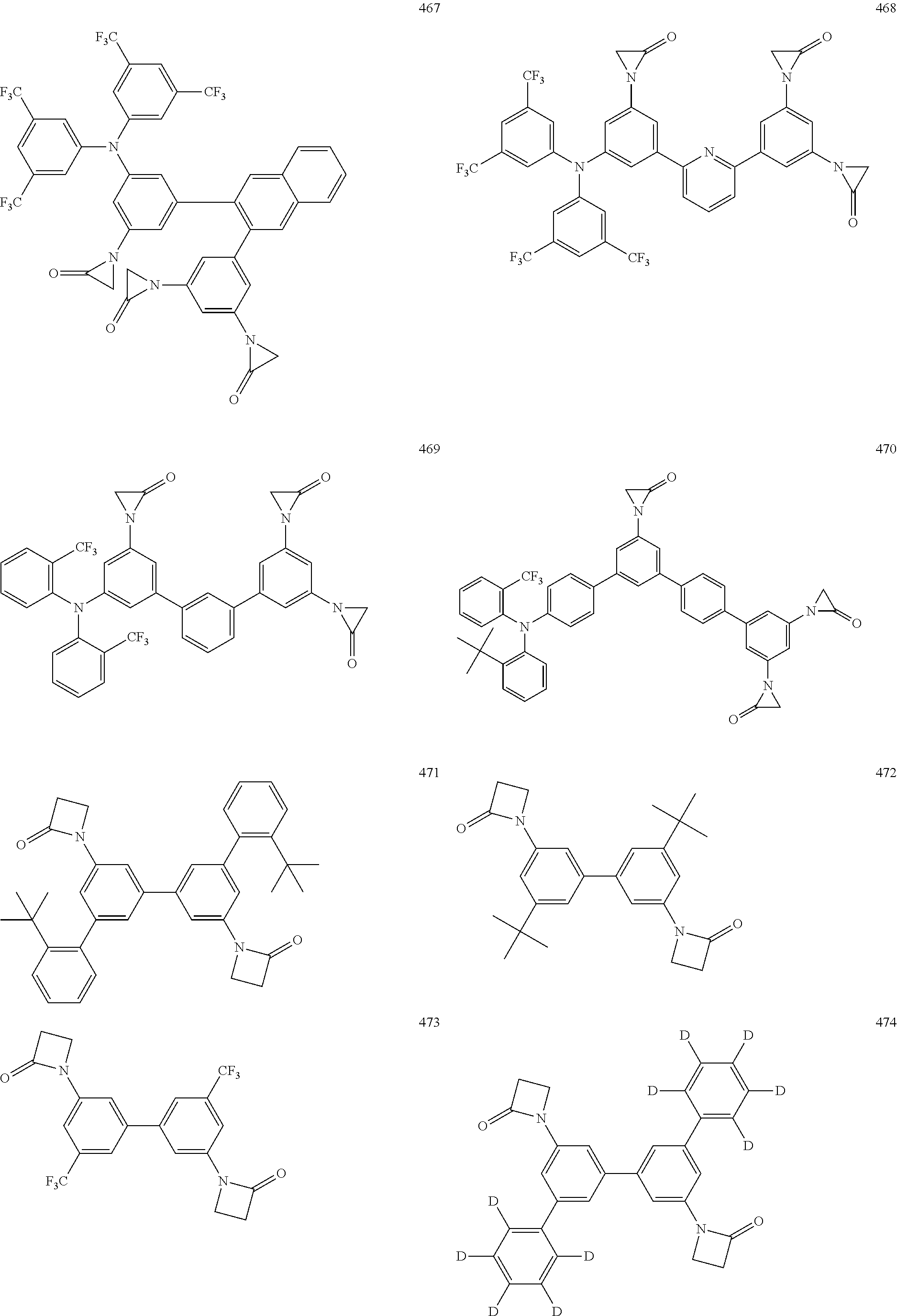

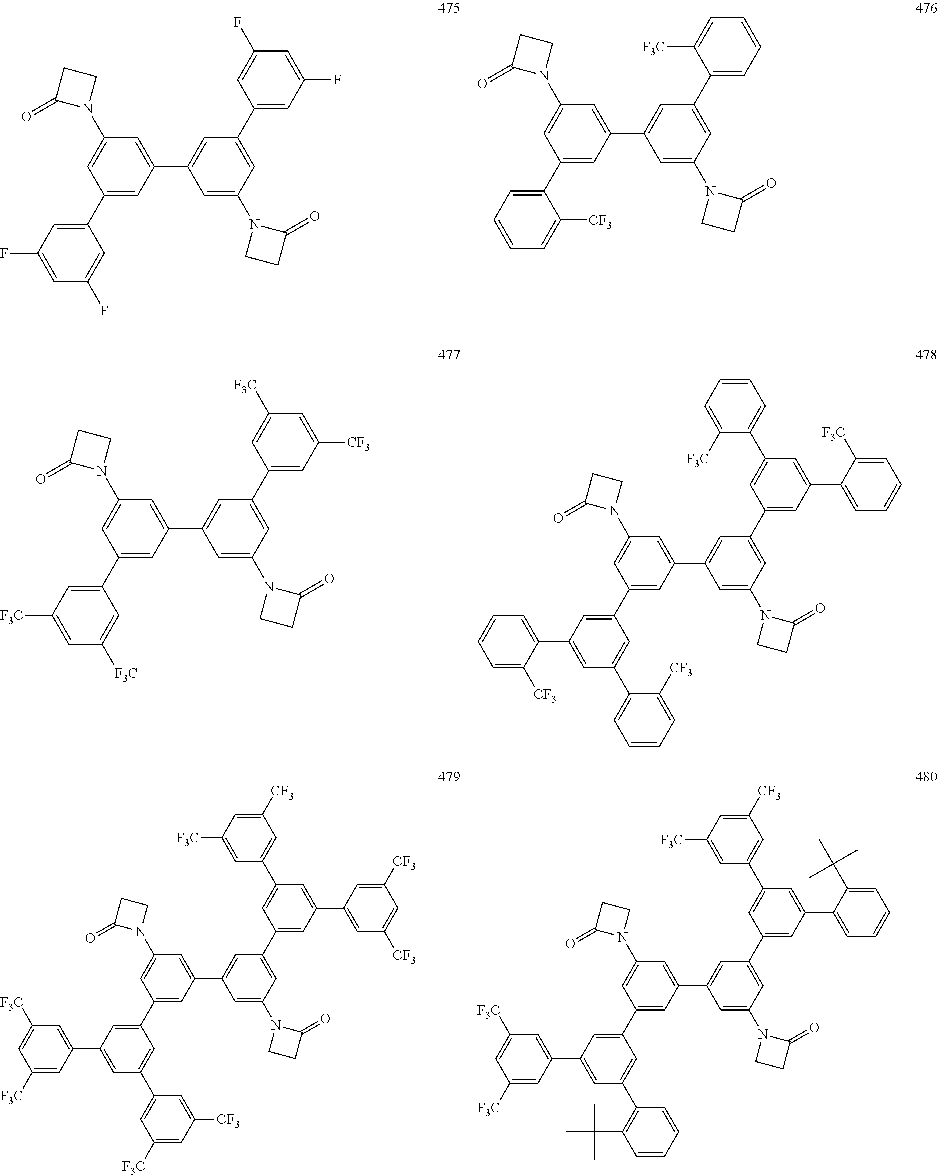

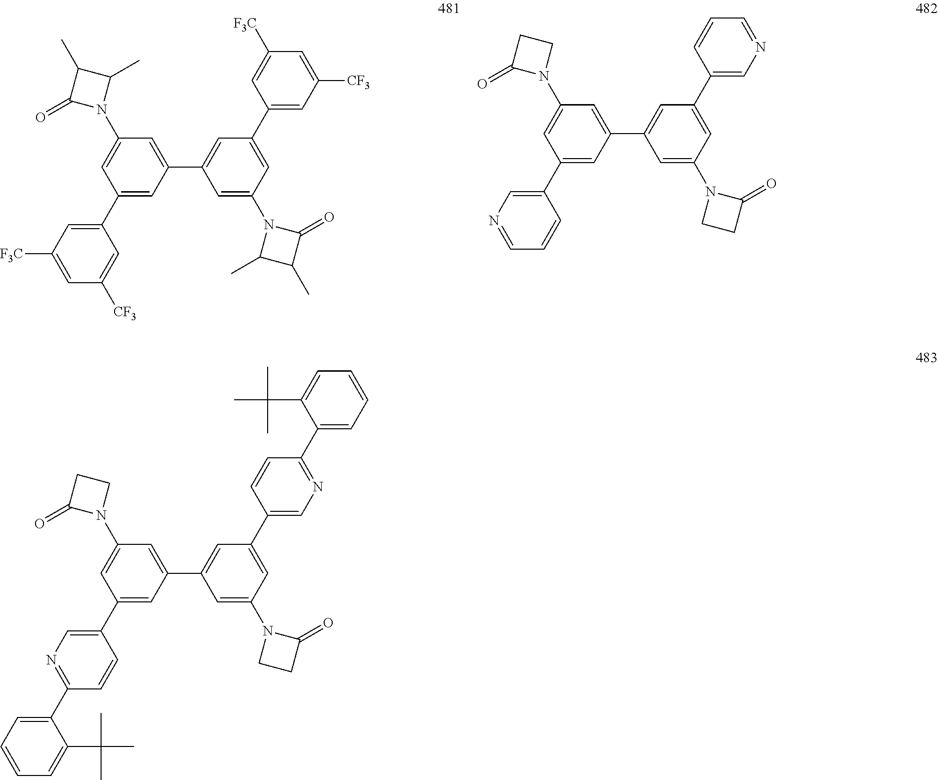 [SMILES: CC1C(=O)N(c2cc(-c3cc(-c4cc(C(F)(F)F)cc(C(F)(F)F)c4)cc(N4C(=O)C(C)C4C)c3)cc(-c3cc(C(F)(F)F)cc(C(F)(F)F)c3)c2)C1C ; O=C1CCN1c1cc(-c2cccnc2)cc(-c2cc(-c3cccnc3)cc(N3CCC3=O)c2)c1 ; CC(C)(C)c1ccccc1-c1ccc(-c2cc(-c3cc(-c4ccc(-c5ccccc5C(C)(C)C)nc4)cc(N4CCC4=O)c3)cc(N3CCC3=O)c2)cn1]

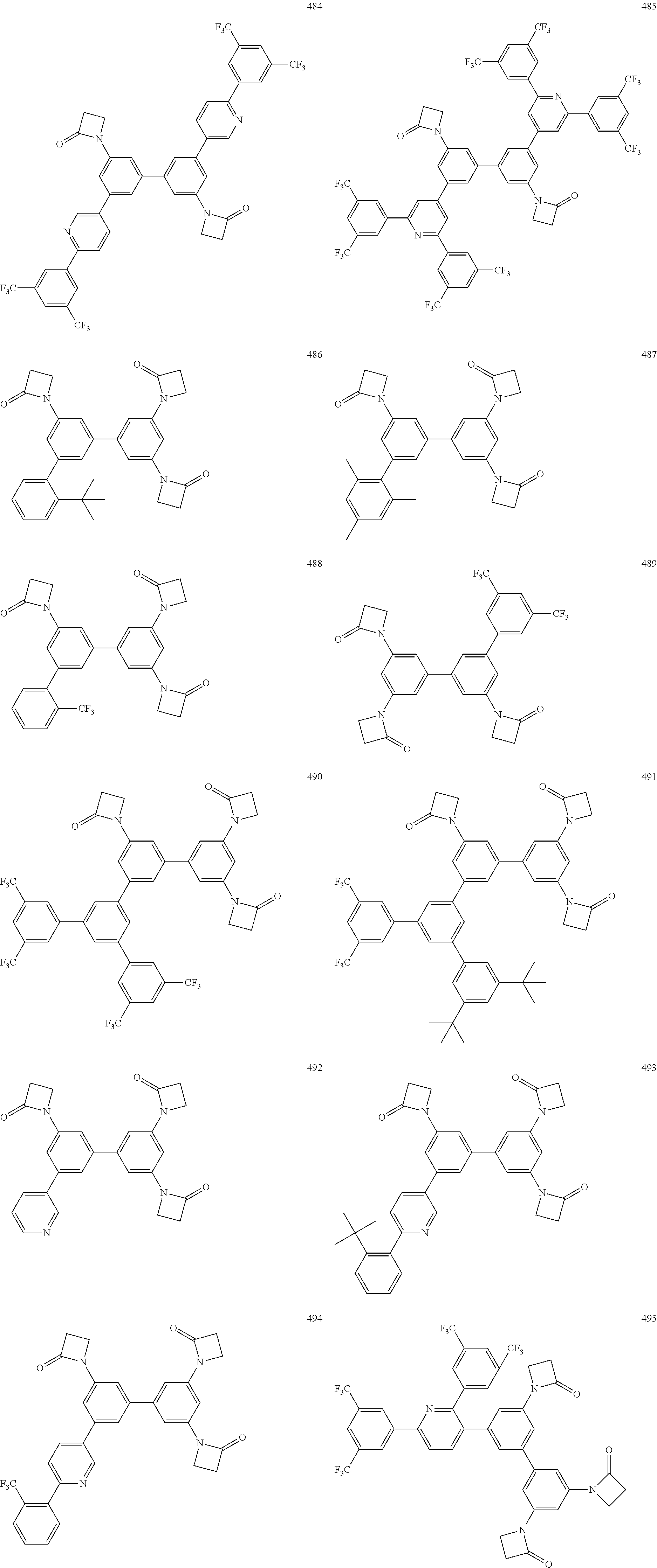

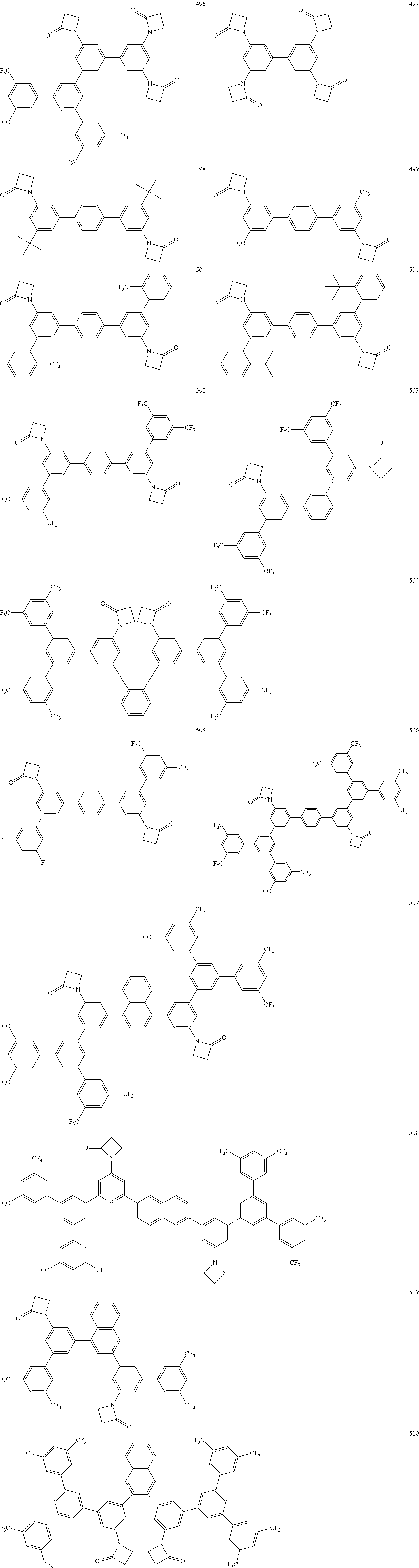

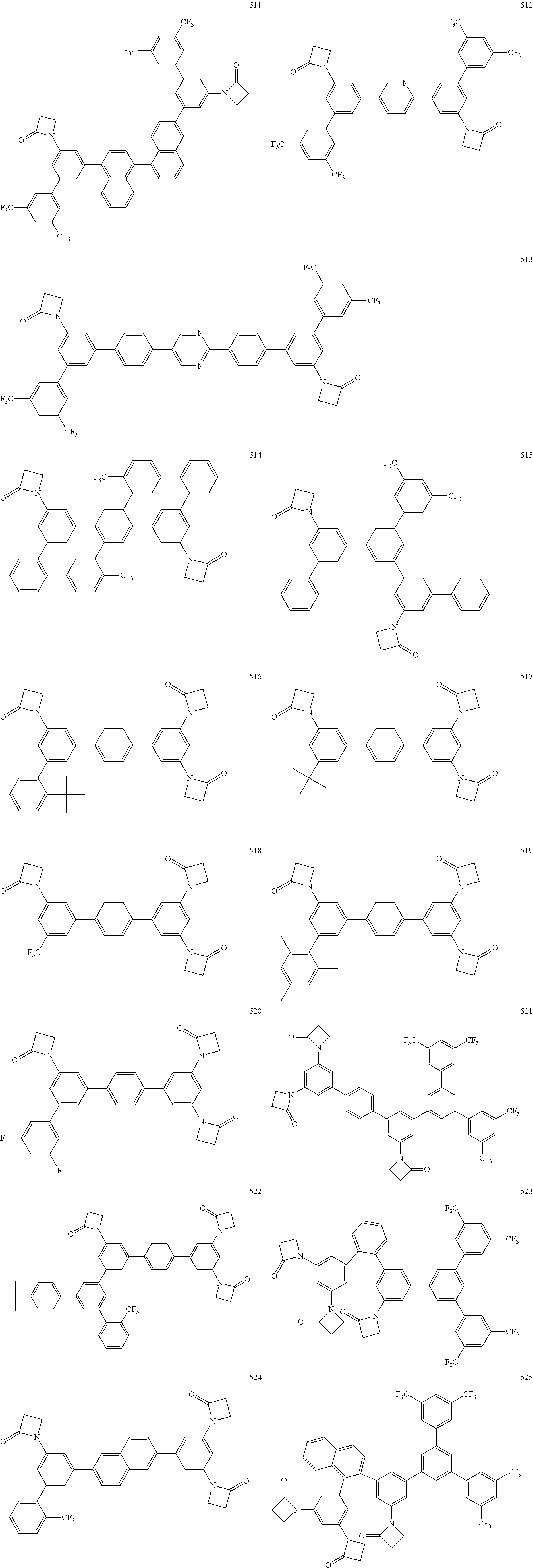

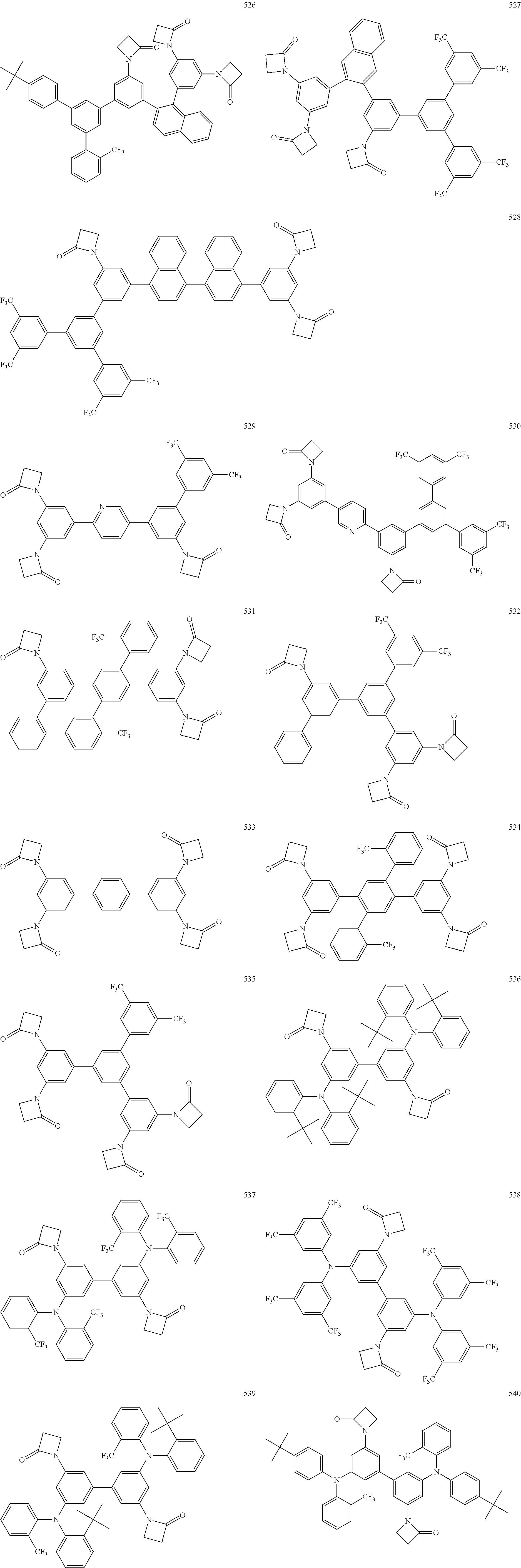

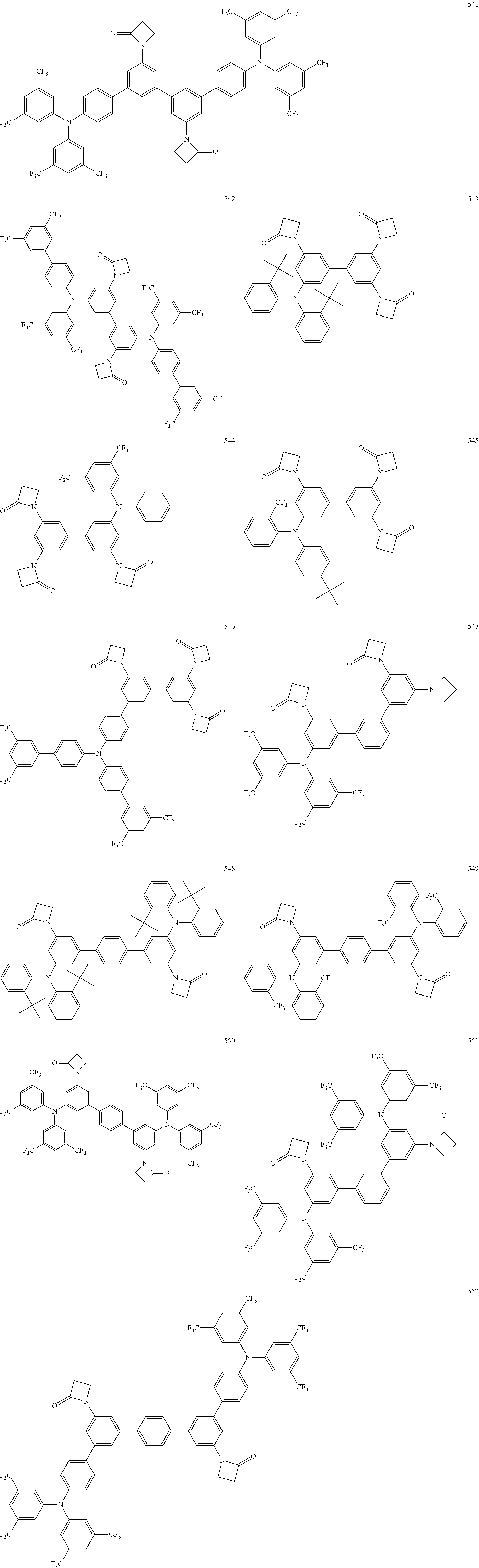

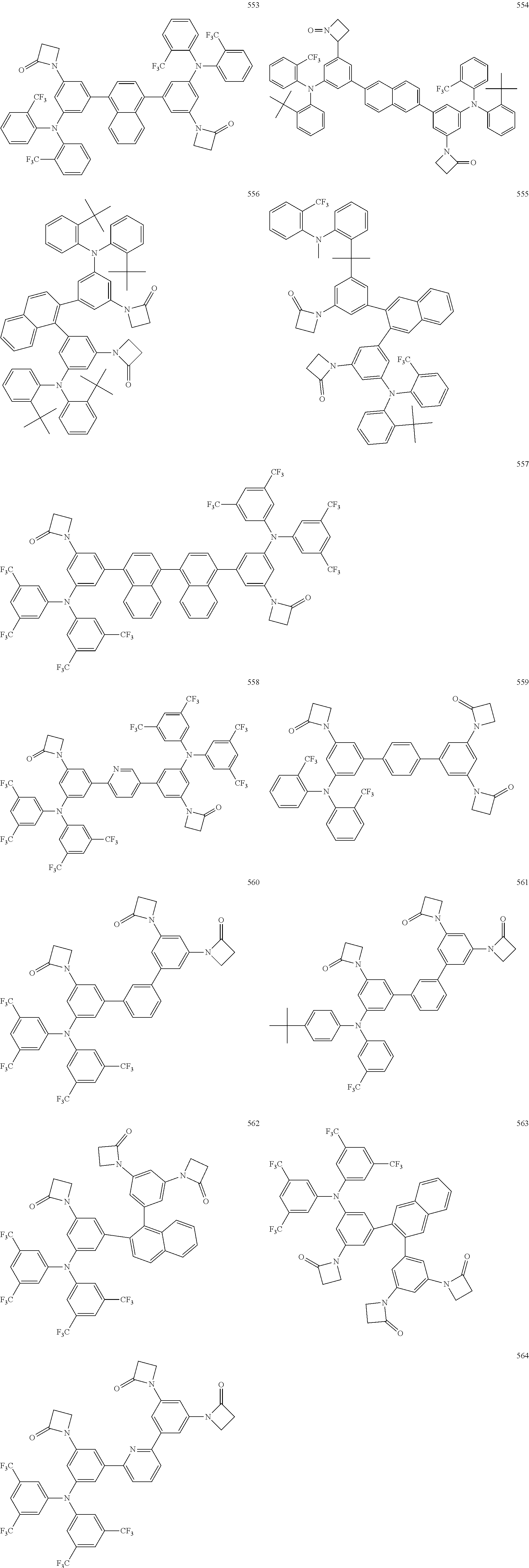

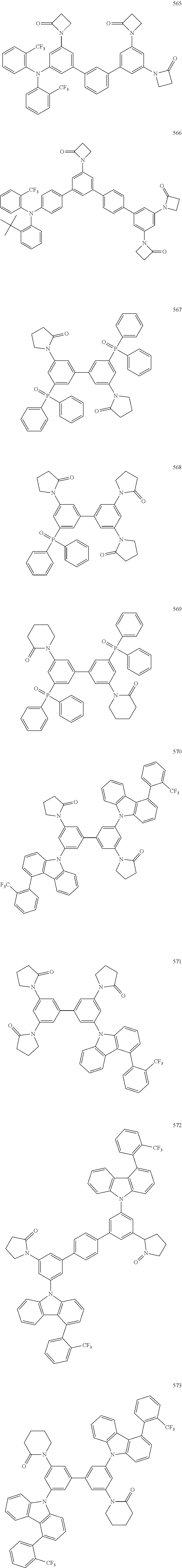

4. An organic light-emitting device comprising a first electrode, a second electrode, and one or more organic layers arranged between the first and second electrodes,
further comprising a light efficiency improving layer (capping layer) formed on at least one side opposite to the organic layer among upper or lower portions of the first electrode and the second electrode,
wherein the light efficiency improving layer includes a compound represented by Formula I above.

5. The organic light-emitting device of claim 4, wherein the light efficiency improving layer is formed on at least one of a lower portion of the first electrode or an upper portion of the second electrode.
